(19) United States
(12) Patent Application Publication
(10) Pub. No.: US 2026/0016446 A1
(43) Pub. Date: Jan. 15, 2026

(54) Diagnostic Device, Diagnostic System Using Same, and Program to be Executed by Computer

(71) Applicant: EXTEND CO., LTD., SAGA (JP)

(72) Inventors: SHINYA OHMAGARI, SAGA (JP); MITSURU KITAICHI, SAGA (JP)

(73) Assignee: EXTEND CO., LTD., SAGA (JP)

(21) Appl. No.: 19/332,832

(22) Filed: Sep. 18, 2025

Related U.S. Application Data
(63) Continuation of application No. PCT/JP2024/044385, filed on Dec. 16, 2024.

(30) Foreign Application Priority Data
Dec. 18, 2023 (JP) 2023-212599

(51) Int. Cl. G01N 27/48; G01N 33/14

(52) U.S. Cl. CPC G01N 27/48; G01N 33/146

(57) ABSTRACT

The diagnostic device calculates sums L(+)_sum, M(+)_sum, H(+)_sum of integral values in a positive predetermined potential section, a sum H(−)_sum of integral values in a negative predetermined potential section, and sums L(all)_sum, M(all)_sum, H(all)_sum of integral values in all predetermined potential sections based on a plurality of integral values ITG_{1_Low} to ITG_{n_Low}, ITG_{1_Middle} to ITG_{n_Middle}, ITG_{1_High} to ITG_{n_High} in a plurality of predetermined potential sections calculated using the current-potential characteristics of three cyclic voltammograms measured while changing the potential at potential scan rates V_{r_Low}, V_{r_Middle}, V_{r_High} to diagnose the taste of analyte.

### CROSS-REFERENCE TO RELATED APPLICATIONS

This application is a bypass continuation of PCT/JP2024/044385 filed on Dec. 16, 2024, which claims the benefit of priority to Japanese patent application No. 2023-212599 filed on Dec. 18, 2023. The entire contents of the foregoing applications are hereby incorporated herein by reference.

### TECHNICAL FIELD

The present invention relates to a diagnostic device, a diagnostic system using the same, and a program for causing a computer to execute the program.

### BACKGROUND ART

In the technology for analyzing solutions of soft drinks, alcoholic beverages, tap water, urine, blood, and the like, analytical methods such as Fourier Transform Infrared Spectroscopy (FTIR), gas chromatography, taste sensors, and Raman spectroscopy are used (NPL 1 to NPL 3).

However, these devices are large and expensive, have problems with portability, and require specialized knowledge to handle the devices due to their complexity.

In addition, although individual sensors for measuring temperature, humidity, total acidity (total acidity of all types of acids in a solution), alcohol content, etc. are capable of on-site measurement (measurement performed on-site), many of them cannot be used alone to judge the state of a solution, and a combination of multiple sensors is required.

Electrochemical sensor can simplify measurement systems, are small, inexpensive, and highly portable, and have great potential as an in situ analytical technique for solutions in general.

### CITATION LIST

### Non Patent Literature

Non Patent Literature 1: H. Yu, Y. Zhang, J. Zhao, and H. Tian, “Taste characteristics of Chinese bayberry juice characterized by sensory evaluation, chromatography analysis, and an electronic tongue,” J Food Sci Technol 55(5), 1624-1631 (2018).
Non Patent Literature 2: G. F. Abreu, F. M. Borem, L. F. C. Oliveira, M. R. Almeida, and A. P. C. Alves, “Raman spectroscopy: A new strategy for monitoring the quality of green coffee beans during storage,” Food Chem 287, 241-248 (2019).
Non Patent Literature 3: R. Ferrer-Gallego, J. M. Hernandez-Hierro, J. C. Rivas-Gonzalo, and M. T. Escribano-Bailon, “Evaluation of sensory parameters of grapes using near infrared spectroscopy,” J Food Eng 118(3), 333-339 (2013).

### SUMMARY OF INVENTION

### Technical Problem

However, it is difficult to diagnose the taste of alcoholic beverages and the like using an electrochemical sensor system.

Therefore, according to an embodiment of the present invention, a diagnostic device capable of diagnosing the taste of alcoholic beverages and the like based on a cyclic voltammogram of the alcoholic beverages and the like is provided.

Furthermore, according to an embodiment of the present invention, a diagnostic system including a diagnostic device capable of diagnosing the taste of alcoholic beverages and the like based on a cyclic voltammogram of the alcoholic beverages and the like is provided.

Furthermore, according to an embodiment of the present invention, a program for causing a computer to execute diagnosis of the taste of alcoholic beverages or the like based on a cyclic voltammogram of the alcoholic beverages or the like is provided.

### Solution to Problem

Aspect 1: According to an embodiment of the present invention, the diagnostic device includes a first calculation unit and a taste diagnostic unit. The first calculation unit calculates a first sum (L(+) sum) which is a sum of first integral values in a positive predetermined potential sections based on a plurality of first integral values in a plurality of predetermined potential sections calculated using a current-potential characteristic of a first cyclic voltammogram measured while changing a potential at a first potential scanning rate, calculates a second sum (M(+)_sum) which is a sum of second integral values in the positive predetermined potential sections based on a plurality of second integral values in the plurality of predetermined potential sections calculated using a current-potential characteristic of a second cyclic voltammogram measured while changing the potential at a second potential scanning rate faster than the first potential scanning rate, calculates a third sum (H(+)_sum) which is a sum of third integral values in the positive predetermined potential sections based on a plurality of third integral values in the plurality of predetermined potential sections calculated using a current-potential characteristic of a third cyclic voltammogram measured while changing the potential at a third potential scanning rate faster than the second potential scanning rate, calculates a fourth sum (H(−)_sum) which is a sum of the third integral values in a negative predetermined potential sections based on the plurality of third integral values in the plurality of predetermined potential sections, calculates a fifth sum (L(all))_sum) which is a sum of the first integral values in all of the predetermined potential sections based on the plurality of first integral values in the plurality of predetermined potential sections, calculates a sixth sum (M(all))_sum) which is a sum of the second integral values in all of the predetermined potential sections based on the plurality of second integral values in the plurality of predetermined potential sections, and calculates a seventh sum (H(all))_sum) which is a sum of the third integral values in all of the predetermined potential sections based on the plurality of third integral values in the plurality of predetermined potential sections. The taste diagnostic unit diagnoses a taste of a first analyte based on the first sum (L(+)_sum), the second sum (M(+)_sum), the third sum (H(+)_sum), the fourth sum (H(−)_sum), the fifth sum (L(all))_sum), the sixth sum (M(all))_sum and the seventh sum (H(all))_sum).

Aspect 2: In aspect 1, the first calculation unit further calculates a first factor (Body index (+)) which is a factor attributable to a diffusion coefficient of a component of the first analyte when a positive potential is applied to the first analyte based on the first sum (L(+)_sum), the second sum (M(+)_sum) and the third sum (H(+)_sum) and calculates a second factor (Body index (all)) which is a factor attributable to the diffusion coefficient of the components of the first analyte when positive and negative potentials are applied to the first analyte based on the fifth sum (L(all))_sum), the sixth sum (M(all))_sum and the seventh sum (H(all))_sum). The taste diagnostic unit diagnoses the “astringency” of the first analyte based on the first factor (Body index (+)), diagnoses the “aftertaste” of the first analyte based on the second factor (Body Index (all)), diagnoses the “sweetness” of the first analyte based on the third sum (H(+)_sum), diagnoses the “aroma” of the first analyte based on the fourth sum (H(−)_sum), and diagnoses the “bitterness” of the first analyte based on the “astringency” of the first analyte and the “sweetness” of the first analyte.

Aspect 3: In aspect 2, the taste diagnostic unit diagnoses the multiplication result obtained by multiplying a coefficient k₁ to the first factor (Body index (+)) as the “astringency” of the first analyte, diagnoses the multiplication result obtained by multiplying a coefficient k₂ to the second factor (Body index (all)) as the “aftertaste” of the first analyte, diagnoses as the “sweetness” of the first analyte the result of multiplying a coefficient k₄ to the result of dividing the third sum (H(+)_sum) by the coefficient k₃, diagnoses as the “aroma” of the first analyte the result of multiplying a coefficient k₆ to the result of dividing the fourth sum (H(−)_sum) by the coefficient k₅, and diagnoses as the “bitterness” of the first analyte the result obtained by subtracting the result of multiplying the coefficient k₈ to the “sweetness” of the first analyte from the result obtained by multiplying the coefficient k₇ to the “astringency” of the first analyte.

Aspect 4: In aspect 3, the taste diagnostic unit performs a regression analysis using the first factor (Body index (+)) as an explanatory variable and “astringency” as a response variable to obtain a regression equation, and determines a value multiplied to the first factor (Body index (+)) in the obtained regression equation as a value of coefficient k₁, performs a regression analysis using the second factor (Body index (all)) as an explanatory variable and the “aftertaste” as a response variable to obtain a regression equation, and determines a value multiplied to the second factor (Body index (all)) which is an explanatory variable in the obtained regression equation as the value of the coefficient k₂, performs a regression analysis using the third sum (H(+)_sum) as an explanatory variable and “sweetness” as a response variable to obtain a regression equation, and determines a value dividing the explanatory variable (=the third sum (H(+) sum)) as the value of coefficient k₃ in the obtained regression equation, and determines a value multiplied to the explanatory variable (=the third sum (H(+)_sum)) as the value of coefficient k₄, performs a regression analysis using the fourth sum (H(−)_sum) as an explanatory variable and “aroma” as a response variable to obtain a regression equation, and determines a value dividing the explanatory variable (the fourth sum (H(−)_sum)) as the value of the coefficient k₅ and determines a value multiplied to the explanatory variable (the fourth sum (H(−)_sum)) as the value of the coefficient k₆ in the obtained regression equation, performs a regression analysis using the “astringency” and the “sweetness” as explanatory variables and the “bitterness” as a response variable to obtain a regression equation, and determines a value multiplied to the “astringency” as the value of the coefficient k₇, and determines a value multiplied to the “sweetness” as the value of the coefficient k₈ in the obtained regression equation.

Aspect 5: In aspect 3, when the taste diagnostic unit diagnosed the “astringency”, the “aftertaste”, the “sweetness”, the “aroma” and the “bitterness” of v (v is an integer of 1 or more) first analytes, the taste diagnostic unit updates the values of the coefficients k₁ to k₈ and diagnoses the “astringency”, the “aftertaste”, the “sweetness”, the “aroma” and the “bitterness” of the first analyte using the updated values of the coefficients k₁ to k₈.

Aspect 6: In aspect 1, the plurality of first integral values are any of n¹₁ (n¹₁ is the number of integral values calculated using the smallest predetermined potential section, and is an addition result obtained by adding “1” to an integer obtained by rounding down the decimal point of a division result obtained by dividing a positive potential section by the smallest predetermined potential section when the decimal point of the division result is not zero) first integral values, n¹₂ (n¹₂<n¹₁) first integral values, n¹₃ (n¹₃<n¹₂) first integral values, . . . , and n¹₁ (n¹_{b}<n¹_{b-1}, b is an integer equal to or greater than 2) first integral values,
the plurality of second integral values are any of n²₁ (n²₁ is the number of integral values calculated using the smallest predetermined potential section, and is an addition result obtained by adding “1” to an integer obtained by rounding down the decimal point of a division result obtained by dividing a positive potential section by the smallest predetermined potential section when the decimal point of the division result is not zero) second integral values, n²₂ (n²₂<n²₁) second integral values, n²₃ (n²₃<n²₂) second integral values, . . . , and n² b (n²_{b}<n²_{b-1}, b is an integer equal to or greater than 2) second integral values, and
the plurality of third integral values are any of n³₁ (n³₁ is the number of integral values calculated using the smallest predetermined potential section, and is composed of an addition result obtained by adding “1” to an integer obtained by rounding down the decimal point of a division result obtained by dividing a positive potential section by the smallest predetermined potential section when the decimal point of the division result is not zero) third integral values, n³₂ (n³₂<n³₁) third integral values, n³₃ (n³₃<n³₂) third integral values, . . . , and n³_{b} (n³_{b}<n³_{b-1}, b is an integer equal to or greater than 2) third integral values.

Aspect 7: In aspect 1, the first calculation unit further calculates an eighth sum (L(−)_sum_th) which is a sum of the first integral values in a plurality of negative predetermined potential sections consisting of negative potentials equal to or lower than a threshold value based on the plurality of first integral values in a plurality of predetermined potential sections calculated using the current-potential characteristic of the first cyclic voltammogram, calculates a ninth sum (M(−)_sum_th) which is a sum of the second integral values in the plurality of negative predetermined potential sections consisting of negative potentials equal to or lower than the threshold value based on the plurality of second integral values in the plurality of predetermined potential sections calculated using the current-potential characteristic of the second cyclic voltammogram, calculates a tenth sum (H(−)_sum_th) which is a sum of the third integral values in the plurality of negative predetermined potential sections consisting of negative potentials equal to or lower than the threshold value based on the plurality of third integral values in the plurality of predetermined potential sections calculated using the current-potential characteristic of the third cyclic voltammogram; calculates a third factor (Body index (−)_th), which is a factor attributable to a diffusion coefficient of a component of the second analyte when a negative potential equal to or lower than the threshold value is applied to the second analyte based on the eighth sum (L(−)_sum_th), the ninth sum (M(−)_sum_th) and the tenth sum (H(−)_sum_th). The taste diagnostic unit further diagnoses the “astringency” of the second analyte based on the third factor (Body index (−)_th).

Aspect 8: In aspect 7, the taste diagnostic unit diagnoses the multiplication result obtained by multiplying a coefficient k₉ to the third factor (Body index (−)_th) as the “astringency” of the second analyte.

Aspect 9: In aspect 8, the taste diagnostic unit performs a regression analysis using the third factor (Body index (−)_th) as an explanatory variable and the “astringency” as a response variable to obtain a regression equation, and determines a value multiplied to the third factor (Body index (−)_th) in the obtained regression equation as the value of coefficient k₉.

Aspect 10: In aspect 8, when the taste diagnostic unit diagnosed the “astringency” of v (v is an integer equal to or greater than 1) of the second analytes, the taste diagnostic unit updates the value of the coefficient k₉ and diagnoses the “astringency” of the second analyte using the updated value of the coefficient k₉.

Aspect 11: In aspect 7, the sum of the first integral values in a plurality of negative predetermined potential sections consisting of negative potentials equal to or less than the threshold value is any one of a sum of w¹₁ (w¹₁ is an addition result obtained by adding “1” to an integer obtained by rounding down a decimal point of a division result obtained by dividing a negative potential section equal to or less than the threshold value by a minimum predetermined potential section when the decimal point of the division result is not zero.) first integral values, a sum of w¹₂ (w¹₂<w¹₁) first integral values, a sum of w¹₃ (w¹₃<w¹₂) first integral values, . . . , and a sum of w¹_{b} (w¹_{b}<w¹_{b-1}, b is an integer equal to or greater than 2) first integral values, the sum of the second integral values in the plurality of negative predetermined potential sections consisting of negative potentials equal to or less than the threshold value is any one of a sum of w²₁ (w²₁ is an addition result obtained by adding “1” to an integer obtained by rounding down a decimal point of a division result obtained by dividing a negative potential section equal to or less than a threshold value by a smallest predetermined potential section when the decimal point of the division result is not zero.) second integral values, a sum of w²₂ (w²₂<w²₁) second integral values, a sum of w²³ (w²₃<w²₂) second integral values, . . . , and a sum of w²_{b} (w²_{b}<w²_{b-1}, b is an integer equal to or greater than 2) second integral values, and the sum of the third integral values in a plurality of negative predetermined potential sections consisting of negative potentials equal to or less than the threshold value is any one of a sum of w³₁ (w³₁ is an addition result obtained by adding “1” to an integer obtained by rounding down a decimal point of a division result obtained by dividing a negative potential section equal to or less than a threshold value by a minimum predetermined potential section when the decimal point of the division result is not zero.) third integral values, a sum of w³₂ (w³₂<w³₁) third integral values, a sum of w³₃ (w³₃<w³₂) third integral values, . . . and a sum of w³_{b} (w³_{b}<w³_{b-1}, b is an integer equal to or greater than 2) third integral values.

Aspect 12: In aspect 1, the diagnostic device further includes a second calculation unit. The second calculation unit calculates a plurality of first integral values in a plurality of predetermined potential sections based on the current-potential characteristics of the first cyclic voltammogram, calculates a plurality of second integral values in a plurality of predetermined potential sections based on the current-potential characteristics of the second cyclic voltammogram, and calculates a plurality of third integral values in a plurality of predetermined potential sections based on the current-potential characteristics of the third cyclic voltammogram. The first calculation unit calculates a first sum (L(+)_sum) and a fifth sum (L(all))_sum based on the plurality of first integral values calculated by the second calculation unit, calculates a second sum (M(+)_sum) and a sixth sum (M(all))_sum based on the plurality of second integral values calculated by the second calculation unit, and calculates a third sum (H(+)_sum), a fourth sum (H(−)_sum) and a seventh sum (H(all))_sum based on the plurality of third integral values calculated by the second calculation unit.

Aspect 13: In aspect 12, the diagnostic device further comprises a creation unit. The creation unit creates, as a feature amount of the first analyte or the second analyte, a curve indicating the dependency of the plurality of sum integral values on the plurality of predetermined potential sections based on the plurality of predetermined potential sections and a plurality of sum integral values respectively corresponding to the plurality of predetermined potential sections. The second calculation unit further calculates the total integral value which is a sum of the first integral value, the second integral value, and the third integral value in one of predetermined potential section for all of the plurality of predetermined potential sections to calculate the plurality of total integral values, and outputs the plurality of predetermined potential sections and the plurality of total integral values respectively associated with the plurality of predetermined potential sections to the creation unit.

Aspect 14: In aspect 13, the calculation data includes a plurality of predetermined potential sections and a plurality of total integral values respectively associated with the plurality of predetermined potential sections. The judgment unit judges whether P (P is an integer equal to or greater than 2) pieces of [a plurality of total integral values] included in P pieces of calculation data are or not different from each other. The creation unit creates P pieces of curves when a judgment unit judges that the P pieces of [plurality of total integral values] are different from each other.

Aspect 15: According to an embodiment of the present invention, a diagnostic system includes the diagnostic device according to any one of aspects 1 to 14.

Aspect 16: Furthermore, according to an embodiment of the present invention, a program causes a computer to execute a first step in which a first calculation unit calculates a first sum (L(+)_sum) which is a sum of first integral values in a positive predetermined potential section based on a plurality of first integral values in a plurality of predetermined potential sections calculated using a current-potential characteristic of a first cyclic voltammogram measured while changing a potential at a first potential scanning rate, calculates a second sum (M(+) sum) which is a sum of second integral values in a positive predetermined potential section based on a plurality of second integral values in a plurality of predetermined potential sections calculated using a current-potential characteristic of a second cyclic voltammogram measured while changing the potential at a second potential scanning rate faster than the first potential scanning rate, calculates a third sum (H(+)_sum) which is a sum of third integral values in a positive predetermined potential section based on a plurality of third integral values in a plurality of predetermined potential sections calculated using a current-potential characteristic of a third cyclic voltammogram measured while changing the potential at a third potential scanning rate faster than the second potential scanning rate, calculates a fourth sum (H(−)_sum) which is a sum of the third integral values in a negative predetermined potential section based on a plurality of third integral values in the plurality of predetermined potential sections, calculates a fifth sum (L(all))_sum) which is a sum of the first integral values in all of the predetermined potential sections based on the plurality of first integral values in the plurality of predetermined potential sections, calculates a sixth sum (M(all))_sum) which is a sum of the second integral values in all of the predetermined potential sections based on the plurality of second integral values in the plurality of predetermined potential sections, and calculates a seventh sum (H(all))_sum) which is a sum of the third integral values in all of the predetermined potential sections based on the plurality of third integral values in the plurality of predetermined potential sections, and a second step in which a taste diagnostic unit diagnoses the taste of the first analyte based on the first sum (L(+)_sum), the second sum (M(+)_sum), the third sum (H(+)_sum), the fourth sum (H(−)_sum), the fifth sum (L(all))_sum), the sixth sum (M(all))_sum and the seventh sum (H(all)_sum).

Aspect 17: In aspect 16, the first calculation unit, in the first step, further calculates a first factor (Body index (+)) which is a factor attributable to the diffusion coefficient of the components of the first analyte when a positive potential is applied to the first analyte based on the first sum (L(+)_sum), the second sum (M(+)_sum), and the third sum (H(+)_sum), and calculates a second factor (Body index (all)) which is a factor attributable to the diffusion coefficient of the components of the first analyte in when positive and negative potentials are applied to the first analyte based on the fifth sum (L(all))_sum), the sixth sum (M(all))_sum), and the seventh sum (H(all))_sum). The taste diagnostic unit, in the second step, diagnoses the “astringency” of the first analyte based on the first factor (Body index (+)), diagnoses the “aftertaste” of the first analyte based on the second factor (Body index (all)), diagnoses the “sweetness” of the first analyte based on the third sum (H(+)_sum), diagnoses the “aroma” of the first analyte based on the fourth sum (H(−)_sum), and diagnoses the “bitterness” of the first analyte based on the “astringency” of the first analyte and the “sweetness” of the first analyte.

Aspect 18: In aspect 17, in the second step, the taste diagnostic unit diagnoses the multiplication result of the first factor (Body index (+)) multiplied by the coefficient k₁ as the “astringency” of the first analyte, diagnoses the multiplication result of the second factor (Body index (all)) multiplied by the coefficient k₂ as the “aftertaste” of the first analyte, diagnoses the result of multiplying k₄ to the result of dividing the third sum (H(+)_sum) by the coefficient k₃ as the “sweetness” of the first analyte, diagnoses the result of multiplying the coefficient k₆ to the result of dividing the fourth sum (H(−)_sum) by the coefficient k₅ as the “aroma” of the first analyte, and diagnoses the result of subtracting the result of multiplying the coefficient k₈ to the “sweetness” of the first analyte from the result of multiplying the coefficient k₇ to the “astringency” of the first analyte as bitterness of the first analyte.

Aspect 19: In aspect 18, in the second step, the taste diagnostic unit performs a regression analysis using the first factor (Body index (+)) as an explanatory variable and “astringency” as a response variable to obtain a regression equation, and determines the value multiplied to the first factor (Body index (+)) in the obtained regression equation as the value of coefficient k₁, performs a regression analysis using the second factor (Body index (all)) as an explanatory variable and “aftertaste” as a response variable to obtain a regression equation, and determines the value multiplied to the second factor (Body index (all)) which is the explanatory variable in the obtained regression equation as the value of coefficient k₂, performs a regression analysis using the third sum (H(+)_sum) as an explanatory variable and “sweetness” as a response variable to obtain a regression equation, and determines the value diving the explanatory variable (=the third sum (H(+)_sum)) in the obtained regression equation as the value of the coefficient k₃ and determines the value multiplied to the explanatory variable (=the third sum (H(+)_sum)) as the value of the coefficient k₄, performs a regression analysis using the fourth sum (H(−)_sum) as an explanatory variable and “aroma” as a response variable to obtain a regression equation, and determines the value diving the explanatory variable (=the fourth sum (H(−)_sum)) in the obtained regression equation as the value of the coefficient k₅ and determines the value multiplied to the explanatory variable (=the fourth sum (H(−)_sum)) as the value of the coefficient k₆, and performs a regression analysis using “the astringency” and “the sweetness” as an explanatory variable and “bitterness” as a response variable to obtain a regression equation, and determines the value multiplied to “the astringency” in the obtained regression equation as the value of the coefficient k₇ and determines the value multiplied to “the sweetness” as the value of the coefficient k₈.

Aspect 20: In aspect 18, in the second step, when the taste diagnostic unit diagnosed the “astringency”, “aftertaste”, “sweetness”, “aroma” and “bitterness” of v (v is an integer greater than or equal to 1) first analytes, the taste diagnostic unit updates the values of coefficients k₁ to k₈ and uses the updated values of coefficients k₁ to k₈ to diagnose the “astringency”, “aftertaste”, “sweetness”, “aroma” and “bitterness” of the first analytes.

Aspect 21: In aspect 16, the plurality of first integral values are any of n¹₁ (n¹₁ is the number of integral values calculated using the smallest predetermined potential section, and is composed of an addition result obtained by adding “1” to an integer obtained by rounding down the decimal point of a division result obtained by dividing a positive potential section by the smallest predetermined potential section when the decimal point of the division result is not zero) first integral values, n¹₂ (n¹₂<n¹₁) first integral values, n¹₃ (n¹₃<n¹₂) first integral values, . . . , and n¹_{b} (n¹_{b}<n¹_{b-1}, b is an integer of 2 or more) first integral values,
the plurality of second integral values are any of n²₁ (n²₁ is the number of integral values calculated using the smallest predetermined potential section, and is composed of an addition result obtained by adding “1” to an integer obtained by rounding down the decimal point of a division result obtained by dividing a positive potential section by the smallest predetermined potential section when the decimal point of the division result is not zero) second integral values, n²₂ (n²₂<n²₁) second integral values, n²₃ (n²₃<n²₂) second integral values, . . . , and n²_{b} (n²_{b}<n²_{b-1}, b is an integer of 2 or more) second integral values, and
the plurality of third integral values are any of n³₁ (n³₁ is the number of integral values calculated using the smallest predetermined potential section, and is composed of an addition result obtained by adding “1” to an integer obtained by rounding down the decimal point of a division result obtained by dividing a positive potential section by the smallest predetermined potential section when the decimal point of the division result is not zero) third integral values, n³₂ (n³₂<n³₁) third integral values, n³₃ (n³₃<n³₂) third integral values, . . . , and n³_{b} (n³_{b}<n³_{b-1}, b is an integer of 2 or more) third integral values.

Aspect 22: In aspect 16, in the first step, the first calculation unit further calculates an eighth sum (L(−)_sum_th) which is a sum of the first integral values in a plurality of negative predetermined potential sections consisting of negative potentials equal to or less than a threshold value based on the plurality of first integral values in a plurality of predetermined potential sections calculated using the current-potential characteristic of the first cyclic voltammogram, calculates an ninth sum (M(−)_sum_th) which is a sum of the second integral values in a plurality of negative predetermined potential sections consisting of negative potentials equal to or less than the threshold value based on the plurality of second integral values in a plurality of predetermined potential sections calculated using the current-potential characteristic of the second cyclic voltammogram, calculates an tenth sum (H(−)_sum_th) which is a sum of the third integral values in a plurality of negative predetermined potential sections consisting of negative potentials equal to or less than the threshold value based on the plurality of third integral values in a plurality of predetermined potential sections calculated using the current-potential characteristic of the third cyclic voltammogram, and calculates a third factor (Body index (−)_th) which is a factor attributable to a diffusion coefficient of a component of the second analyte when a negative potential equal to or less than the threshold is applied to the second analyte based on the eighth sum (L(−)_sum_th), the ninth sum (M(−)_sum_th) and the tenth sum (H(−)_sum_th), and in the second step, the taste diagnostic unit further diagnoses the “astringency” of the second analyte based on the third factor (Body index (−)_th).

Aspect 23: In aspect 22, in the second step, the taste diagnostic unit diagnoses the multiplication result obtained by multiplying the coefficient k₉ to the third factor (Body index (−)_th) as the “astringency” of the second analyte.

Aspect 24: In aspect 23, in the second step, the taste diagnostic unit performs regression analysis using the third factor (Body index (−)_th) as the explanatory variable and “astringency” as the objective variable to obtain a regression equation, and determines the value multiplied to the third factor (Body index (−)_th) in the obtained regression equation as the value of coefficient k₉.

Aspect 25: In aspect 23, in the second step, when the taste diagnostic unit has diagnosed the “astringency” of v (v is an integer greater than or equal to 1) second analytes, the taste diagnostic unit updates the value of coefficient k₉ and diagnoses the “astringency” of the second analytes using the updated value of coefficient k₉.

Aspect 26: In aspect 22, the sum of the first integral values in a plurality of negative predetermined potential sections consisting of negative potentials equal to or below the threshold is any one of a sum of w¹₁ (w¹₁ is the result of adding “1” to the integer obtained by truncating the number equal to or below the decimal point of the division result when number equal to or below the decimal point of the division result of dividing a negative potential section equal to or below the threshold by the minimum specified potential section is not zero.) first integral values, a sum of w¹₂(w¹₂<w¹₁) first integral values, a sum of w¹₃(w¹₃<w¹₂) first integral values, . . . , and a sum of w¹_{b} (w¹_{b}<w¹_{b-1}, b is an integer equal to or greater than 2) first integral values,
the sum of the second integral values in a plurality of negative predetermined potential sections consisting of negative potentials equal to or below the threshold is any one of a sum of w²₁ (w²₁ is the result of adding “1” to the integer obtained by truncating the number equal to or below the decimal point of the division result when number equal to or below the decimal point of the division result of dividing a negative potential section equal to or below the threshold by the minimum specified potential section is not zero.) second integral values, a sum of w²₂ (w²₂<w²₁) second integral values, a sum of w²₃ (w²₃<w²₂) second integral values, . . . , and a sum of w²_{b} (w²_{b}<w²_{b-1}, b is an integer equal to or greater than 2) second integral values, and
the sum of the third integral values in a plurality of negative predetermined potential sections consisting of negative potentials equal to or below the threshold is any one of a sum of w³₁ (w³₁ is the result of adding “1” to the integer obtained by truncating the number equal to or below the decimal point of the division result when number equal to or below the decimal point of the division result of dividing a negative potential section equal to or below the threshold by the minimum specified potential section is not zero.) third integral values, a sum of w³₂ (w³₂<w³₁) third integral values, a sum of w³₃ (w³₃<w³₂) third integral values, . . . , and a sum of w³_{b} (w³_{b}<w³_{b-1}, b is an integer equal to or greater than 2) third integral values.

Aspect 27: In aspect 16, the program causes a computer to execute further a third step in which a second calculation unit calculates a plurality of first integral values in a plurality of predetermined potential sections based on the current-potential characteristics of the first cyclic voltammogram, calculates a plurality of second integral values in a plurality of predetermined potential sections based on the current-potential characteristics of the second cyclic voltammogram, and calculates a plurality of third integral values in a plurality of predetermined potential sections based on the current-potential characteristics of the third cyclic voltammogram, and
in the first step, the first calculation unit calculates the first sum (L(+)_sum) and the fifth sum (L(all))_sum) based on the plurality of first integral values calculated by the second calculation unit, calculates the second sum (M(+)_sum) and the sixth sum (M(all))_sum) based on the plurality of second integral values calculated by the second calculation unit, and calculates the third sum (H(+)_sum), the fourth sum (H(−)_sum) and the seventh sum (H(all))_sum) based on the plurality of third integral values calculated by the second calculation unit.

Aspect 28: In aspect 27, the program causes a computer to execute further a fourth step in which the creation unit creates a curve showing the dependence of the plurality of sum integral values on the plurality of predetermined potential sections based on the plurality of predetermined potential sections and the plurality of sum integral values respectively corresponding to the plurality of predetermined potential sections, as a feature amount of the first analyte or the second analyte, and
in the third step, the second calculation unit further execute, for all of the plurality of predetermined potential sections, to calculate a total integral value which is the sum of the first integral value, the second integral value and the third integral value in one predetermined potential section to calculate a plurality of total integral values, and outputs the plurality of predetermined potential sections and the plurality of total integral values respectively corresponding to the plurality of predetermined potential sections to the creation unit.

Aspect 29: In aspect 28, calculation data includes the plurality of predetermined potential sections and the plurality of total integral values respectively corresponding to the plurality of predetermined potential sections,
the program causes the computer to execute further a fifth step in which a judgment unit judges whether P (P is an integer equal to or greater than 2) pieces of [a plurality of sum integral values] included in P pieces of calculation data are or not different from each other, and
the creation unit creates P pieces of curves in the fourth step when the judgment unit judges in the fifth step that the P pieces of [plurality of multiple sum integral values] are different from each other.

### Advantageous Effects of Invention

According to the embodiment of the present invention, the diagnostic device can diagnose the taste of alcoholic beverages and the like based on a cyclic voltammogram of the alcoholic beverages and the like.

### BRIEF DESCRIPTION OF DRAWINGS

FIG. 1 is a schematic diagram of a diagnostic system according to a first embodiment of the present invention.

FIG. 2 is a schematic diagram of the sensor device 1 shown in FIG. 1.

FIG. 3 is a perspective view of the measuring instrument 12 shown in FIG. 2.

FIG. 4 is a schematic diagram of the measuring instrument 12 shown in FIG. 2.

FIG. 5 is a schematic diagram showing a timing chart of the potential supplied to the sensor 11 shown in FIG. 2.

FIG. 6 is a schematic diagram of the measurement data MRS.

FIG. 7 is a schematic diagram of the diagnostic device 2 shown in FIG. 1.

FIG. 8 s a schematic diagram of the analysis/diagnostic unit 21 shown in FIG. 7.

FIG. 9 is a first conceptual diagram for explaining a method for calculating an integral value.

FIG. 10 is a second conceptual diagram for explaining a method for calculating an integral value.

FIG. 11 is a figure showing a part of a cyclic voltammogram.

FIG. 12 is a diagram for explaining a method for creating a curve CUR showing the relationship between a plurality of classes Cls and a plurality of integral values ITG.

FIG. 13 is a conceptual diagram showing the class dependency of the integral value created based on cyclic voltammograms measured while changing a potential scanning rate to the potential scanning rate V_{r_Low}, V_{r_Middle}, and V_{r_High}.

FIG. 14 is a conceptual diagram of the teacher data.

FIG. 15 is a conceptual diagram of a correspondence table showing the correspondence between coefficients and teacher data.

FIG. 16 is a conceptual diagram of updated teacher data.

FIG. 17 is a conceptual diagram of a correspondence table TBL1_up1 obtained by updating the correspondence table TBL1 shown in FIG. 15.

FIG. 18 is a conceptual diagram for diagnosing the taste (astringency, aftertaste, sweetness, aroma, bitterness) of shochu.

FIG. 19 is a diagram showing the integral value spectra for the shochu of samples No. 1 to No. 3 shown in Table 2.

FIG. 20 is a diagram showing the integral value spectra for the shochu of samples No. 4 to No. 6 shown in Table 2.

FIG. 21 is a diagram showing the integral value spectra for the shochu of samples No. 7 to No. 9 shown in Table 2.

FIG. 22 is a diagram showing the integral value spectra for the shochu of samples No. 10 to No. 12 shown in Table 2.

FIG. 23 is a diagram showing the integral value spectra for the shochu of samples No. 12 to No. 14 shown in Table 2.

FIG. 24 is a diagram showing the integral value spectra for the shochu of samples No. 15 to No. 16 shown in Table 2.

FIG. 25 is a diagram showing the integral value spectra for the shochu of samples No. 17 to No. 20 shown in Table 2.

FIG. 26 is a first diagram showing judgment results on whether the curves k1 to k20 shown in FIGS. 19 to 25 are or not different from one another.

FIG. 27 is a second diagram showing judgment results on whether the curves k1 to k20 shown in FIGS. 19 to 25 are or not different from one another.

FIG. 28 is a third diagram showing judgment results on whether the curves k1 to k20 shown in FIGS. 19 to 25 are or not different from one another.

FIG. 29 is a diagram showing the diagnosis results of “astringency,” “aftertaste,” “sweetness,” “aroma,” and “bitterness” for Shochu of No. 1 to No. 20 shown in Table 2.

FIG. 30 is a diagram showing other diagnosis results of the “astringency”, “aftertaste”, “sweetness”, “aroma” and “bitterness” for Shochu of No. 1 to No. 20 shown in Table 2.

FIG. 31 is a diagram showing the differences between the respective values of the diagnosis results for Shochu of No. 1 to No. 20 shown in FIG. 29 and the respective values of the diagnosis results for Shochu of No. 1 to No. 20 shown in FIG. 30.

FIG. 32 is a diagram showing integral spectra for Crimson Seedless (skin+flesh), Green Seedless (skin+flesh) and Shine Muscat (skin+flesh).

FIG. 33 is a diagram showing integral spectra for Crimson Seedless (flesh), Green Seedless (flesh), and Shine Muscat (flesh).

FIG. 34 is a diagram showing the result of judgment on whether the curves k21 to k26 shown in FIG. 32 and FIG. 33 are or not different from each other.

FIG. 35 is a diagram showing the diagnosis results of “astringency” for Green Seedless (flesh only), Crimson Seedless (flesh only), Shine Muscat (flesh only), Green Seedless (flesh+skin), Crimson Seedless (flesh+skin), and Shine Muscat (flesh+skin).

FIG. 36 is a diagram showing integral value spectra for the shochu of samples No. 1 to No. 3 shown in Table 2, in the case where the number of integral values is 138.

FIG. 37 is a diagram showing integral value spectra for the shochu of samples No. 4 to No. 6 shown in Table 2, in the case where the number of integral values is 138.

FIG. 38 is a diagram showing integral value spectra for the shochu of samples No. 7 to No. 9 shown in Table 2, in the case where the number of integral values is 138.

FIG. 39 is a diagram showing integral value spectra for the shochu of samples No. 10 to No. 12 shown in Table 2, in the case where the number of integral values is 138.

FIG. 40 is a diagram showing integral value spectra for the shochu of samples No. 12 to No. 14 shown in Table 2, in the case where the number of integral values is 138.

FIG. 41 is a diagram showing integral value spectra for the shochu of samples No. 15 to No. 16 shown in Table 2, in the case where the number of integral values is 138.

FIG. 42 is a diagram showing integral value spectra for the shochu of samples No. 17 to No. 20 shown in Table 2, in the case where the number of integral values is 138.

FIG. 43 is a first diagram showing the result of judgment on whether the curves k27 to k46 shown in FIGS. 36 to 42 are or not different from each other.

FIG. 44 is a second diagram showing the result of judgment on whether the curves k27 to k46 shown in FIGS. 36 to 42 are or not different from each other.

FIG. 45 is a third diagram showing the result of judgment on whether the curves k27 to k46 shown in FIGS. 36 to 42 are or not different from each other.

FIG. 46 is a diagram showing integral value spectra for the shochu of samples No. 1 to No. 3 shown in Table 2, in the case where the number of integral values is 70.

FIG. 47 is a diagram showing integral value spectra for the shochu of samples No. 4 to No. 6 shown in Table 2, in the case where the number of integral values is 70.

FIG. 48 is a diagram showing integral value spectra for the shochu of samples No. 7 to No. 9 shown in Table 2, in the case where the number of integral values is 70.

FIG. 49 is a diagram showing integral value spectra for the shochu of samples No. 10 to No. 12 shown in Table 2, in the case where the number of integral values is 70.

FIG. 50 is a diagram showing integral value spectra for the shochu of samples No. 12 to No. 14 shown in Table 2, in the case where the number of integral values is 70.

FIG. 51 is a diagram showing integral value spectra for the shochu of samples No. 15 to No. 16 shown in Table 2, in the case where the number of integral values is 70.

FIG. 52 is a diagram showing integral value spectra for the shochu of samples No. 17 to No. 20 shown in Table 2, in the case where the number of integral values is 70.

FIG. 53 is a first diagram showing the result of judgment on whether the curves k47 to k66 shown in FIGS. 46 to 52 are or not different from each other.

FIG. 54 is a second diagram showing the result of judgment on whether the curves k47 to k66 shown in FIGS. 46 to 52 are or not different from each other.

FIG. 55 is a third diagram showing the result of judgment on whether the curves k47 to k66 shown in FIGS. 46 to 52 are or not different from each other.

FIG. 56 is a diagram showing integral value spectra for the shochu of samples No. 1 to No. 3 shown in Table 2, in the case where the number of integral values is 36.

FIG. 57 is a diagram showing integral value spectra for the shochu of samples No. 4 to No. 6 shown in Table 2, in the case where the number of integral values is 36.

FIG. 58 is a diagram showing integral value spectra for the shochu of samples No. 7 to No. 9 shown in Table 2, in the case where the number of integral values is 36.

FIG. 59 is a diagram showing integral value spectra for the shochu of samples No. 10 to No. 12 shown in Table 2, in the case where the number of integral values is 36.

FIG. 60 is a diagram showing integral value spectra for the shochu of samples No. 12 to No. 14 shown in Table 2, in the case where the number of integral values is 36.

FIG. 61 is a diagram showing integral value spectra for the shochu of samples No. 15 to No. 16 shown in Table 2, in the case where the number of integral values is 36.

FIG. 62 is a diagram showing integral value spectra for the shochu of samples No. 17 to No. 20 shown in Table 2, in the case where the number of integral values is 36.

FIG. 63 is a first diagram showing the result of judgment on whether the curves k67 to k86 shown in FIGS. 56 to 62 are or not different from each other.

FIG. 64 is a second diagram showing the result of judgment on whether the curves k67 to k86 shown in FIGS. 56 to 62 are or not different from each other.

FIG. 65 is a third diagram showing the result of judgment on whether the curves k67 to k86 shown in FIGS. 56 to 62 are or not different from each other.

FIG. 66 is a diagram showing integral value spectra for the shochu of samples No. 1 to No. 3 shown in Table 2, in the case where the number of integral values is 18.

FIG. 67 is a diagram showing integral value spectra for the shochu of samples No. 4 to No. 6 shown in Table 2, in the case where the number of integral values is 18.

FIG. 68 is a diagram showing integral value spectra for the shochu of samples No. 7 to No. 9 shown in Table 2, in the case where the number of integral values is 18.

FIG. 69 is a diagram showing integral value spectra for the shochu of samples No. 10 to No. 12 shown in Table 2, in the case where the number of integral values is 18.

FIG. 70 is a diagram showing integral value spectra for the shochu of samples No. 12 to No. 14 shown in Table 2, in the case where the number of integral values is 18.

FIG. 71 is a diagram showing integral value spectra for the shochu of samples No. 15 to No. 16 shown in Table 2, in the case where the number of integral values is 18.

FIG. 72 is a diagram showing integral value spectra for the shochu of samples No. 17 to No. 20 shown in Table 2, in the case where the number of integral values is 18.

FIG. 73 is a first diagram showing the result of judgment on whether the curves k87 to k106 shown in FIGS. 66 to 72 are or not different from each other.

FIG. 74 is a second diagram showing the result of judgment on whether the curves k87 to k106 shown in FIGS. 66 to 72 are or not different from each other.

FIG. 75 is a third diagram showing the result of judgment on whether the curves k87 to k106 shown in FIGS. 66 to 72 are or not different from each other.

FIG. 76 is a diagram showing the correspondence between the classes and integral values when the number of integral values is 138 and the classes and integral values when the number of integral values is 276.

FIG. 77 is a diagram showing integral value spectra of Crimson Seedless (skin+flesh), Green Seedless (skin+flesh) and Shine Muscat (skin+flesh) in when the number of integral values in a specified potential section in the range of −1362 mV to −2501 mV is 74.

FIG. 78 is a diagram showing integral value spectra of Crimson Seedless (only flesh), Green Seedless (only flesh) and Shine Muscat (only flesh) when the number of integral values in a specified potential section in the range of −1362 mV to −2501 mV is 74.

FIG. 79 is a diagram showing the result of judgment on whether the curves k107 to k112 shown in FIG. 77 and FIG. 78 are or not different from each other.

FIG. 80 is a diagram showing integral value spectra of Crimson Seedless (skin+flesh), Green Seedless (skin+flesh) and Shine Muscat (skin+flesh) in when the number of integral values in a specified potential section in the range of −1362 mV to −2501 mV is 37.

FIG. 81 is a diagram showing integral value spectra of Crimson Seedless (only flesh), Green Seedless (only flesh) and Shine Muscat (only flesh) in when the number of integral values in a specified potential section in the range of −1362 mV to −2501 mV is 37.

FIG. 82 is a diagram showing the result of judgment on whether the curves k113 to k118 shown in FIG. 80 and FIG. 81 are or not different from each other.

FIG. 83 is a diagram showing integral value spectra of Crimson Seedless (skin+flesh), Green Seedless (skin+flesh) and Shine Muscat (skin+flesh) in when the number of integral values in a specified potential section in the range of −1362 mV to −2501 mV is 19.

FIG. 84 is a diagram showing integral value spectra of Crimson Seedless (only flesh), Green Seedless (only flesh) and Shine Muscat (only flesh) in when the number of integral values in a specified potential section in the range of −1362 mV to −2501 mV is 19.

FIG. 85 is a diagram showing the result of judgment on whether the curves k119 to k124 shown in FIG. 83 and FIG. 84 are or not different from each other.

FIG. 86 is a diagram showing integral value spectra of Crimson Seedless (skin+flesh), Green Seedless (skin+flesh) and Shine Muscat (skin+flesh) in when the number of integral values in a specified potential section in the range of −1362 mV to −2501 mV is 9.

FIG. 87 is a diagram showing integral value spectra of Crimson Seedless (only flesh), Green Seedless (only flesh) and Shine Muscat (only flesh) in when the number of integral values in a specified potential section in the range of −1362 mV to −2501 mV is 9.

FIG. 88 is a diagram showing the result of judgment on whether the curves k125 to k130 shown in FIG. 86 and FIG. 87 are or not different from each other.

FIG. 89 is a diagram showing the correspondence between the classes and integral values in when the number of integral values is 37 and the classes and integral values in when the number of integral values is 74.

FIG. 90 is a flowchart for explaining the operation of the diagnostic system 10 shown in FIG. 1.

FIG. 91 is a flowchart for explaining the detailed operation of step S7 in FIG. 90.

FIG. 92 is a flowchart for explaining the detailed operation of step S73 in FIG. 91.

FIG. 93 is a flowchart for explaining the detailed operation of step S72 in FIG. 91.

FIG. 94 is a flowchart for explaining the detailed operation of step S75 in FIG. 91.

FIG. 95 is a flowchart for explaining the detailed operation of step S76 in FIG. 91.

FIG. 96 is a flowchart for explaining the detailed operation of step S77 in FIG. 91.

FIG. 97 is a flowchart for explaining the detailed operation of step S8 in FIG. 90.

FIG. 98 is a flowchart for explaining the detailed operation of step S82 in FIG. 97.

FIG. 99 is a flowchart for explaining the detailed operation of step S830 in FIG. 98.

FIG. 100 is another flowchart for explaining the detailed operation of step S830 in FIG. 98.

FIG. 101 is a flowchart for explaining the detailed operation of step S83 in FIG. 97.

FIG. 102 is another flowchart for explaining the detailed operation of step S83 in FIG. 97.

FIG. 103 is a diagram showing the correspondence relationship of classes in a positive predetermined potential section when the number of integral values in when the analyte is “shochu” is 18, 20, 32, 36, 56, 70, 92, 138 and 276.

FIG. 104 is a diagram showing the correspondence relationship of classes in a negative predetermined potential section when the number of integral values in when the analyte is “shochu” is 18, 20, 32, 36, 56, 70, 92, 138 and 276.

FIG. 105 is a diagram showing the correspondence relationship of classes when the number of integral values in when the analyte is “grapes” is 7, 9, 11, 13, 15, 19, 25, 37 and 74.

FIG. 106 is a conceptual diagram of a judgment result indicating whether two curves CURᵢ, CURⱼ of _{P}C₂ pairs are or not different.

FIG. 107 is a conceptual diagram showing the update from the analytical data ALY_Dᵤₙᵢ to the index data IDXᵤₙᵢ in step S74 of FIG. 91.

FIG. 108 is a conceptual diagram showing the updating of P pieces of analysis data ALY_D₁ to ALY_D_{P} to P pieces of index data IDX₁ to IDX_{P} in step S78 of FIG. 91.

FIG. 109 is a conceptual diagram showing the results of a taste diagnosis.

FIG. 110 is a schematic diagram of a diagnostic system according to the second embodiment.

FIG. 111 is a schematic diagram of the terminal device 3 shown in FIG. 110.

FIG. 112 is a schematic diagram of the diagnostic device 2A shown in FIG. 110.

FIG. 113 is a schematic diagram of the analysis/diagnostic unit 21A shown in FIG. 112.

FIG. 114 is a first flowchart for explaining the operation of the diagnostic system 10A shown in FIG. 110.

FIG. 115 is a second flowchart for explaining the operation of the diagnostic system 10A shown in FIG. 110.

FIG. 116 is a diagram for explaining the locations of the sensor device 1, the terminal device 3, and the diagnostic device 2A.

FIG. 117 is a schematic diagram of a diagnostic system according to the third embodiment.

FIG. 118 is a schematic diagram of the terminal device 3A shown in FIG. 117.

FIG. 119 is a schematic diagram of the analysis device 2C shown in FIG. 117.

FIG. 120 is a schematic diagram of the diagnostic device 2B shown in FIG. 117.

FIG. 121 is a first flowchart for explaining the operation of the diagnostic system 10B shown in FIG. 117.

FIG. 122 is a second flowchart for explaining the operation of the diagnostic system 10B shown in FIG. 117.

FIG. 123 is a diagram for explaining the locations of the sensor device 1, the terminal device 3A, the analysis device 2C, and the diagnostic device 2B.

FIG. 124 is another schematic diagram of the measuring instrument 12 shown in FIG. 4.

FIG. 125 is another schematic diagram of the diagnostic device 2 shown in FIG. 7.

FIG. 126 is a schematic diagram of the analysis/diagnostic circuit 21B shown in FIG. 125.

FIG. 127 is another schematic diagram of the terminal device 3 shown in FIG. 111.

FIG. 128 is another schematic diagram of the diagnostic device 2A shown in FIG. 112.

FIG. 129 is another schematic diagram of the analysis/diagnostic unit 21A shown in FIG. 113.

FIG. 130 is another schematic diagram of the terminal device 3A shown in FIG. 118.

FIG. 131 is another schematic diagram of the analysis device 2C shown in FIG. 119.

FIG. 132 is another schematic diagram of the diagnostic device 2B shown in FIG. 120.

### DESCRIPTION OF EMBODIMENTS

Embodiments of the present invention will be described in detail in conjunction with the accompanying drawings. Note that the same or corresponding portions in the drawings are denoted by the same reference numerals and their descriptions will not be repeated.

### First Embodiment

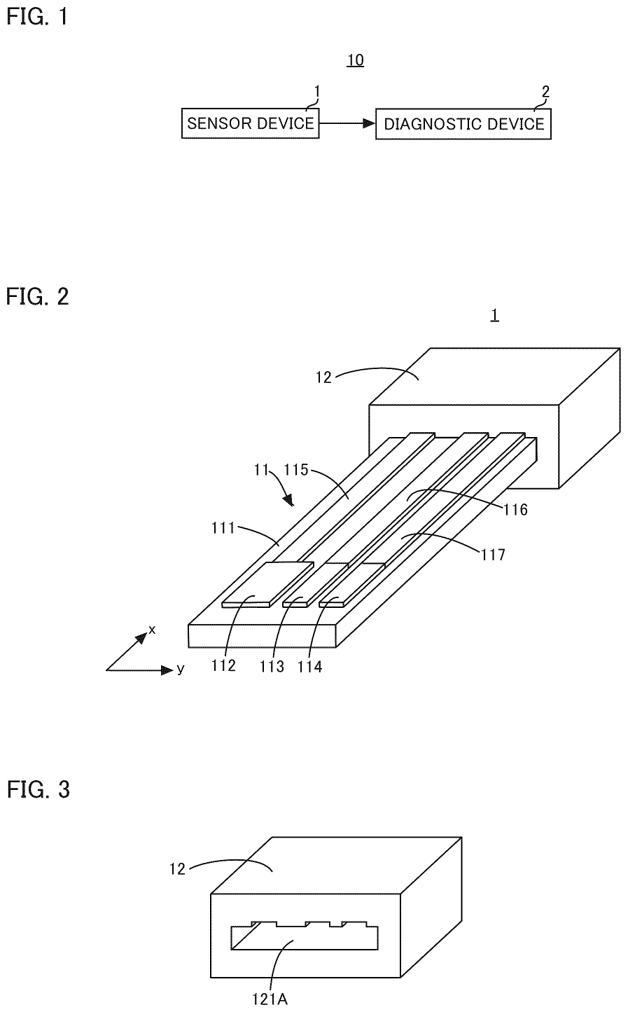

FIG. 1 is a schematic diagram of a diagnostic system according to a first embodiment of the present invention. Referring to FIG. 1, a diagnostic system 10 according to a first embodiment of the present invention includes a sensor device 1 and a diagnostic device 2.

The diagnostic system 10 is placed, for example, in restaurants such as Japanese restaurants, Chinese restaurants, and Western restaurants, sake breweries that brew shochu, liquor stores that sell shochu, ant etc.

The sensor device 1 measures measurement data of cyclic voltammogram of an analyte consisting of, for example, shochu or grapes (grape juice) by a cyclic voltammetry method, and transmits the measurement data of cyclic voltammogram to the diagnostic device 2 by wireless communication or wired communication.

The cyclic voltammetry method (CV) is a measurement method in which electrodes are placed in a stationary solution, analyzes the current-potential curve (cyclic voltammogram CVG) obtained by analyzing the current-potential curve (cyclic voltammogram CVG) obtained by measuring the current that flows when the potential is repeatedly swept to examine redox properties, etc.

The cyclic voltammogram CVG is a current-potential curve measured by cyclic voltammetry method, and the measurement data of the cyclic voltammogram CVG includes current-potential characteristics (I-V) that have associated the current I with the potential V.

When the sensor device 1 transmits the measurement data of the cyclic voltammogram CVG to the diagnostic device 2 by wireless communication, the sensor device 1 transmits the measurement data of the cyclic voltammogram CVG to the diagnostic device 2 by wireless communication, for example, via Bluetooth (registered trademark).

Furthermore, when the sensor device 1 transmits the measurement data of the cyclic voltammogram CVG to the diagnostic device 2 by wired communication, the sensor device 1 is connected to the diagnostic device 2 by a cable, and transmits the measurement data to the diagnostic device 2 via the cable.

The diagnostic device 2 receives measurement data of the cyclic voltammogram CVG from the sensor device 1 by wireless communication or wired communication. The diagnostic device 2 then calculates, for all of the predetermined potential sections, an integral value in a predetermined potential section of the current-potential characteristic (I-V) included in the measurement data based on the measurement data of the cyclic voltammogram CVG using a method described below, thereby calculating a plurality of integral values in the plurality of predetermined potential sections, and creates a curve CUR indicating the dependency of the integral value on the predetermined potential section based on the calculated plurality of integral values in the plurality of predetermined potential sections as [an index curve which is a curve that serves as an index when identifying the analyte], and diagnoses the taste of shochu or grapes (grape juice) based on the plurality of integral values in the plurality of predetermined potential sections, and displays the diagnosis result.

FIG. 2 is a schematic diagram of the sensor device 1 shown in FIG. 1. FIG. 3 is a perspective view of the measuring instrument 12 shown in FIG. 2.

Referring to FIG. 2, the sensor device 1 includes a sensor 11 and a measuring instrument 12. The sensor 11 includes a substrate 111, a working electrode 112, a counter electrode 113, a reference electrode 114, and wires 115-117.

In FIG. 2, an x-y plane is defined. The substrate 111 has, for example, a flat plate shape and is disposed along the x-y plane.

The wirings 115 to 117 are placed on the upper surface of the substrate 111 along the x-axis direction (first direction). The wiring 116 is disposed along the x-axis direction (first direction) at a predetermined interval (for example, 2 to 3 mm) from the wiring 115 in the y-axis direction (second direction perpendicular to the first direction). Moreover, the wiring 117 is disposed along the x-axis direction (first direction) at a predetermined interval (for example, 2 to 3 mm) from the wiring 116 in the y-axis direction (second direction perpendicular to the first direction).

The working electrode 112 is disposed on one end of the wiring 115 opposite the measuring instrument 12 side of the wiring 115 and is electrically connected to the wiring 115. The counter electrode 113 is disposed on one end of the wiring 116 opposite the measuring instrument 12 side of the wiring 116 and is electrically connected to the wiring 116. The reference electrode 114 is disposed on one end of the wiring 117 opposite the measuring instrument 12 side of the wiring 117 and is electrically connected to the wiring 117.

The substrate 111 is, for example, a printed circuit board (PCB: Printed Circuit Board), a plastic plate, or a glass epoxy substrate, and has, for example, a width of 12 mm, a length of 80 mm, and a thickness of 1 mm. The working electrode 112 is made of, for example, any one of boron (B) doped diamond (BDD), a carbon electrode, glassy carbon (glassy diamond), gold (Au) and platinum (Pt). The counter electrode 113 is made of, for example, gold (Au). The reference electrode 114 is made of, for example, gold (Au) or Ag/AgCl.

When the working electrode 12 is made of diamond, the diamond may be either single crystal diamond or polycrystalline diamond, but the polycrystalline diamond is preferred. In this case, the polycrystalline diamond is further preferable that the dangling bonds on the outermost surface of the polycrystalline diamond are terminated with hydrogen.

The working electrode 112 has, for example, a rectangular planar shape with an area of 3×3 mm², the counter electrode 113 has, for example, a rectangular planar shape with an area of 3×3 mm², and the reference electrode 114 has, for example, a rectangular planar shape with an area of 1×2 mm².

When the working electrode 112 is made of diamond or gold, the working electrode 112 has a planar shape that is, for example, a circular shape with a diameter of 3.5 mm.

Furthermore, when the working electrode 112 is made of glassy carbon, the cyclic voltammogram CVG can be measured over a wide range.

The working electrode 112 is an electrode that transfers electrons to and from the analyte. The counter electrode 113 is an electrode that returns to the system a current value that is the same as the current value generated at the working electrode 112. The reference electrode 114 is an electrode that serves as a reference when determining the potential of the working electrode 112.

An analyte consisting of shochu or grapes (grape juice) is supplied to a region in which a working electrode 112, a counter electrode 113 and a reference electrode 114 are arranged.

Referring to FIG. 3, measuring instrument 12 has a recess 121A into which a portion of the other end side of sensor 11 is inserted.

When the sensor 11 is electrically connected to the measuring instrument 12, a portion of the other end side of the sensor 11 in the x-axis direction (first direction) is inserted into a recess 121 A of the measuring instrument 12. As a result, the wiring 115 to 117 of the sensor 11 is electrically connected to the measuring instrument 12. Furthermore, when the sensor 11 is not electrically connected to the measuring instrument 12, a portion of the other end side of the sensor 11 in the x-axis direction (first direction) is pulled out from the recess 121 A of the measuring instrument 12.

Therefore, by attaching and detaching a portion of the other end of the sensor 11 in the x-axis direction (first direction) to the recess 121A of the measuring instrument 12, the sensor 11 can be electrically connected to the measuring instrument 12 or electrically disconnected from the measuring instrument 12.

In the embodiment of the present invention, the sensor 11 is used to measure the cyclic voltammogram CVG of the analyte, is attachable to and detachable from a measuring instrument 12 that measures the cyclic voltammogram CVG, and is the sensor discarded each time a measurement of the cyclic voltammogram CVG is performed. In other words, the sensor 11 is a disposable sensor.

In this way, the sensor 11 is discarded each time a cyclic voltammogram measurement is performed, so there is no need to regenerate the electrodes (working electrode 112, counter electrode 113 and reference electrode 114) by physical polishing or the like, or to pretreat the sample (analyte).

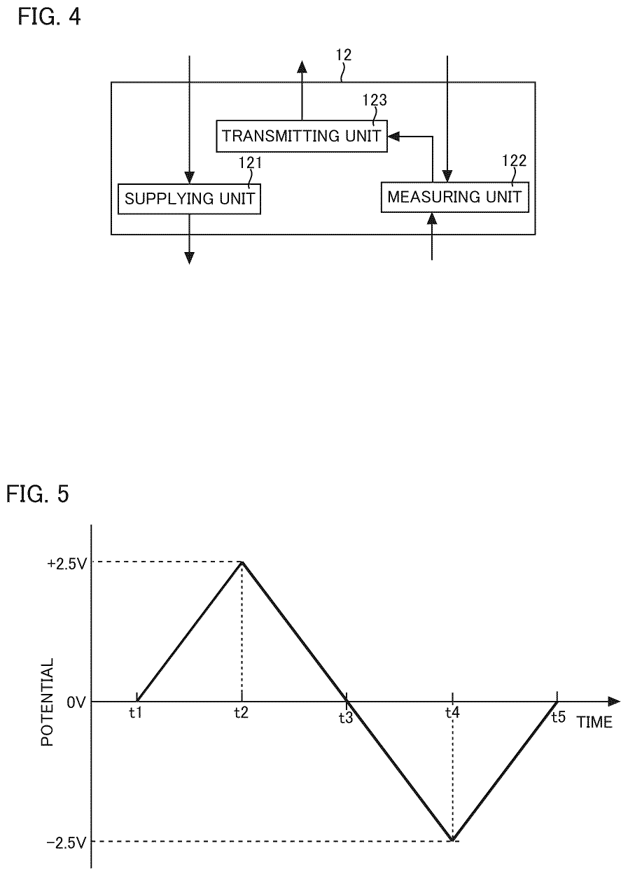

FIG. 4 is a schematic diagram of the measuring instrument 12 shown in FIG. 2. FIG. 5 is a schematic diagram showing a timing chart of the potential supplied to the sensor 11 shown in FIG. 2.

Referring to FIG. 4, the measuring instrument 12 includes a supplying unit 121, a measuring unit 122, and a transmitting unit 123.

The supply unit 121 is electrically connected to the working electrode 112 by a wiring 115. The supply unit 121 accepts the potential scanning range and the potential scanning speed input by the user of the sensor device 1. The supply unit 121 supplies a potential within a potential scanning range to the working electrode 112 via the wiring 115 while changing the potential at a predetermined scanning speed.

Here, users of the sensor device 1 are, for example, staff at restaurants such as Japanese restaurants, Chinese restaurants, and Western restaurants, master brewers at sake breweries, and staff at liquor stores.

The measurement unit 122 is electrically connected to the working electrode 112, the counter electrode 113, and the reference electrode 114 by wiring 115, 116, and 117, respectively, and measures the potential V of the working electrode 112 based on the potential of the reference electrode 114 as a reference, and also measures the current value I from the counter electrode 113, and creates measurement data MRS including current-potential characteristics (I-V) that mutually correspond the measured potential V and current value I.

The potential scanning speed is, for example, 0.3 V/sec, 0.5 V/sec, or 0.6 V/sec, and the potential scanning range is, for example, −2.5 V to +2.5 V.

Referring to FIG. 5, in a period from time t1 to time t2, the supply unit 121 supplies a potential V in the range of 0 V to +2.5 V to the working electrode 112 while changing the potential V at a predetermined scanning speed.

Thereafter, during a period from time t2 to time t3, the supply unit 121 supplies a potential V in the range of +2.5 V to 0 V to the working electrode 112 while changing the potential V at a predetermined scanning speed.

Subsequently, in the period from time t3 to time t4, the supply unit 121 supplies a potential V in the range of 0 V to −2.5 V to the working electrode 112 while changing the potential V at a predetermined scanning speed.

Furthermore, in the period from time t4 to time t5, the supply unit 121 supplies a potential V in the range of −2.5 V to 0 V to the working electrode 112 while changing the potential V at a predetermined scanning speed.

In this manner, the supply unit 121 supplies the potential V with triangular wave to the working electrode 112.

In the embodiment of the present invention, the supply unit 121 supplies a potential V in a potential scanning range of −2.5 V to +2.5 V to the working electrode 112 while changing the potential V at a scan speed of 0.3 V/sec, and the measurement unit 122 measures the potential V of the working electrode 112 by using the potential of the reference electrode 114 as a criterion, measures the current value I from the counter electrode 113, and creates measurement data MRS_Low including a current-potential characteristic (I-V)_Low that the measured potential V and current value I are corresponded with each other.

In addition, the supply unit 121 supplies a potential V in a potential scanning range of −2.5 V to +2.5 V to the working electrode 112 while changing the potential V at a scan speed of 0.5 V/sec, and the measurement unit 122 measures the potential V of the working electrode 112 by using the potential of the reference electrode 114 as a criterion, measures the current value I from the counter electrode 113, and creates measurement data MRS_Middle including a current-potential characteristic (I-V)_Middle that the measured potential V and current value I are corresponded with each other.

Furthermore, the supply unit 121 supplies a potential V in a potential scanning range of −2.5 V to +2.5 V to the working electrode 112 while changing the potential V at a scan speed of 0.6 V/sec, and the measurement unit 122 measures the potential V of the working electrode 112 by using the potential of the reference electrode 114 as a criterion, measures the current value I from the counter electrode 113, and creates measurement data MRS_High including a current-potential characteristic (I-V) High that the measured potential V and current value I are corresponded with each other.

Then, the measurement unit 122 creates measurement data MRS including the measurement data MRS_Low, the measurement data MRS_Middle, and the measurement data MRS_High, and outputs the created measurement data MRS to the transmitting unit 123.

The transmitting unit 123 receives the measurement data MRS from the measurement unit 122, and transmits the received measurement data MRS to the diagnostic device 2 by wireless communication or wired communication.

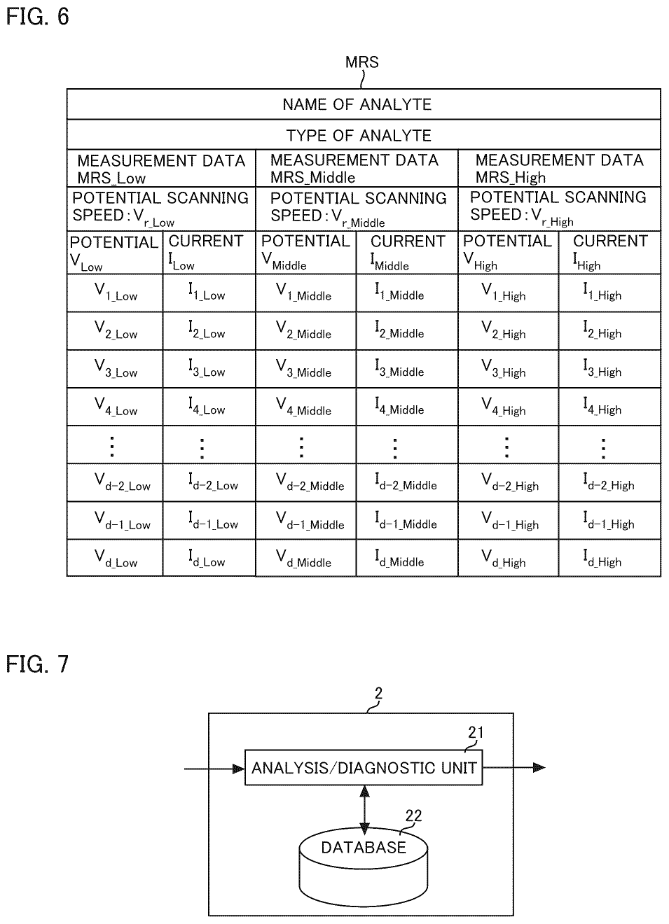

FIG. 6 is a schematic diagram of the measurement data MRS. Referring to FIG. 6, the measurement data MRS includes a name of the analyte, a type of the analyte, and the measurement data MRS_Low, MRS_Middle, and MRS_High.

The name of the analyte is composed of, for example, shochu or grapes. When the name of the analyte is shochu, the types of the analyte are composed of, for example, sweet potato shochu, barley shochu, and rice shochu etc. Furthermore, when the name of the analyte is “grape”, the types of the analyte include, for example, green seedless, crimson seedless, and shine muscat etc.

The measurement data MRS_Low includes a potential scanning speed V_{r_Low} and a current-potential characteristic (I_{Low}-V_{Low}). The current-potential characteristic (I_{Low}-V_{Low}) consists of a configuration in which the potential V_{Low} corresponds to the current value I_{Low}.

The potential V_{Low} consistes of V_{1_Low} to V_{d_Low}, and the current value I_{Low} consists of I_{1_Low} to I_{d_Low}. The current values I_{1_Low} to I_{d_Low} are corresponded to the potentials V_{1_Low} to V_{d_Low}, respectively.

The potentials V_{1_Low} to V_{d_Low} are the potentials of the working electrode 112 measured based on the potential of the reference electrode 114 when the potential scanning speed is “V_{r_Low}”, and the current values I_{1_Low} to I_{d_Low} are the current values I from the counter electrode 113 in when the potential scanning speed is “V_{r_Low}”.

The measurement data MRS_Middle includes a potential scanning speed V_{r_Middle} and a current-potential characteristic (I_{Middle}-V_{Middle}). The current-potential characteristic (I_{Middle}-V_{Middle}) is configuration which a potential V_{Middle} is corresponded to a current value I_{Middle}.

The potential V_{Middle} is composed of V_{1_Middle} to V_{d_Middle}, and the current value I_{Middle} is composed of from I_{1_Middle} to I_{d_Middle}. The current values I_{1_Middle} to I_{d_Middle} are corresponded to potentials V_{1_Middle} to V_{d_Middle}, respectively.

The potentials V_{1_Middle} to V_{d_Middle} are the potentials of the working electrode 112 measured based on the potential of the reference electrode 114 when the potential scanning speed is “V_{r_Middle}”, and the current values I_{1_Middle} to I_{d_Middle} are the current values I from the counter electrode 113 when the potential scanning speed is “V_{r_Middle}”.

The measurement data MRS_High includes a potential scanning speed V_{r_High} and a current-potential characteristic (I_{High}-V_{High}). The current-potential characteristic (I_{High}-V_{High}) consists of a correspondence between the potential V_{High} and the current value I_{High}.

The potential V_{High} ranges from V_{1_High} to V_{d_High}, and the current value I_{High} ranges from I_{1_High} to I_{d_High}. The current values I_{1_High} to Id High correspond to the potentials V_{1_High} to V_{d_High}, respectively.

The potentials V_{1_High} to V_{d_High} are the potentials of the working electrode 112 measured based on the potential of the reference electrode 114 when the potential scanning speed is “V_{r_High}”, and the current values I_{1_High} to Id High are the current values I from the counter electrode 113 when the potential scanning speed is “V_{r_High}”.

When the scanning range of the potential V_{Low} is −2.5V to +2.5V, the potentials V_{1_Low}, V_{2_Low}, V_{3_Low}, V_{4_Low}, . . . , V_{d-2_Low}, V_{d-1_Low}, and V_{d_Low} are respectively 0V, 1 mV, 2 mV, 3 mV, . . . , 2499 mV, 2500 mV, 2499 mV, . . . , 2 mV, 1 mV, 0 mV, −1 mV, −2 mV, . . . , −2499 mV, −2500 mV, −2499 mV, . . . , −2 mV, −1 mV, and 0V. That is, the potentials V_{1_Low}, V_{2_Low}, V_{3_Low}, V_{4_Low}, . . . , V_{d-2_Low}, V_{d-1_Low}, and V_{d_Low} are made up of potentials that are changed by unit potential (=1 mV). As a result, d represents twice the total number of unit potentials in the scanning range of the potential V.

The same is applied to the potentials V_{1_Middle} to V_{d_Middle} and the potentials V_{1_High} to V_{d_High}.

The measurement unit 122 receives the name of the analyte, the type of analyte, and the potential scanning speeds V_{r_Low}, V_{r_Middle}, and V_{r_High} from the user of the sensor device 1, and measures the current-potential characteristics (I_{Low}-V_{Low}) at the potential scanning speed V_{r_Low}, measures the current-potential characteristics (I_{Middle}-V_{Middle}) at the potential scanning speed V_{r_Middle}, and measures the current-potential characteristics (I_{High}-V_{High}) at the potential scanning speed Vᵣ_High. Then, the measurement unit 122 creates measurement data MRS including the name of the analyte, the type of analyte, the potential scanning speeds V_{r_Low}, V_{r_Middle}, V_{r_High}, and the current-potential characteristics (I_{Low}-V_{Low}), (I_{Middle}-V_{Middle}), and (I_{High}-V_{High}), and outputs the created measurement data MRS to the transmitting unit 123.

The transmitting unit 123 receives the measurement data MRS from the measurement unit 122, and transmits the received measurement data MRS to the diagnostic device 2 by wired communication or wireless communication.

When transmitting the measurement data MRS to the diagnostic device 2 by wireless communication, the transmitting unit 123 transmits the measurement data MRS to the diagnostic device 2 by, for example, Bluetooth (registered trademark).

Furthermore, when the transmitting unit 123 transmits the measurement data MRS to the diagnostic device 2 by wired communication, the transmitting unit 123 is connected to the diagnostic device 2 by a cable.

When the sensor device 1 measures P cyclic voltammograms of P (P is an integer greater than or equal to 2) analytes using the cyclic voltammetry method, the measurement unit 122 of the sensor device 1 creates each of the P pieces of measurement data MRS_1 to MRS_P using the method described above, and the transmitting unit 123 of the sensor device 1 transmits the P pieces of measurement data MRS_1 to MRS_P created by the measurement unit 122 to the diagnostic device 2 via wired or wireless communication. In this case, each of the P pieces of measurement data MRS_1 to MRS_P has the same configuration as the measurement data MRS shown in FIG. 6.

FIG. 7 is a schematic diagram of the diagnostic device 2 shown in FIG. 1. Referring to FIG. 7, the diagnostic device 2 includes an analysis/diagnostic unit 21 and a database 22.

The analysis/diagnostic unit 21 receives the measurement data MRS from the measuring instrument 12 (transmitting unit 123) of the sensor device 1 by wireless communication or wired communication.

Then, the analysis/diagnostic unit 21 diagnoses the taste of the analyte (shochu or grapes) based on the measurement data MRS using a method described below, stores the diagnosed taste diagnosis result in the database 22 in association with the analyte (shochu or grapes), and displays the taste diagnosis result of the analyte (shochu or grapes).

The database 22 stores the taste diagnosis results in association with the analyte (shochu or grapes).

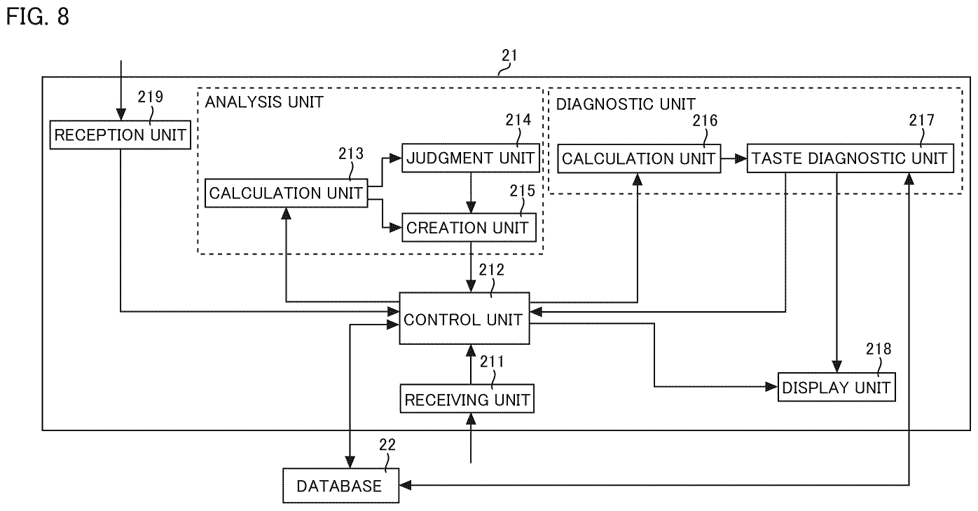

FIG. 8 is a schematic diagram of the analysis/diagnostic unit 21 shown in FIG. 7. Referring to FIG. 8, the analysis/diagnostic unit 21 includes a receiving unit 211, a control unit 212, calculation units 213 and 216, a judgment unit 214, a creation unit 215, a taste diagnostic unit 217, a display unit 218, and a reception unit 219.

The receiving unit 211 receives the measurement data MRS from the measuring instrument 12 (transmitting unit 123) of the sensor device 1 by wireless communication or wired communication, and outputs the received measurement data MRS to the control unit 212.

Here, the measurement data MRS may be one piece of measurement data or may be a plurality of pieces of measurement data.

The control unit 212 includes a timer. When the control unit 212 receives one piece of measurement data MRS_uni from the receiving unit 211, the control unit 212 refers to the timer to detect the time tᵤₙᵢ when the measurement data MRS_uni was received, and issues identification information IDᵤₙᵢ for identifying the measurement data MRS_uni. The measurement data MRS_uni has the same structure as the measurement data MRS shown in FIG. 6.

Then, the control unit 212 detects the name ALY_Naᵤₙᵢ of the analyte, the type ALY_Kdᵤₙᵢ of the analyte, the potential scanning speed V_{r_Low_uni}, the current-potential characteristics (I_{Low}-V_{Low})ᵤₙᵢ, the potential scanning speed V_{r_Middle_uni}, the current-potential characteristics (I_{Middle}-V_{Middle})ᵤₙᵢ, the potential scanning speed V_{r_High_uni}, and the current-potential characteristics (I_{High}-V_{High})ᵤₙᵢ from the measurement data MRS_uni.

Thereafter, the control unit 212 generates measurement data MRS_Low_uni={V_{r_Low_uni}:(I_{Low}-V_{Low})ᵤₙᵢ} in which the potential scanning speed V_{r_Low_uni} and the current-potential characteristic (I_{Low}-V_{Low})ᵤₙᵢ are corresponded each other, measurement data MRS_Middle_uni={V_{r_middle_uni}:(I_{middle}-V_{middle})ᵤₙᵢ} in which the potential scanning speed V_{r_middle_uni} and the current-potential characteristic (I_{middle}-V_{middle})ᵤₙᵢ are corresponded each other, and measurement data MRS_High_uni={V_{r_High_uni}:(I_{High}-V_{High})ᵤₙᵢ} in which the potential scanning speed V_{r_High_uni} and the current-potential characteristic (I_{High}-V_{High})ᵤₙᵢ are corresponded each other.

Then, the control unit creates analysis data ALY_Dᵤₙᵢ=[tᵤₙᵢ/IDᵤₙᵢ/ALY_Naᵤₙᵢ/ALY_Kdᵤₙᵢ/MRS_Low_uni/MRS_Middle_uni/MRS_High_uni] in which the time tᵤₙᵢ, the identification information IDᵤₙᵢ, the name ALY_Naᵤₙᵢ of analyte, the kind ALY_Kdᵤₙᵢ of the analyte, the measurement data MRS_Low_uni={V_{r_Low_uni}: (I_{Low}-V_{Low})ᵤₙᵢ}, the measurement data MRS_Middle_uni={V_{r_Middle_uni}: (I_{Middle}-V_{Middle})ᵤₙᵢ}, and the measurement data MRS_High_uni={V_{r_High_uni}: (I_{High}-V_{High})ᵤₙᵢ} are associated with each other.

Then, the control unit 212 stores the analysis data ALY_Dᵤₙᵢ in the database 22 and outputs the analysis data ALY_Dᵤₙᵢ to the calculation unit 213.

In addition, when the control unit 212 have received P pieces of measurement data MRS_1 to MRS_P (i.e., multiple measurement data) from the receiving unit 211, the control unit 212 refers to the timer to detect the times t₁ to t_{P} at when the P pieces of measurement data MRS_1 to MRS_P were received, respectively, and issues P pieces of identification information ID₁ to ID_{P} for identifying the P pieces of measurement data MRS_1 to MRS_P, respectively. Here, each of the P pieces of measurement data MRS_1 to MRS_P has the same configuration as the measurement data MRS shown in FIG. 6.

Then, the control unit 212 executes to detecting the name ALY_Naₚ of the analyte, the kind ALY_Kdₚ of the analyte, the potential scanning speed V_{r_Low_p}, the current-potential characteristic (I_{Low}-V_{Low})ₚ, the potential scanning speed V_{r_Middle_p}, the current-potential characteristic (I_{Middle}-V_{Middle})ₚ, the potential scanning speed V_{r_High_p}, and the current-potential characteristic (I_{High}-V_{High})ₚ from the measurement data MRS_p (p is any of 1 to P) for all of P pieces of measurement data MRS_1 to MRS_P (i.e., multiple measurement data).

Thereafter, the control unit 212 executes for all of p=1 to P generating measurement data MRS_Low_p={V_{r_Low_p}: (I_{Low}-V_{Low})ₚ} in which the potential scanning speed V_{r_Low_p} and the current-potential characteristic (I_{Low}-V_{Low})ₚ are corresponded to each other, measurement data MRS_Middle_p={V_{r_Middle_p}: (I_{Middle}-V_{Middle})ₚ} in which the potential scanning speed V_{r_Middle_p} and the current-potential characteristic (I_{Middle}-V_{Middle})ₚ are corresponded to each other, and measurement data MRS_High_p={V_{r_High_p}: (I_{High}-V_{High})ₚ} in which the potential scanning speed V_{r_High_p} and the current-potential characteristic (I_{High}-V_{High})ₚ are corresponded to each other, and the control unit 212 creates P pieces of measurement data MRS_Low_={V_{r_Low_1}: (I_{Low}-V_{Low})₁} to MRS_Low_P={V_{r_Low_P}: (I_{Low}-V_{Low})_{P}}, P pieces of measurement data MRS_Middle_1={V_{r_Middle_1}: (I_{Middle}-V_{Middle})₁} to MRS_Middle_P={V_{r_Middle_P}: (I_{Middle}-V_{Middle})_{P}}, and P pieces of measurement data MRS_High_1={V_{r_High_1}: (I_{High}-V_{High})₁}-MRS_High_P={V_{r_High_P}: (I_{High}-V_{High})_{P}}.

Then, the control unit 212 executes for all of p=1 to P creating analysis data ALY_Dₚ=[tₚ/IDₚ/ALY_Naₚ/ALY_Kdₚ/measurement data MRS_Low_p/measurement data MRS_Middle_p/measurement data MRS_High_p] in which the time tₚ, the identification information IDₚ, the name ALY_Naₚ of the analyte, the type ALY_Kdₚ of the analyte, the measurement data MRS_Low_p={V_{r_Low_p}: (I_{Low}-V_{Low})ₚ}, the measurement data MRS_Middle_p={V_{r_Middle_p}: (I_{Middle}-V_{Middle})ₚ}. And the control unit 212 creates P pieces of analysis data ALY_D₁=[t₁/ID₁/ALY_Na₁/ALY_Kd₁/measurement data MRS_Low_1/measurement data MRS_Middle_1/measurement data MRS_High_1] to ALY_D_{P}=[tₚ/ID_{P}/ALY_Naₚ/ALY_Kdₚ/measurement data MRS_Low_P/measurement data MRS_Middle_P/measurement data MRS_High_P].

Then, the control unit 212 stores the P pieces of analysis data ALY_D₁ to ALY_D_{P} in the database 22, and outputs the P pieces of analysis data ALY_D₁ to ALY_D_{P} to the calculation unit 213.

Furthermore, after outputting the analysis data ALY_Dᵤₙᵢ to the calculation unit 213, the control unit 212 receives from the creation unit 215 an analysis result ALY_RLSᵤₙᵢ=[IDᵤₙᵢ/CALᵤₙᵢ/CURᵤₙᵢ] in which the identification information IDᵤₙᵢ, the calculation data CALᵤₙᵢ, and the curve CURᵤₙᵢ are corresponded to each other, and then detects the identification information IDᵤₙᵢ, the calculation data CALᵤₙᵢ, and the curve CURᵤₙᵢ from the analysis result ALY_RLSᵤₙᵢ, and reads out from the database 22 the analysis data ALY_Dᵤₙᵢ having the same identification information as the detected identification information IDᵤₙᵢ.

Then, the control unit 212 stores the calculation data CALᵤₙᵢ and the curve CURᵤₙᵢ in the analysis data ALY_Dᵤₙᵢ read out from the database 22 to update the analysis data ALY_Dᵤₙᵢ to the index data IDXᵤₙᵢ, and outputs the updated index data IDXᵤₙᵢ to the calculation unit 216.

Thereafter, the control unit 212 stores the index data IDXᵤₙᵢ in the database 22 in place of the analysis data ALY_Dᵤₙᵢ.

Furthermore, the control unit 212 receives from the create unit 215 the P analysis results ALY_RLS₁=[ID₁/CAL₁/CUR₁] to ALY_RLS_{P}=[ID_{P}/CAL_{P}/CUR_{P}] in which the P pieces of identification information ID₁ to ID_{P}, the P pieces of calculation data CAL₁ to CAL_{P}, and the P pieces of curves CUR₁ to CUR_{P} are associated with each other and a judgment result JDGR indicating whether the P curves CUR₁ to CUR_{P} are or not different from each other after the control unit 212 outputs the P pieces of analysis data ALY_D₁ to ALY_D_{P} to the calculation unit 213. After that, the control unit 212 detects the identification information IDₚ, calculation data CAL_{P}, and curve CURₚ from the analysis result ALY_RLSₚ=[IDₚ/CALₚ/CURₚ] (p is any of 1 to P) and executes for all of p=1 to P reading out the analysis data ALY_Dₚ having the same identification information as the detected identification information IDₚ from the database 22.

Then, the control unit 212 stores the calculation data CALₚ and the curve CURₚ in the analysis data ALY_Dₚ read out from the database 22 and executes for all of p=1 to P updating the analysis data ALY_Dₚ to the index data IDXₚ.

Thereafter, the control unit 212 outputs the P index data IDX₁ to IDXₚ to the calculation unit 216.

Then, the control unit 212 stores the P index data IDX₁ to IDX_{P} in the database 22 in place of the P analysis data ALY_D₁ to ALY_D_{P}, respectively.

Then, the control unit 212 stores the judgment result JDGR in the database 22 in association with the P index data IDX₁ to IDX_{P}.

Furthermore, after outputting the index data IDXᵤₙᵢ to the calculation unit 216, the control unit 212 receives a diagnosis result JDRᵤₙᵢ which is the result of diagnosing the taste (astringency, aftertaste, sweetness, aroma and bitterness) of the analyte (shochu or grapes) from the taste diagnostic unit 217, and stores the received diagnosis result JDRᵤₙᵢ in the database 22 in correspondence with the index data IDXᵤₙᵢ.

Furthermore, the control unit 212 receives from the taste diagnostic unit 217 P diagnosis results JDR₁ to JDR_{P} which are the results of diagnosing the taste (astringency, aftertaste, sweetness, aroma and bitterness) of the analyte (shochu or grapes) after outputting the P pieces of index data IDX₁ to IDX_{P} to the calculation unit 216. Then, the control unit 212 stores the received P diagnosis results JDR₁ to JDR_{P} in the database 22 in association with P index data IDX1 to IDXP, respectively.

Furthermore, when the control unit 212 have received a request RQTᵤₙᵢ that displays the name ALY_Naᵤₙᵢ of the analyte and the diagnosis result JDRᵤₙᵢ associated with the name ALY_Naᵤₙᵢ from the reception unit 219, the control unit 212 detects the diagnosis result JDRᵤₙᵢ associated with the name ALY_Naᵤₙᵢ of the analyte from the database 22 based on the name ALY_Naᵤₙᵢ of the analyte, and outputs the name ALY_Naᵤₙᵢ of the analyte and the diagnosis result JDRᵤₙᵢ to the display unit 218.

Furthermore, the control unit 212 receives from the reception unit 219 a request RQT_{q} to display q (where q is an integer satisfying 1≤q≤P) names ALY_Na₁ to ALY_Na_{q} in the P names ALY_Na₁ to ALY_Na_{P} of the P analytes and q diagnosis results JDR₁ to JDR_{q} associated with the q names ALY_Na₁ to ALY_Na_{q}, respectively. Then, the control unit 212 detects q diagnosis results JDR₁ to JDR_{q} associated with q names ALY_Na₁ to ALY_Na_{q} of q analytes from database22 based on the q names ALY_Na₁ to ALY_Na_{q}, and outputs the q names ALY_Na₁ to ALY_Na_{q} of q analytes and q diagnosis results JDR₁ to JDR_{q} to the display unit 218.

In this case, in the request RQTq, q types ALY_Kd₁ to ALY_Kd_{q} of q analytes may be used instead of q names ALY_Na₁ to ALY_Na_{q} of q analytes.

The calculation unit 213 receives the analysis data ALY_Dᵤₙᵢ=[tᵤₙᵢ/IDᵤₙᵢ/ALY_Naᵤₙᵢ/ALY_Kdᵤₙᵢ/MRS_Low_uni={Low_uni:(I_{LOW}-V_{Low})ᵤₙᵢ}/MRS_Middle_uni={Middle_uni:(I_{Middle}-V_{Middle})ᵤₙᵢ}/MRS_High_uni={High_uni:(I_{High}-V_{High})ᵤₙᵢ}] from the control unit 212.

Then, the calculation unit 213 executes, for all of predetermined potential section V_ITV, calculating the integral value ITG of the cyclic voltammogram CVG for each predetermined potential section V_ITV based on the current-potential characteristics (I_{Low}-V_{Low})ᵤₙᵢ, (I_{Middle}-V_{Middle})ᵤₙᵢ, (I_{High}-V_{High})ᵤₙᵢ using a method described below based on the current-potential characteristics (I_{Low}-V_{Low}), (I_{Middle}-V_{Middle}), (I_{High}-V_{High}) in the analysis data ALY_Dᵤₙᵢ=[tᵤₙᵢ/IDᵤₙᵢ/ALY_Naᵤₙᵢ/ALY_Kdᵤₙᵢ/MRS_Low_uni={Low_uni: (I_{Low}-V_{Low})ᵤₙᵢ}/MRS_Middle_uni={Middle_uni: (I_{Middle}-V_{Middle})ᵤₙᵢ}/MRS_High_uni={High_uni:(I_{High}-V_{High})ᵤₙᵢ}].

Then, when the number of predetermined potential sections V_ITV is n, the calculation unit 213 sets the first predetermined potential section V_ITV to “class Cls₁”, sets the second predetermined potential section V_ITV to “class Cls₂”, . . . , and similarly sets the (n−1)th predetermined potential section V_ITV to “class Clsₙ₋₁” and sets the nth predetermined potential section V_ITV to “class Clsₙ”.

Then, based on the current-potential characteristic (I_{Low}-V_{Low})ᵤₙᵢ of the measurement data MRS_Low_uni={Low_uni:(I_{Low}-V_{Low})ᵤₙᵢ}, the calculation unit 213 sets the integral value in the first predetermined potential section V_ITV to ITG_{1_Low}, sets the integral value in the second predetermined potential section V_ITV to ITG_{2_Low}, . . . , similarly sets the integral value in the (n−1)th predetermined potential section V_ITV to ITG_{(n-1)_Low} and sets the integral value in the nth predetermined potential section V_ITV to ITG_{n_Low}.

In addition, based on the current-potential characteristic (I_{Middle}-V_{Middle})ᵤₙᵢ of the measurement data MRS_Middle_uni={Middle_uni:(I_{Middle}-V_{Middle})ᵤₙᵢ}, the calculation unit 213 sets the integral value in the first predetermined potential section V_ITV to ITG_{1_Middle}, sets the integral value in the second predetermined potential section V_ITV to ITG_{2_Middle}, . . . , similarly sets the integral value in the (n−1)th predetermined potential section V_ITV to ITG_{(n-1)_Middle}, and sets the integral value in the nth predetermined potential section V_ITV to ITG_{n_Middle}.

Furthermore, based on the current-potential characteristic (I_{High}-V_{High})ᵤₙᵢ of the measurement data MRS_High_uni={High_uni:(I_{High}-V_{High})ᵤₙᵢ}, the calculation unit 213 sets the integral value in the first predetermined potential section V_ITV to ITG_{1_High}, sets the integral value in the second predetermined potential section V_ITV to ITG_{2_High}, . . . , similarly sets the integral value in the (n−1)th predetermined potential section V_ITV to ITG_{(n-1)_High} and sets the integral value in the nth predetermined potential section V_ITV to ITG_{n_High}.

Then, the calculation unit 213 adds the integral values ITG_{1_Low}, ITG_{1_Middle}, and ITG_{1_High} in class Cls₁ to calculate the sum ITG_{1_Low}+ITG_{1_Middle}+ITG_{1_High} of the integral values, adds the integral values ITG_{2_Low}, ITG_{2_Middle}, and ITG_{2_High} in class Cls₂ to calculate the sum ITG_{2_Low}+ITG_{2_Middle}+ITG_{2_High} of the integral values, similarly, adds the integral values ITG_{n_Low}, ITG_{n_Middle}, and ITG_{n_High} in class ClSₙ to calculate the sum ITG_{n_Low}+ITG_{n_Middle}+ITG_{n_High} of the integral values.

Then, the calculation unit 213 creates calculation data CALᵤₙᵢ which includes n classes Cls₁ to Clsₙ, n integral values ITG_{1_Low} to ITG_{n_Low}, ITG_{1_Middle} to ITG_{n_Middle}, and ITG_{1_High} to ITG_{n_High} which are respectively associated with the n classes Cls₁ to Clsₙ, and the sum (ITG_{1_Low}+ITG_{1_Middle}+ITG_{1_High}) to (ITG_{n_Low}+ITG_{n_Middle}+ITG_{n_High}) of the n integral values which are respectively associated with the n classes Cls₁ to Clsₙ.

Thereafter, the calculation unit 213 generates a calculation result CAL_RLSᵤₙᵢ[IDᵤₙᵢ/CALᵤₙᵢ] corresponding the calculation data CALᵤₙᵢ to the identification information IDᵤₙᵢ. Then, the calculation unit 213 outputs the calculation result CAL_RLSᵤₙᵢ=[IDᵤₙᵢ/CALᵤₙᵢ] and a signal S_u indicating that the calculation data is one to the creation unit 215.

The calculation unit 213 also receives the P pieces of analysis data ALY_D₁=[t₁/ID₁/ALY_Na₁/ALY_Kd₁/MRS_Low_1={Low_1:(I_{Low}-V_{Low})₁}/MRS_Middle_1={Middle_1: (I_{Middle}-V_{Middle})₁}/MRS_High_1={High_1:(I_{High}-V_{High})₁}] to ALY_D_{P}=[t_{P}/ID_{P}/ALY_Na_{P}/ALY_Kd_{P}/MRS_Low_P={Low_P:(I_{Low}-V_{Low})}/MRS_Middle_P={Middle_P: (I_{Middle}-V_{Middle})_{P}}/{MRS_High_P=High_P:(I_{High}-V_{High})_{P}}] from control unit 212.

Then, the calculation unit 213 performs the following processing in the same manner as when the analysis data ALY_Dᵤₙᵢ=[tᵤₙᵢ/IDᵤₙᵢ/ALY_Naᵤₙᵢ/ALY_Kdᵤₙᵢ/MRS_Low_uni={Low_uni: (I_{Low}-V_{Low})ᵤₙᵢ}/MRS_Middle_uni={Middle_uni: (I_{Middle}-V_{Middle})ᵤₙᵢ}/MRS_High_uni={High_uni: (I_{High}-V_{High})ᵤₙᵢ}] is received from the control unit 212. Based on the current-potential characteristics {(I_{Low}-V_{Low})ₚ, (I_{Middle}-V_{Middle})ₚ, (I_{High}-V_{High})ₚ} (p is any one of 1 to P) of the analysis data ALY_Dₚ=[tₚ/IDₚ/ALY_Naₚ/ALY_Kdₚ/MRS_Low_p={Low_p: (I_{Low}-V_{Low})ₚ}/MRS_Middle_p={Middle_p: (I_{Middle}-V_{Middle})ₚ}/{MRS_High_p=High_p: (I_{High}-V_{High})ₚ}], the calculation unit 213 executes for all of P pieces of analysis data ALY_D₁ to ALY_D_{P} creating the calculation data CALₚ that associates n classes Cls₁ to Clsₙ, the n integral values ITG_{1_Low} to ITG_{n_Low}, ITG_{1_Middle} to ITG_{n_Middle}, and ITG_{1_High} to ITG_{n_High}, and the sum of the n integral values (ITG_{1_Low}+ITG_{1_Middle}+ITG_{1_High}) to (ITG_{n_Low}+ITG_{n_Middle}+ITG_{n_High}) to create P calculation data CAL₁ to CAL_{P}.

Then, the calculation unit 213 outputs P calculation results CAL_RLS₁=[ID₁/CAL₁] to CAL_RLS_{P}=[ID_{P}/CAL_{P}] in which the P pieces of identification information ID₁ to ID_{P} correspond to the P pieces of calculation data CAL₁ to CAL_{P}, respectively, to the judgment unit 214 and the creation unit 215.

In this way, when the calculation unit 213 has created one calculation data CALᵤₙᵢ, the calculation unit 213 outputs the calculation result CAL_RLSᵤₙᵢ=[IDᵤₙᵢ/CALᵤₙᵢ] to only the creation unit 215, and when the calculation unit 213 has created P calculation results CAL_RLS₁=[ID₁/CAL₁] to CAL_RLS_{P}=[ID_{P}/CAL_{P}] (i.e., multiple calculation results), the calculation unit 213 outputs P calculation results CAL_RLS₁ to CAL_RLS_{P} (i.e., multiple calculation results) to the judgment unit 214 and the creation unit 215.

As described above, the calculation data CALᵤₙᵢ is configured by associating n classes Cls₁ to Clsₙ with n integral values ITG_{1_Low} to ITG_{n_Low}, ITG_{1_Middle} to ITG_{n_Middle}, and ITG_{1_High} to ITG_{n_High}, and the sum of the n integral values (ITG_{1_Low}+ITG_{1_Middle}+ITG_{1_High}) to (ITG_{n_Low}+ITG_{n_Middle}+ITG_{n_High}), but since each of the n classes Cls₁ to Clsₙ is made up of a predetermined potential section V_ITV, the calculation data CALᵤₙᵢ is configured by associating n predetermined potential sections V_ITV₁ to V_ITVₙ with n integral values ITG_{1_Low} to ITG_{n_Low}, ITG_{1_Middle} to ITG_{n_Middle}, and ITG_{1_High} to ITG_{n_High}, and the sum of the n integral values (ITG_{1_Low}+ITG_{1_Middle}+ITG_{1_High}) to (ITG_{n_Low}+ITG_{n_Middle}+ITG_{n_High}). The same is true for each of the P pieces of calculation data CAL₁ to CAL_{P}.

The judgment unit 214 receives P calculation results CAL_RLS₁ to CAL_RLS_{P} (that is, a plurality of calculation results) from the calculation unit 213. Then, the judgment unit 214 detects the P pieces of calculation data CAL₁ to CAL_{P} contained in the P pieces of calculation results CAL_RLS₁ to CAL_RLS_{P}.

Then, the judgment unit 214 detects two calculation data CALᵢ, CALⱼ(i≠j) of _{P}C₂ sets based on the P pieces of calculation data CAL₁ to CAL_{P}. Here, _{P}C₂ is the number of combinations of two different pieces of calculation data CALᵢ, CALⱼ(i≠j) when two different pieces of calculation data CALᵢ, CALⱼ(i≠j) are extracted from the P pieces of calculation data CAL₁ to CAL_{P}.

Thereafter, the judgment unit 214 calculates differences between the multiple integral values included in the calculation data CALᵢ and the multiple integral values included in the calculation data CALⱼ for each class Cls based on two calculation data CALᵢ and CALⱼ(i≠j), and calculates the standard deviation of the calculated differences for all of the two pieces of calculation data CALᵢ, CALⱼ(i≠j) of _{P}C₂ sets.

The judgment unit 214 calculates the differences in each class Cls between the multiple integral values included in the calculation data CALᵢ and the multiple integral values included in the calculation data CALⱼ by the following method.

The judgment unit 214 calculates a difference DFₖ between the integral value ITG_{k_i} and the integral value ITG_{k_j} in one class Clsₖ using the following equation, based on the multiple integral values ITG_{1_i} to ITG_{n_i} included in the calculation data CALᵢ and the multiple integral values ITG_{1_j} to ITG_{n_j} included in the calculation data CALⱼ.

$$[ Equation 1 ]$$

$${DF}_{k} = [ \frac{( \vert {ITG}_{k \_ i} \vert - \vert {ITG}_{k \_ j} \vert )}{( {ITG}_{k \_ i} + {ITG}_{{k}_{-} j} ) / 2} ] \times 100 \qquad ( 1 )$$

The unit of the difference DFₖ calculated by the equation (1) is “%”.

Then, the judgment unit 214 executes the calculation of the difference DFₖ of the integral value in one class Clsₖ using the equation (1) for all of the n classes Cls₁ to Clsₙ to calculate n differences DF₁ to DFₙ.

Then, the judgment unit 214 calculates the standard deviation σ_{DF} of the n differences DF₁ to DFₙ.

Then, the judgment unit 214 judges that the two pieces of calculation data CALᵢ and CALⱼ(i≠j) are different when the standard deviation GDF of the differences is greater than a threshold σₜₕ (=6%), and the judgment unit 214 judges that the two pieces of calculation data CALᵢ and CALⱼ(i≠j) are not different when the standard deviation GDF of the differences is equal or smaller than the threshold σₜₕ (=6%). The judgment unit 214 holds the threshold value σₜₕ (=6%) in advance.

The judgment unit 214 executes the above-mentioned method to judge whether or not the two pieces of calculation data CALᵢ, CALⱼ(i≠j) differ for all of the two pieces of calculation data CALᵢ, CALⱼ(i≠j) in the _{P}C₂ set, and judges whether or not the two pieces of calculation data CALᵢ, CALⱼ (i≠j) in each set of the two pieces of calculation data CALᵢ, CALⱼ(i≠j) in the _{P}C₂ set differ.

In addition, judging that the two pieces of calculation data CALᵢ, CALⱼ(i≠j) are not different corresponds to judging that the two pieces of calculation data CALᵢ, CALⱼ(i≠j) cannot be distinguished from each other, and judging that two pieces of calculation data CALᵢ, CALⱼ(i≠j) are different corresponds to judging that the two pieces of calculation data CALᵢ, CALⱼ(i≠j) can be distinguished from each other.

The judgment unit 214 judges whether or not the two pieces of calculation data CALᵢ, CALⱼ(i≠j) of each of the two sets of _{P}C₂ are different using the method described above, and creates the judgment results shown in Table 1. Then, the judgment unit 214 outputs the judgment result shown in Table 1 to the creation unit 215.

TABLE 1

| | CAL₁ | CAL₂ | CAL₃ | . . . | CAL_{P} |
|---|---|---|---|---|---|
| CAL₁ | | ∘ | ∘ | . . . | ∘ |
| | | or | or | | or |
| | | x | x | | x |
| CAL₂ | ∘ | | ∘ | . . . | ∘ |
| | or | | or | | or |
| | x | | x | | x |
| CAL₃ | ∘ | ∘ | | . . . | ∘ |
| | or | or | | | or |
| | x | x | | | x |
| . | . | . | . | | ∘ |
| . | . | . | . | | or |
| . | . | . | . | | x |
| CAL_{P} | ∘ | ∘ | ∘ | ∘ | |
| | or | or | or | or | |
| | x | x | x | x | |

∘: indicates that the two calculation data CALᵢ and CALᵢ are different.
x: indicates that the two calculation data CALᵢ and CALᵢ are not different.

Judging that the P pieces of calculation data CAL₁ to CAL_{P} are not different from one another corresponds to judging that the P pieces of calculation data CAL₁ to CAL_{P} cannot be distinguished from one another, and judging that the P pieces of calculation data CAL₁ to CAL_{P} are different from one another corresponds to judging that the P pieces of calculation data CAL₁ to CAL_{P} can be distinguished from one another.

Upon receiving one calculation result CAL_RLSᵤₙᵢ and a signal S_u indicating that there is one calculation data from the calculation unit 213, the creation unit 215 creates a curve CURᵤₙᵢ based on the calculation data CALᵤₙᵢ by a method described later.

In addition, when the creation unit 215 receives P calculation results CAL_RLS₁ to CAL_RLS_{P} (i.e., multiple calculation results) from the calculation unit 213 and a judgment result JDGR (judgment result shown in Table 1) indicating whether the P calculation data CAL₁ to CAL_{P} are different or not from the judgment unit 214, the creation unit 215 creates P curves CUR₁ to CUR_{P} based on the P calculation data CAL₁ to CAL_{P} using a method described below.

When the creation unit 215 created one curve CURᵤₙᵢ, the creation unit 215 adds the curve CURᵤₙᵢ to the calculation result CAL_RLSᵤₙᵢ to create an analysis result ALY_RLSᵤₙᵢ=[IDᵤₙᵢ/CALᵤₙᵢ/CURᵤₙᵢ] and outputs the created analysis result ALY_RLSᵤₙᵢ=[IDᵤₙᵢ/CALᵤₙᵢ/CURᵤₙᵢ] to the control unit 212.

On the other hand, when the creation unit 215 created P curves CUR₁ to CUR_{P}, the creation unit 215 adds the P curves CUR₁ to CUR_{P} to the P calculation results CAL_RLS₁ to CAL_RLS_{P}, respectively, to create P analysis results ALY_RLS₁=[ID₁/CAL₁/CUR₁] to ALY_RLSₚ=[ID_{P}/CAL_{P}/CUR_{P}], and outputs the P analysis results ALY_RLS₁ to ALY_RLSₚ and the judgment result JDGR (the judgment result shown in Table 1) to the control unit 212.

The calculation unit 216 receives the index data IDXᵤₙᵢ from the control unit 212. Then, the calculation unit 216 detects the calculation data CALᵤₙᵢ from the index data IDXᵤₙᵢ, and calculates the Body index (+)ᵤₙᵢ, Body index (all)ᵤₙᵢ, Body index (−)_thᵤₙᵢ, the sum of the integral values H(+)_sumᵤₙᵢ, and the sum of the integral values H(−)_sumuni based on the detected calculation data CALᵤₙᵢ using a method described below.

Then, the calculation unit 216 creates index data INDX_Dᵤₙᵢ=[ALY_Naᵤₙᵢ/Body index (+)ᵤₙᵢ/Body index (all)ᵤₙᵢ/Body index (−)_thᵤₙᵢ/H(+)_sumᵤₙᵢ/H(−)_sumᵤₙᵢ] in which the name ALY_Naᵤₙᵢ of the analyte, Body index (+)ᵤₙᵢ, Body index (all)ᵤₙᵢ, Body index (−)_thᵤₙᵢ, the sum H(+)_sumuni of the integral values, and the sum H(−)_sumᵤₙᵢ of the integral values are corresponded to each other, and the calculation unit 216 outputs the created index data INDX_Dᵤₙᵢ=[ALY_Naᵤₙᵢ/Body index (+)ᵤₙᵢ/Body index (all)ᵤₙᵢ/Body index (−)_thᵤₙᵢ/H(+)_sumᵤₙᵢ/H(−)_sumᵤₙᵢ] to the taste diagnostic unit 217.

The calculation unit 216 also receives P index data IDX₁ to IDXₚ from the control unit 212. Then, the calculation unit 216 detects the P pieces of calculation data CAL₁ to CAL_{P} from the P pieces of index data IDX₁ to IDX_{P}, respectively.

Then, the calculation unit 216 calculates Body index (+)ₚ Body index (all)ₚ, Body index (−)_thₚ, the sum H(+)_sumₚ of the integral values and the sum H(−)_sumₚ (p=1 to P) of the integral values based on the calculation data CALₚ using a method described below.

The calculation unit 216 then executes, for all of p=1 to P, creating index data INX_Dₚ[ALY_Naₚ/Body index (+)ₚ/Body index (all)ₚ/Body index (−)_thₚ/H(+)_sumₚ/H(−)_sumₚ] in which the name ALY_Naₚ of the analyte, Body index (+)ₚ, Body index (all)ₚ, Body index (−)_thₚ, the sum H(+)_sumₚ of the integral values, and the sum H(−)_sumₚ of the integral values are corresponded to each other and the calculation unit 216 creates P pieces of index data INDX_D₁=[ALY_Na₁/Body index (+)₁/Body index (all)₁/Body index (−)_th₁/H(+)_sum₁/H(−)_sum₁] to INDX_Dₚ=[ALY_Naₚ/Body index (+)_{P}/Body index (all)_{P}/Body index (−)_thₚ/H(+)_sump/H(−)_sumₚ].

Then, the calculation unit 216 outputs P pieces of index data INDX_D₁=[ALY_Na₁/Body index (+)₁/Body index (all)₁/Body index (−)_th₁/H(+)_sum₁/H(−)_sum₁] to INDX_Dₚ=[ALY_Naₚ/Body index (+)_{P}/Body index (all)ₚ/Body index (−)_thₚ/H(+)_sumₚ/H(−)_sumₚ] to the taste diagnostic unit 217.

The taste diagnostic unit 217 receives index data INDX_Dᵤₙᵢ=[ALY_Naᵤₙᵢ/Body index (+)ᵤₙᵢ/Body index (all)ᵤₙᵢ/Body index (−)_thᵤₙᵢ/H(+)_sumᵤₙᵢ/H(−)_sumᵤₙᵢ] from the calculation unit 216.

The taste diagnostic unit 217 detects name ALY_Naᵤₙᵢ of the analyte, Body index (+)ᵤₙᵢ, Body index (all)ᵤₙᵢ, Body index (−)_thᵤₙᵢ, sum H(+)_sumᵤₙᵢ of integral value, and sum H(−)_sumᵤₙᵢ of integral value from the index data INDX_Dᵤₙᵢ.

Then, the taste diagnostic unit 217 diagnoses that the “astringency” of the analyte having the name ALY_Naᵤₙᵢ is the index value ASTGᵤₙᵢ by a method described later based on the Body index (+)ᵤₙᵢ.

Furthermore, the taste diagnostic unit 217 diagnoses that the “aftertaste” of the analyte having the name ALY_Naᵤₙᵢ is an index value LNGSᵤₙᵢ by a method described later based on the Body index (all)ᵤₙᵢ.

Furthermore, the taste diagnostic unit 217 diagnoses that the “sweetness” of the analyte having the name ALY_Naᵤₙᵢ is the index value SWTᵤₙᵢ by a method described later based on the sum H(+)_sumᵤₙᵢ of the integral values.

Furthermore, the taste diagnostic unit 217 diagnoses that the “aroma” of the analyte having the name ALY_Naᵤₙᵢ is the index value SCTᵤₙᵢ by a method described later based on the sum H(−)_sumᵤₙᵢ of the integral values.

Furthermore, the taste diagnostic unit 217 diagnoses that the “bitterness” of the analyte having the name ALY_Naᵤₙᵢ is the index value BITᵤₙᵢ by a method described later based on the index value ASTGᵤₙᵢ for “astringency” and the index value SWTᵤₙᵢ for “sweetness”.

Then, the taste diagnostic unit 217 creates a taste diagnosis result JDRᵤₙᵢ=[ALY_Naᵤₙᵢ/ASTGᵤₙᵢ/LNGSᵤₙᵢ/SWTᵤₙᵢ/SCTᵤₙᵢ/BITᵤₙᵢ] in which the name ALY_Naᵤₙᵢ of the analyte, the index value ASTGᵤₙᵢ for “astringency”, the index value LNGSᵤₙᵢ for “aftertaste”, the index value SWTᵤₙᵢ for “sweetness”, the index value SCTᵤₙᵢ for “aroma”, and the index value BITᵤₙᵢ for “bitterness” are associated with each other and outputs the created taste diagnosis result JDR₁=[ALY_Naᵤₙᵢ/ASTGᵤₙᵢ/LNGSᵤₙᵢ/SWTᵤₙᵢ/SCTᵤₙᵢ/BITᵤₙᵢ] to the control unit 212 and the display unit 218.

In addition, when the name ALY_Naᵤₙᵢ of the analyte is “grapes”, the taste diagnostic unit 217 diagnoses that the “astringency” of the analyte having the name ALY_Naᵤₙᵢ (=grapes) is the index value ASTG_{th_uni} by a method described below based on the Body index (−)_thᵤₙᵢ.

Then, the taste diagnostic unit 217 creates a diagnosis result JDR_{th_uni}=[ALY_Naᵤₙᵢ (=grapes)/ASTG_{th_uni}] in which the name ALY_Naᵤₙᵢ (=grapes) of the analyte is associated with the index value ASTG_{th_uni} of “astringency”, and outputs the created diagnosis result JDR_{th_uni}=[ALY_Naᵤₙᵢ (=grape)/ASTG_{th_uni}] to the control unit 212 and the display unit 218.

On the other hand, when the taste diagnostic unit 217 receives P pieces of index data INDX_D₁=[ALY_Na₁/Body index (+)₁/Body index (all)₁/Body index (−)_th₁/H(+)_sum₁/H(−)_sum₁] to INDX_D_{P}=[ALY_Naₚ/Body index (+)_{P}/Body index (all)_{P}/Body index (−)_th_{P}/H(+)_sumₚ/H(−)_sumₚ] from the calculation unit 216, the taste diagnostic unit 217 detects the name ALY_Naₚ of the analyte, Body index (+)ₚ, Body index (all)ₚ, Body index (−)_thₚ, the sum H(+)_sumₚ of the integral values and the sum H(−)_sumₚ of the integral values from the index data INDX_Dₚ=[ALY_Naₚ/Body index (+)ₚ/Body index (all)ₚ/Body index (−)_thₚ/H(+)_sumₚ/H(−)_sumₚ].

Then, the taste diagnostic unit 217 diagnoses that the “astringency” of the analyte having the name ALY_Naₚ of the analyte is the index value ASTGₚ by a method described below based on the Body index (+)ₚ.

Furthermore, the taste diagnostic unit 217 diagnoses that the “aftertaste” of the analyte having the name ALY_Naₚ of the analyte is the index value LNGSₚ by a method described below based on the Body index (all)ₚ.

Furthermore, the taste diagnostic unit 217 diagnoses that the “sweetness” of the analyte having the name ALY_Naₚ of the analyte is the index value SWTₚ by a method described below based on the sum H(+)_sumₚ of the integral values.

Furthermore, the taste diagnostic unit 217 diagnoses that the “aroma” of the analyte having the name ALY_Naₚ of the analyte is the index value SCTₚ by a method described below based on the sum H(−)_sumₚ of the integral values.

Furthermore, the taste diagnostic unit 217 diagnoses the “bitterness” of the analyte having the name ALY_Naₚ of the analyte be an index value BITₚ by a method described below based on the index value ASTRₚ of “astringency” and the index value SWTₚ of “sweetness”.

Then, the taste diagnostic unit 217 creates a taste diagnosis result JDR_{P} [ALY_Naₚ/ASTGₚ/LNGSₚ/SWTₚ/SCTₚ/BITₚ] that corresponds to the name ALY_Naₚ of the analyte, the index value ASTGₚ for “astringency”, the index value LNGSₚ for “aftertaste”, the index value SWTₚ for “sweetness”, the index value SCTₚ for “aroma”, and the index value BITₚ for “bitterness”.

The taste diagnostic unit 217 executes the above-mentioned operation for all of p=1 to P to generate P taste diagnosis results JDR₁=[ALY_Na₁/ASTG₁/LNGS₁/SWT₁/SCT₁/BIT₁] to JDR_{P}=[ALY_Naₚ/ASTGₚ/LNGS_{P}/SWTₚ/SCTₚ/BITₚ], and outputs the created P taste diagnosis results JDR₁=[ALY_Na₁/ASTG₁/LNGS₁/SWT₁/SCT₁/BIT₁] to JDR_{P}=[ALY_Naₚ/ASTGₚ/LNGSₚ/SWTₚ/SCTₚ/BITₚ] to the control unit 212 and the display unit 218.

In addition, when the name ALY_Naₚ of the analyte is “grapes”, the taste diagnostic unit 217 diagnoses that the “astringency” of the analyte having the name ALY_Naₚ (=grapes) is the index value ASTG_{th_p} based on the Body index (−)_thₚ by a method described below.

Then, the taste diagnostic unit 217 executes, for all of p=1 to P, creating a diagnosis result JDR_{th_p}=[ALY_Naₚ(=grapes)/ASTG_{th_p}] in which the name ALY_Naₚ (=grapes) of the analyte is associated with the index value ASTG_{th_p} of “astringency”, and creates P taste diagnosis results JDR_{th_1}=[ALY_Na₁(=grapes)/ASTG_{th_1}] to JDR_{th_P}=[ALY_Naₚ (=grapes)/ASTG_{th_P}], and outputs the P taste diagnosis results JDR_{th_1}=[ALY_Na₁ (=grapes)/ASTG_{th_1}] to JDR_{th_p}=[ALY_Naₚ (=grapes)/ASTG_{th_P}] to the control unit 212 and display unit 218.

When the display unit 218 receives the diagnosis result JDRᵤₙᵢ=[ALY_Naᵤₙᵢ/ASTGᵤₙᵢ/LNGSᵤₙᵢ/SWTᵤₙᵢ/SCTᵤₙᵢ/BITᵤₙᵢ] from the taste diagnostic unit 217, the display unit 218 displays the received diagnosis result JDRᵤₙᵢ[ALY_Naᵤₙᵢ/ASTGᵤₙᵢ/LNGSᵤₙᵢ/SWTᵤₙᵢ/SCTᵤₙᵢ/BITᵤₙᵢ].

Furthermore, when the display unit 218 receives the diagnosis result JDR_{th_uni}=[ALY_Naᵤₙᵢ (=grapes)/ASTG_{th_uni}] from the taste diagnostic unit 217, the display unit 218 displays the received diagnosis result JDR_{th_uni}=[ALY_Naᵤₙᵢ (=grapes)/ASTG_{th_uni}].

Furthermore, when the display unit 218 receives P diagnosis results JDR₁=[ALY_Na₁/ASTG₁/LNGS₁/SWT₁/SCT₁/BIT₁] to JDR_{P}=[ALY_Naₚ/ASTG_{P}/LNGS_{P}/SWT_{P}/SCT_{P}/BIT_{P}] from the taste diagnostic unit 217, the display unit 218 displays the received P diagnosis results JDR₁=[ALY_Na₁/ASTG₁/LNGS₁/SWT₁/SCT₁/BIT₁] to JDR_{P}=[ALY_Na_{P}/ASTG_{P}/LNGS_{P}/SWT_{P}/SCT_{P}/BIT_{P}].

Furthermore, when the display unit 218 receives P taste diagnosis results JDR_{th_1}=[ALY_Na₁ (grapes)/ASTG_{th_1}] to JDR_{th_P}=[ALY_Naₚ (=grapes)/ASTG_{th_P}] from the taste diagnostic unit 217, the display unit 218 displays the received P taste diagnosis results JDR_{th_1}=[ALY_Na₁(=grapes)/ASTG_{th_1}] to JDR_{th_P}=[ALY_Naₚ (=grapes)/ASTG_{th_P}].

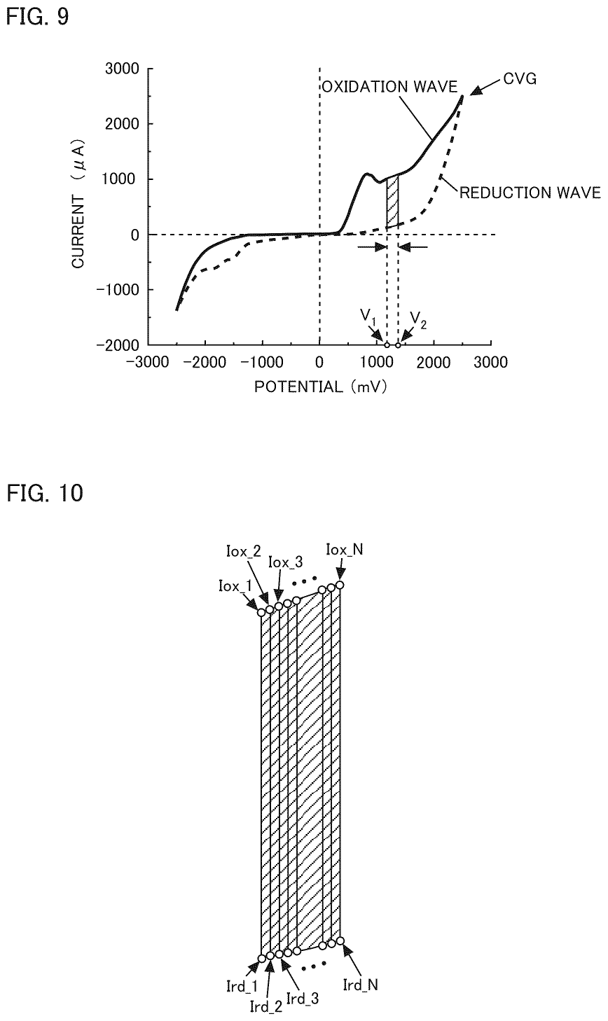

The reception unit 219 receives requests RQTᵤₙᵢ or RQT_{q} from staff at restaurants such as Japanese restaurants, Chinese restaurants, and Western restaurants, master brewers at sake breweries, and staff at liquor stores, and outputs the received requests RQTᵤₙᵢ or RQT_{q} to the control unit 212.

[Calculation of integral value] FIG. 9 and FIG. 10 are first and second conceptual diagrams for explaining a method for calculating an integral value, respectively.

Referring to FIG. 9, the cyclic voltammogram CVG is measured, for example, by scanning the potential V from 0 V to +2500 mV at a predetermined scanning speed, then scanning the potential V from +2500 mV to 0 V at a predetermined scanning speed, further scanning the potential V from 0 V to −2500 mV at a predetermined scanning speed, and further scanning the potential V from −2500 mV to 0 V at a predetermined scanning speed.

As a result, in the cyclic voltammogram CVG, the solid line portion represents the current value I in when the potential V is scanned in the positive direction, and the dotted line portion represents the current value I in when the potential V is scanned in the negative direction.

Therefore, in the cyclic voltammogram CVG, the solid line portion represents the current value I of the oxidation wave, and the dotted line portion represents the current value I of the reduction wave.

The predetermined scanning speed is, for example, any one of 0.3 V/sec, 0.5 V/sec, and 0.6 V/sec.

When calculating the integral value ITG of the cyclic voltammogram CVG, the predetermined potential section V_ITV is, for example, [0 to 100 mV], [101 to 200 mV], [201 to 300 mV], . . . , [2301 to 2400 mV], [2401 to 2500 mV], [0 to −100 mV], [−101 to −200 mV], . . . , [−2301 to −2400 mV], [−2401 to −2500 mV].

FIG. 10 shows an enlarged view of a portion at a predetermined potential section [V₁ to V₂] of the cyclic voltammogram CVG shown in FIG. 9.

Referring to FIG. 10, when the calculation unit 213 calculates the integral value ITG in a predetermined potential section [V₁ to V₂], the calculation unit 213 detects the current value Iox_1 of the oxidation wave and the current value Ird_1 of the reduction wave at potential V₁, and calculates the difference (Iox_1−Ird_1) between the current value Iox_1 and the current value Ird_1 to calculate the intensity (Iox_1−Ird_1) of the cyclic voltammogram CVG at potential V₁.

Then, the calculation unit 213 detects current value Iox_2 of the oxidation wave and current value Ird_2 of the reduction wave at a potential V₁+1 obtained by adding a unit potential (=1 mV) to the potential V₁, and calculates the difference (=Iox_2−Ird_2) between the current value Iox_2 and the current value Ird_2 to calculate the intensity (Iox_2−Ird_2) of the cyclic voltammogram CVG at the potential V₁+1.

Furthermore, the calculation unit 213 detects the oxidation wave current value Iox_3 and the reduction wave current value Ird_3 at a potential V₁+2 obtained by adding a unit potential (=1 mV) to the potential V₁+1, and calculates the difference (Iox_3−Ird_3) between the current value Iox_3 and the current value Ird_3 to calculate the intensity (Iox_3−Ird_3) of the cyclic voltammogram CVG at the potential V₁+2.

Similarly, the calculation unit 213 detects the oxidation wave current value Iox_N and the reduction wave current value Ird_N at potential V₂ which is obtained by adding a unit potential (=1 mV) to potential V₂-1, and calculates the difference (Iox_N−Ird_N) between the current value Iox_N and the current value Ird_N to calculate the intensity (Iox_N−Ird_N) of the cyclic voltammogram CVG at potential V₂.

Then, the calculation unit 213 calculates the integral value ITG in the predetermined potential section [V₁ to V₂] by the following formula.

$$[ Equation 2 ]$$

$$ITG ( {V}_{1} - {V}_{2} ) = ( Iox\_ 1 - Ird\_ 1 ) + ( Iox\_ 2 - Ird\_ 2 ) + ⋯ + ( Iox\_N - Ird\_N ) \qquad ( 2 )$$

That is, the calculation unit 213 calculates multiple intensities ((Iox_1−Ird_1), (Iox_2−Ird_2), . . . , (Iox_N−Ird_N)) of the cyclic voltammogram CVG for each unit potential (=1 mV) in the predetermined potential section [V₁ to V₂], and adds up the multiple calculated intensities ((Iox_1−Ird_1), (Iox_2−Ird_2), . . . , (Iox_N−Ird_N)) to calculate an integral value ITG of the cyclic voltammogram CVG in the predetermined potential section [V₁ to V₂].

Here, in the predetermined potential section [V₁ to V₂], calculating the difference (lox 1−Ird_1) at the potential V₁, calculating the difference (Iox_2−Ird_2) at the potential V₁+1, calculating the difference (Iox_3−Ird_3) at the potential V₁+2, . . . , and calculating the difference (Iox_N−Ird_N) at the potential V₂ corresponds to calculating a plurality of intensities in one specified potential section by executing for all of unit potential in one specified potential section calculating the intensity of the cyclic voltammogram at one unit potential by performing a subtraction process of subtracting the current value of the reduction wave from the current value of the oxidation wave of a cyclic voltammogram at one unit potential in one specified potential section for all unit potentials in one specified potential section.

Calculating the integral value ITG in a predetermined potential section [V₁ to V₂] using equation (2) is equivalent to calculating the sum of the calculated intensities as the area of a cyclic voltammogram in one predetermined potential section.

Furthermore, calculating the difference (Iox_1−Ird_1) at potential V₁, calculating the difference (Iox_2−Ird_2) at potential V₁+1, calculating the difference (Iox_3−Ird_3) at potential V₁+2, . . . , calculating the difference (Iox_N−Ird_N) at potential V₂ each corresponds to calculating the intensity of a cyclic voltammogram at one unit potential by subtracting the current value of the reduction wave from the current value of the oxidation wave of a cyclic voltammogram at one unit potential in one specified potential range.

In addition, the calculation unit 213 may calculate a sum SUM_Iox of the current values Iox_1 to Iox_N of the N oxidation waves and a sum SUM_Ird of the current values Ird_1 to Ird_N of the N reduction waves in a predetermined potential section [V₁ to V₂], and subtract the sum SUM_Ird from the sum SUM_Iox to calculate an integral value ITG of the cyclic voltammogram CVG in the predetermined potential range [V₁ to V₂].

The calculation unit 213 calculates a plurality of integral values ITG₁, ITG₂, ITG₃, . . . , ITG₂₄, ITG₂₅, ITG₂₆, ITG₂₇, . . . , ITG₄₉, and ITG₅₀ in a plurality of predetermined potential sections [0 to 100 mV], [101 to 200 mV], [201 to 300 mV], . . . , [2301 to 2400 mV], [2401 to 2500 mV], [0 to −100 mV], [−101 to −200 mV], . . . , [−2301 to −2400 mV], and [−2401 to −2500 mV] by the method of calculating the integral value ITG of the cyclic voltammogram CVG in the predetermined potential section [V₁ to V₂] described above.

By calculating multiple integral values ITG₁ to ITG₅₀ using the methods shown in FIGS. 9 and 10 described above, it is possible to correct the characteristic variations between the sensors used to measure the cyclic voltammogram CVG, and clearly show the signal intensity differences between the solutions of the analyte.

In the above-mentioned formula (2), (Iox_1−Ird_1), (Iox_2−Ird_2), . . . , (Iox_N−Ird_N) each represent the current value in the oxidation reaction and reduction reaction between the electrode (working electrode 112) and the analyte at each unit potential.

Furthermore, the sum (=integral value) of the subtraction results (lox-Ird) in one specified potential section represents the total number of electrons in the oxidation reaction and reduction reaction between the electrode (working electrode) and the analyte in one specified potential section.

Furthermore, a curve CUR showing the class dependence of multiple integral values ITG₁, ITG₂, ITG₃, . . . , ITG₂₄, ITG₂₅, ITG₂₆, ITG₂₇, . . . , ITG₄₉, and ITG₅₀ represents the dependence of the total number of electrons (=integral value) in the oxidation reaction and reduction reaction between the electrode (working electrode) and the analyte on a specified potential section.

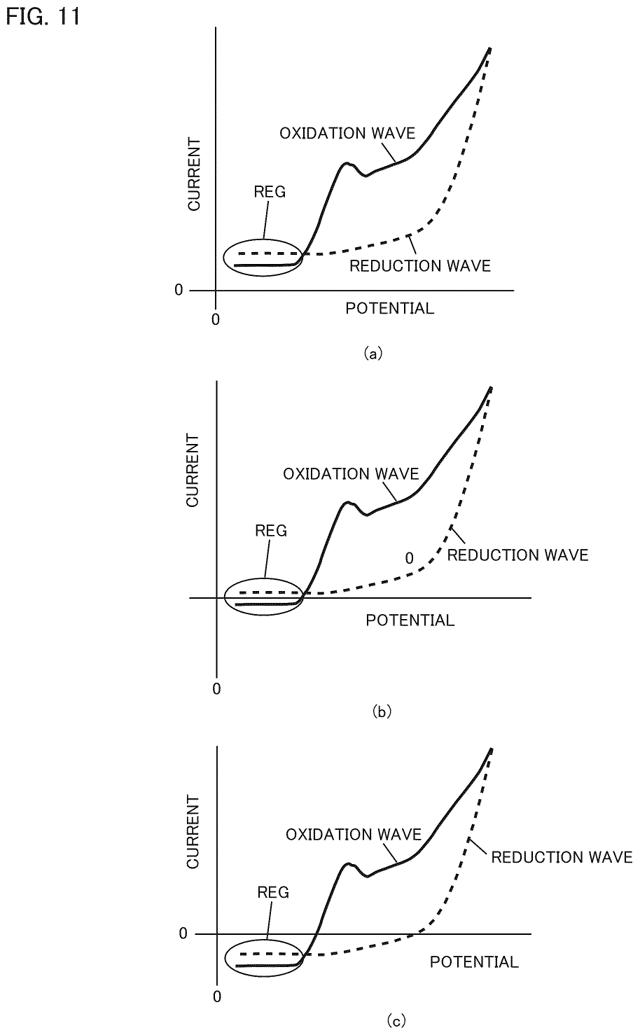

FIG. 11 is a diagram showing a part of a cyclic voltammogram. FIG. 11 shows a cyclic voltammogram with a region REG where the oxidation wave lies below the reduction wave. FIG. 11(a) shows the case where region REG is located in the positive current region, FIG. 11(b) shows the case where, in region REG, the reduction wave is located in the positive current region and the oxidation wave is located in the negative current region, and FIG. 11(c) shows the case where region REG is located in the negative current region.

Here, the current values of the oxidation wave at the unit potential of region REG are Iox_1_REG, Iox_2_REG, . . . , Iox_N′_REG (N′ is the total number of unit potentials in region REG), and the current values of the reduction wave at the unit potential of region REG are Ird_1_REG, Ird_2_REG, . . . Ird_N′_REG.

Referring to (a) of FIG. 11, when calculating the integral value of a specified potential section in region REG, the oxidation wave current values Iox_1_REG, Iox_2_REG, . . . , Iox_N′_REG are respectively smaller than the reduction wave current values Ird_1_REG, Ird_2_REG, . . . , Ird_N′_REG, and therefore the integral value ITG of the specified potential section in region REG becomes a negative value.

Referring to (b) of FIG. 11, when calculating the integral value of a specified potential section in region REG, each of the oxidation wave current values Iox_1_REG, Iox_2_REG, . . . , Iox_N′_REG is a negative current value, and each of the reduction wave current values Ird_1_REG, Ird_2_REG, . . . , Ird_N′_REG is a positive current value, so that the integral value ITG of the specified potential section in region REG is a negative value.

Referring to (c) of FIG. 11, when calculating the integral value of a predetermined potential section in the region REG, each of the oxidation wave current values Iox_1_REG, Iox_2_REG, . . . , Iox_N′_REG is negative current values, each of the reduction wave current values Ird_1_REG, Ird_2_REG, . . . , Ird_N′_R is negative current values, and since the absolute values |I_{ox_1_REG}|, I_{ox_2_REG}|, . . . , I_{ox_N′_REG}| of the oxidation wave current values I_{ox_1_REG}, I_{ox_2_REG}, . . . , I_{rd_N′_REG} are greater than the absolute values |I_{rd_1_REG}|, |I_{rd_2_REG}|, . . . , |I_{rd_N′_R}| of the reduction wave current values I_{rd_1_REG}, I_{rd_2_REG}, . . . , I_{rd_N′_R}, respectively, the integral value ITG of a predetermined potential section in the region REG becomes a negative value.

Therefore, in embodiment of the present invention, the integral value in a predetermined potential section of a cyclic voltammogram having a region REG where the oxidation wave is located below the reduction wave becomes a negative value in the region REG.

The calculation unit 213 may calculate a plurality of integral values in a plurality of predetermined potential sections by a method different from the above-mentioned method.

For example, the calculation unit 213 may use the potential V as an explanatory variable and the current I as a target variable to calculate a regression curve RC1 (the curve indicated by the solid line) showing the oxidation wave of the cyclic voltammogram CVG in FIG. 9 and a regression curve RC2 (the curve indicated by the dashed line) showing the reduction wave, calculate an integral value ITG_RC1 of the regression curve RC1 and an integral value ITG_RC2 of the regression curve RC2 in a predetermined potential section [V₁-V₂], and executes for all predetermined potential sections calculating an integral value of the cyclic voltammogram CVG shown in FIG. 9 in the predetermined potential section [V₁-V₂] by subtracting the integral value ITG_RC2 from the integral value ITG_RC1, thereby may calculate a plurality of integral values in a plurality of predetermined potential sections.

The calculation unit 213 may calculate the multiple integral values in the multiple predetermined potential sections by any method as long as the method can calculate the multiple integral values in the multiple predetermined potential sections.

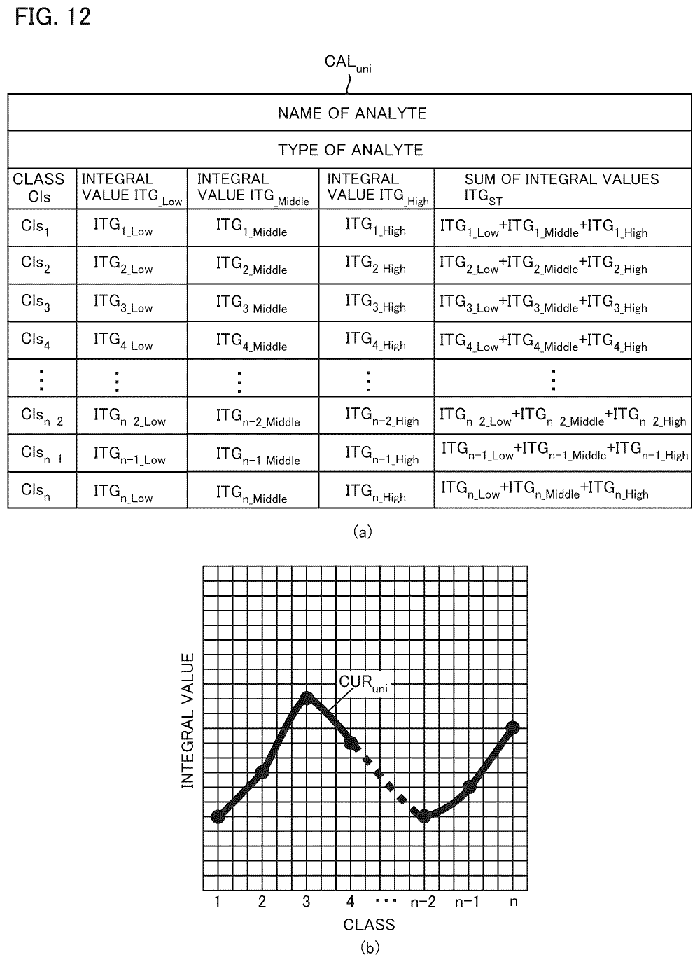

FIG. 12 is a diagram for explaining a method for creating a curve CUR showing the relationship between a plurality of classes Cls and a plurality of integral values ITG.

FIG. 12(a) shows one piece of calculation data CALᵤₙᵢ, and FIG. 12(b) shows a curve CURᵤₙᵢ that represents the relationship between the integral value and the class.

Referring to (a) of FIG. 12, the calculation data CALᵤₙᵢ includes a name ALY_Naᵤₙᵢ of the analyte, a type ALY_Kdᵤₙᵢ of the analyte, a class Cls, integral values ITG__{Low}, ITG__{Middle}, ITG__{High}, and a sum ITG_{ST} of the integral values.

The integral value ITG__{Low} is an integral value calculated by the above-mentioned method based on the cyclic voltammogram CVG measured by changing the potential V at a potential scanning rate V_{r_Low} (e.g., 0.3 V/sec), the integral value ITG__{Middle} is an integral value calculated by the above-mentioned method based on the cyclic voltammogram CVG measured by changing the potential V at a potential scanning rate V_{r_Middle} (e.g., 0.5 V/sec), and the integral value ITG__{High} is an integral value calculated by the above-mentioned method based on the cyclic voltammogram CVG measured by changing the potential V at a potential scanning rate V_{r_High} (e.g., 0.6 V/sec).

The class Cls consists of n classes Cls₁ to Clsₙ, the integral value ITG__{Low} consists of n integral values ITG_{1_Low} to ITG_{n_Low}, the integral value ITG__{Middle} consists of n integral values ITG_{1_Middle} to ITG_{n_Middle}, and the integral value ITG__{High} consists of n integral values ITG_{1_High} to ITG_{n_High}. Here, n represents the total number of predetermined potential sections, when the potential scanning range is [−Vₛ₁ to +Vₛ₂] and one predetermined potential section is V_{PTS}, n=(|−Vₛ₁|+|+Vₛ₂|)/V_{PTS}.

The n integral values ITG_{1_Low} to ITG_{n_Low} are integral values calculated by the above-mentioned method based on the potentials V_{1_Low} to V_{d_Low} and the current values I_{1_Low} to I_{d_Low} shown in FIG. 6, the n integral values ITG_{1_Middle} to ITG_{n_Middle} are integral values calculated by the above-mentioned method based on the potentials V_{1_Middle} to V_{d_Middle} and the current values I_{1_Middle} to I_{d_Middle} shown in FIG. 6, and the n integral values ITG_{1_High} to ITG_{n_High} are integral values calculated by the above-mentioned method based on the potentials V_{1_High} to V_{d_High} and the current values I_{1_High} to I_{d_High} shown in FIG. 6.

The sum of the integral values ITG_{ST} consists of the sum [ITG_{1_Low}+ITG_{1_Middle}+ITG_{1_High}] to [ITG_{n_Low}+ITG_{n_Middle}+ITG_{n_High}] of n integral values.

Here, the sum [ITG_{1_Low}+ITG_{1_Middle}+ITG_{1_High}] to [ITG_{n_Low}+ITG_{n_Middle}+ITG_{n_High}] of n integral values is expressed as “the sum ITG__{SMT1} to ITG__{SMTn} of n integral values”.

The n integral values ITG_{1_Low} to ITG_{n_Low}, the n integral values ITG_{1_Middle} to ITG_{n_Middle}, the n integral values ITG_{1_High} to ITG_{n_High}, and the sums ITG__{SMT1} to ITG__{SMTn} of the n integral values are associated with n classes Cls₁ to Clsₙ, respectively.

When the creation unit 215 receives one piece of calculation data CALᵤₙᵢ and a signal S_u indicating that the calculation data is one from the calculation unit 213, the creation unit 215 judges that the calculation data CAL calculated by the calculation unit 213 is one based on the signal S_u.

Then, the creation unit 215 detects a set of (class Cls₁, sum ITG__{SMT1} of integral values)_that corresponded to each other from the calculation data CALᵤₙᵢ, then detects a set of (class Cls₂, sum ITG__{SMT2} of integral values) from the calculation data CALᵤₙᵢ, and similarly detects a set of (class Clsₙ−1, sum ITG__{SMTn-1} of integral values) from the calculation data CALᵤₙᵢ, and detects a set of (class Clsₙ, sum ITG__{SMTn} of integral values) from the calculation data CALᵤₙᵢ.

Then, the creation unit 215 plots one set (class Cls₁, sum ITG__{SMT1} of integral values), one set (class Cls₂, sum ITG__{SMT2} of integral values), one set (class Cls₃, sum ITG__{SMT3} of integral values), . . . , one set (Cls_{n_2}, sum ITG__{SMTn-2} of integral values), one set (class Clsₙ₋₁, sum ITG__{SMTn-1} of integral values), and one set (class Clsₙ, sum ITG__{SMTn} of integral values) on a graph with the horizontal axis representing classes and the vertical axis representing integral values.

Thereafter, the creation unit 215 creates a curve CURᵤₙᵢ by connecting the n plotted points. In this case, the n plotted points are plotted for each class Cls, so that the creation unit 215 can create a curve CURᵤₙᵢ consisting of a smooth curve by connecting the n plotted points.

The creation unit 215 may plot n points and then create the curve CURᵤₙᵢ by determining a regression curve using the class Cls as an explanatory variable and the sum ITG__{SMT} of the integral values as a response variable.

Then, the creation unit 215 sets the curve CURᵤₙᵢ as [an index curve which is a curve that serves as an index when identifying the analyte].

In addition, the creation unit 215 receives P calculation results CAL_RLS₁ to CAL_RLS_{P} (i.e., multiple calculation results) from the calculation unit 213, and also receives, from the judgment unit 214, a judgment result JDGR (judgment result shown in Table 1) indicating whether the P calculation data CAL₁ to CAL_{P} are or not different.

Then, the creation unit 215 executes, for all P pieces of calculation data CAL₁ to CAL_{P}, creating a curve CURₚ showing the class dependency of the sum ITG__{SMT} of integral values based on one calculation data CALₚ (p is any of 1 to P) using the method described with reference to FIG. 12, thereby creating P curves CUR₁ to CUR_{P} (i.e., multiple curves CUR).

In this case, each of the P pieces of calculation data CAL₁ to CAL_{P} (that is, a plurality of calculation data) has the same configuration as the calculation data CALᵤₙᵢ shown in FIG. 12(a).

The curve CURᵤₙᵢ represents the dependency of [the sum ITG__{SMT1} to ITG__{SMTn} of n integral values] on [n classes Cls₁ to Clsₙ]. Since each of the n classes Cls₁ to Clsₙ consists of a predetermined potential section, the curve CURᵤₙᵢ is a curve that represents the dependency of the integral value on the predetermined potential section. Similarly, each of the P curves CUR₁ to CUR_{P} is also a curve that indicates the dependency of the integral value on a predetermined potential section.

When the creation unit 215 have created one curve CURᵤₙᵢ, the creation unit 215 adds the curve CURᵤₙᵢ to the calculation result CAL_RLSᵤₙᵢ to create an analysis result ALY_RLSᵤₙᵢ[IDᵤₙᵢ/CALᵤₙᵢ/CURᵤₙᵢ] and outputs the created analysis result ALY_RLSᵤₙᵢ=[IDᵤₙᵢ/CALᵤₙᵢ/CURᵤₙᵢ] to the control unit 212.

On the other hand, when the creation unit 215 have created P curves CUR₁ to CUR_{P}, the creation unit 215 adds the P curves CUR₁ to CUR_{P} to the P calculation results CAL_RLS₁ to CAL_RLS_{P}, respectively, to create P analysis results ALY_RLS₁=[ID₁/CAL₁/CUR₁] to ALY_RLS_{P}=[ID_{P}/CAL_{P}/CUR_{P}], and outputs the P analysis results ALY_RLS₁ to ALY_RLS_{P} and the judgment results (the judgment results shown in Table 1) to the control unit 212.

### [Taste Diagnosis] (I) Taste Diagnosis of Shochu

When the calculation unit 216 receives the index data IDXᵤₙᵢ from the control unit 212, the calculation unit 216 detects the calculation data CALᵤₙᵢ (see FIG. 12) from the index data IDXᵤₙᵢ. Then, the calculation unit 216 detects the integral value ITG__{Low} (ITG_{1_Low} to ITG_{n_Low}), the integral value ITG__{Middle} (ITG_{1_Middle} to ITG_{n_Middle}) and the integral value ITG__{High} (ITG_{1_High} to ITG_{n_High}) from the calculation data CALᵤₙᵢ (see FIG. 12).

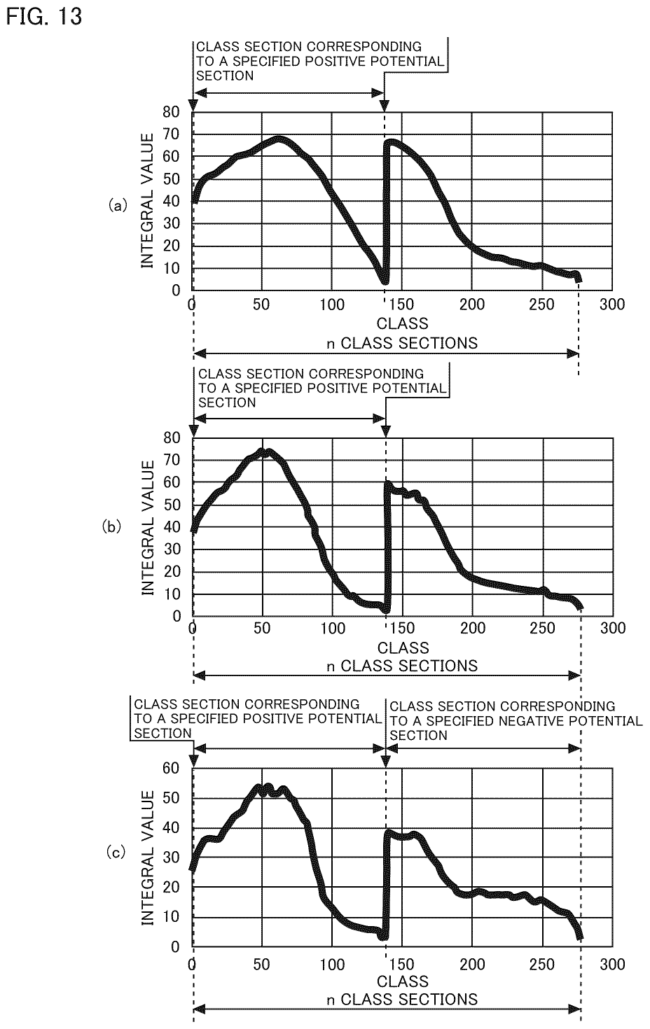

FIG. 13 is a conceptual diagram showing the class dependency of the integral value created based on cyclic voltammograms measured while changing the potential scanning rate to the potential scanning V_{r_Low}, V_{r_Middle}, and V_{r_High}.

FIG. 13(a) is a conceptual diagram showing the dependency of the integral value on the class created based on a cyclic voltammogram measured with the potential scanning rate set to “V_{r_Low}”, FIG. 13(b) is a conceptual diagram showing the dependency of the integral value on the class created based on a cyclic voltammogram measured with the potential scanning rate set to “V_{r_Middle}”, and FIG. 13(c) is a conceptual diagram showing the dependency of the integral value on the class created based on a cyclic voltammogram measured with the potential scanning rate set to “V_{r_High}”.

Based on the integral value ITG Low (ITG_{1_Low} to ITG_{n_Low}), the calculation unit 216 detects x integral values ITG_{1_Low(+)} to ITG_{x_Low(+)} in x (x is an integer satisfying x=INT(n/2)) classes (see FIG. 13(a)) corresponding to a predetermined positive potential section, and calculates a sum SMT__{Low(+)} of the detected x integral values ITG_{1_Low(+)} to ITG_{x_Low(+)}. Here, “INT(n/2)” is the result converting the result of dividing n by 2 into an integer.

In addition, the calculation unit 216 detects x integral values ITG_{1_Middle(+)} to ITG_{x_Middle(+)} in x classes (see (b) of FIG. 13) corresponding to a predetermined positive potential section based on the integral value ITG__{Middle} (ITG_{1_Middle} to ITG_{n_Middle}), and calculates the sum SMT__{Middle(+)} of the detected x integral values ITG_{1_Middle}(+) to ITG_{x_Middle(+)}.

Furthermore, the calculation unit 216 detects x integral values ITG_{1_High(+)} to ITG_{x_High(+)} in x classes (see (c) of FIG. 13) corresponding to a predetermined positive potential range based on the integral value ITG__{High} (ITG_{1_High} to ITG_{n_High}), and calculates the sum SMT__{High(+)} of the detected x integral values ITG_{1_High(+)} to ITG_{x_High(+)}.

Then, the calculation unit 216 calculates the Body index (+) based on the sums SMT__{Low(+)}, SMT__{Middle(+)}, and SMT__{High(+)} according to the following formula:

$$[ Equation 3 ]$$

$$Body index ( + ) = \frac{{SMT}_{\_ Middle ( + )}}{{SMT}_{\_ High ( + )}} + \frac{{SMT}_{\_ Low ( + )}}{{SMT}_{\_ High}} \qquad ( 3 )$$

The Body Index (+) is a factor based on the diffusion coefficient of the components of the analyte when a positive potential is applied to the analyte.

In addition, the calculation unit 216 detects n integral values ITG_{1_Low(all)} to ITG_{n_Low(all)} in n class sections (see (a) of FIG. 13) corresponding to n predetermined potential sections based on the integral value ITG__{Low} (ITG_{1_Low} to ITG_{n_Low}), and calculates the sum SMT__{Low(all)} of the detected n integral values ITG_{1_Low(all)} to ITG_{n_Low(all)}.

Next, the calculation unit 216 detects n integral values ITG_{1_Middle(all)} to ITG_{n_Middle(all)} in n class sections (see (b) of FIG. 13) corresponding to n predetermined potential sections based on the integral value ITG__{Middle} (ITG_{1_Middle} to ITG_{n_Middle}), and calculates the sum SMT__{Middle(all)} of the detected n integral values ITG_{1_Middle(all)} to ITG_{n_Middle(all)}.

Furthermore, the calculation unit 216 detects n integral values ITG_{1_High(all)} to ITG_{n_High(all)} in n class sections (see (c) of FIG. 13) corresponding to n predetermined potential sections based on the integral value ITG__{High} (ITG_{1_High} to ITG_{n_High}), and calculates the sum SMT__{High(all)} of the detected n integral values ITG_{1_High(all)} to ITG_{n_High(all)}.

Then, the calculation unit 216 calculates the Body index (all) based on the sums SMT__{Low(all)}, SMT__{Middle(all)}, and SMT__{High(all)} by the following formula.

$$[ Equation 4 ]$$

$$Body index ( all ) = \frac{{SMT}_{\_ Middle ( all )}}{{SMT}_{\_ High ( all )}} + \frac{{SMT}_{\_ Low ( all )}}{{SMT}_{\_ High ( all )}} \qquad ( 4 )$$

The Body Index (all) is a factor based on the diffusion coefficients of the components of the analyte when positive and negative potentials are applied to the analyte.

Furthermore, the calculation unit 216 sets the sum SMT__{High(+)} used in the calculation of the Body index(+) to H(+)_sum (=SMT__{High(+)}).

Furthermore, based on the integral value ITG__{High} (ITG_{1_High} to ITG_{n_High}), the calculation unit 216 detects x integral values ITG_{1_High(-)} to ITG_{x_High(-)} in x classes (see (c) of FIG. 13) corresponding to a negative predetermined potential section, and calculates the sum SMT__{High(-)} of the detected x integral values ITG_{1_High(-)} to ITG_{x_High(-)} as H(−)_sum.

Then, the calculation unit 216 outputs the Body index (+), the Body index (all), H(+)_sum (=SMT__{High(+)}), and H(−)_sum (=SMT__{High(-)}) to the taste diagnostic unit 217.

The taste diagnostic unit 217 receives the Body index (+), the Body index (all), H(+)_sum (=SMT__{High(+)}), and H(−)_sum (=SMT__{High(-)}) from the calculation unit 216.

### (1) Diagnosis of “Astringency”

When the taste diagnostic unit 217 receives the Body index (+) from the calculation unit 216, the taste diagnostic unit 217 substitutes the Body index (+) into the following formula to calculate the result (=ASTG), and diagnoses the result as the astringency ASTG of the shochu being analyte.

$$[ Equation 5 ]$$

$$ASTG = Body index ( + ) \times {k}_{1} \qquad ( 5 )$$

In the equation (5), the coefficient k₁ is, for example, 1.5.

### (2) Diagnosis of “Aftertaste”

When the taste diagnostic unit 217 receives the Body index (all) from the calculation unit 216, the taste diagnostic unit 217 substitutes the Body index (all) into the following formula to calculate and diagnoses the calculation result (=LNGS) as the aftertaste LNGS of the shochu being analyte.

$$[ Equation 6 ]$$

$$LNGS = Body index ( all ) \times {k}_{2} \qquad ( 6 )$$

In the equation (6), the coefficient k₂ is, for example, 2.

When the taste diagnostic unit 217 receives H(+)_sum (=SMT__{High(+)}) from the calculation unit 216, the taste diagnostic unit 217 diagnoses the calculation result (=SWT) calculated by substituting H(+)_sum (=SMT__{High(+)}) to the following equation to diagnose the calculation result (=SWT) as the “sweetness” of the shochu, which is the analyte.

$$[ Equation 7 ]$$

$$SWT = [ H ( + ) \_sum / {k}_{3} ] \times {k}_{4} \qquad ( 7 )$$

In the equation (7), the coefficient k₃ is, for example, 3000 and the coefficient k4 is, for example, 1.5.

### (4) Diagnosis of “Aroma”

When the taste diagnostic unit 217 receives H(−)_sum (=SMT__{High(-)}) from the calculation unit 216, the taste diagnostic unit 217 substitutes H(−)_sum (=SMT__{High(-)}) into the following equation to diagnose the calculation result (=SCT) as the “aroma” of shochu, which is the analyte.

$$[ Equation 8 ]$$

$$SCT = [ H ( - ) \_sum / {k}_{5} ] \times {k}_{6} \qquad ( 8 )$$

In the equation (8), the coefficient k₅ is, for example, 2000 and the coefficient k₆ is, for example, 1.5.

### (5) Diagnosis of “Bitterness”

The taste diagnostic unit 217 substitutes the “astringency” (=ASTG in equation (5)) and “sweetness” (=SWT in equation (7)) diagnosed by the above-mentioned method into the following formula to calculate the calculation result (=BIT), and diagnoses the calculation result (=BIT) as the “bitterness” of the shochu, which is the analyte.

$$[ Equation 9 ]$$

$$BIT = ASTG \times {k}_{7} - SWT \times {k}_{8} \qquad ( 9 )$$

In the equation (9), the coefficient k7 is, for example, 1.5 and the coefficient k₅ is, for example, 0.4.

The coefficient k₁ in equation (5), the coefficient k₂ in equation (6), the coefficients k₃ and k₄ in equation (7), the coefficients k₅ and k₆ in equation (8), and the coefficients k7 and k₅ in equation (9) are determined using multiple shochu as teacher data whose “astringency,” “aftertaste,” “sweetness,” “aroma,” and “bitterness” are known.

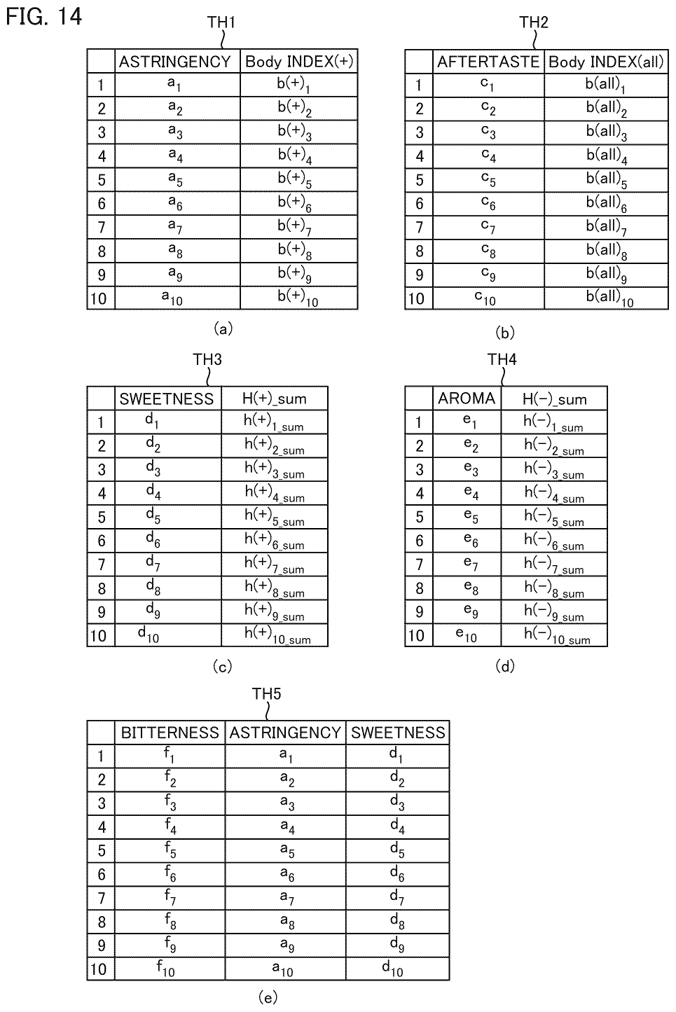

FIG. 14 is a conceptual diagram of the teacher data. Referring to FIG. 14, the teacher data consists of teacher data TH1 to TH5. The teacher data TH1 to TH5 are stored in database 22 in advance.

The teacher data TH1 is teacher data for determining the value of the coefficient k₁ in the formula (5), the teacher data TH2 is teacher data for determining the value of the coefficient k₂ in the formula (6), the teacher data TH3 is teacher data for determining the values of the coefficients k₃, k₄ in the formula (7), the teacher data TH4 is teacher data for determining the values of the coefficients k₅, k₆ in the formula (8), the teacher data TH5 is teacher data for determining the values of the coefficients k₇, k₅ in the formula (9).

When determining the values of the coefficients k₁ to k₅, the taste diagnostic unit 217 reads out the teacher data TH1 to TH5 from the database 22.

### (A) Determination of Coefficient k₁

The training data TH1 includes astringency and Body index (+). The astringency is, for example, made up of ten numbers of a₁ to a₁₀, and the Body index (+) is, for example, made up of ten numbers of b(+)₁ to b(+)₁₀. The ten of a₁ to a₁₀ are known.

The b(+)_{y}(y=1 to 10) is obtained as follows.
(i) The sensor device 1 measures cyclic voltammograms CVG__{Low_y}, CVG__{Middle_y}, and CVG__{High_y} for one shochu y by changing the potential scanning rate to V_{r_Low}, V_{r_Middle}, and V_{r_High}, respectively, and transmits the measured cyclic voltammograms CVG__{Low_y}, CVG__{Middle_y}, and CVG__{High_y} to personal computer PC by wireless communication or wired communication.
(ii) The personal computer PC obtains the correspondence relationship [(Cls₁-ITG₁) to (Clsₙ-ITGₙ)]__{Low_y} between the classes Cls and the integral values ITG based on the cyclic voltammogram CVG__{Low_y} by the method described above.
(iii) The personal computer PC obtains the correspondence relationship [(Cls₁-ITG₁) to (Clsₙ-ITGₙ)]__{Middle_y} between the class Cls and the integral value ITG based on the cyclic voltammogram CVG__{Middle_y} by the method described above.
(iv) The personal computer PC obtains the correspondence relationship [(Cls₁-ITG₁) to (Clsₙ-ITGₙ)]__{High_y} between the class Cls and the integral value ITG based on the cyclic voltammogram CVG__{High_y} by the method described above.
(v) The personal computer PC detects (n/2) integral values associated with the class corresponding to the positive predetermined potential section based on the correspondence relationship [(Cls₁-ITG₁) to (Clsₙ−ITGₙ)]__{Low_y}, calculates the sum of the detected (n/2) integral values, and obtains [Sum of Low(+)]__{y}.
(vi) The personal computer PC detects (n/2) integral values associated with the class corresponding to the positive predetermined potential section based on the correspondence relationship [(Cls₁-ITG₁) to (Clsₙ−ITGₙ)]__{Middle_y}, calculates the sum of the detected (n/2) integral values, and obtains [Sum of Middle(+)]__{y}.
(vii) The personal computer PC detects (n/2) integral values associated with the class corresponding to the positive predetermined potential section based on the correspondence relationship [(Cls₁-ITG₁) to (Clsₙ−ITGₙ)]__{High_y}, calculates the sum of the detected (n/2) integral values, and obtains [Sum of High(+)]__{y}.
(viii) The personal computer PC calculates b(+)_{y} by substituting [Sum of Low(+)]__{y}, [Sum of Middle(+)]__{y}, and [Sum of High(+)]__{y} into SMT__{Low(+)}, SMT__{Middle(+)}, and SMT__{High(+)} in equation (3), respectively.
(ix) The personal computer PC executes the above steps (i) to (viii) for all of y=1 to 10 to obtain b(+)₁ to b(+)₁₀.
(x) When the personal computer PC acquires b(+)₁ to b(+)₁₀, the personal computer PC associates the acquired b(+)₁ to b(+)₁₀ with a₁ to a₁₀, respectively, and stores them in the “Body Index (+)” column of the teacher data TH1.

Then, based on (a₁, b(+)₁), (a₂, b(+)₂), . . . , (a₉, b(+)₉), (a₁₀, b(+)₁₀) of the teacher data TH1, the personal computer PC executes a regression analysis with “Body index (+)” as an explanatory variable and “astringency” as a response variable to obtain a regression equation, and determines the value multiplied to the explanatory variable “Body index (+)” in the obtained regression equation as the value of coefficient k₁ in equation (5).

### (B) Determination of Coefficient k₂

The teacher data TH2 includes aftertaste and Body index (all). The aftertaste is made up of, for example, ten elements c₁ to c₁₀, and the Body index (all) is made up of, for example, ten elements b(all)₁ to b(all)₁₀. Ten of c₁ to c₁₀ are known.

b(all)_{y} (y=1 to 10) is obtained as follows.
(xi) The personal computer PC calculates the sum SUM_{all_Low_y} of all the integral values ITG₁ to ITGₙ based on the correspondence relationship [(Cls₁-ITG₁) to (Clsₙ−ITGₙ)]__{Low_y} obtained in (ii) above.
(xii) The personal computer PC calculates the sum SUM_{all_Middle_y} of all the integral values ITG₁ to ITGₙ based on the correspondence relationship [(Cls₁-ITG₁) to (Clsₙ−ITGₙ)]__{Middle_y} obtained in (iii) above.
(xiii) The personal computer PC calculates the sum SUM_{all_High_y} of all the integral values ITG₁ to ITGₙ based on the correspondence relationship [(Cls₁-ITG₁) to (Clsₙ−ITGₙ)]__{High_y} obtained in (iv) above.
(xiv) The personal computer PC calculates b(all)_{y} by substituting the sum SUM_{all_Low_y}, the sum SUM_{all_Middle_y}, and the sum SUM_{all_High_y} into SMT__{Low(all)}, SMT__{Middle(all)}, and SMT__{High(all)} in equation (4), respectively.
(xv) The personal computer PC executes the above steps (xi) to (xiv) for all of y=1 to 10 to obtain b(all)₁ to b(all)₁₀.
(xvi) When the personal computer PC obtains b(all)₁ to b(all)₁₀, the personal computer PC associates the obtained b(all)₁ to b(all)₁₀ with c₁ to c₁₀ of the teacher data TH2, respectively, to store the obtained b(all)₁ to b(all)₁₀ in the “Body index (all)” column of the teacher data TH2.

Then, the personal computer PC executes a regression analysis using the “Body index (all)” as an explanatory variable and the “aftertaste” as a response variable based on (c₁, b(all)₁), (c₂, b(all)₂), . . . , (c₉, b(all)₉), (c₁₀, b(all)₁₀) of the teacher data TH2 to obtain a regression equation, and determines the value multiplied to the explanatory variable “Body index (all)” in the obtained regression equation as the value of the coefficient k₂ in equation (6).

(C) Determination of Coefficients k₃, k₄
The teacher data TH3 includes sweetness and H(+)_sum. Sweetness is, for example, made up of ten elements d₁ to d₁₀, and H(+)_sum is, for example, made up often elements h(+)_{1_sum} to h(+)_{10_sum}. Ten values d₁ to d₁₀ are known.

h(+)_{y_sum} (y=1 to 10) is obtained as follows.
(xvii) The personal computer PC executes the above (vii) for all of y=1 to 10 to obtain h(+)_{1_sum} to h(+)_{10_sum}.
(xviii) When the personal computer PC obtains h(+)_{1_sum} to h(+)_{10_sum}, the personal computer PC associates the obtained h(+)_{1_sum} to h(+)_{10_sum} with d₁ to d₁₀ of the teacher data TH3, respectively, and stores them in the “H(+)_sum” column of the teacher data TH3.

Then, based on (d₁, h(+)_{1_sum}), (d₂, h(+)_{2_sum}), . . . , (d₉, h(+)_{9_sum}), and (d₁₀, h(+)_{10_sum}) of the teacher data TH3, the personal computer PC performs a regression analysis with “H(+)_sum” as the explanatory variable and “sweetness” as the target variable to obtain a regression equation, determines the value by which the explanatory variable “H(+)_sum” is divided in the obtained regression equation as the value of coefficient k₃ in equation (7), and determines the value multiplied to the explanatory variable “H(+)_sum” as the value of coefficient k₄ in equation (7).

(D) Determination of Coefficients k₅, k₆
The teacher data TH4 includes an aroma and H(−)_sum. The aroma is made up of, for example, ten elements e₁ to e₁₀, and H(−)_sum is made up of, for example, ten elements h(−)_{1_sum} to h(−)_{10_sum}. Ten of e₁ to e₁₀ are known.

h(−)_{y_sum} (y=1 to 10) is obtained as follows.
(xix) The personal computer PC detects (n/2) integral values associated with the class corresponding to the predetermined negative potential section based on the correspondence relationship [(Cls₁-ITG₁) to (Clsₙ−ITGₙ)]__{High_y} obtained in (iv) above, and calculates the sum of the detected (n/2) integral values to obtain h(−)_{y_sum}.
(xx) The personal computer PC executes (xix) for all values of y=1 to 10 to obtain h(−)_{1_sum} to h(−)_{10_sum}.
(xxi) When the personal computer PC acquires h(−)_{1_sum} to h(−)_{10_sum}, the personal computer PC associates the acquired h(−)_{1_sum} to h(−)_{10_sum} with e₁ to e₁₀ of the teacher data TH4, respectively, and stores them in “H(−)_sum” of the teacher data TH4.

Then, based on (e₁, h(−)_{1_sum}), (e₂, h(−)_{2_sum}), . . . , (e₉, h(−)_{9_sum}), (e₁₀, h(−)_{10_sum}) of the teacher data TH4, the personal computer PC performs regression analysis with “H(−)_sum” as the explanatory variable and “aroma” as the target variable to obtain a regression equation, determines the value by which the explanatory variable “H(−)_sum” is divided in the obtained regression equation as the value of coefficient k₅ in equation (8), and determines the value multiplied to the explanatory variable “H(−)_sum” as the value of coefficient k₆ in equation (8).

(E) Determination of Coefficients k₇, k₈
The teacher data TH5 includes “bitterness”, “astringency” and “sweetness”. “bitterness” is made up of, for example, ten elements f₁ to f₁₀. “Astringency” is made up of elements a₁ to a₁₀ included in the teacher data TH1. “Sweetness” is made up of elements d₁ to d₁₀ included in the teacher data TH3.
(xxii) The personal computer PC performs regression analysis with “astringency” and “sweetness” as explanatory variables and “bitterness” as the objective variable to obtain a regression equation, and determines the value multiplied to “astringency” (=ASTG) in the obtained regression equation as the value of coefficient k₇ in equation (9), and determines the value multiplied to “sweetness” (=SWT) as the value of coefficient k₈ in equation (9).

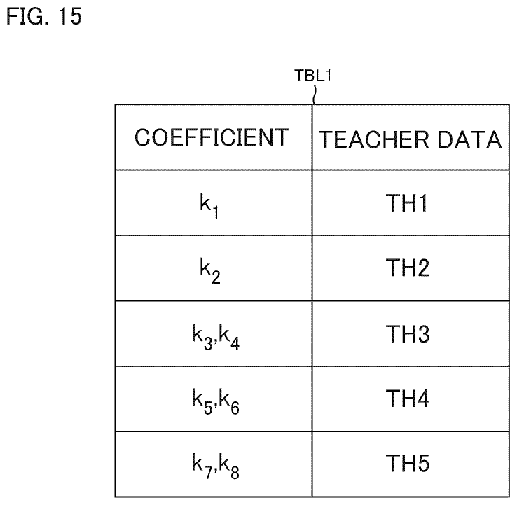

FIG. 15 is a conceptual diagram of a correspondence table showing the correspondence between coefficients and teacher data. Referring to FIG. 15, a correspondence table TBL1 includes coefficients and teacher data. The coefficients and the teacher data are associated with each other.

The coefficients are made of k₁ to k₈, and the teacher data is made of TH1 to TH5. The teacher data TH1 is associated with the coefficient k₁, the teacher data TH2 is associated with the coefficient k₂, the teacher data TH3 is associated with the coefficients k₃ and k₄, the teacher data TH4 is associated with the coefficients k₅ and k₆, and the teacher data TH5 is associated with the coefficients k₇ and k₈.

As a result, the correspondence table TBL1 indicates that coefficient k₁ is determined based on teacher data TH1, coefficient k₂ is determined based on teacher data TH2, coefficients k₃ and k₄ are determined based on teacher data TH3, coefficients k₅ and k₆ are determined based on teacher data TH4, and coefficients k₇ and k₈ are determined based on teacher data TH5.

After determining the values of the coefficients k₁ to k₈ by the above-mentioned method, the personal computer PC creates the correspondence table TBL1 and transmits the created correspondence table TBL1 to the diagnostic device 2.

The receiving unit 211 of the diagnostic device 2 receives the correspondence table TBL 1 from the personal computer PC, and outputs the received correspondence table TBL 1 to the control unit 212.

When the control unit 212 has received the correspondence table TBL 1 from the receiving unit 211, the control unit 212 stores the received correspondence table TBL 1 in the database 22.

When diagnosing “astringency”, “aftertaste”, “sweetness”, “aroma” and “bitterness”, the taste diagnostic unit 217 reads out the coefficients k₁ to k₈ of the correspondence table TBL1 stored in the database 22, and uses the read-out coefficients k₁ to k₈ to diagnose “astringency”, “aftertaste”, “sweetness”, “aroma” and “bitterness” by the method described above.

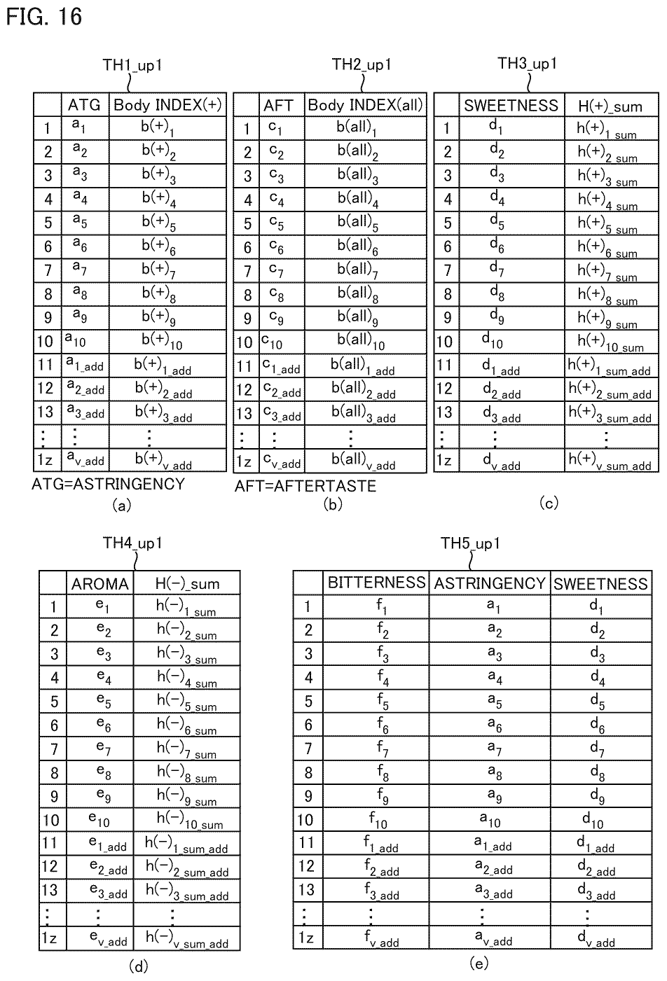

FIG. 16 is a conceptual diagram of updated teacher data. FIG. 16(a) shows teacher data TH1_up1 obtained by updating teacher data TH1 shown in FIG. 14(a). FIG. 16(b) shows teacher data TH2_up1 obtained by updating teacher data TH2 shown in FIG. 14(b). FIG. 16(c) shows teacher data TH3_up1 obtained by updating teacher data TH3 shown in FIG. 14(c). FIG. 16(d) shows teacher data TH4_up1 obtained by updating teacher data TH4 shown in FIG. 14(d). FIG. 16(e) shows teacher data TH5_up1 obtained by updating teacher data TH5 shown in FIG. 14(e).

In the teacher data TH1_up1 to TH5_up1, the “1” in “up1” represents the number of updated times which the teacher data TH1 to TH5 have been updated.

With referring to FIG. 16(a), the teacher data TH1_up1 is obtained by adding “astringency a_{1_add} to a_{v_add}” and “Body index (+): b(+)_{1_add} to b(+)_{v_add}” to the teacher data TH1 shown in FIG. 14(a), where v is an integer equal to or greater than 1.

With referring to FIG. 16(b), the teacher data TH2_up1 is obtained by adding “aftertaste c_{1_add} to c_{v_add}” and “Body index (all): b(all)_{1_add} to b(all)_{v_add}” to the teacher data TH2 shown in FIG. 14(b).

With referring to FIG. 16(c), the teacher data TH3_up1 is obtained by adding “sweetness d_{1_add} to d_{v_add}” and “H(+)_sum: h(+)_{1_sum} add to h(+)_{v_sum_add}” to the teacher data TH3 shown in FIG. 14(c).

With referring to FIG. 16(d), the teacher data TH4_up1 is obtained by adding “aroma e_{1_add} to e_{v_add}” and “H(−)_sum: h(−)_{1_sum_add} to h(−)_{v_sum_add}” to the teacher data TH4 shown in FIG. 14(d).

With referring to FIG. 16(e), the teacher data TH5_up1 is obtained by adding “bitterness f_{1_add} to f, add”, “astringency: a_{1_add} to a_{v_add}”, and “sweetness: d_{1_add} to d, add” to the teacher data TH5 shown in FIG. 14(e).

The taste diagnostic unit 217 uses the coefficients k₁ to k₈ to diagnose the “astringency”, “aftertaste”, “sweetness”, “aroma” and “bitterness” of v shochus using the method described above, and then reads out teacher data TH1 to TH5 from the database 22.

Then, the taste diagnostic unit 217 adds “astringency a_{1_add} to a_{v_add}” and “Body index (+): b(+)_{1_add} to b(+)_{v_add}” to the teacher data TH1, and updates the teacher data TH1 to teacher data TH1_up1.

In addition, the taste diagnostic unit 217 adds “aftertaste c₁_add to c_{v_add}” and “Body index (all): b(all)_{1_add} to b(all), add” to the teacher data TH2, and updates the teacher data TH2 to teacher data TH2_up1.

Furthermore, the taste diagnostic unit 217 adds “sweetness d_{1_add} to d, add” and “H(+)_sum: h(+)_{1_sum} add to h(+)_{v_sum_add}” to the teacher data TH3, and updates the teacher data TH3 to teacher data TH3_up1.

Furthermore, the taste diagnostic unit 217 adds “aroma e_{1_add} to e, add” and “H(−)_sum: h(−)_{1_sum_add} to h(−)_{v_sum_add}” to the teacher data TH4, and updates the teacher data TH4 to teacher data TH4_up1.

Furthermore, the taste diagnostic unit 217 adds “bitterness f_{1_add} to f, add”, “astringency a_{1_add} to a_{v_add}” and “sweetness: d_{1_add} to d, add” to the teacher data TH5, and updates the teacher data TH5 to teacher data TH5_up1.

Then, the taste diagnostic unit 217 determines the value of coefficient k₁ based on the updated teacher data TH1_up1 using the method described above in “(A) Determination of coefficient k₁”, and updates the value of coefficient k₁ to the determined value.

In addition, the taste diagnostic unit 217 determines the value of coefficient k₂ based on the updated teacher data TH2_up1 using the method described above in “(B) Determination of coefficient k₂”, and updates the value of coefficient k₂ to the determined value.

Furthermore, the taste diagnostic unit 217 determines the values of coefficients k₃ and k₄ based on the updated teacher data TH3_up1 using the method described above in “(C) Determination of coefficients k₃ and k₄”, and updates the values of coefficients k₃ and k₄ to the determined values.

Furthermore, the taste diagnostic unit 217 determines the values of coefficients k₅ and k₆ based on the updated teacher data TH4_up1 using the method described above in “(D) Determination of coefficients k₅ and k₆,” and updates the values of coefficients k₅ and k₆ to the determined values.

Furthermore, the taste diagnostic unit 217 determines the values of coefficients k₇ and k₈ based on the updated teacher data TH5_up1 using the method described above in “(E) Determination of coefficients k₇ and k₈”, and updates the values of coefficients k₇ and k₈ to the determined values.

Then, after updating the values of the coefficients k₁ to k₈, the taste diagnostic unit 217 stores the updated teacher data TH1_up1 to TH5_up1 in the database 22.

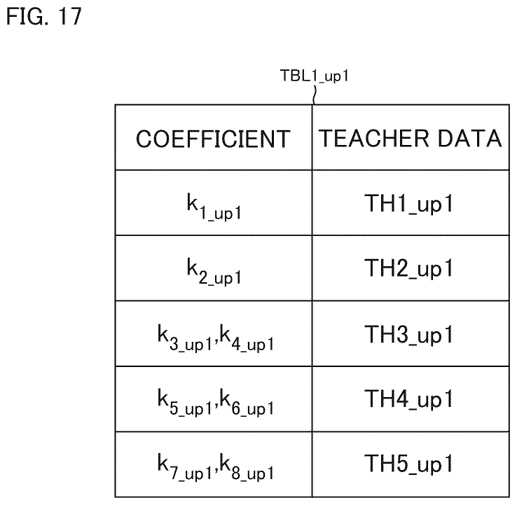

FIG. 17 is a conceptual diagram of a correspondence table TBL1_up1 obtained by updating the correspondence table TBL1 shown in FIG. 15.

Referring to FIG. 17, the correspondence table TBL1_up1 includes coefficients k_{1_up1} to k_{8_up1} and teacher data TH1_up1 to TH5_up1. The coefficients k_{1_up1} to k_{8_up1} are updated coefficients, and the teacher data TH1_up1 to TH5_up1 are updated teacher data.

The teacher data TH1_up1 is associated with the coefficient k_{1_up1}, the teacher data TH2_up1 is associated with the coefficient k_{2_up1}, the teacher data TH3_up1 is associated with the coefficients k_{3_up1} and k_{4_up1}, the teacher data TH4_up1 is associated with the coefficients k_{5_up1} and k_{6_up1}, and the teacher data TH5_up1 is associated with the coefficients k_{7_up1} and k_{8_up1}.

When the taste diagnostic unit 217 updates the values of the coefficients k₁ to k₅ using the method described above, the taste diagnostic unit 217 updates the correspondence table TBL1 to the correspondence table TBL1_up1 based on the updated values of the coefficients k₁ to k₈, and stores the updated correspondence table TBL1_up1 in the database 22.

Then, the taste diagnostic unit 217 uses the coefficients k_{1_up1} to k_{8_up1} stored in the correspondence table TBL1_up1 to diagnose the “astringency,” “aftertaste,” “sweetness,” “aroma,” and “bitterness” of one or more new shochu using the method described above.

Thereafter, the taste diagnostic unit 217 repeatedly updates the teacher data TH1 to TH5, updates the values of the coefficients k₁ to k₈ using the updated teacher data TH1 to TH5, and repeatedly diagnoses the “astringency,” “aftertaste,” “sweetness,” “aroma,” and “bitterness” of one or more new shochu samples.

### (II) Taste Diagnosis of Grape

When the calculation unit 216 has received calculation data CAL (=calculation data having the same configuration as the calculation data CALᵤₙᵢ shown in FIG. 12) from the control unit 212, the calculation unit 216 detects the integral value ITG Low (ITG_{1_Low} to ITG_{n_Low}), the integral value ITG__{Middle} (ITG_{1_Middle} to ITG_{n_Middle}), and the integral value ITG__{High} (ITG₁_High to ITG_{n_High}) from the calculation data CAL.

Then, the calculation unit 216 detects a plurality of integral values ITG_{U_Low_th} to ITG_{n_Low_th} corresponding to a predetermined potential section (=class Clsₜₕ) including a potential V equal to or lower than the threshold value Vₜₕ, among the predetermined negative potential sections, based on the classes Cls₁ to Clsₙ and the integral value ITG__{Low} (ITG_{1_Low} to ITG_{n_Low}). U indicates the class Clsₜₕ corresponding to the predetermined potential section including the potential V corresponding to the threshold value Vₜₕ.

Furthermore, the calculation unit 216 detects a plurality of integral values ITG_{U_Middle_th} to ITG_{n_Middle_th} corresponding to a predetermined potential section (=class Clsₜₕ) equal to or lower than the threshold value Vₜₕ, among the predetermined negative potential sections, based on the classes Cls₁ to Clsₙ and the integral value ITG__{Middle} (ITG_{1_Middle} to ITG_{n_Middle}).

Furthermore, the calculation unit 216 detects a plurality of integral values ITG_{U_High_th} to ITG_{n_High_th} corresponding to a predetermined potential section (=class Cls) equal to or lower than the threshold value Vₜₕ, out of a predetermined negative potential section, based on the classes Cls₁ to Clsₙ and the integral value ITG__{High} (ITG_{1_High} to ITG_{n_High}).

The subscript “U” in the multiple integral values ITG_{U_Low_th} to ITG_{n_Low_th}, ITG_{U_Middle_th} to ITG_{n_Middle_th}, and ITG_{U_High_th} to ITG_{n_High_th} represents a predetermined potential section including the potential V corresponding to the threshold value Vₜₕ, so each of the integral values ITG_{U_Low_th}, ITG_{U_Middle_th}, and ITG_{U_High_th} is an integral value in a predetermined potential section including the potential V corresponding to the threshold value Vₜₕ.

When the calculation unit 216 has detected multiple integrated values ITG_{U_Low_th} to ITG_{n_Low_th}, ITG_{U_Middle_th} to ITG_{n_Middle_th}, and ITG_{U_High_th} to ITG_{n_High_th}, the calculation unit 216 calculates a sum L(−)_sum_th of the multiple integrated values ITG_{U_Low_th} to ITG_{n_Low_th}, calculates a sum M(−)_sum_th of the multiple integrated values ITG_{U_Middle_th} to ITG_{n_Middle_th}, and calculates a sum H(−)_sum_th of the multiple integrated values ITG_{U_High_th} to ITG_{n_High_th}.

Then, the calculation unit 216 substitutes the sums L(−)_sum_th, M(−)_sum_th, and H(−)_sum_th of the integral values, M(−)_sum_th, and H(−)_sum_th into the following equation to calculate the Body index (−)_th.

$$[ Equation 10 ]$$

$$Body index ( - ) \_th = \frac{M ( - ) \_sum \_th}{H ( - ) \_sum \_th} + \frac{L ( - ) \_sum \_th}{H ( - ) \_sum \_th} \qquad ( 10 )$$

After calculating Body index (−)_th, the calculation unit 216 outputs the calculated Body index (−)_th to the taste diagnostic unit 217.

When the taste diagnostic unit 217 has received Body index (−)_th from calculation unit 216, the taste diagnostic unit 217 substitutes Body index (−)_th and coefficient k9 into the following equation to calculate value ASTG_GRP of “astringency” of the grape, and diagnoses the calculated value ASTG_GRP as the “astringency” of the grape.

$$[ Equation 11 ]$$

$$ASTG\_GRP = Body index ( - ) \_th \times {k}_{9} \qquad ( 11 )$$

In equation 11, the coefficient k₉, for example, is 1.5.

Then, the taste diagnostic unit 217 determines the value of the coefficient k₉ by the same manner as the above-mentioned method for determining the value of the coefficient k₁.

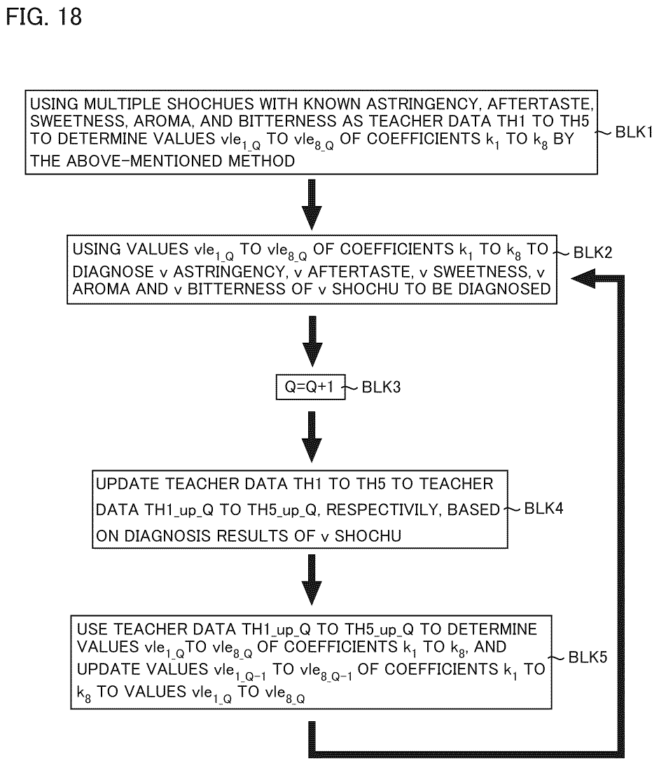

FIG. 18 is a conceptual diagram for diagnosing the taste (astringency, aftertaste, sweetness, aroma, bitterness) of shochu. Referring to FIG. 18, the taste diagnostic unit 217 uses multiple shochu with known “astringency,” “aftertaste,” “sweetness,” “aroma,” and “bitterness” as teacher data TH1 to TH5 to determine the values vle_{1_Q} to vle_{8_Q} of the coefficients k₁ to k₈ by the above-mentioned method (block BLK1).

Then, the taste diagnostic unit 217 uses the values vle_{1_Q} to vle_{8_Q} (Q=0, 1, 2, . . . ) of the coefficients k₁ to k₅ to diagnose the v “astringency,” v “aftertaste,” v “sweetness,” v “aroma,” and v “bitterness” of the v shochu to be diagnosed by the method described above (block BLK2).

Thereafter, the taste diagnostic unit 217 sets Q=Q+1 (block BLK3), and updates the teacher data TH1-TH5 to teacher data TH1_up_Q to TH5_up_Q, respectively, based on the diagnosis results of the v Shochus (block BLK4).

Next, the taste diagnostic unit 217 uses the teacher data TH1_up_Q to TH5_up_Q to determine the values vle_{1_Q} to vle_{8_Q} of the coefficients k₁ to k₈ by the method described above, and updates the values vle_{1_Q-1} to vle_{8_Q-1} of the coefficients k₁ to k₈ to the values vle_{1_Q} to vle_{8_Q}, respectively (block BLK5).

After block BLK5, the taste diagnostic unit 217 repeatedly executes blocks BLK2 to BLK5.

When moving from block BLK1 to block BLK2, since the values vle_{1_Q} to vle_{8_Q} of coefficients k₁ to k₈ are set to values vle_{1_0} to vle_{8_0}, respectively, the taste diagnostic unit 217 uses, in block BLK2, the values vle_{1_0} to vle_{8_0} of coefficients k₁ to k₈ to diagnose the v “astringency,” v “aftertaste,” v “sweetness,” v “aroma,” and v “bitterness” of the v shochu to be diagnosed. In other words, the taste diagnostic unit 217 diagnoses v “astringency,” v “aftertaste,” v “sweetness,” v “aroma,” and v “bitterness” of the v shochu to be diagnosed using the values vle_{1_0} to vle_{8_0} of the coefficients k₁ to k₈ determined using multiple shochu whose “astringency,” “aftertaste,” “sweetness,” “aroma,” and “bitterness” are known as teacher data TH1 to TH5.

After block BLK2, the taste diagnostic unit 217 sets Q=Q+1 (block BLK3) and then executes block BLK4.

In this case, since Q=Q+1=0+1=1 is set in block BLK3, the taste diagnostic unit 217 updates the teacher data TH1 to TH5 to teacher data TH1_up_1 to TH5_up_1, respectively, in block BLK4.

Then, in block BLK5, the taste diagnostic unit 217 determines the values vle_{1_1} to vle_{8_1} of the coefficients k₁ to k₈ using the updated teacher data TH1_up_1 to TH5_up_1 by the method described above, and updates the values vle_{1_0} to vle_{8_0} of the coefficients k₁ to k₈ to the values vle_{1_1} to vle_{8_1}, respectively.

After block BLK5, the taste diagnostic unit 217 uses the values vle_{1_1} to vle_{8_1} of the coefficients k₁ to k₈ to diagnose the v “astringency,” v “aftertaste,” v “sweetness,” v “aroma,” and v “bitterness” of the v shochu to be diagnosed by the method described above (block BLK2). In other words, in block BLK2, the taste diagnostic unit 217 uses the values vle_{1_1} to vle_{8_1} of the coefficients k₁ to k₈ determined using the updated teacher data TH1_up_1 to TH5_up_1 to diagnose the v “astringency,” v “aftertaste,” v “sweetness,” v “aroma,” and v “bitterness” of the v shochu to be diagnosed.

After block BLK2, the taste diagnostic unit 217 sets Q=Q+1 (block BLK3) and then executes block BLK4.

In this case, since Q=Q+1=1+1=2 is set in block BLK3, the taste diagnostic unit 217 updates the teacher data TH1_up_1 to TH5_up_1 to teacher data TH1_up_2 to TH5_up_2, respectively, in block BLK4.

Then, in block BLK5, the taste diagnostic unit 217 determines the values vle_{1_2} to vle_{8_2} of the coefficients k₁ to k₈ using the updated teacher data TH1_up_2 to TH5_up_2 by the method described above, and updates the values vle_{1_1} to vle_{8_1} of the coefficients k₁ to k₈ to the values vle_{1_2} to vle_{8_2}, respectively.

After block BLK5, the taste diagnostic unit 217 uses the values vle_{1_2} to vle_{8_2} of the coefficients k₁ to k₈ to diagnose the v “astringency,” v “aftertaste,” v “sweetness,” v “aroma,” and v “bitterness” of the v shochu to be diagnosed by the method described above (block BLK2). In other words, in block BLK2, the taste diagnostic unit 217 uses the values vle_{1_2} to vle_{8_2} of the coefficients k₁ to k₈ determined using the updated teacher data TH1_up_2 to TH5_up_2 to diagnose the v “astringency,” v “aftertaste,” v “sweetness,” v “aroma,” and v “bitterness” of the v shochu to be diagnosed.

After block BLK2, the taste diagnostic unit 217 sets Q=Q+1 (block BLK3) and then executes block BLK4.

In this case, since Q=Q+1=2+1=3 is set in block BLK3, the taste diagnostic unit 217 updates the teacher data TH1_up_2 to TH5_up_2 to teacher data TH1_up_3 to TH5_up_3, respectively, in block BLK4.

Thereafter, the taste diagnostic unit 217 repeatedly executes blocks BLK2 to BLK5.

Each time blocks BLK2 to BLK5 are one time executed, v pieces of “astringency” and v pieces of “Body index (+)” are added to teacher data TH1, v pieces of “aftertaste” and v pieces of “Body index (all)” are added to teacher data TH2, v pieces of “sweetness” and v pieces of “H(+)_sum” are added to teacher data TH3, v pieces of “aroma” and v pieces of “H(−)_sum” are added to teacher data TH4, and v pieces of “bitterness”, v pieces of “astringency”, and v pieces of “sweetness” are added to teacher data TH5.

Therefore, as the number of times blocks BLK2 to BLK5 are repeatedly executed increases, it is possible to set to higher value the accuracy of the values vle₁ to vle₈ of the coefficients k₁ to k₈ determined by regression analysis in block BLK5.

In addition, in FIG. 18, when blocks BLK2 to BLK5 are executed the gth time (g is an integer equal to or greater than 1), v may be set to v_{Q} (v_{Q} is an integer equal to or greater than 1) to sequentially execute blocks BLK2 to BLK5, and when blocks BLK2 to BLK5 are executed the (g+1)th time, v may be set to v_{R} (v_{R} is an integer equal to or greater than 1 and different from v_{Q}) to sequentially execute blocks BLK2 to BLK5. In other words, the number (v=v_{R}) of “shochu” diagnosed in the (g+1)th time may be different from the number of “shochu” diagnosed in the gth time (v=v_{Q}).

Also, when the taste diagnostic unit 217 diagnoses the “astringency” of grapes, the taste diagnostic unit 217 executes blocks BLK1 to BLK5 shown in FIG. 18 to diagnose the “astringency” of grapes while updating the teacher data and the value of the coefficient k₉.

In this case, the number (v=v_{R}) of “grapes” diagnosed in the (g+1)th time may be different from the number (v=v_{Q}) of “grapes” diagnosed in the gth time.

### Example

The curve CUR generated by the diagnostic device 2 will be described for the case where the analyte is shochu and grapes.

### (1) Shochu

The shochu used to create the curve CUR are shown in Table 2.

TABLE 2

| Name | Alcohol content(%) | Alcoholic beverages |
|---|---|---|
| Sample No. 1 | 25% | Wheat |
| Sample No. 2 | 25% | Wheat |
| Sample No. 3 | 25% | Wheat |
| Sample No. 4 | 25% | Wheat |
| Sample No. 5 | 25% | Wheat |
| Sample No. 6 | 25% | Wheat |
| Sample No. 7 | 25% | Potato |
| Sample No. 8 | 25% | Potato |
| Sample No. 9 | 25% | Potato |
| Sample No. 10 | 25% | Potato |
| Sample No. 11 | 25% | Potato |
| Sample No. 12 | 25% | Potato |
| Sample No. 13 | 25% | Potato |
| Sample No. 14 | 25% | Potato |
| Sample No. 15 | 25% | Rice |
| Sample No. 16 | 25% | Rice |
| Sample No. 17 | 25% | Sake lees |
| Sample No. 18 | 40% | Barrel-stored wheat |
| Sample No. 19 | 36% | Barrel-stored wheat |
| Sample No. 20 | 40% | Barrel-stored wheat |

The measurement conditions for the cyclic voltammograms CVG for the shochu of samples No. 1 to No. 20 shown in Table 2 are shown in Table 3.

TABLE 3

| Working electrode | Diamond electrode (diameter: 3 mm) | |
|---|---|---|
| Counter electrode | Gold(rod-shaped electrode) | |
| Reference electrode | Gold(rod-shaped electrode) | |
| Potential scanning range | ±2.5 | V |
| Integral value extraction potential | 18.1 | mV |

| | |
|---|---|
| Potential scanning speeds | 300 mV/s, 500 mV/s, 600 mV/s |

As shown in Table 3, the working electrode was made of diamond having a circular planar shape, and the counter electrode and reference electrode were made of rod-shaped gold. The potential scanning range in when measuring the cyclic voltammogram is −2.5 V to +2.5 V, the predetermined potential section (integral value extraction potential) in when calculating the integral value is 18.1 mV, and the potential scanning speeds are 300 mV/s, 500 mV/s, and 600 mV/s.

In terms of the potential scanning speed, 300 mV/s corresponds to a potential scanning speed V_{r_Low}, 500 mV/s corresponds to a potential scanning speed V_{r_Middle}, and 600 mV/s corresponds to a potential scanning speed V_{r_High}.

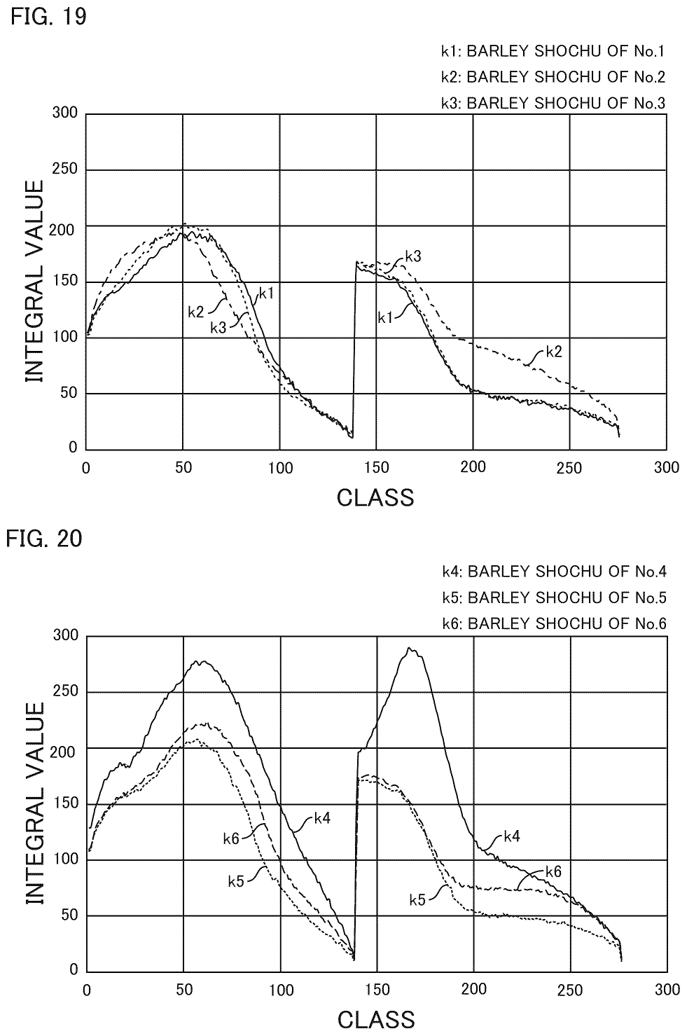

FIG. 19 is a diagram showing the integral value spectra for the shochu of samples No. 1 to No. 3 shown in Table 2.

Referring to FIG. 19, each of the curves k₁ to k₃ is a curve CUR (index curve) generated by the creation unit 215 of the diagnostic device 2 using the method described above. Curve k1 shows the integral value spectrum of the barley shochu of sample No. 1, curve k₂ shows the integral value spectrum of the barley shochu of sample No. 2, and curve k₃ shows the integral value spectrum of the barley shochu of sample No. 3.

FIG. 20 is a diagram showing the integral value spectra for the shochu of samples No. 4 to No. 6 shown in Table 2.

Referring to FIG. 20, each of the curves k₄ to k₆ is a curve CUR (index curve) created by the creation unit 215 of the diagnostic device 2 using the method described above. Curve k₄ shows the integral value spectrum of the barley shochu of sample No. 4, curve k₅ shows the integral value spectrum of the barley shochu of sample No. 5, and curve k₆ shows the integral value spectrum of the barley shochu of sample No. 6.

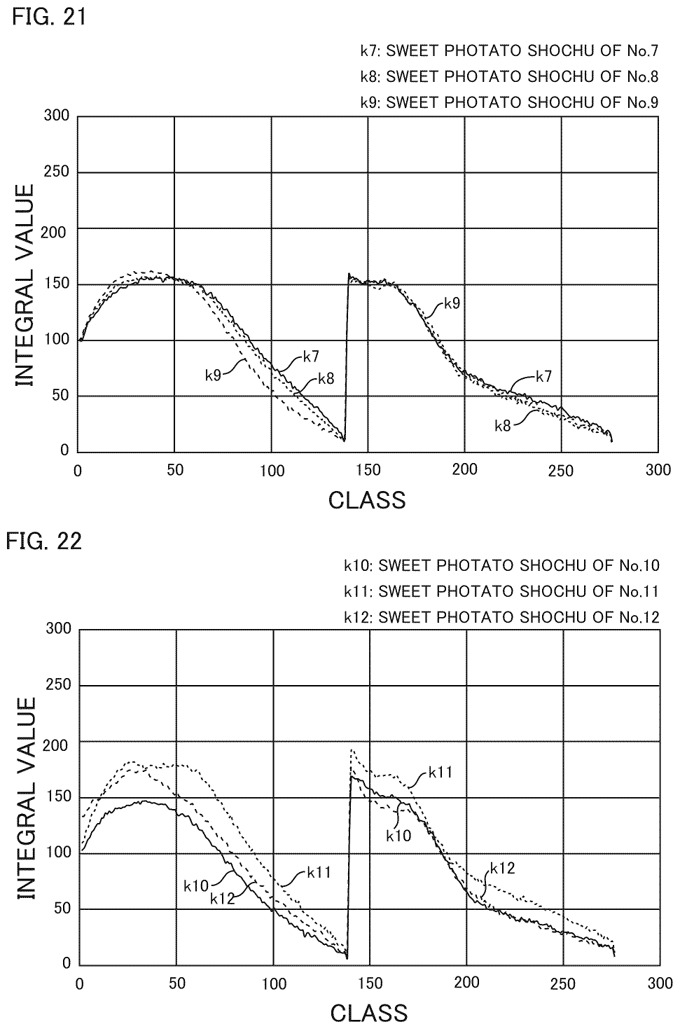

FIG. 21 is a diagram showing the integral value spectra for the shochu samples No. 7 to No. 9 shown in Table 2.

Referring to FIG. 21, each of the curves k₇ to k₉ is a curve CUR (index curve) created by the creation unit 215 of the diagnostic device 2 using the method described above. Curve k₇ shows the integral value spectrum of the sweet potato shochu of sample No. 7, curve k₈ shows the integral value spectrum of the sweet potato shochu of sample No. 8, and curve k₉ shows the integral value spectrum of the sweet potato shochu of sample No. 9.

FIG. 22 is a diagram showing integral value spectra for the shochu of samples No. 10 to No. 12 shown in Table 2.

Referring to FIG. 22, each of the curves k10 to k12 is a curve CUR (index curve) created by the creation unit 215 of the diagnostic device 2 using the method described above. A curve k10 indicates the integral value spectrum of the sweet potato shochu of sample No. 10, a curve k11 indicates the integral value spectrum of the sweet potato shochu of sample No. 11, and a curve k12 indicates the integral value spectrum of the sweet potato shochu of sample No. 12.

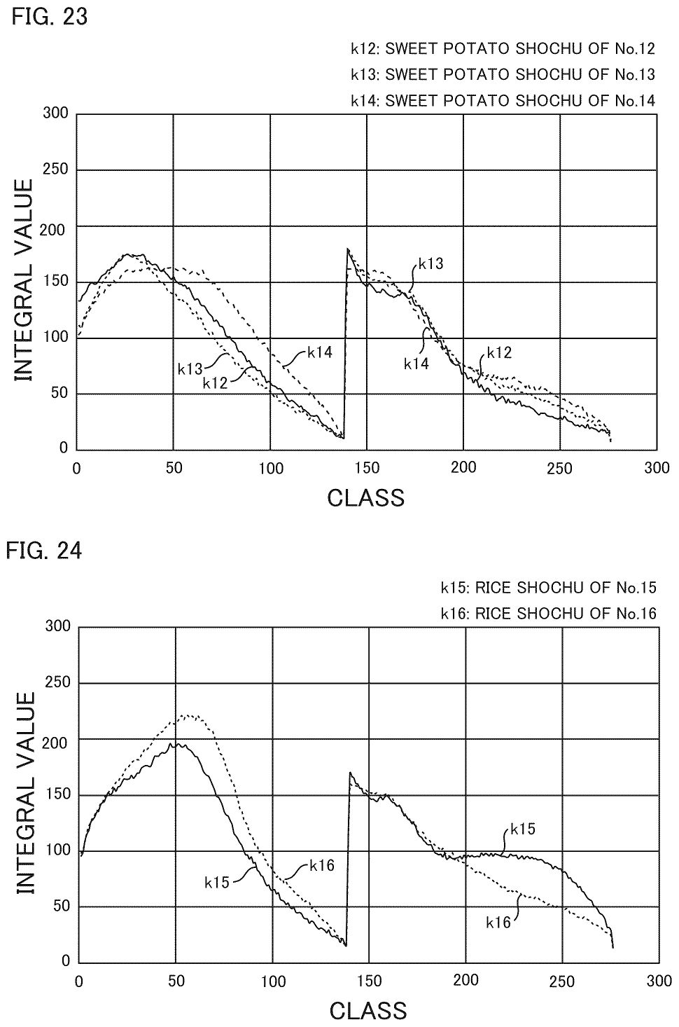

FIG. 23 is a diagram showing integral value spectra for the shochu of samples No. 12 to No. 14 shown in Table 2.

Referring to FIG. 23, each of the curves k12 to k14 is a curve CUR (index curve) created by the creation unit 215 of the diagnostic device 2 using the method described above. Curve k12 shows the integral value spectrum of the sweet potato shochu of sample No. 12, curve k13 shows the integral value spectrum of the sweet potato shochu of sample No. 13, and curve k14 shows the integral value spectrum of the sweet potato shochu of sample No. 14.

FIG. 24 is a diagram showing integral value spectra for the shochu of samples No. 15 to No. 16 shown in Table 2.

Referring to FIG. 24, each of the curves k15 and k16 is a curve CUR (index curve) created by the creation unit 215 of the diagnostic device 2 using the above-mentioned method. The curve k15 shows the integral value spectrum of the rice shochu of sample No. 15, and the curve k16 shows the integral value spectrum of the rice shochu sample No. 16.

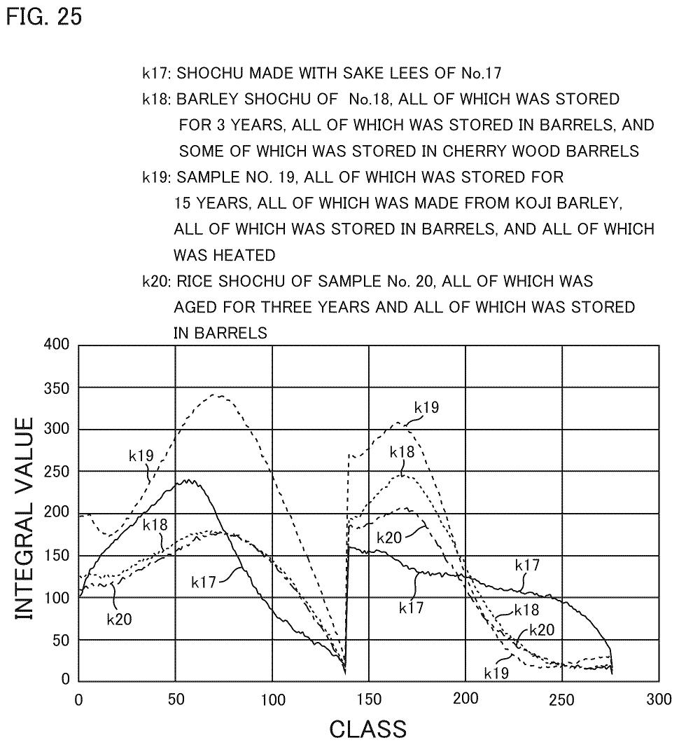

FIG. 25 is a diagram showing integral value spectra for the shochu of samples No. 17 to No. 20 shown in Table 2.

Referring to FIG. 25, each of the curves k17 to k20 is a curve CUR (index curve) created by the creation unit 215 of the diagnostic device 2 using the method described above. Curve k17 shows the integral value spectrum of shochu made with sake lees of sample No. 17. Curve k18 shows the integral value spectrum of barley shochu of sample No. 18, all of which was stored for 3 years, all of which was stored in barrels, and some of which was stored in cherry wood barrels. Curve k19 shows the integral value spectrum of sample No. 19, all of which was stored for 15 years, all of which was made from koji barley, all of which was stored in barrels, and all of which was heated. Curve k20 shows rice shochu of sample No. 20, all of which was aged for three years and all of which was stored in barrels.

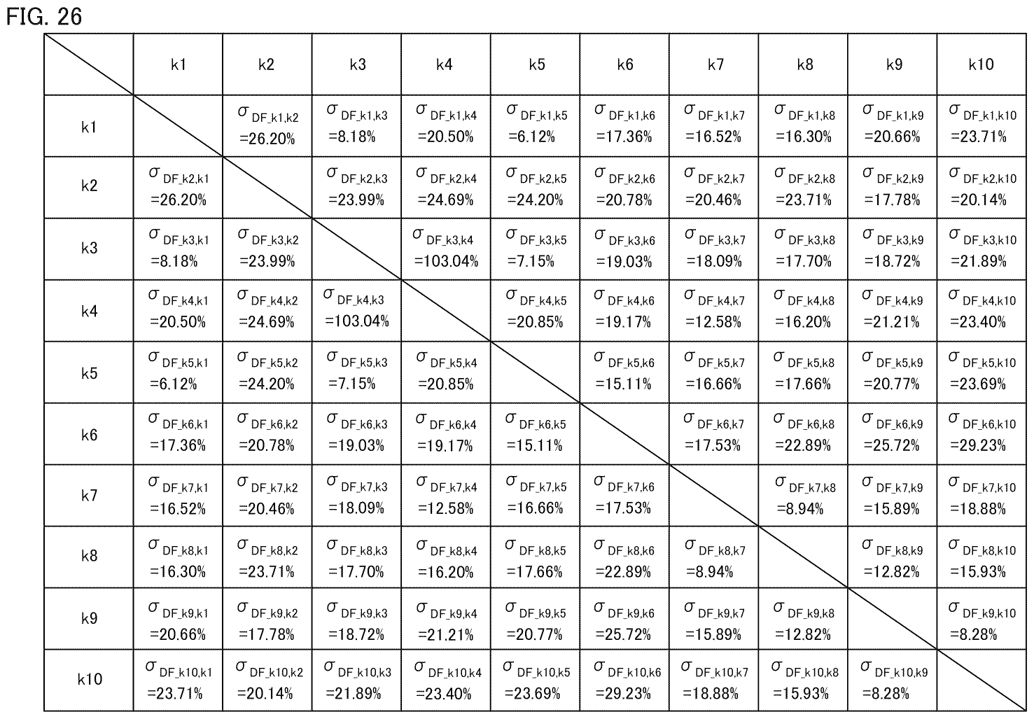

FIGS. 26 to 28 are first to third diagrams respectively showing the results of judging whether or not the curves k1 to k20 shown in FIGS. 19 to 25 are different from one another.

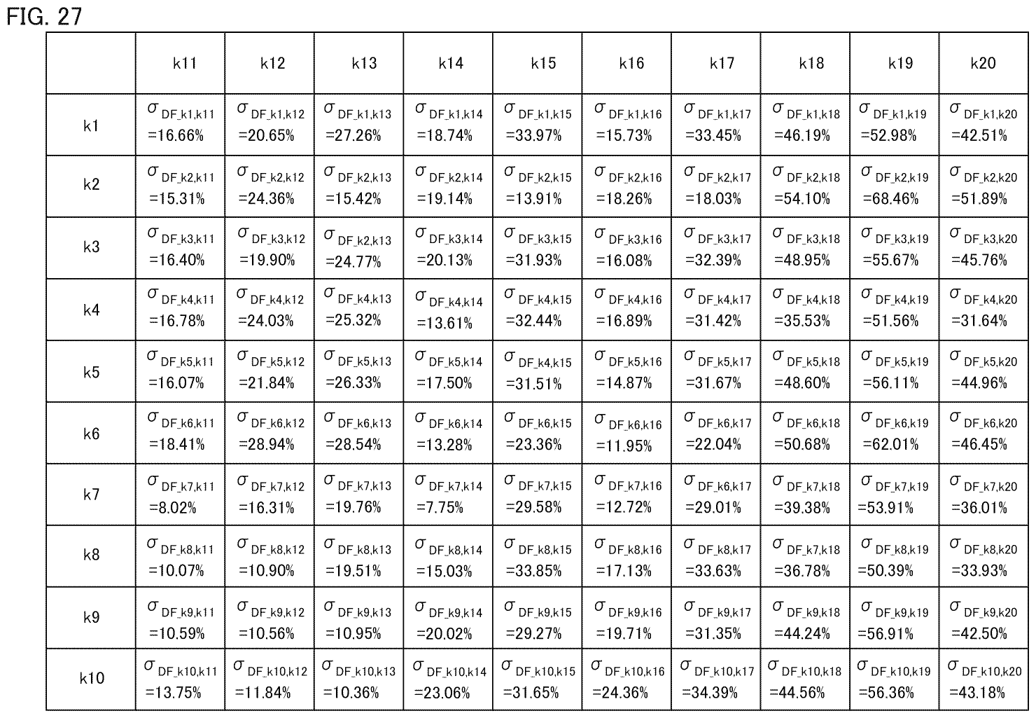

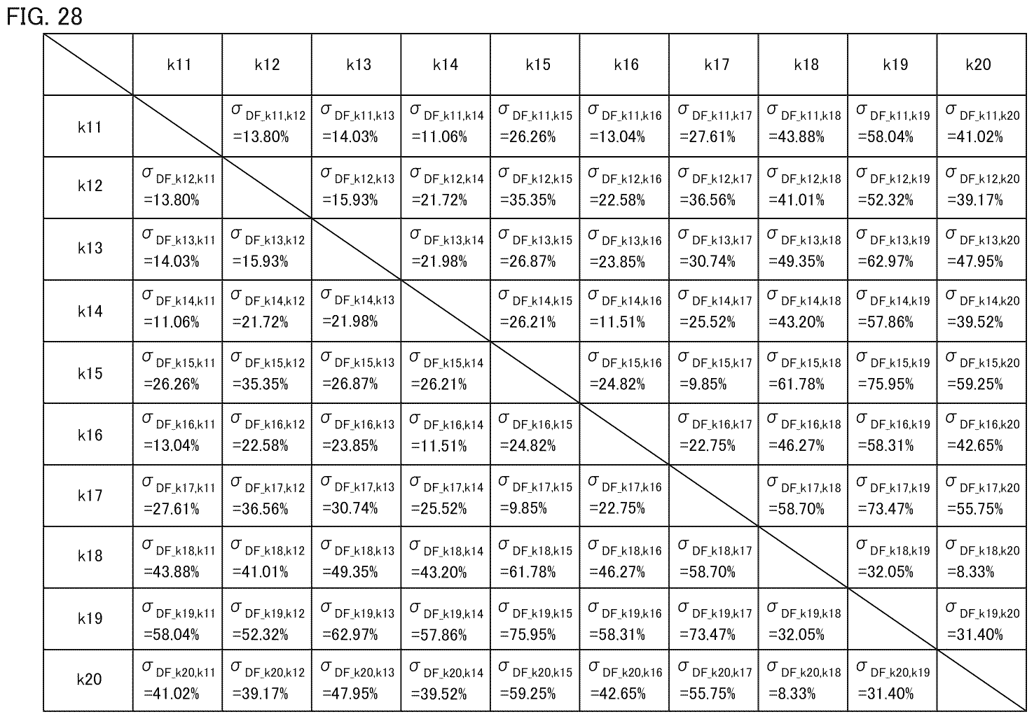

FIG. 26 shows the results of judging whether or not ten curves k1 to k10 are different from one another. FIG. 27 shows the results of judging whether or not ten curves k1 to k10 and ten curves k11 to k20 are different from each other. FIG. 28 shows the results of judging whether or not ten curves k11 to k20 are different from one another.

The judging of whether the 20 curves k1 to k20 are or not different from each other is performed by judging whether the two curves are or not different for all combinations (₂₀C₂=190) in when selecting two different curves from the 20 curves k1 to k20.

Then, it is executed by the following method to judge whether two different curves CURᵢ, CURⱼ (i≠j) are or not different. A difference DFₖ between an integral value ITG_{k_i} and an integral value ITG_{k_j} in one class Clsₖ is calculated using equation (1) based on a plurality of integral values ITG_{1_i} to ITG_{n_j} in the curve CURᵢ and a plurality of integral values ITG_{1_j} to ITG_{n_j} in the curve CUR, and this is performed for all classes Cls₁ to Clsₙ to calculate n differences DF₁ to DFₙ, and a judging whether two different curves CURᵢ, CUⱼ(i≠j) are different is performed by judging is made as to whether the standard deviation σ_{DFk} of the calculated n differences DF₁ to DFₙ is or not greater than a threshold value σₜₕ (=6%).

Referring to FIG. 26, when two different curves are selected from 10 curves k1 to k10, all of the 45 standard deviations σ_{DFk1, k2} to σ_{DFk9, k10} of the differences for all 45 combinations of two different curves are greater than the threshold value σₜₕ (=6%).

Referring to FIG. 27, when two different curves are selected from 10 curves k1 to k10 and 10 curves k11 to k20, all of the 100 standard deviations σ_{DFk1, k11} to σ_{DFk10, k20} of the differences for all 100 combinations of two different curves are greater than the threshold value σₜₕ (=6%).

Referring to FIG. 28, when two different curves are selected from 10 curves k11 to k20, all of the 45 standard deviations σ_{DFk11, k12} to σDF_{k19, k20} of the differences for all 45 combinations of two different curves are greater than the threshold value σₜₕ (=6%).

Therefore, when two different curves are selected from the 20 curves k1 to k20, all of the 190 standard deviations σ_{DFk1, k2} to σ_{DFk19, k20} of the differences for all of 190 combinations of two different curves are greater than the threshold value σₜₕ (=6%).

Therefore, the 20 curves k1 to k20 are mutually different curves. When it is judged that the 20 curves k1 to k20 are mutually different, the curves k1 to k20 are curves for uniquely identifying Shochu No. 1 to Shochu No. 20, respectively. The curves k1 to k20 are also fingerprints that represent feature amounts based on integral values for Shochu No. 1 to Shochu No. 20, respectively.

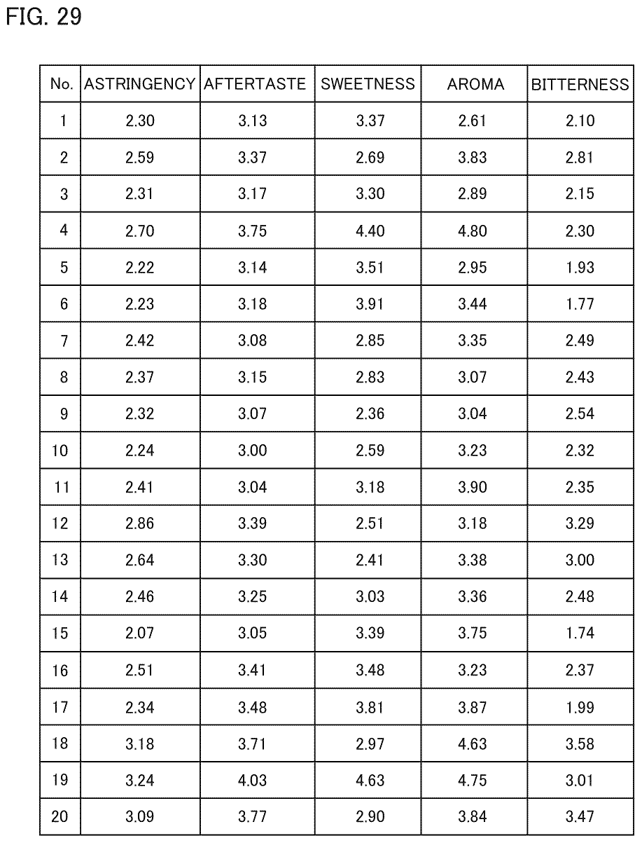

FIG. 29 is a diagram showing the results of diagnosis of “astringency,” “aftertaste,” “sweetness,” “aroma,” and “bitterness” for Shochu No. 1 to No. 20 shown in Table 2.

The values of “astringency”, “aftertaste”, “sweetness”, “aroma” and “bitterness” for Shochu No. 1 to No. 20 shown in FIG. 29 are the diagnosis results obtained by the taste diagnostic unit 217 using coefficients k₁=1.5, k₂=2, k₃=3000, k₄=1.5, k₅=2000, k₆=1.5, k₇=1.5 and k₈=0.4.

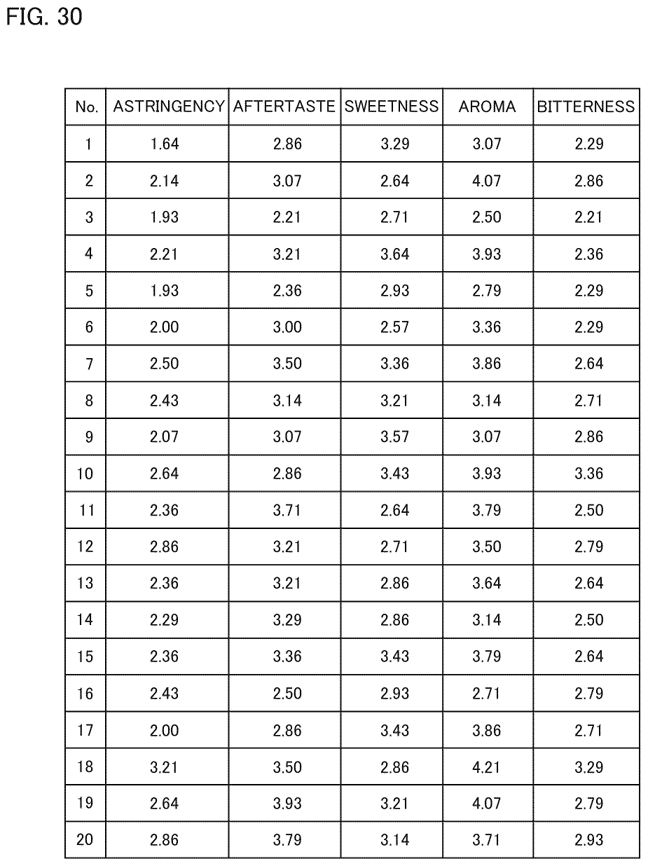

FIG. 30 is a diagram showing other diagnosis results of the “astringency”, “aftertaste”, “sweetness”, “aroma” and “bitterness” of Shochu No. 1 to No. 20 shown in Table 2.

The values of “astringency”, “aftertaste”, “sweetness”, “aroma” and “bitterness” for Shochu No. 1 to No. 20 shown in FIG. 30 are the average values of “astringency”, “aftertaste”, “sweetness”, “aroma” and “bitterness” judged by seven people.

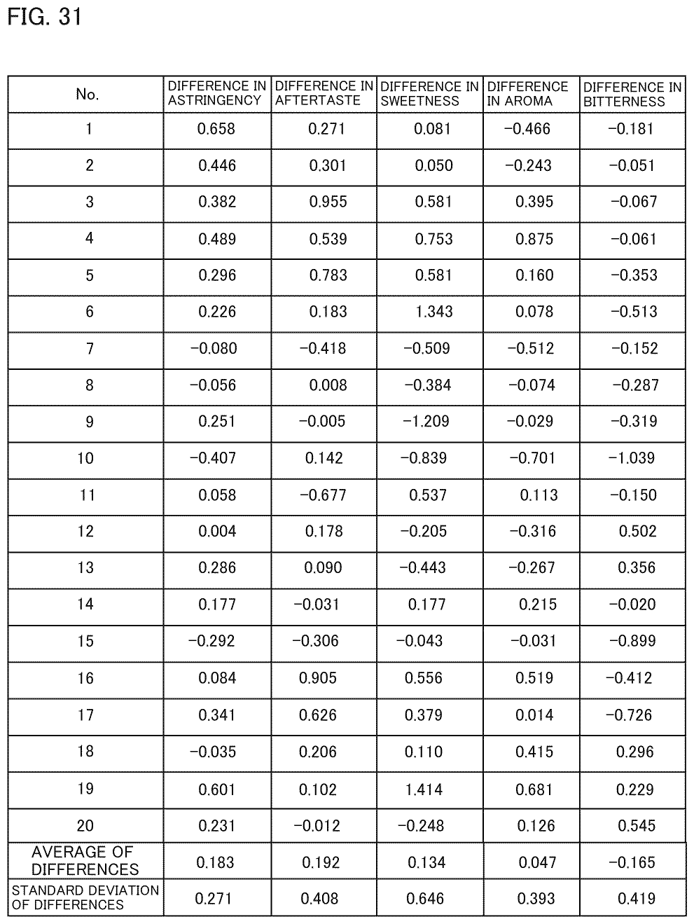

FIG. 31 is a diagram showing the differences between the respective values of the diagnosis results for Shochu No. 1 to No. 20 shown in FIG. 29 and the respective values of the diagnosis results for Shochu No. 1 to No. 20 shown in FIG. 30.

The differences between “astringency”, “aftertaste”, “sweetness”, “aroma” and “bitterness” shown in FIG. 31 consist of the subtraction results obtained by subtracting each of values of “astringency”, “aftertaste”, “sweetness”, “aroma” and “bitterness” shown in FIG. 30 from each of values of “astringency”, “aftertaste”, “sweetness”, “aroma” and “bitterness” shown in FIG. 29.

Therefore, in FIG. 31, when the difference is a positive value, it indicates that each value of “astringency”, “aftertaste”, “sweetness”, “aroma” and “bitterness” diagnosed by the taste diagnostic unit 217 is greater than each value of “astringency”, “aftertaste”, “sweetness”, “aroma” and “bitterness” diagnosed by a human and when the difference is a negative value, it indicates that the values of “astringency”, “aftertaste”, “sweetness”, “aroma” and “bitterness” diagnosed by the taste diagnostic unit 217 are smaller than the values of “astringency”, “aftertaste”, “sweetness”, “aroma” and “bitterness” diagnosed by a human.

Referring to FIG. 31, for Shochu of No. 1 to No. 20, the average value of the difference in “astringency” is 0.183, the average value of the difference in “aftertaste” is 0.192, the average value of the difference in “sweetness” is 0.134, the average value of the difference in “aroma” is 0.047, and the average value of the difference in “bitterness” is −0.165.

In addition, for Shochu of No. 1 to No. 20, the standard deviation of the difference in “astringency” is 0.271, the standard deviation of the difference in “aftertaste” is 0.408, the standard deviation of the difference in “sweetness” is 0.646, the standard deviation of the difference in “aroma” is 0.393, and the standard deviation of the difference in “bitterness” is 0.419.

As a result, the average deviation of the “astringency”, “aftertaste”, “sweetness”, “aroma” and “bitterness” diagnosed by the taste diagnostic unit 217 from the “astringency”, “aftertaste”, “sweetness”, “aroma” and “bitterness” diagnosed by humans was 19.2% or less, and the variation from the average deviation was 0.646 or less.

Therefore, it was found that the taste diagnostic unit 217 can diagnose the “astringency”, “aftertaste”, “sweetness”, “aroma” and “bitterness” of shochu in the same manner as a human being, by using the above-mentioned method.

### (2) Grape

The grapes used to create the curve CUR were Crimson Seedless (skin+body), Green Seedless (skin+body), Shine Muscat (skin+body), Crimson Seedless (body), Green Seedless (body) and Shine Muscat (body).

Each of Crimson Seedless (skin+flesh), Green Seedless (skin+flesh) and Shine Muscat (skin+flesh) are the juice of grapes that have been pressed together with the skin and flesh, while each of Crimson Seedless (flesh), Green Seedless (flesh) and Shine Muscat (flesh) are the juice of grapes that have been pressed only with the flesh.

In addition, the measurement conditions of the cyclic voltammograms CVG for Crimson Seedless (skin+flesh), Green Seedless (skin+flesh), Shine Muscat (skin+flesh), Crimson Seedless (flesh), Green Seedless (flesh) and Shine Muscat (flesh) are the same as the measurement conditions shown in Table 3.

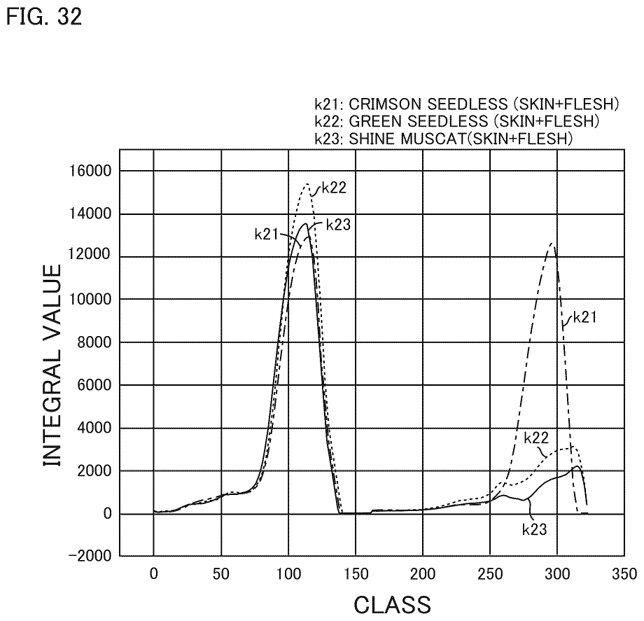

FIG. 32 is a diagram showing integral value spectra about Crimson Seedless (skin+flesh), Green Seedless (skin+flesh) and Shine Muscat (skin+flesh).

Referring to FIG. 32, each of the curves k21 to k23 is a curve CUR (index curve) which the diagnostic device 2 created by the above-mentioned method. The curve k21 shows the integral value spectrum of Crimson Seedless (skin+flesh), the curve k22 shows the integral value spectrum of Green Seedless (skin+flesh), and the curve k23 shows the integral value spectrum of Shine Muscat (skin+flesh).

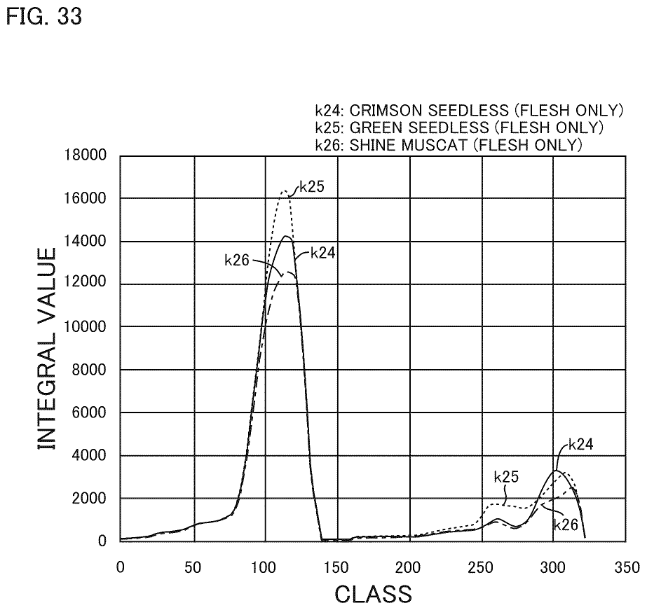

FIG. 33 is a diagram showing integral value spectra for Crimson Seedless (flesh only), Green Seedless (flesh only), and Shine Muscat (flesh only).

Referring to FIG. 33, each of the curves k24 to k26 is a curve CUR (index curve) which the diagnostic device 2 created by the above-mentioned method. The curve k24 shows the integral value spectrum of Crimson Seedless (flesh only), the curve k25 shows the integral value spectrum of Green Seedless (flesh only), and the curve k26 shows the integral value spectrum of Shine Muscat (flesh only).

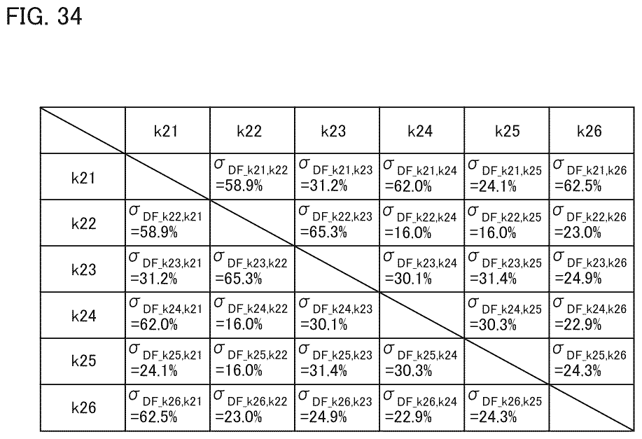

FIG. 34 is a diagram showing the result of judgment as to whether or not the curves k21 to k26 shown in FIG. 32 and FIG. 33 are different from each other.

Whether or not the curves k21 to k26 are different from one another is judged by executing a judgment as to whether or not any two different curves among the curves k21 to k26 are different for all combinations of two different curves among the curves k21 to k26.

There are 15 combinations of two different curves among the six curves k21 to k26: (k21, k22), (k21, k23), (k21, k24), (k21, k25), (k21, k26), (k22, k23), (k22, k24), (k22, k25), (k22, k26), (k23, k24), (k23, k25), (k23, k26), (k24, k25), (k24, k26), and (k25, k26).

Referring to FIG. 34, the standard deviation σ_{DF_k21, k22} of the difference between the multiple integral values on the curve k21 and the multiple integral values on the curve k22 is σ_{DF_k21, k22}=58.9%, the standard deviation σ_{DF_k21, k23} of the difference between the multiple integral values on the curve k21 and the multiple integral values on the curve k23 is σ_{DF_k21, k23}=31.2%, the standard deviation σ_{DF_k21, k24} of the difference between the multiple integral values on the curve k21 and the multiple integral values on the curve k24 is σ_{DF_k21, k24}=62.0%, the standard deviation σ_{DF_k21, k25} of the difference between the multiple integral values on the curve k21 and the multiple integral values on the curve k25 is σ_{DF_k21, k25}=24.1%, and the standard deviation σ_{DF_k21, k26} of the difference between the multiple integral values on the curve k21 and the multiple integral values on the curve k26 is σ_{DF_k21, k25}=62.5%.

Furthermore, the standard deviation σ_{DF_k22, k23} of the difference between the multiple integral values on the curve k22 and the multiple integral values on the curve k23 is σ_{DF_k22, k23}=65.3%, the standard deviation σ_{DF_k22, k24} of the difference between the multiple integral values on the curve k22 and the multiple integral values on the curve k24 is σ_{DF_k22, k24}=16.0%, the standard deviation σ_{DF_k22, k25} of the difference between the multiple integral values on the curve k22 and the multiple integral values on the curve k25 is σ_{DF_k22, k25}=16.0%, and the standard deviation σ_{DF_k22, k26} of the difference between the multiple integral values on the curve k22 and the multiple integral values on the curve k26 is σ_{DF_k22, k26}=23.0%.

Furthermore, the standard deviation σ_{DF_k23, k24} of the difference between the multiple integral values on the curve k23 and the multiple integral values on the curve k24 is σ_{DF_k23, k24}=30.1%, the standard deviation σ_{DF_k23, k25} of the difference between the multiple integral values on the curve k23 and the multiple integral values on the curve k25 is σ_{DF_k23, k25}=31.4%, and the standard deviation σ_{DF_k23, k26} of the difference between the multiple integral values on the curve k23 and the multiple integral values on the curve k26 is σ_{DF_k23, k26}=24.9%.

Furthermore, the standard deviation σ_{DF_k24, k25} of the difference between the multiple integral values on the curve k24 and the multiple integral values on the curve k25 is σ_{DF_k24, k25}=30.3%, and the standard deviation σ_{DF_k24, k26} of the difference between the multiple integral values on the curve k24 and the multiple integral values on the curve k26 is σ_{DF_k24, k26}=22.9%.

Furthermore, the standard deviation σ_{DF_k25, k26} of the difference between the multiple integral values on the curve k25 and the multiple integral values on the curve k26 is σ_{DF_k25, k26}=24.3%.

As a result, the standard deviation σ_{DF_k21, k22} (=58.9%) of the differences, the standard deviation σ_{DF_k21, k23} (=31.2%) of the differences, the standard deviation σ_{DF_k21, k24} (=62.0%) of the differences, the standard deviation σ_{DF_k21, k25} (=24.1%) of the differences, the standard deviation σ_{DF_k21, k26} (=62.5%) of the differences, the standard deviation σ_{DF_k22, k23} (=65.3%) of the differences, the standard deviation σ_{DF_k22, k24} (=16.0%) of the differences, the standard deviation σ_{DF_k22, k25} (=16.0%) of the differences, the standard deviation σ_{DF_k22,k26} (=23.0%) of differences, the standard deviation σ_{DF_k23,k24} (=30.1%) of differences, the standard deviation σ_{DF_k23,k25} (=31.4%) of differences, the standard deviation σ_{DF_k23,k26} (=24.9%) of differences, the standard deviation σ_{DF_k24,k25} (=30.3%) of differences, the standard deviation σDF_k24,k26 (=22.9%) of differences, and the standard deviation σ_{DF_k25,k26} (=24.3%) of differences are all greater than the threshold value σₜₕ (=15%).

Therefore, the two curves k21, k22 are different, the two curves k21, k23 are different, the two curves k21, k24 are different, the two curves k21, k25 are different, the two curves k21, k26 are different, the two curves k22, k23 are different, the two curves k22, k24 are different, the two curves k22, k25 are different, the two curves k22, k26 are different, the two curves k23, k24 are different, the two curves k23, k25 are different, the two curves k23, k26 are different, the two curves k24, k25 are different, the two curves k24, k26 are different, and the two curves k25, k26 are different. Therefore, the curves k21 to k26 are mutually different curves.

When it is judged that the six curves k21 to k26 are different from each other, the curves k21 to k26 are curves for uniquely identifying Crimson Seedless (flesh+skin), Green Seedless (flesh+skin), Shine Muscat (flesh+skin), Crimson Seedless (flesh only), Green Seedless (flesh only) and Shine Muscat (flesh only), respectively. And, the curves k21 to k26 are fingerprints representing the feature amounts by integral values for Crimson Seedless (flesh+skin), Green Seedless (flesh+skin), Shine Muscat (flesh+skin), Crimson Seedless (flesh only), Green Seedless (flesh only) and Shine Muscat (flesh only), respectively.

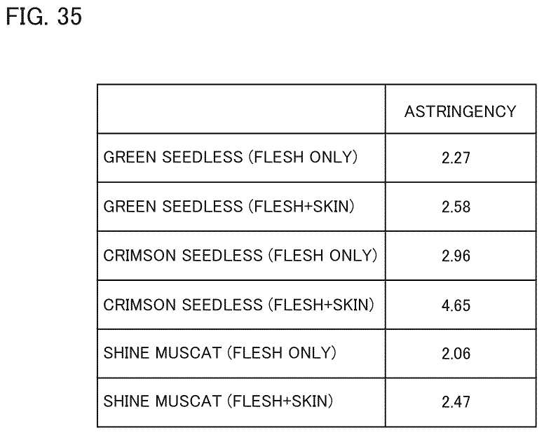

FIG. 35 is a diagram showing the diagnosis results of “astringency” for Green Seedless (flesh only), Crimson Seedless (flesh only), Shine Muscat (flesh only), Green Seedless (flesh+skin), Crimson Seedless (flesh+skin), and Shine Muscat (flesh+skin).

Note that the “Astringency” shown in FIG. 35 is calculated using L(−)_sum_th, M(−)_sum_th, and H(−)_sum_th calculated based on the integral value in a specified potential section (=class) in the range from −1362 mV to −2501 mV, with the threshold value Vₜₕ of the above-mentioned potential V being set to “−1362 mV.”

Referring to FIG. 35, the astringency of green seedless (flesh only) is 2.27, and the astringency of green seedless (flesh+skin) is 2.58.

Additionally, the astringency of Crimson Seedless (flesh only) is 2.96, ang the astringency of Crimson Seedless (flesh+skin) is 4.65.

Furthermore, the astringency of Shine Muscat (flesh only) is 2.06, and the astringency of Shine Muscat (flesh+skin) is 2.47.

As a result, the “astringency” value increases in the order of Shine Muscat (flesh only), Green Seedless (flesh only), Shine Muscat (flesh+skin), Green Seedless (flesh+skin), Crimson Seedless (flesh only), and Crimson Seedless (flesh+skin).

In addition, the astringency of Green Seedless (flesh only) is less than that of Green Seedless (flesh+skin), the astringency of Crimson Seedless (flesh only) is less than that of Crimson Seedless (flesh+skin), and the astringency of Shine Muscat (flesh only) is less than that of Shine Muscat (flesh+skin).

Therefore, “the astringency” of Green Seedless (flesh only), Crimson Seedless (flesh only) and Shine Muscat (flesh only), which are juices made by squeezing grape flesh, is less than the astringency of Green Seedless (flesh+skin), Crimson Seedless (flesh+skin) and Shine Muscat (flesh+skin), which are juices made by squeezing grape flesh and skin.

Therefore, by diagnosing the astringency of grapes using the taste diagnostic unit 217, it was found that the grape skin is a factor that increases “the astringency”.

### [Relationship Between Taste Diagnosis and Number of Integral Values for Shochu] (I) Number of Integral Values: 138

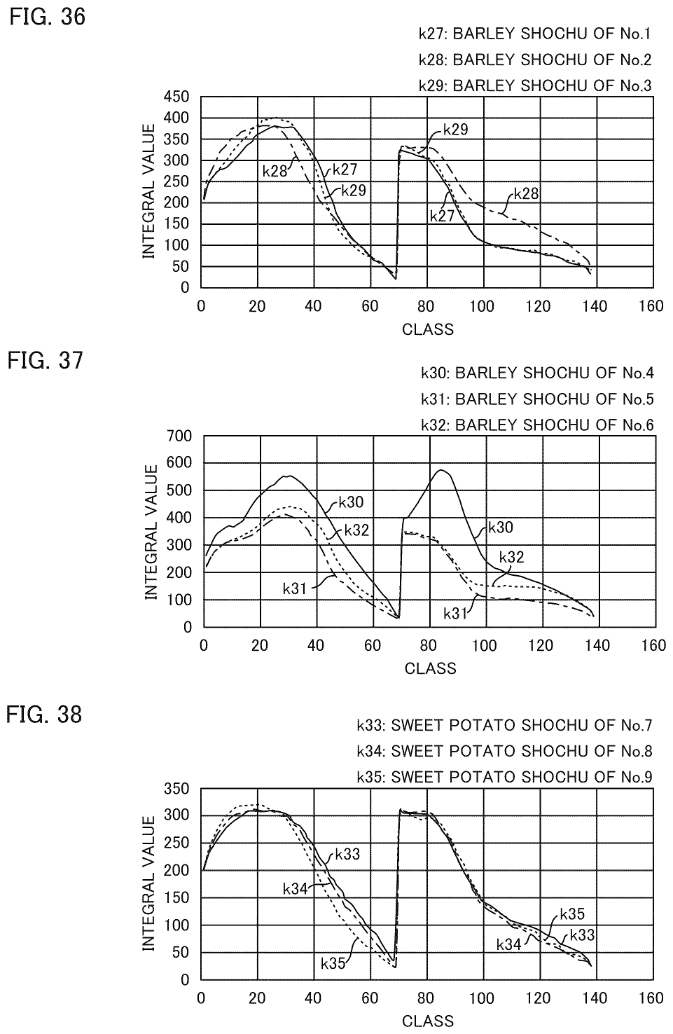

FIG. 36 is a diagram showing integral value spectra for the shochu with samples No. 1 to No. 3 shown in Table 2, in the case where the number of integral values is 138.

Referring to FIG. 36, each of the curves k27 to k29 is a curve CUR (index curve) created by the creation unit 215 of the diagnostic device 2 using the method described above. Curve k27 shows the integral value spectrum of the barley shochu of sample No. 1, curve k28 shows the integral value spectrum of the barley shochu of sample No. 2, and curve k29 shows the integral value spectrum of the barley shochu of sample No. 3.

FIG. 37 is a diagram showing integral value spectra for the shochu of samples No. 4 to No. 6 shown in Table 2, in the case where the number of integral values is 138.

Referring to FIG. 37, each of the curves k30 to k32 is a curve CUR (index curve) created by the creation unit 215 of the diagnostic device 2 using the method described above. Curve k30 shows the integral value spectrum of the barley shochu of sample No. 4, curve k31 shows the integral value spectrum of the barley shochu of sample No. 5, and curve k32 shows the integral value spectrum of the barley shochu of sample No. 6.

FIG. 38 is a diagram showing integral value spectra for the shochu of samples No. 7 to No. 9 shown in Table 2 when the number of integral values is 138.

Referring to FIG. 38, each of the curves k33 to k35 is a curve CUR (index curve) created by the creation unit 215 of the diagnostic device 2 using the method described above. A curve k33 indicates the integral value spectrum of the sweet potato shochu of sample No. 7, a curve k34 indicates the integral value spectrum of the sweet potato shochu of sample No. 8, and a curve k35 indicates the integral value spectrum of the sweet potato shochu of sample No. 9.

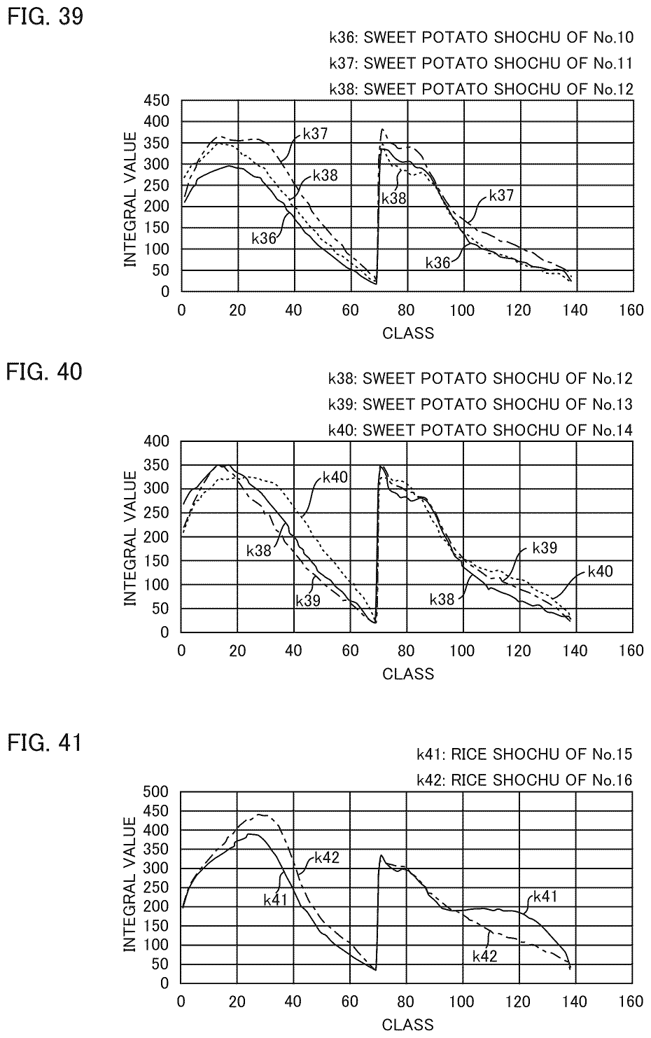

FIG. 39 is a diagram showing integral value spectra for the shochu of samples No. 10 to No. 12 shown in Table 2, in the case where the number of integral values is 138.

Referring to FIG. 39, each of the curves k36 to k38 is a curve CUR (index curve) created by the creation unit 215 of the diagnostic device 2 using the above-mentioned method. The curve k36 shows the integral value spectrum of the sweet potato shochu of sample No. 10, the curve k37 shows the integral value spectrum of the sweet potato shochu of sample No. 11, and the curve k38 shows the integral value spectrum of the sweet potato shochu of sample No. 12.

FIG. 40 is a diagram showing integral value spectra for the shochu of samples No. 12 to No. 14 shown in Table 2 when the number of integrals is 138.

Referring to FIG. 40, each of the curves k38 to k40 is a curve CUR (index curve) created by the creation unit 215 of the diagnostic device 2 using the above-mentioned method. The curve k38 shows the integral value spectrum of the sweet potato shochu of sample No. 12, the curve k39 shows the integral value spectrum of the sweet potato shochu of sample No. 13, and the curve k40 shows the integral value spectrum of the sweet potato shochu of sample No. 14.

FIG. 41 is a diagram showing integral value spectra for the shochu of samples No. 15 and No. 16 shown in Table 2, in the case where the number of integral values is 138.

Referring to FIG. 41, each of the curves k41 and k42 is a curve CUR (index curve) created by the creation unit 215 of the diagnostic device 2 using the above-mentioned method. The curve k41 shows the integral value spectrum of the rice shochu of sample No. 15, and the curve k42 shows the integral value spectrum of the rice shochu of sample No. 16.

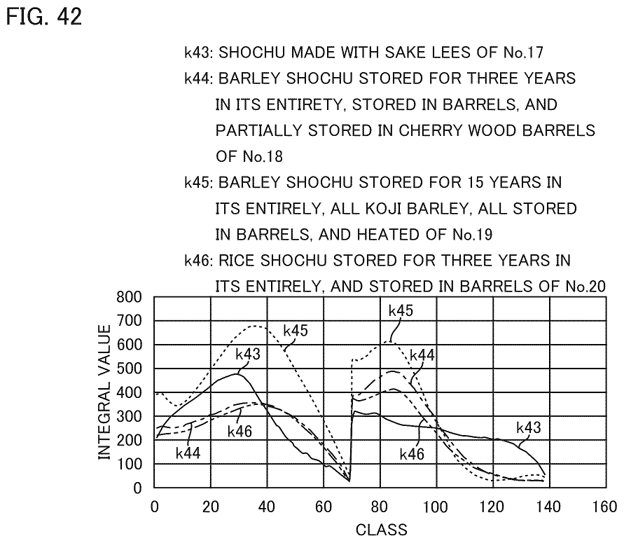

FIG. 42 is a diagram showing integral value spectra for the shochu of samples No. 17 to No. 20 shown in Table 2, in the case where the number of integral values is 138.

Referring to FIG. 42, each of the curves k43 to k46 is a curve CUR (index curve) created by the creation unit 215 of the diagnostic device 2 by the above-mentioned method. The curve k43 shows the integral value spectrum of the shochu made with sake lees of sample No. 17, the curve k44 shows the integral value spectrum of the barley shochu stored for three years in its entirety, stored in barrels, and partially stored in cherry wood barrels of sample No. 18, the curve k45 shows the integral value spectrum of the barley shochu stored for 15 years in its entirety, all koji barley, all stored in barrels, and heated, of sample No. 19, and the curve k46 shows the integral value spectrum of the rice shochu stored for three years in its entirety, and stored in barrels of sample No. 20.

In FIG. 36 to FIG. 42, each of the classes 1 to 138 is composed of a predetermined potential section (integral value extraction potential) of 36.2 mV. Classes 1 to 69 are classes in the positive predetermined potential section, and classes 70 to 138 are classes in the negative predetermined potential section.

As a result, it is possible to calculate the sums SMT_Low(+), SMT__{Middle}(+), and SMT__{High(+)} of the integral values in the positive predetermined potential sections can be calculated based on the integral value spectra (curves CUR__{Low_k27} to CUR__{Low_k46}, CUR__{Middle_k27} to CUR__{Middle_k46}, and CUR__{High_k27} to CUR__{High_k46} similar to curves k27 to k46) in which all classes are composed of a predetermined potential section (integral value extraction potential) of 36.2 mV. Therefore, it is possible to calculate Body Index (+) based on the integral value spectra (curves CUR__{Low_k27} to CUR__{Low_k46}, CUR__{Middle_k27} to CUR__{Middle_k46}, and CUR__{High_k27} to CUR__{High_k46} similar to curves k27 to k46) in which all classes are composed of a predetermined potential section (integral value extraction potential) of 36.2 mV to diagnose the astringency ASTG of the shochu using equation (5). In addition, it is possible to calculate H(+)_sum (=SMT__{High(+)}) based on the integral value spectra (curves CUR__{Low_k27} to CUR__{Low_k46}, CUR__{Middle_k27} to CUR__{Middle_k46}, and CUR__{High_k27} to CUR__{High_k46} similar to curves k27 to k46) in which all classes are composed of a predetermined potential section (integral value extraction potential) of 36.2 mV to diagnose the sweetness SWT of the shochu using equation (7). Furthermore, it is possible to calculate H(−)_sum (=SMT__{High(+)}) based on the integral value spectra (curves CUR__{Low_k27} to CUR__{Low_k46}, CUR__{Middle_k27} to CUR__{Middle_k46}, and CUR__{High_k27} to CUR__{High_k46} similar to curves k27 to k46) in which all classes are composed of a predetermined potential section (integral value extraction potential) of 36.2 mV to diagnose the aroma SCT of the shochu using equation (8). As a result, the bitterness BIT of shochu can be diagnosed by equation (9).

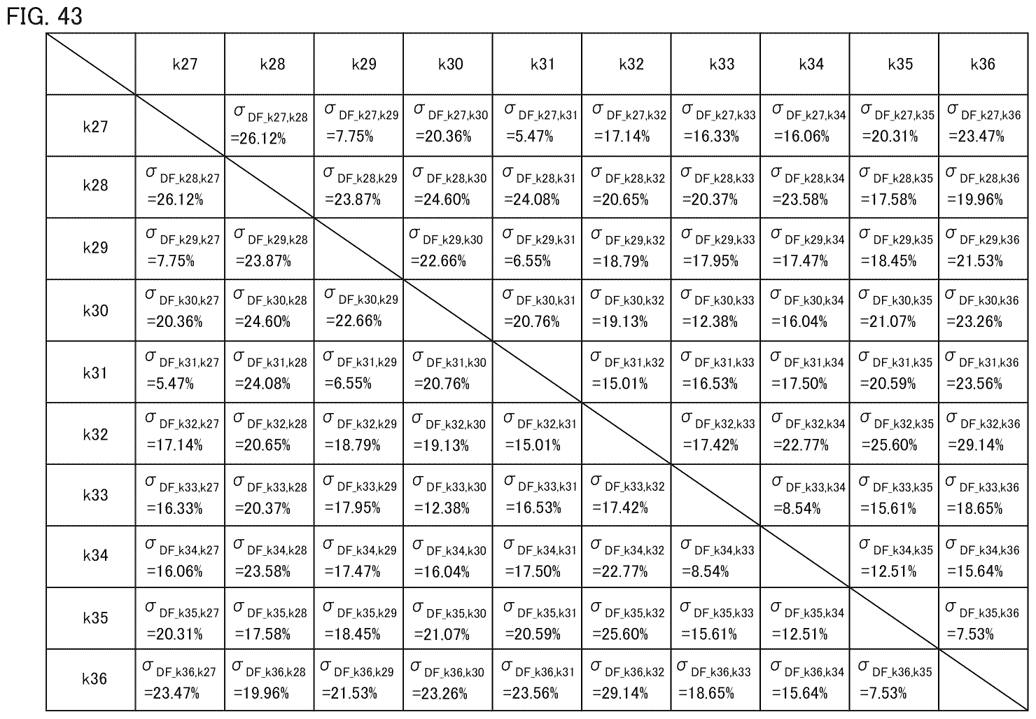

FIGS. 43 to 45 are first to third diagrams respectively showing the results of judging whether or not the curves k27 to k46 shown in FIGS. 36 to 42 are different from one another.

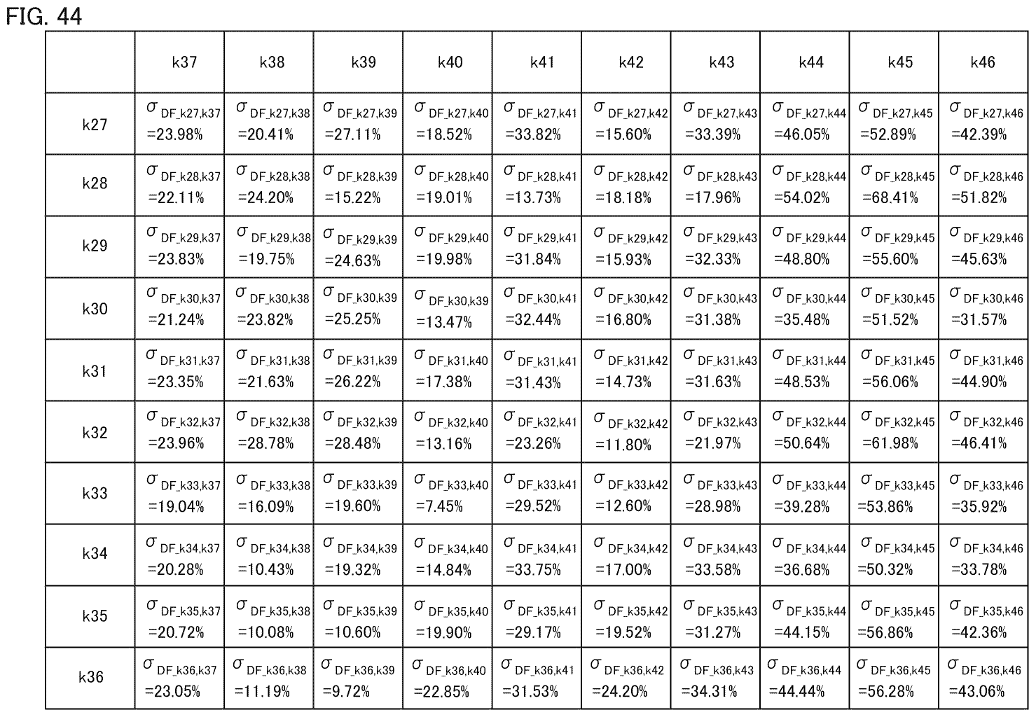

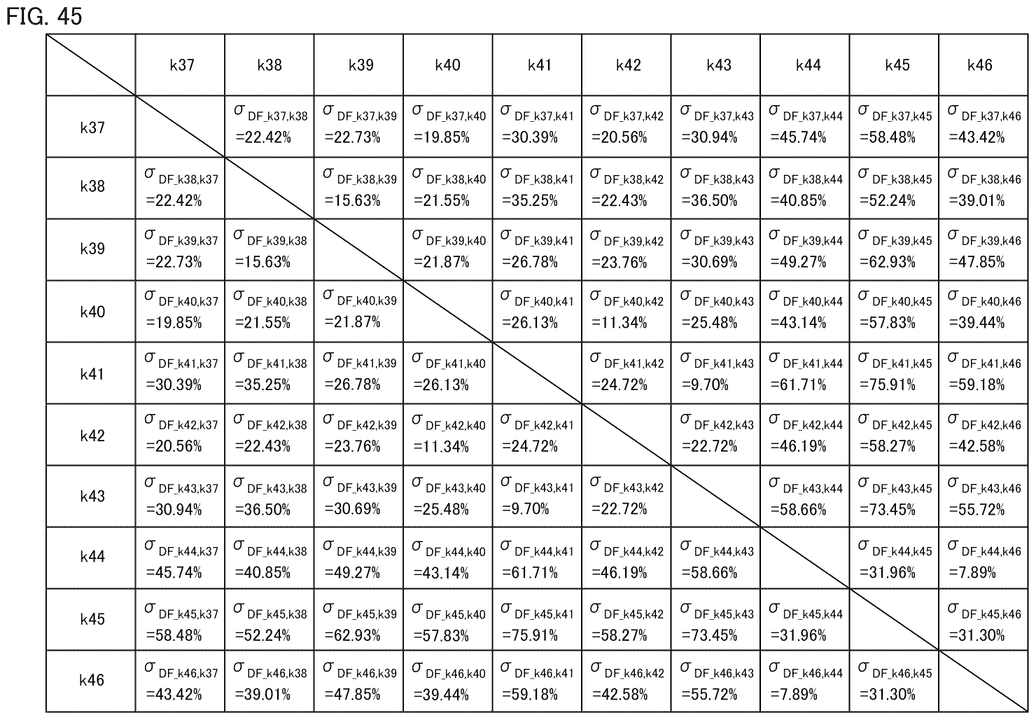

FIG. 43 shows the results of the judging as to whether or not ten curves k27 to k36 are different from each other, FIG. 44 shows the results of the judging as to whether or not ten curves k27 to k36 and ten curves k37 to k46 are different from each other, and FIG. 45 shows the results of the judging as to whether or not ten curves k37 to k46 are different from each other.

The judgment of whether the 20 curves k27 to k46 are or not different from each other is performed by judging whether the two curves are or not different for all combinations (₂₀C₂=190) in when selecting two different curves from the 20 curves k27 to k46.

Referring to FIG. 43, when two different curves are selected from the 10 curves k27 to k36, all of the 45 standard deviations σ_{DF_k27, k28} to σ_{DF_k35, k36} of the differences for all of 45 combinations of two different curves are greater than the threshold value σₜₕ (=5%).

Referring to FIG. 44, when two different curves are selected from 10 curves k27 to k36 and 10 curves k37 to k46, all of the 100 standard deviations σ_{DF_k27, k37} to σ_{DF_k36, k46} of the differences or all of 100 combinations of two different curves are greater than the threshold value σₜₕ (=5%).

Referring to FIG. 45, when two different curves are selected from 10 curves k37 to k46, all of the 45 standard deviations σ_{DF_k37, k38} to σ_{DF_k45, k46} of the differences for all of 45 combinations of two different curves are greater than the threshold value σₜₕ (=5%).

Therefore, when two different curves are selected from the 20 curves k27 to k46, all of the 190 standard deviations σ_{DF_k27, k28} to σ_{DF_k45, k46} of the differences for all of 190 combinations of two different curves are greater than the threshold value σₜₕ (=5%).

Therefore, the 20 curves k27 to k46 are mutually different curves. When it is judged that the 20 curves k27 to k46 are mutually different, the curves k27 to k46 are curves for uniquely identifying the shochu No. 1 to No. 20, respectively. When the number of integral values is 138, the curves k27 to k46 are fingerprints representing feature amounts by integral values for the shochu No. 1 to No. 20, respectively.

### (II) Number of Integral Values: 70

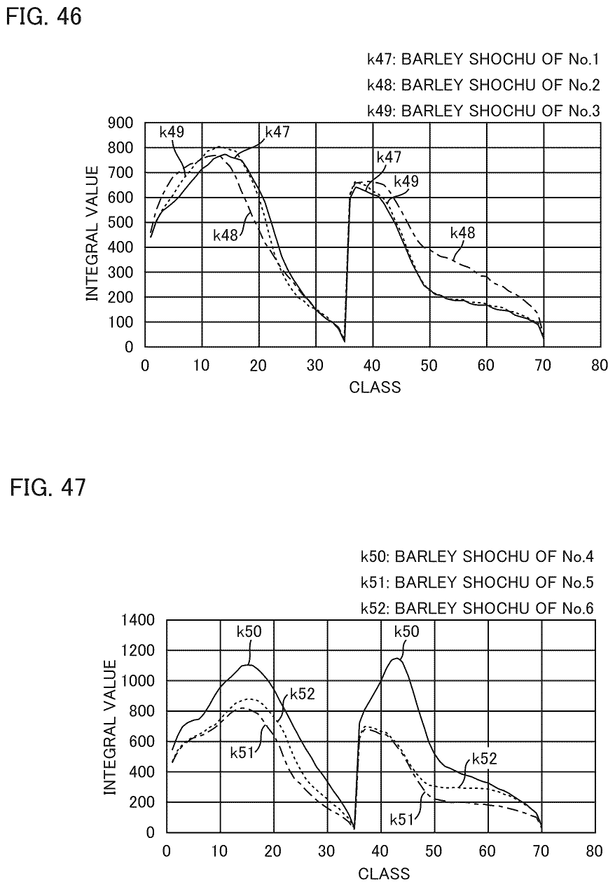

FIG. 46 is a diagram showing integral value spectra for the shochu of samples No. 1 to No. 3 shown in Table 2, in the case where the number of integral values is 70.

Referring to FIG. 46, each of the curves k47 to k49 is a curve CUR (index curve) created by the creation unit 215 of the diagnostic device 2 using the above-mentioned method. The curve k47 shows the integral value spectrum of the barley shochu of sample No. 1, the curve k48 shows the integral value spectrum of the barley shochu of sample No. 2, and the curve k49 shows the integral value spectrum of the barley shochu of sample No. 3.

FIG. 47 is a diagram showing integral value spectra for the shochu of samples No. 4 to No. 6 shown in Table 2, in the case where the number of integral values is 70.

Referring to FIG. 47, each of the curves k50 to k52 is a curve CUR (index curve) created by the creation unit 215 of the diagnostic device 2 using the above-mentioned method. The curve k50 shows the integral value spectrum of the barley shochu of sample No. 4, the curve k51 shows the integral value spectrum of the barley shochu of sample No. 5, and the curve k52 shows the integral value spectrum of the barley shochu of sample No. 6.

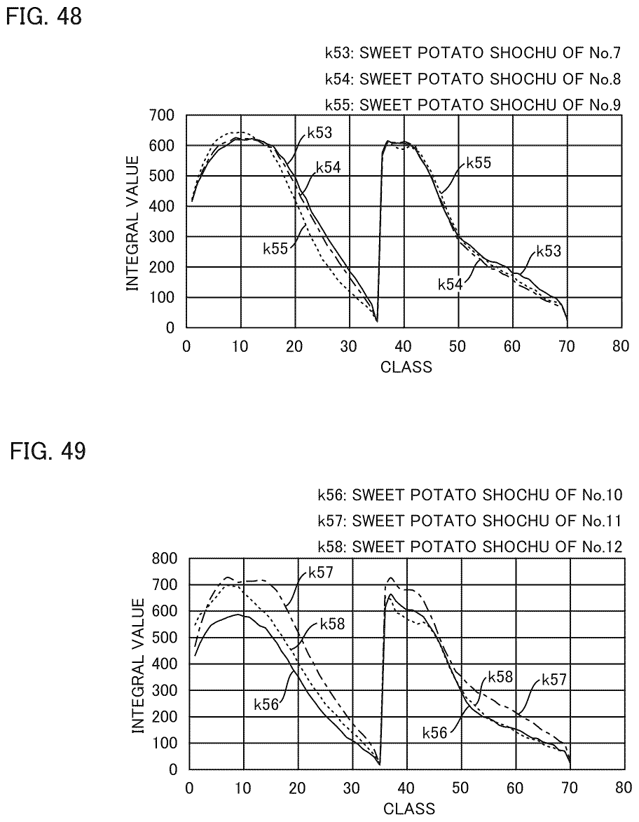

FIG. 48 is a diagram showing integral value spectra for the shochu of samples No. 7 to No. 9 shown in Table 2, in the case where the number of integral values is 70.

Referring to FIG. 48, each of the curves k53 to k55 is a curve CUR (index curve) created by the creation unit 215 of the diagnostic device 2 using the above-mentioned method. The curve k53 shows the integral value spectrum of the sweet potato shochu of sample No. 7, the curve k54 shows the integral value spectrum of the sweet potato shochu of sample No. 8, and the curve k55 shows the integral value spectrum of the sweet potato shochu of sample No. 9.

FIG. 49 is a diagram showing integral value spectra for the shochu of samples No. 10 to No. 12 shown in Table 2, in the case where the number of integral values is 70.

Referring to FIG. 49, each of the curves k56 to k58 is a curve CUR (index curve) created by the creation unit 215 of the diagnostic device 2 using the above-mentioned method. A curve k56 indicates the integral value spectrum of the sweet potato shochu of sample No. 10, a curve k57 indicates the integral value spectrum of the sweet potato shochu of sample No. 11, and a curve k58 indicates the integral value spectrum of the sweet potato shochu of sample No. 12.

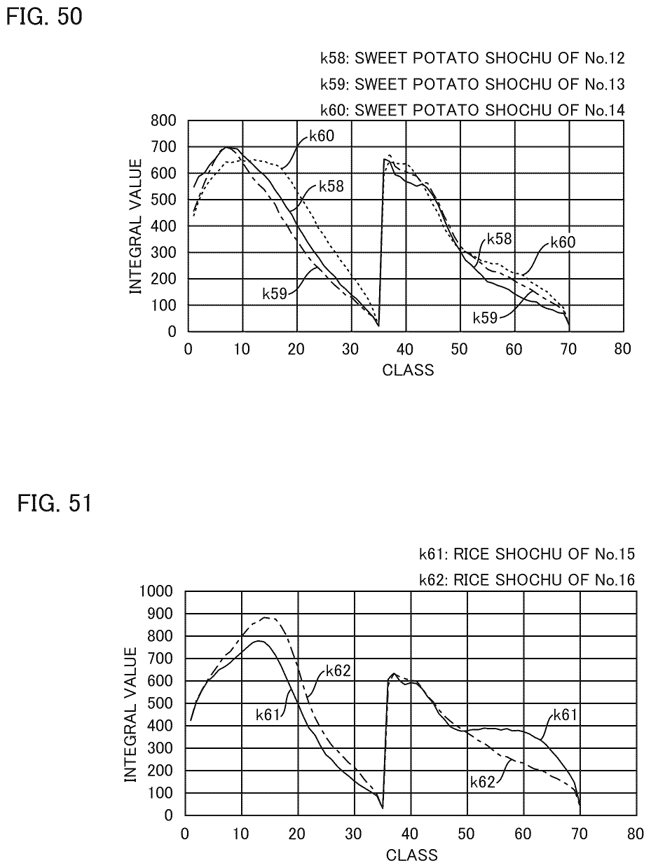

FIG. 50 is a diagram showing integral value spectra for the shochu of samples No. 12 to No. 14 shown in Table 2, in the case where the number of integral values is 70.

Referring to FIG. 50, each of the curves k58 to k60 is a curve CUR (index curve) created by the creation unit 215 of the diagnostic device 2 using the above-mentioned method. The curve k58 shows the integral value spectrum of the sweet potato shochu of sample No. 12, the curve k59 shows the integral value spectrum of the sweet potato shochu of sample No. 13, and the curve k60 shows the integral value spectrum of the sweet potato shochu of sample No. 14.

FIG. 51 is a diagram showing integral value spectra for the shochu of samples No. 15 and No. 16 shown in Table 2, in the case where the number of integral values is 70.

Referring to FIG. 51, each of the curves k61, k62 is a curve CUR (index curve) created by the creation unit 215 of the diagnostic device 2 using the above-mentioned method. The curve k61 shows the integral value spectrum of the rice shochu of sample No. 15, and the curve k62 shows the integral value spectrum of the rice shochu of sample No. 16.

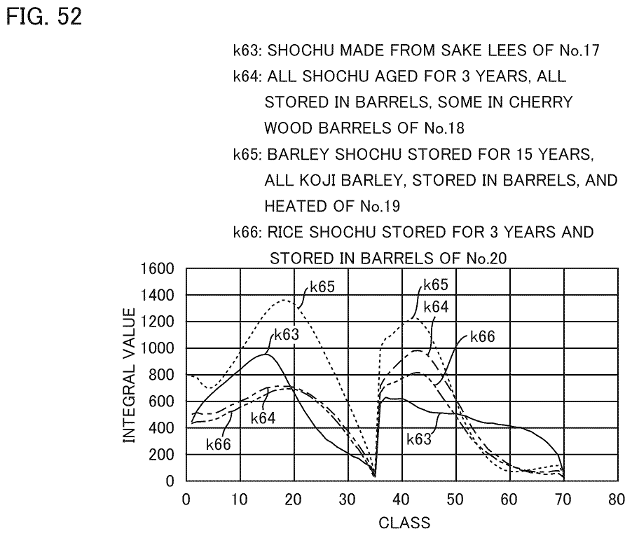

FIG. 52 is a diagram showing integral value spectra for the shochu of samples No. 17 to No. 20 shown in Table 2, in the case where the number of integral values is 70.

Referring to FIG. 52, each of the curves k63 to k66 is a curve CUR (index curve) created by the creation unit 215 of the diagnostic device 2 using the above-mentioned method. Curve k63 shows the integral value spectrum of shochu made from sake lees with sample No. 17, curve k64 shows the integral value spectrum of barley shochu for all shochu aged for 3 years, all stored in barrels, some in cherry wood barrels with sample No. 18, curve k65 shows the integral value spectrum of barley shochu stored for 15 years, all koji barley, stored in barrels, and heated with sample No. 19, and curve k66 shows rice shochu stored for 3 years and stored in barrels with sample No. 20.

In from FIG. 46 to FIG. 52, each of classes 1 to 34 has a predetermined potential section (integral value extraction potential) of 72.4 mV, class 35 has a predetermined potential section (integral value extraction potential) of 36.2 mV, each of classes 36 to 69 has a predetermined potential section (integral value extraction potential) of 72.4 mV, and class 70 has a predetermined potential section (integral value extraction potential) of 36.2 mV. Classes 1 to 35 are classes in the positive potential section, and classes 36 to 70 are classes in the negative potential section.

The reason why class 35 has a predetermined potential section (integral value extraction potential) of 36.2 mV, which is smaller than the predetermined potential section (integral value extraction potential) of 72.4 mV in each of classes 1 to 34, is to make the class 35 the “last class in the positive predetermined potential section.”

In other words, when class 35 is composed of the same predetermined potential section (integral value extraction potential) as the predetermined potential section (integral value extraction potential) of 72.4 mV in each of classes 1 to 34, the integral value in class 35 is the sum of the integral value in the positive predetermined potential section and the integral value in the negative predetermined potential section, and it is not possible to calculate the sums SMT__{Low(+)}, SMT__{Middle(+)}, and SMT__{High(+)} of the integral values in the positive predetermined potential section based on an integral value spectrum in which all classes are composed of the predetermined potential section (integral value extraction potential) of 72.4 mV. As a result, it is not possible to diagnose the astringency ASTG of shochu using formula (5) by calculating the Body Index (+) based on the integral value spectrum consisting of a predetermined potential section (integral value extraction potential) of 72.4 mV for all classes, it is not possible to diagnose the sweetness SWT of shochu using formula (7) by calculating H(+)_sum (=SMT_High(+)) based on the integral value spectrum consisting of a predetermined potential section (integral value extraction potential) of 72.4 mV for all classes, it is not possible to diagnose the aroma SCT of shochu using formula (8) by calculating H(−)_sum (=SMT_High(−)) based on the integral value spectrum consisting of a predetermined potential section (integral value extraction potential) of 72.4 mV for all classes, and as a result, it is not possible to diagnose the bitterness BIT of shochu using formula (9). Therefore, the class 35 has a predetermined potential section (integral value extraction potential) of 36.2 mV.

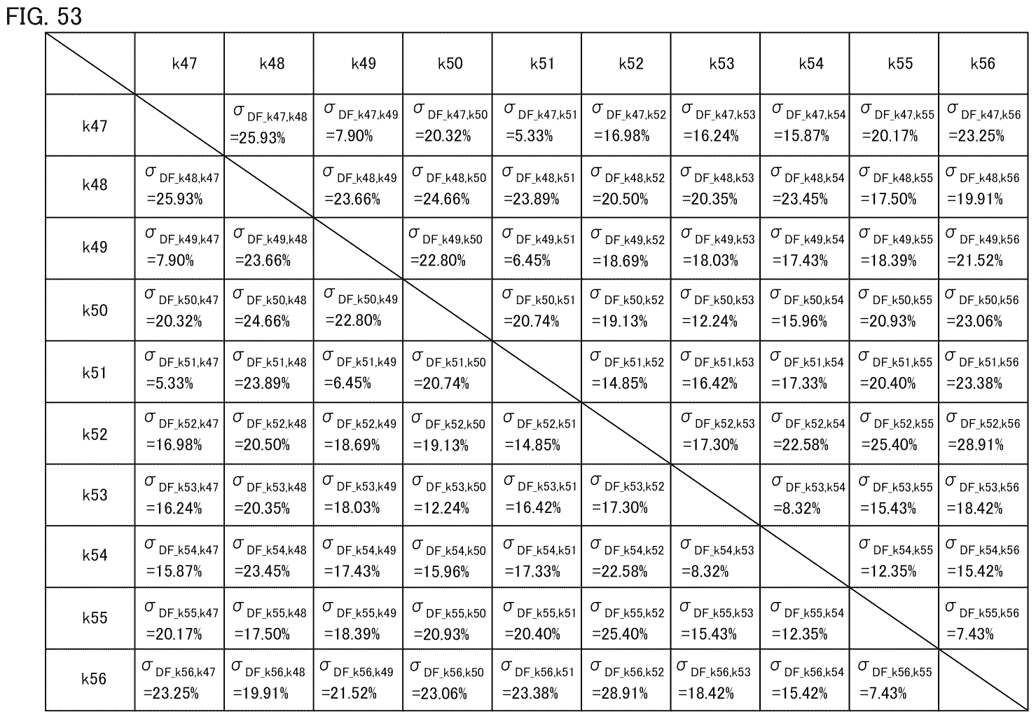

FIGS. 53 to 55 are first to third diagrams respectively showing the results of judging whether or not the curves k47 to k66 shown in FIGS. 46 to 52 are different from one another, respectively.

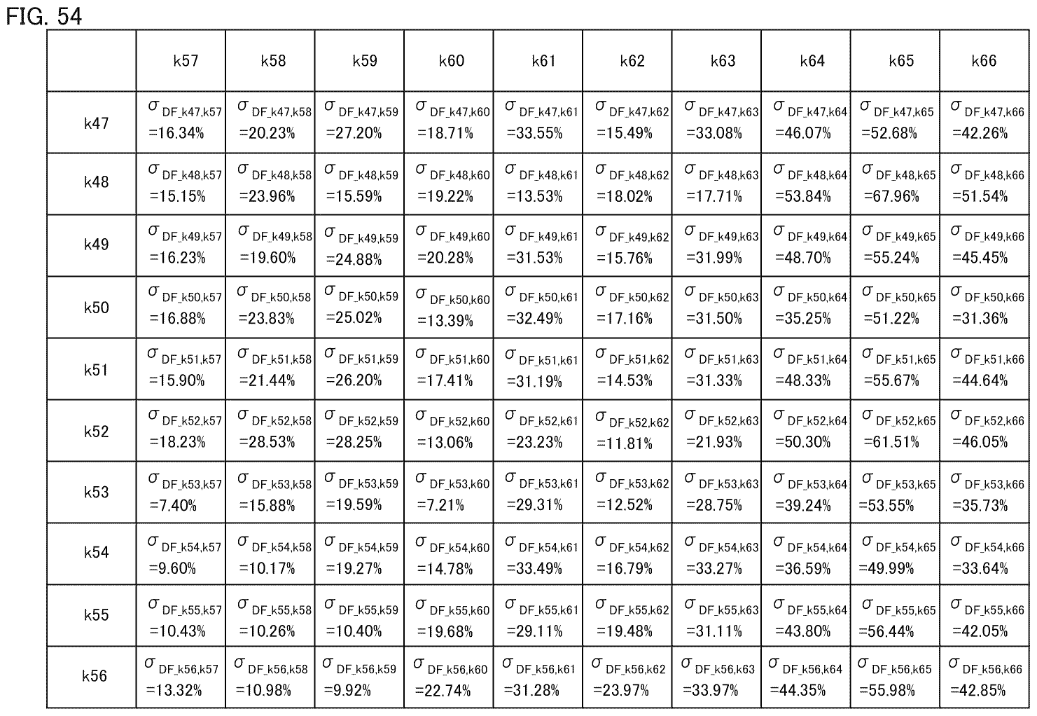

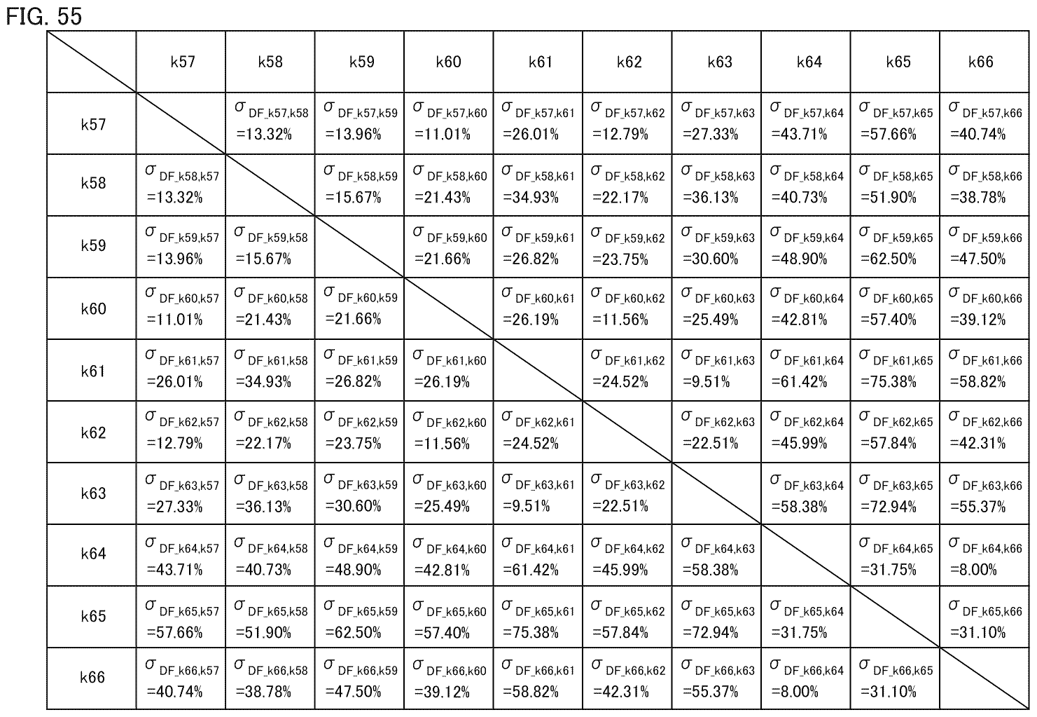

FIG. 53 shows the results of a judging as to whether or not ten curves k47 to k56 are different from each other, FIG. 54 shows the results of a judging as to whether or not ten curves k47 to k56 and ten curves k57 to k66 are different from each other, and FIG. 55 shows the results of a judging as to whether or not ten curves k57 to k66 are different from each other.

The judgment of whether the 20 curves k47 to k66 are or not different from each other is performed by judging whether the two different curves are or not different for all combinations (₂₀C₂=190) on when selecting two different curves from the 20 curves k47 to k66.

Referring to FIG. 53, when two different curves are selected from the ten curves k47 to k56, all of the 45 standard deviations σ_{DF_k47, k48} to σ_{DF_k55, k56} of the differences for all 45 combinations of two different curves are greater than the threshold value σₜₕ (=5%).

Referring to FIG. 54, when two different curves are selected from 10 curves k47 to k56 and 10 curves k57 to k66, all of the 100 standard deviations σ_{DF_k47, k57} to σ_{DF_k56, k66} of the differences for all of 100 combinations of two different curves are greater than the threshold value σₜₕ (=5%).

Referring to FIG. 55, when two different curves are selected from the ten curves k57 to k66, all of the 45 standard deviations σ_{DF_k57, k58} to σ_{DF_k65, k66} of the differences for all of 45 combinations of two different curves are greater than the threshold value σₜₕ (=5%).

Therefore, when two different curves are selected from the 20 curves k47 to k66, all of the 190 standard deviations σ_{DF_k47, k48} to σ_{DF_k65, k66} of the differences for all 190 combinations of two different curves are greater than the threshold value σₜₕ (=5%).

Therefore, the 20 curves k47 to k66 are mutually different curves. When it is judged that the 20 curves k47 to k66 are mutually different, the curves k47 to k66 are curves for uniquely identifying the shochu No. 1 to No. 20, respectively. When the number of integral values is 70, the curves k47 to k66 are fingerprints representing feature amounts based on integral values for the shochu No. 1 to No. 20, respectively.

Other explanations for the case where the number of integral values is 70 are the same as those for the case where the number of integral values is 138.

### (III) Number of Integral Values: 36

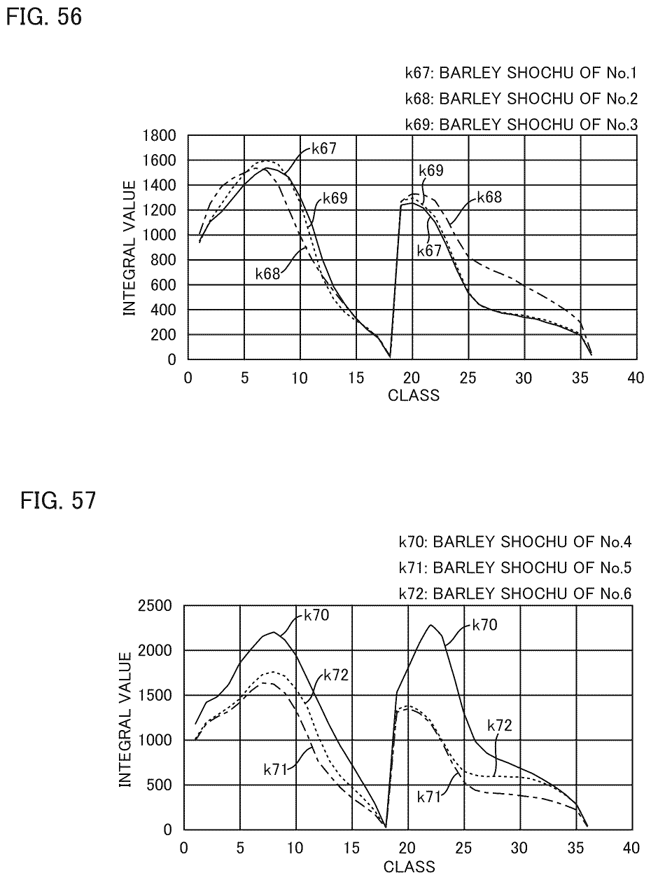

FIG. 56 is a diagram showing integral value spectra for the shochu of samples No. 1 to No. 3 shown in Table 2, in the case where the number of integral values is 36.

Referring to FIG. 56, each of the curves k67 to k69 is a curve CUR (index curve) created by the creation unit 215 of the diagnostic device 2 using the above-mentioned method. The curve k67 shows the integral value spectrum of the barley shochu of sample No. 1, the curve k68 shows the integral value spectrum of the barley shochu of sample No. 2, and the curve k69 shows the integral value spectrum of the barley shochu of sample No. 3.

FIG. 57 is a diagram showing integral value spectra for the shochu of samples No. 4 to No. 6 shown in Table 2, in the case where the number of integral values is 36.

Referring to FIG. 57, each of the curves k70 to k72 is a curve CUR (index curve) created by the creation unit 215 of the diagnostic device 2 using the above-mentioned method. The curve k70 shows the integral value spectrum of the barley shochu of sample No. 4, the curve k71 shows the integral value spectrum of the barley shochu of sample No. 5, and the curve k72 shows the integral value spectrum of the barley shochu of sample No. 6.

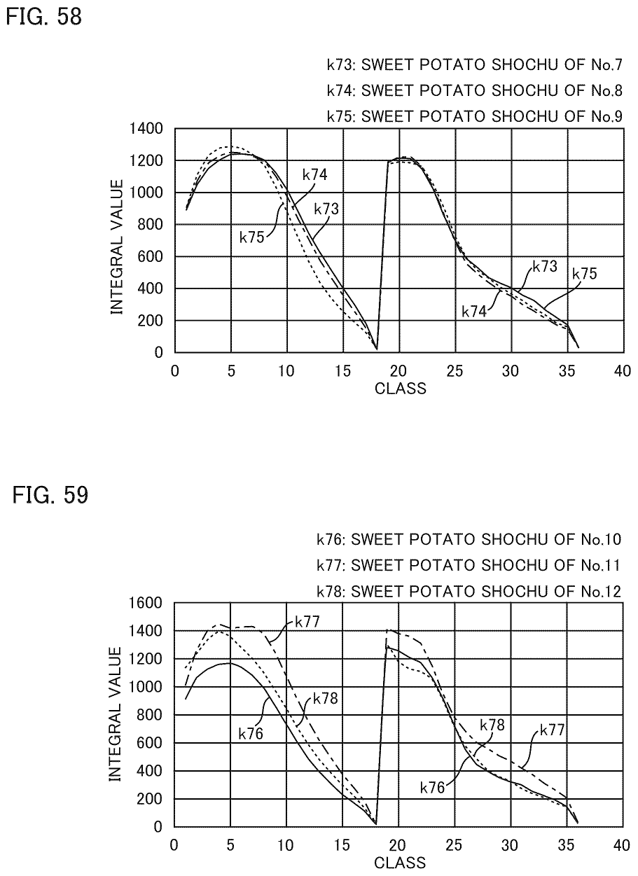

FIG. 58 is a diagram showing integral value spectra for the shochu of samples No. 7 to No. 9 shown in Table 2, in the case where the number of integral values is 36.

Referring to FIG. 58, each of the curves k73 to k75 is a curve CUR (index curve) created by the creation unit 215 of the diagnostic device 2 using the above-mentioned method. The curve k73 shows the integral value spectrum of the sweet potato shochu of sample No. 7, the curve k74 shows the integral value spectrum of the sweet potato shochu of sample No. 8, and the curve k75 shows the integral value spectrum of the sweet potato shochu of sample No. 9.

FIG. 59 is a diagram showing integral value spectra for the shochu of samples No. 10 to No. 12 shown in Table 2, in the case where the number of integral values is 36.

Referring to FIG. 59, each of the curves k76 to k78 is a curve CUR (index curve) created by the creation unit 215 of the diagnostic device 2 using the above-mentioned method. The curve k76 shows the integral value spectrum of the sweet potato shochu of sample No. 10, the curve k77 shows the integral value spectrum of the sweet potato shochu of sample No. 11, and the curve k78 shows the integral value spectrum of the sweet potato shochu of sample No. 12.

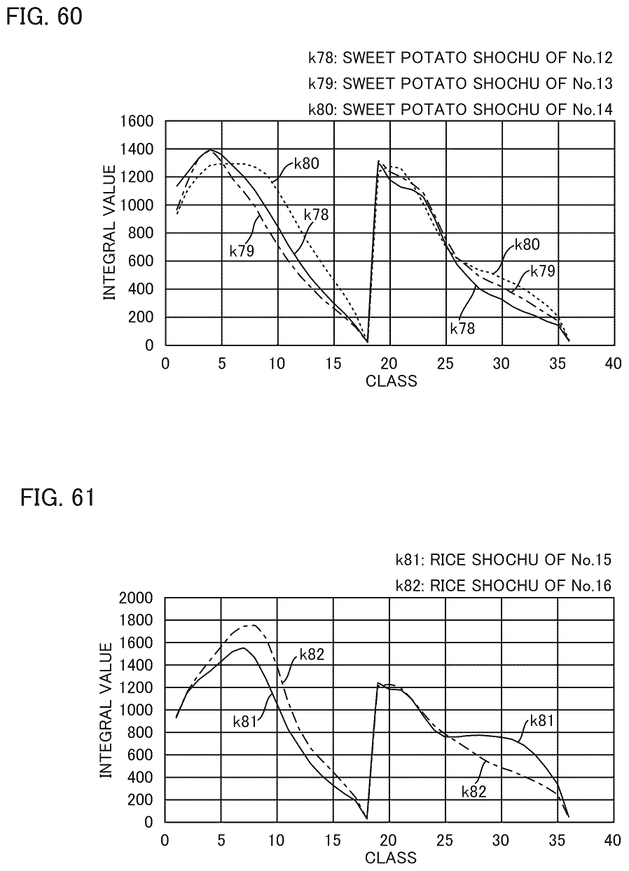

FIG. 60 is a diagram showing integral value spectra for the shochu of samples No. 12 to No. 14 shown in Table 2, in the case where the number of integral values is 36.

Referring to FIG. 60, each of the curves k78 to k80 is a curve CUR (index curve) created by the creation unit 215 of the diagnostic device 2 using the above-mentioned method. The curve k78 shows the integral value spectrum of the sweet potato shochu of sample No. 12, the curve k79 shows the integral value spectrum of the sweet potato shochu of sample No. 13, and the curve k80 shows the integral value spectrum of the sweet potato shochu of sample No. 14.

FIG. 61 is a diagram showing integral value spectra for the shochu of samples No. 15 to No. 16 shown in Table 2, in the case where the number of integral values is 36.

Referring to FIG. 61, each of the curves k81 to k82 is a curve CUR (index curve) created by the creation unit 215 of the diagnostic device 2 using the above-mentioned method. The curve k81 shows the integral value spectrum of the rice shochu of sample No. 15, and the curve k82 shows the integral value spectrum of the rice shochu of sample No. 16.

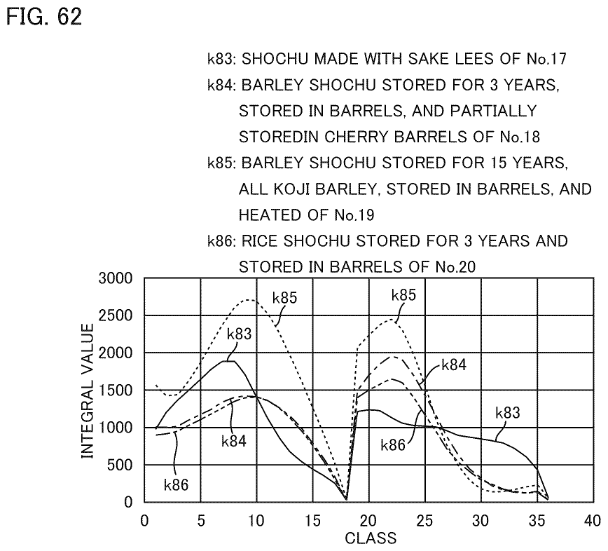

FIG. 62 is a diagram showing integral value spectra for the shochu of samples No. 17 to No. 20 shown in Table 2, in the case where the number of integral values is 36.

Referring to FIG. 62, each of the curves k83 to k86 is a curve CUR (index curve) created by the creation unit 215 of the diagnostic device 2 using the above-mentioned method. The curve k83 shows the integral value spectrum of shochu made with sake lees in sample No. 17, the curve k84 shows the integral value spectrum of sample No. 18, barley shochu stored for 3 years, stored in barrels, and partially stored in cherry wood barrels, the curve k85 shows the integral value spectrum of sample No. 19, barley shochu stored for 15 years, all koji barley, stored in barrels, and heated, and the curve k86 shows the integral value spectrum of sample No. 20, rice shochu stored for 3 years and stored in barrels.

In FIG. 56 to FIG. 62, each of classes 1 to 17 has a predetermined potential section (integral value extraction potential) of 144.8 mV, class 18 has a predetermined potential section (integral value extraction potential) of 36.2 mV, each of classes 19 to 35 has a predetermined potential section (integral value extraction potential) of 144.8 mV, and class 36 has a predetermined potential section (integral value extraction potential) of 36.2 mV. Classes 1 to 18 are classes in the positive potential section, and classes 19 to 36 are classes in the negative potential section.

The reason why class 18 has a predetermined potential section (integral value extraction potential) of 36.2 mV, which is smaller than the predetermined potential section (integral value extraction potential) of 144.8 mV in each of classes 1 to 17, is to make class 18 the “last class in the positive predetermined potential section.”

In other words, when class 18 is composed of the same predetermined potential section (integral value extraction potential) as the predetermined potential section (integral value extraction potential) of 144.8 mV in each of classes 1 to 17, the integral value in class 18 is the sum of the integral value in the positive predetermined potential section and the integral value in the negative predetermined potential section, and it is not possible to calculate the sums SMT__{Low(+)}, SMT__{Middle(+)}, and SMT__{High(+)} of the integral values in the positive predetermined potential section based on an integral value spectrum in which all classes are composed of the predetermined potential section (integral value extraction potential) of 144.8 mV. As a result, it is not possible to diagnose the astringency ASTG of shochu using formula (5) by calculating the Body index(+) based on the integral value spectrum consisting of a predetermined potential section (integral value extraction potential) of 144.8 mV for all classes, it is not possible to diagnose the sweetness SWT of shochu using formula (7) by calculating H(+)_sum (=SMT__{High(+)}) based on the integral value spectrum consisting of a predetermined potential section (integral value extraction potential) of 144.8 mV for all classes, it is not possible to diagnose the aroma SCT of shochu using formula (8) by calculating H(−)_sum (=SMT__{High(-)}) based on the integral value spectrum consisting of a predetermined potential section (integral value extraction potential) of 144.8 mV for all classes, and as a result, it is not possible to diagnose the bitterness BIT of shochu using formula (9). Therefore, the class 35 has a predetermined potential section (integral value extraction potential) of 36.2 mV.

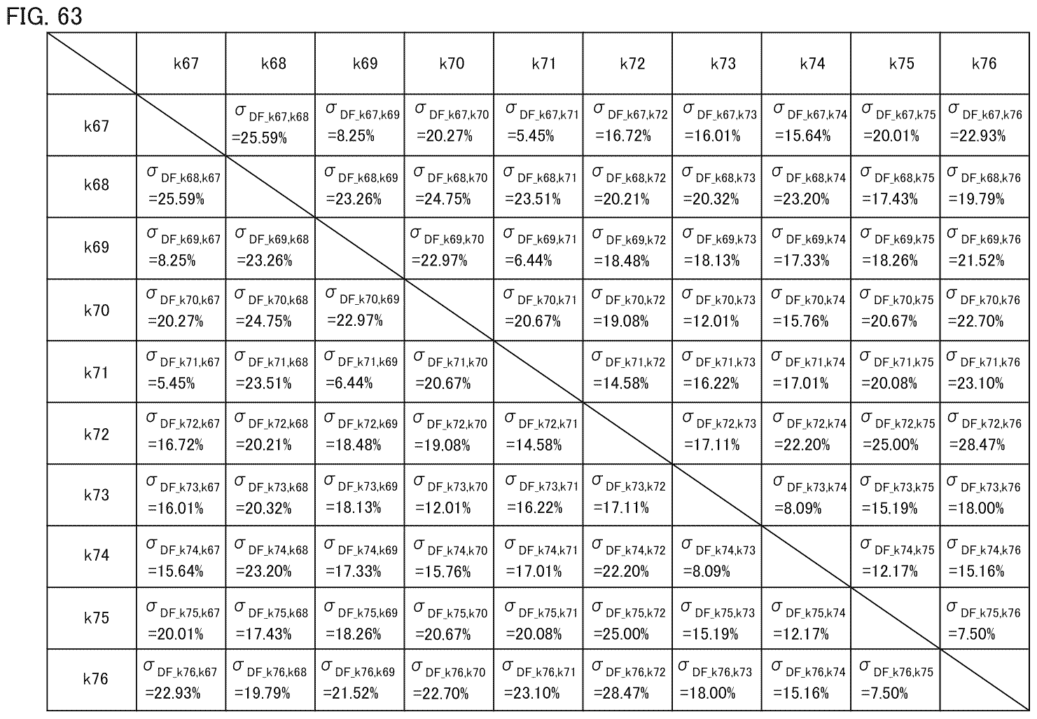

FIGS. 63 to 65 are first to third diagrams respectively showing the results of judging whether the curves k67 to k86 shown in FIGS. 56 to 62 are or not different from one another.

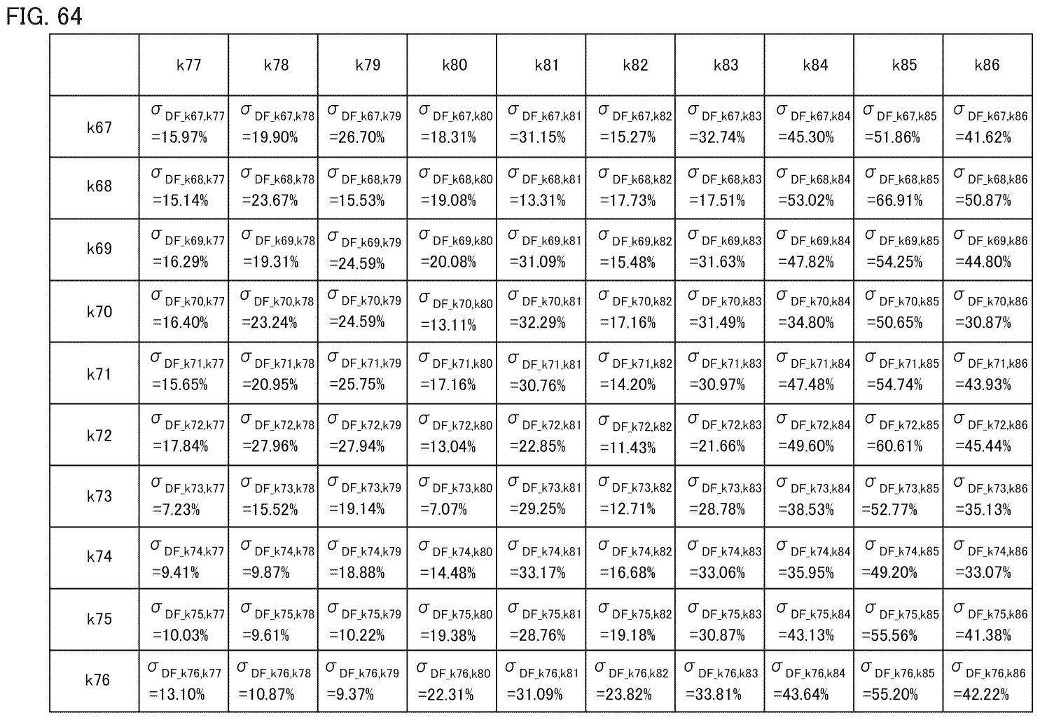

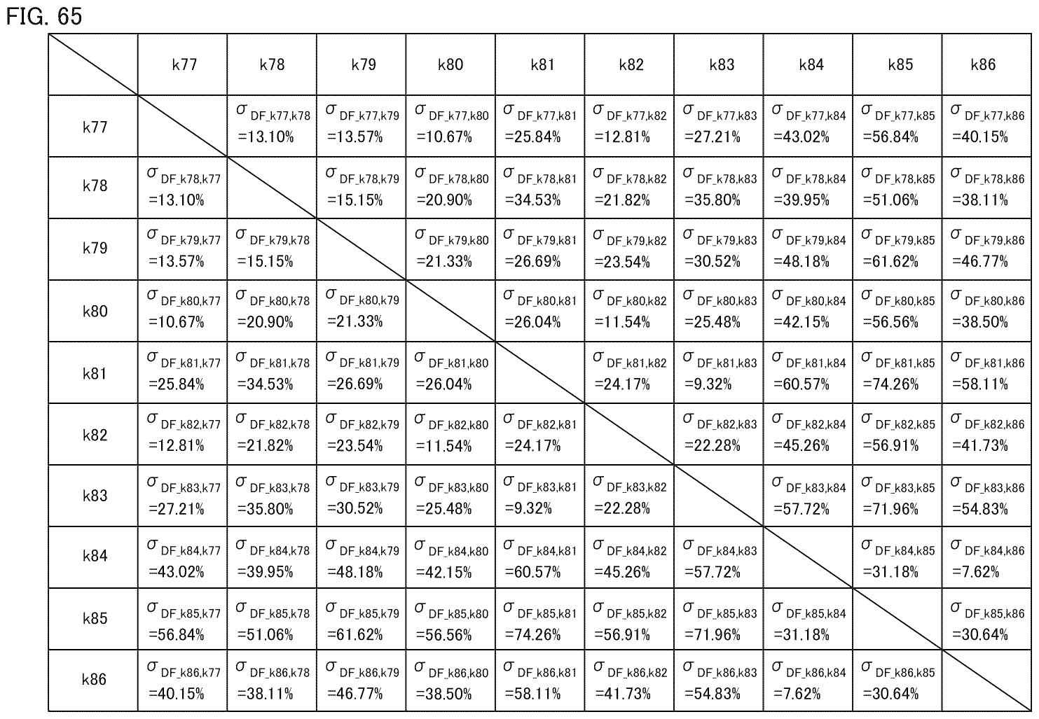

FIG. 63 shows the results of the judgment as to whether or not ten curves k67 to k76 are different from each other, FIG. 64 shows the results of the judgment as to whether or not ten curves k67 to k76 and ten curves k77 to k86 are different from each other, and FIG. 65 shows the results of the judgment as to whether or not ten curves k77 to k86 are different from each other.

The judgment of whether the 20 curves k67 to k86 are or not different from each other is performed by judging whether the two different curves are or not different for all combinations (₂₀C₂=190) when selecting two different curves from the 20 curves k67 to k86.

Referring to FIG. 63, when two different curves are selected from the ten curves k67 to k76, all of the 45 standard deviations σ_{DF_k67, k68} to σ_{DF_k75, k76} of the differences for all 45 combinations of two different curves are greater than the threshold value σₜₕ (=5%).

Referring to FIG. 64, when two different curves are selected from 10 curves k67 to k76 and 10 curves k77 to k86, all of the 100 standard deviations σ_{DF_k67, k77} to σ_{DF_k76, k86} of the differences for all of 100 combinations of two different curves are greater than the threshold value σₜₕ (=5%).

Referring to FIG. 65, when two different curves are selected from the ten curves k77 to k86, all of the 45 standard deviations σ_{DF_k77, k78} to σ_{DF_k85, k86} of the differences for all of 45 combinations of two different curves are greater than the threshold value σₜₕ (=5%).

Therefore, when two different curves are selected from the 20 curves k67 to k86, all of the 190 standard deviations σ_{DF_k67, k68} to σ_{DF_k85, k86} of the differences for all of 190 combinations of two different curves are greater than the threshold value σₜₕ (=5%).

Therefore, the 20 curves k67 to k86 are mutually different curves. When it is judged that the 20 curves k67 to k86 are mutually different, the curves k67 to k86 are curves for uniquely identifying the shochu No. 1 to No. 20, respectively. When the number of integral values is 36, the curves k67 to k86 are fingerprints representing feature amounts based on integral values for the shochu of No. 1 to No. 20, respectively.

Other explanations for the case where the number of integral values is 36 are the same as explanations for the case where the number of integral values is 138.

### (IV) Number of Integral Values: 18

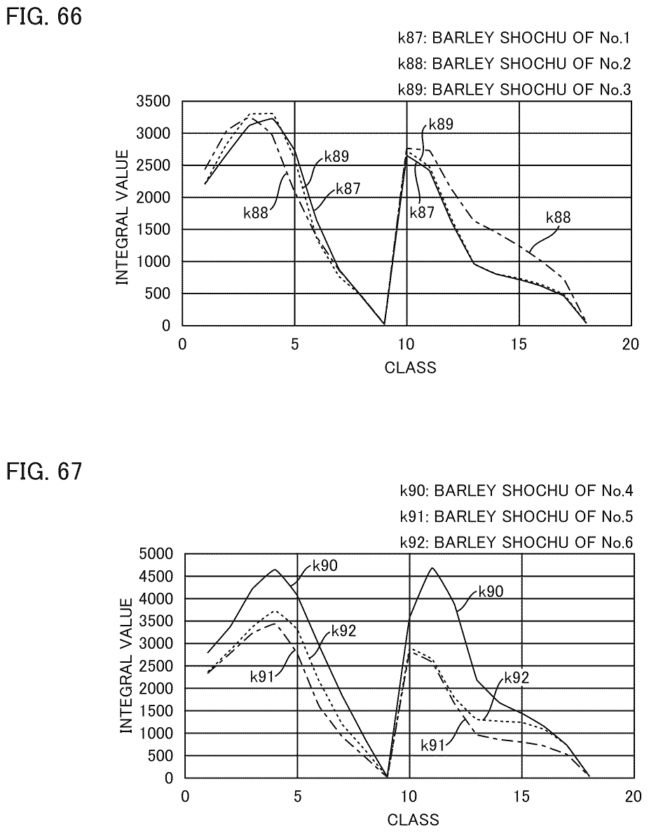

FIG. 66 is a diagram showing integral value spectra for the shochu of samples No. 1 to No. 3 shown in Table 2, in the case where the number of integral values is 18.

Referring to FIG. 66, each of the curves k87 to k89 is a curve CUR (index curve) created by the creation unit 215 of the diagnostic device 2 using the above-mentioned method. The curve k87 shows the integral value spectrum of the barley shochu of sample No. 1, the curve k88 shows the integral value spectrum of the barley shochu of sample No. 2, and the curve k89 shows the integral value spectrum of the barley shochu of sample No. 3.

FIG. 67 is a diagram showing integral value spectra for the shochu of samples No. 4 to No. 6 shown in Table 2, in the case where the number of integral values is 18.

Referring to FIG. 67, each of the curves k90 to k92 is a curve CUR (index curve) created by the creation unit 215 of the diagnostic device 2 using the above-mentioned method. The curve k90 shows the integral value spectrum of the barley shochu of sample No. 4, the curve k91 shows the integral value spectrum of the barley shochu of sample No. 5, and the curve k92 shows the integral value spectrum of the barley shochu of sample No. 6.

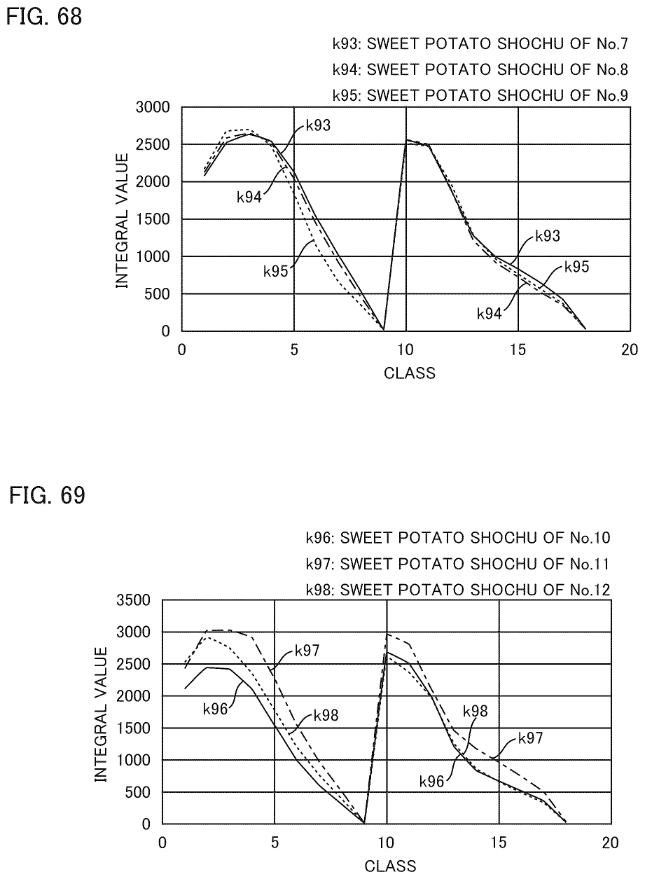

FIG. 68 is a diagram showing integral value spectra for the Shochu of samples No. 7 to No. 9 shown in Table 2 when the number of integral values is 18.

Referring to FIG. 68, each of the curves k93 to k95 is a curve CUR (index curve) created by the creation unit 215 of the diagnostic device 2 using the above-mentioned method. The curve k93 shows the integral value spectrum of the sweet potato shochu of sample No. 7, the curve k94 shows the integral value spectrum of the sweet potato shochu of sample No. 8, and the curve k95 shows the integral value spectrum of the sweet potato shochu of sample No. 9.

FIG. 69 is a diagram showing integral value spectra for the shochu of samples No. 10 to No. 12 shown in Table 2 when the number of integral values is 18.

Referring to FIG. 69, each of the curves k96 to k98 is a curve CUR (index curve) created by the creation unit 215 of the diagnostic device 2 using the above-mentioned method. The curve k96 shows the integral value spectrum of the sweet potato shochu of sample No. 10, the curve k97 shows the integral value spectrum of the sweet potato shochu of sample No. 11, and the curve k98 shows the integral value spectrum of the sweet potato shochu of sample No. 12.

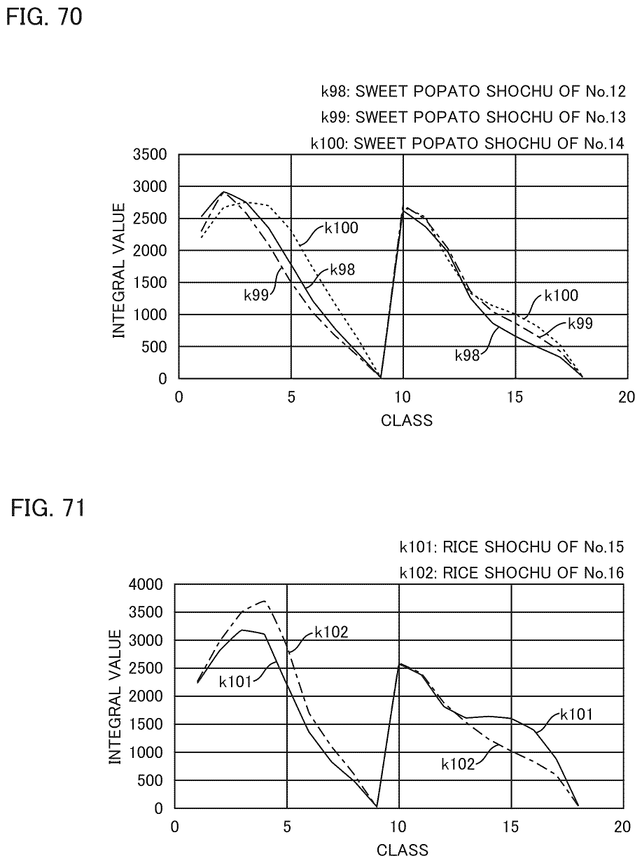

FIG. 70 is a diagram showing integral value spectra for the shochu of samples No. 12 to No. 14 shown in Table 2 when the number of integral values is 18.

Referring to FIG. 70, each of the curves k98 to k100 is a curve CUR (index curve) created by the creation unit 215 of the diagnostic device 2 using the above-mentioned method. The curve k98 shows the integral value spectrum of the sweet potato shochu of sample No. 12, the curve k99 shows the integral value spectrum of the sweet potato shochu of sample No. 13, and the curve k100 shows the integral value spectrum of the sweet potato shochu of sample No. 14.

FIG. 71 is a diagram showing integral value spectra for the shochu of samples No. 15 and No. 16 shown in Table 2 when the number of integral values is 18.

Referring to FIG. 71, each of curves k101 and k102 is a curve CUR (index curve) created by the creation unit 215 of the diagnostic device 2 using the above-mentioned method. The curve k101 shows the integral value spectrum of the rice shochu of sample No. 15, and the curve k102 shows the integral value spectrum of the rice shochu of sample No. 16.

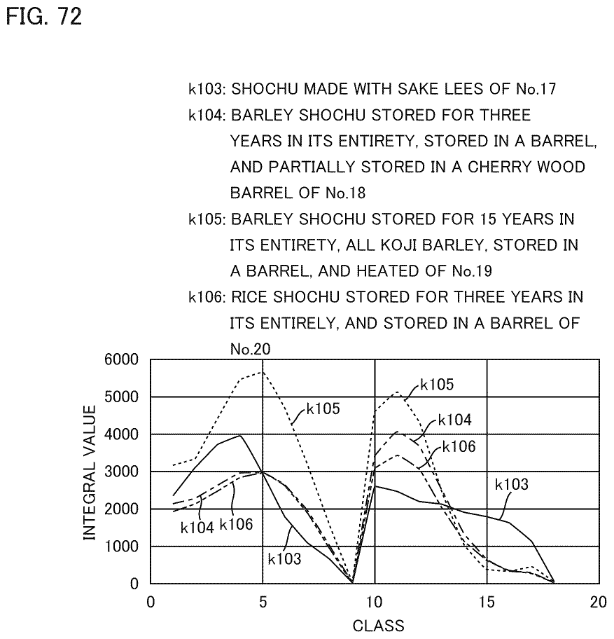

FIG. 72 is a diagram showing integral value spectra for the shochu of samples No. 17 to No. 20 shown in Table 2 when the number of integral values is 18.

Referring to FIG. 72, each of the curves k103 to k106 is a curve CUR (index curve) created by the creation unit 215 of the diagnostic device 2 by the above-mentioned method. The curve k103 shows the integral value spectrum of the shochu made with sake lees of sample No. 17, the curve k104 shows the integral value spectrum of the barley shochu stored for three years in its entirety, stored in a barrel, and partially stored in a cherry wood barrel of sample No. 18, the curve k105 shows the integral value spectrum of the barley shochu stored for 15 years in its entirety, all koji barley, stored in a barrel, and heated, of sample No. 19, and the curve k106 shows the integral value spectrum of the rice shochu stored for three years in its entirety, and stored in a barrel of sample No. 20.

In FIGS. 66 to 72, each of classes 1 to 8 has a predetermined potential section (integral value extraction potential) of 307.7 mV, class 9 has a predetermined potential section (integral value extraction potential) of 36.2 mV, each of classes 10 to 17 has a predetermined potential section (integral value extraction potential) of 307.7 mV, and class 18 has a predetermined potential section (integral value extraction potential) of 36.2 mV. Classes 1 to 9 are classes in the positive potential section, and classes 10 to 18 are classes in the negative potential section.

The reason why class 9 has a predetermined potential section (integral value extraction potential) of 36.2 mV, which is smaller than the predetermined potential section (integral value extraction potential) of 307.7 mV in each of classes 1 to 8, is to make class 9 the “last class in the positive predetermined potential section.”

In other words, when class 9 is composed of the same predetermined potential section (integral value extraction potential) as the predetermined potential section (integral value extraction potential) of 307.7 mV in each of classes 1 to 8, the integral value in class 9 is the sum of the integral value in the positive predetermined potential section and the integral value in the negative predetermined potential section, and it is not possible to calculate the sums SMT__{Low(+)}, SMT__{Middle(+)}, and SMT__{High(+)} of the integral values in the positive predetermined potential section based on an integral value spectrum in which all classes are composed of the predetermined potential section (integral value extraction potential) of 307.7 mV. As a result, it is not possible to diagnose the astringency ASTG of shochu using formula (5) by calculating the Body index(+) based on the integral value spectrum consisting of a predetermined potential section (integral value extraction potential) of 307.7 mV for all classes, it is not possible to diagnose the sweetness SWT of shochu using formula (7) by calculating H(+)_sum (=SMT__{High(+)}) based on the integral value spectrum consisting of a predetermined potential section (integral value extraction potential) of 307.7 mV for all classes, it is not possible to diagnose the aroma SCT of shochu using formula (8) by calculating H(−)_sum (=SMT_High(−)) based on the integral value spectrum consisting of a predetermined potential section (integral value extraction potential) of 307.7 mV for all classes, and as a result, it is not possible to diagnose the bitterness BIT of shochu using formula (9). Therefore, the class 9 has a predetermined potential section (integral value extraction potential) of 36.2 mV.

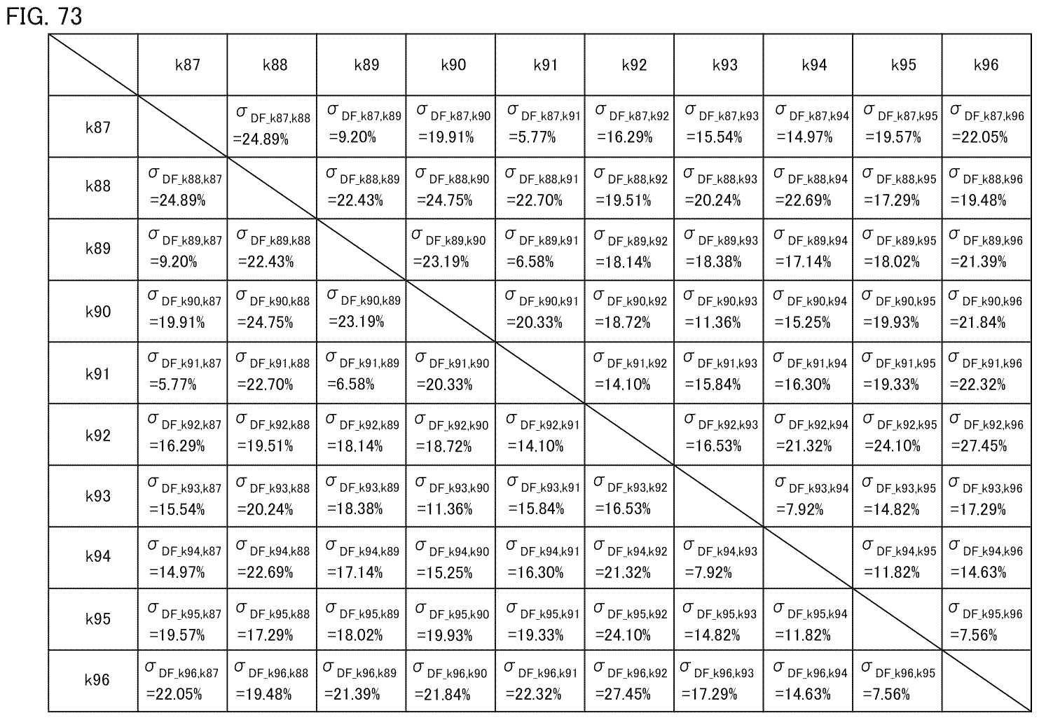

FIGS. 73 to 75 are first to third diagrams respectively showing the results of judging whether the curves k87 to k106 shown in FIGS. 66 to 72 are or not different from one another.

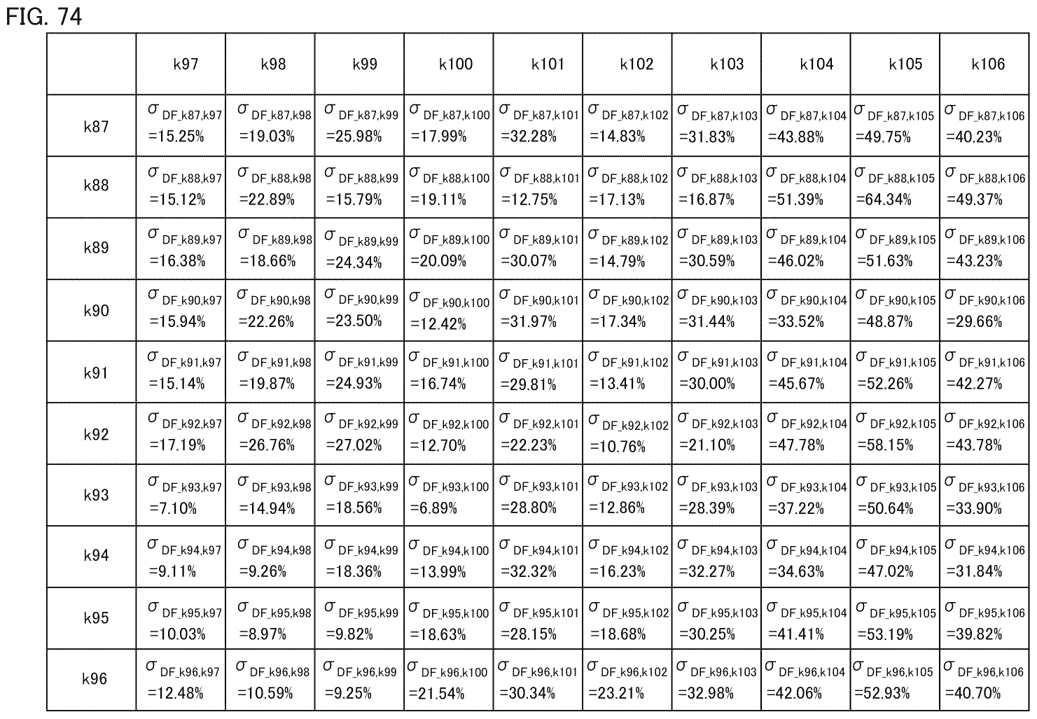

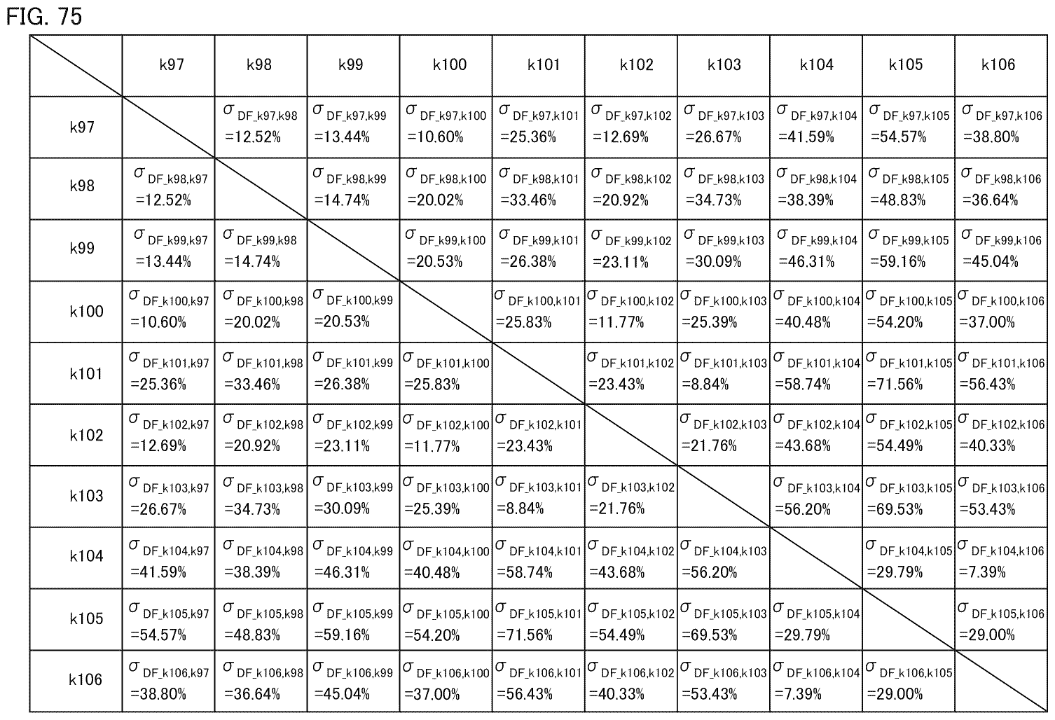

FIG. 73 shows the results of judging whether ten curves k87 to k96 are or not different from each other, FIG. 74 shows the results of judging whether ten curves k87 to k96 and ten curves k97 to k106 are or not different from each other, and FIG. 75 shows the results of judging whether ten curves k97 to k106 are or not different from each other.

The judgment of whether the 20 curves k87 to k106 are or not different from each other is executed by judging whether the two different curves are or not different for all combinations (₂₀C₂=190) in when selecting two different curves from the 20 curves k87 to k106.

Referring to FIG. 73, when two different curves are selected from the ten curves k87 to k96, all of the 45 standard deviations σ_{DF_k87, k88} to σ_{DF_k95, k96} of the differences for all of 45 combinations of two different curves are greater than the threshold value σₜₕ (=5%).

Referring to FIG. 74, when two different curves are selected from ten curves k87 to k96 and ten curves k97 to k106, all of the 100 standard deviations σ_{DF_k87, k97} to σ_{DF_k96, k106} of the differences for all of 100 combinations of two different curves are greater than the threshold value σₜₕ (=5%).

Referring to FIG. 75, when two different curves are selected from the ten curves k97 to k106, all of the 45 standard deviations σ_{DF_k97, k98} to σ_{DF_k105, k106} of the differences for all 45 combinations of two different curves are greater than the threshold value σₜₕ (=5%).

Therefore, when two different curves are selected from the 20 curves k87 to k106, all of the 190 standard deviations σ_{DF_k87, k88} to σ_{DF_k105, k106} of the differences for all 190 combinations of two different curves are greater than the threshold value σₜₕ (=5%).

Therefore, the 20 curves k87 to k106 are mutually different curves. When it is judged that the 20 curves k87 to k106 are mutually different, the curves k87 to k106 are curves for uniquely identifying shochu No. 1 to No. 20, respectively. When the number of integral values is 18, the curves k87 to k106 are fingerprints representing feature amounts based on integral values for shochu No. 1 to No. 20, respectively.

Although not shown in the figure, it was confirmed that when the number of integral values was 92, 56, 32, and 20, the standard deviation of the differences was greater than the threshold value 0th (5%) for all combinations (₂₀C₂=190) when selecting two different curves from 20 curves.

Other explanations regarding the case where the number of integral values is 18 are the same as those regarding the case where the number of integral values is 138.

As described above, the index curve for uniquely identifying Shochu No. 1 is made up of a plurality of curves k1, k27, k47, k67, and k87 each having a different number of integral values, the index curve for uniquely identifying Shochu No. 2 is made up of a plurality of curves k2, k28, k48, k68, and k88 each having a different number of integral values, the index curve for uniquely identifying Shochu No. 3 is made up of a plurality of curves k3, k29, k49, k69, and k89 each having a different number of integral values, the index curve for uniquely identifying Shochu No. 4 is made up of a plurality of curves k4, k30, k50, k70, and k90 each having a different number of integral values, and the index curve for uniquely identifying Shochu No. 5 is made up of a plurality of curves k5, k31, k51, k71, and k91 each having a different number of integral values.

In addition, the index curve for uniquely identifying Shochu No. 6 is made up of a plurality of curves k6, k32, k52, k72, and k92 each having a different number of integral values, the index curve for uniquely identifying Shochu No. 7 is made up of a plurality of curves k7, k33, k53, k73, and k93 each having a different number of integral values, the index curve for uniquely identifying Shochu No. 8 is made up of a plurality of curves k8, k34, k54, k74, and k94 each having a different number of integral values, the index curve for uniquely identifying Shochu No. 9 is made up of a plurality of curves k9, k35, k55, k75, and k95 each having a different number of integral values, and the index curve for uniquely identifying Shochu No. 10 is made up of a plurality of curves k10, k36, k56, k76, and k96 each having a different number of integral values.

Furthermore, the index curve for uniquely identifying Shochu No. 11 is made up of a plurality of curves k11, k37, k57, k77, and k97 having different numbers of integral values, the index curve for uniquely identifying Shochu No. 12 is made up of a plurality of curves k12, k38, k58, k78, and k98 having different numbers of integral values, the index curve for uniquely identifying Shochu No. 13 is made up of a plurality of curves k13, k39, k59, k79, and k99 having different numbers of integral values, the index curve for uniquely identifying Shochu No. 14 is made up of a plurality of curves k14, k40, k60, k80, and k100 having different numbers of integral values, and the index curve for uniquely identifying Shochu No. 15 is made up of a plurality of curves k15, k41, k61, k81, and k101 each having a different number of integral values.

Furthermore, the index curve for uniquely identifying Shochu No. 16 is made up of a plurality of curves k16, k42, k62, k82, and k102 having different numbers of integral values, the index curve for uniquely identifying Shochu No. 17 is made up of a plurality of curves k17, k43, k63, k83, and k103 having different numbers of integral values, the index curve for uniquely identifying Shochu No. 18 is made up of a plurality of curves k18, k44, k64, k84, and k104 having different numbers of integral values, the index curve for uniquely identifying Shochu No. 19 is made up of a plurality of curves k19, k45, k65, k85, and k105 having different numbers of integral values, and the index curve for uniquely identifying Shochu No. 20 is made up of a number of curves k20, k46, k66, k86, and k106 each having a different number of integral values.

In taste diagnosis of shochu, the taste diagnostic unit 217 diagnoses the astringency ASTG of the shochu using formula (5), diagnoses the aftertaste LNGS of the shochu using formula (6), diagnoses the sweetness SWT of the shochu using formula (7), diagnoses the aroma SCT of the shochu using formula (8), and diagnoses the bitterness BIT of the shochu using formula (9).

The taste diagnostic unit 217 uses the Body index (+) shown in equation (3) when diagnosing the astringency ASTG of shochu using equation (5), uses the Body index (all) when diagnosing the aftertaste LNGS of shochu using equation (6), uses H(+)_sum when diagnosing the sweetness SWT of shochu using equation (7), uses H(−)_sum when diagnosing the aroma SCT of shochu using equation (8), and uses the bitterness ASTG of shochu and the sweetness SWT of shochu when diagnosing the bitterness BIT of shochu using equation (9).

The taste diagnostic unit 217 calculates, in equation (3), the Body Index (+) using the sum SMT__{Low(+)}Of integral values in the positive potential section of the integral value spectrum created based on the cyclic voltammogram measured using the potential scanning speed V_{r_Low}, the sum SMT__{Middle(+)} of integral value in the positive potential section of the integral value spectrum created based on the cyclic voltammogram measured using the potential scanning speed V_{r_Middle}, and the sum SMT__{High(+)} of integral value in the positive potential section of the integral value spectrum created based on the cyclic voltammogram measured using the potential scanning speed V_{r_High}.

In addition, the taste diagnostic unit 217 calculates, in equation (4), the Body Index (all) using the sum SMT__{Low(all)}Of integral values in all potential sections of the integral value spectrum created based on the cyclic voltammogram measured using the potential scanning speed V_{r_Low}, the sum SMT__{Middle(all)} of integral values in all potential sections of the integral value spectrum created based on the cyclic voltammogram measured using the potential scanning speed V_{r_Middle}, and the sum SMT__{High(all)} of integral values in all potential sections of the integral value spectrum created based on the cyclic voltammogram measured using the potential scanning speed V_{r_High}.

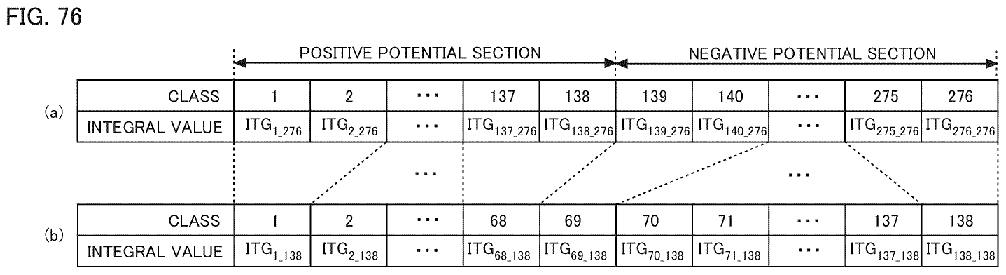

FIG. 76 is a diagram showing the correspondence between the classes and integral values when the number of integral values is 138 and the classes and integral values when the number of integral values is 276.

Referring to FIGS. 76 (a) and (b), when the number of integral value (=the number of classes) is 138, the integral value ITG_{1_138} in class 1 is the sum of the integral values ITG_{1_276} and ITG_{2_276} in classes 1 and 2 when the number of integral value (=the number of classes) is 276, the integral value ITG_{2_138} in class 2 is the sum of the integral values ITG_{3_276} and ITG_{4_276} in classes 3 and 4 when the number of integral value (=the number of classes) is 276, . . . , the integral value ITG_{68_138} in class 68 is the sum of the integral values ITG_{137_276} and ITG_{138_276} in classes 137 and 138 when the number of integral values (=the number of classes) is 276, the integral value ITG_{69_138} in class 69 is the sum of the integral values ITG_{139_276} and ITG_{140_276} in classes 139 and 140 when the number of integral values (=the number of classes) is 276, . . . , the integral value ITG_{138_138} in class 138 is the sum of the integral values ITG_{275_276} and ITG_{276_276} in classes 275 and 276 when the number of integral values (=the number of classes) is 276.

In FIG. 76 (a), classes 1 to 138 are classes in the positive potential section, integral values ITG_{1_276} to ITG_{138_276} are integral values in the positive potential section, classes 139 to 276 are classes in the negative potential section, and integral values ITG_{139_276} to ITG_{276_276} are integral values in the negative potential section.

As a result, in (b) of FIG. 76, classes 1 to 68 are classes in the positive potential section, integral values ITG_{1_138} to ITG_{68_138} are integral values in the positive potential section, classes 69 to 138 are classes in the negative potential section, and integral values ITG_{69_138} to ITG_{138_138} are integral values in the negative potential section.

In this case, the sum of the integral values ITG_{1_138} to ITG_{68_138} in classes 1 to 68 in FIG. 76 (b) is equal to the sum of the integral values ITG_{1_276} to ITG_{138_276} in classes 1 to 138 in FIG. 76 (a).

In addition, the sum of the integral values ITG_{69_138} to ITG_{138_138} in classes 69 to 138 in FIG. 76 (b) is equal to the sum of the integral values ITG_{139_276} to ITG_{276_276} in classes 139 to 276 in FIG. 76 (a).

Furthermore, the sum of the integral values ITG_{1_138} to ITG_{138_138} in classes 1 to 138 in FIG. 76 (b) is equal to the sum of the integral values ITG_{1_276} to ITG_{276_276} in classes 1 to 276 in FIG. 76 (a).

Therefore, the sum SMT__{Low(+)_138} of integral values in the positive potential section of the integral value spectrum consisting of 138 integral values created based on the cyclic voltammogram measured using the potential scanning speed V_{r_Low} is equal to the sum SMT__{Low(+)_276} of integral values in the positive potential section of the integral value spectrum consisting of 276 integral values created based on the cyclic voltammogram measured using the potential scanning speed V_{r_Low}.

The same is true for the sum SMT__{Middle(+)_138} of integral values in the positive potential section of an integral value spectrum consisting of 138 integral values created based on a cyclic voltammogram measured using a potential scanning speed V_{r_Middle}, and the sum SMT__{High(+)_138} of integral values in the positive potential section of an integral value spectrum consisting of 138 integral values created based on a cyclic voltammogram measured using a potential scanning speed V_{r_High}.

As a result, in equation (3), Body Index (+)_₁₃₈ calculated based on the sums of the integral values SMT__{Low(+)_138}, SMT__{Middle(+)_138}, and SMT__{High(+)_138} is equal to Body Index (+)_₂₇₆ calculated based on the sums SMT__{Low(+)_276}, SMT__{Middle(+)_276}, and SMT__{High(+)_276} of the integral values.

In addition, the sum SMT__{Low(all)_138} of the integral values in all potential sections of the integral value spectrum consisting of 138 integral values created based on the cyclic voltammogram measured using the potential scanning speed V_{r_Low} is equal to the sum SMT__{Low(all)_276} of the integral values in all potential sections of the integral value spectrum consisting of 276 integral values created based on the cyclic voltammogram measured using the potential scanning speed V_{r_Low}.

The same is true for the sum SMT__{Middle(all)_138} of the integral values over all potential sections of an integral value spectrum consisting of 138 integral values created based on a cyclic voltammogram measured using a potential scanning speed V_{r_Middle}, and the sum SMT__{High(all)_138} of the integral values over all potential sections of an integral value spectrum consisting of 138 integral values created based on a cyclic voltammogram measured using a potential scanning speed V_{r_High}.

As a result, in equation (4), Body index (all)_₁₃₈ calculated using the sums SMT__{Low(all)_138}, SMT__{Middle(all)_138}, and SMT__{High(all)_138} of the integral values is equal to Body index (all)_₂₇₆ calculated using the sums SMT__{Low(all)_276}, SMT__{Middle(all)_276}, and SMT__{High(all)_276} of the integral values.

Furthermore, H(+)__{sum_138} which is the sum SMT_High(+) of the integral values in the positive potential section of an integral value spectrum consisting of 138 integral values created based on a cyclic voltammogram measured using a potential scanning speed V_{r_High}, is equal to H(+)__{sum_276} which is the sum SMT__{High(+)} of the integral values in the positive potential section of an integral value spectrum consisting of 276 integral values created based on a cyclic voltammogram measured using a potential scanning speed V_{r_High}.

Furthermore, H(−)_sum_₁₃₈ which is the sum SMT_High(−) of the integral values in the negative potential section of an integral value spectrum consisting of 138 integral values created based on a cyclic voltammogram measured using a potential scanning speed V_{r_High}, is equal to H(−)__{sum_276} which is the sum SMT__{High(-)} of the integral values in the negative potential section of an integral value spectrum consisting of 276 integral values created based on a cyclic voltammogram measured using a potential scanning speed V_{r_High}.

Even if the number of integral values changes, since the coefficients k1 to k5 in formulas (5) to (9) do not change, in equation (5), the astringency ASTG_₁₃₈ of shochu calculated using the Body index (+)_₁₃₈ is equal to the astringency ASTG_₂₇₆ of shochu calculated using the Body index (+)_₂₇₆, in formula (6), the aftertaste LNGS_₁₃₈ of the shochu calculated using the Body index (all)_₁₃₈ is equal to the aftertaste LNGS_₂₇₆ of the shochu calculated using the Body index (all)_₂₇₆, in formula (7), the sweetness SWT 138 of shochu calculated using H(+)_sum_₁₃₈ (=SMT__{High(+)_138}) is equal to the sweetness SWT 276 of shochu calculated using H(+)__{sum_276} (=SMT__{High(+)_276}), in formula (8), the aroma SCT 138 of shochu calculated using H(−)_sum_₁₃₈ (=SMT__{High(-)_138}) is consistent with the aroma SCT 276 of shochu calculated using H(−)_sum_₂₇₆ (=SMT__{High(-)_276}), and in formula (9), the bitterness BIT_₁₃₈ of shochu calculated using the astringency ASTG_₁₃₈ and sweetness SWT_₁₃₈ of shochu is equal to the bitterness BIT 276 of shochu calculated using the astringency ASTG_₂₇₆ and sweetness SWT_₂₇₆ of shochu.

The same is applied to the cases where the number of integral values is any of 92, 70, 56, 36, 32, 20, and 18.

Therefore, even if the number of integral values in the integral value spectrum which is the fingerprint for Shochu No. 1 to Shochu No. 20 changes, the results of the taste diagnosis of Shochu No. 1 to Shochu No. 20 will match the “astringency”, “aftertaste”, “sweetness”, “aroma” and “bitterness” shown in FIG. 29.

As a result, the diagnostic device 2 may perform the taste diagnosis for Shochu No. 1 to Shochu No. 20 by the above-mentioned method based on the integral value spectrum ITG__{SPC_n} in which the number of integral values is any one of 18, 20, 32, 36, 56, 70, 92, 138, and 276.

In this case, the integral value spectrum ITG__{SPC_n} is composed of an integral value spectrum consisting of integral values ITG_{1_Low} to ITG_{n_Low}, an integral value spectrum consisting of integral values ITG_{1_Middle} to ITG_{n_Middle}, and an integral value spectrum consisting of integral values ITG_{1_High} to ITG_{n_High}, where n is any of 18, 20, 32, 36, 56, 70, 92, 138, and 276.

[Relationship between taste diagnosis and number of integral values in grapes] As described above, in the taste diagnosis of Crimson Seed (skin+flesh), Green Seedless (skin+flesh), Shine Muscat (skin+flesh), Crimson Seedless (flesh only), Green Seedless (flesh only) and Shine Muscat (flesh only), the threshold value Vth of the potential V is set to −1362 mV, and L(−)_sum_th, M(−)_sum_th and H(−)_sum_th calculated based on the integral value in a specified potential section (=class) in the range of −1362 mV to −2501 mV are used.

Therefore, an integral value spectrum in which the number of integral values in a predetermined potential section (=class) in the range of −1362 mV to −2501 mV used for taste diagnosis is changed will be explained.

### (I) Number of Integral Values: 74

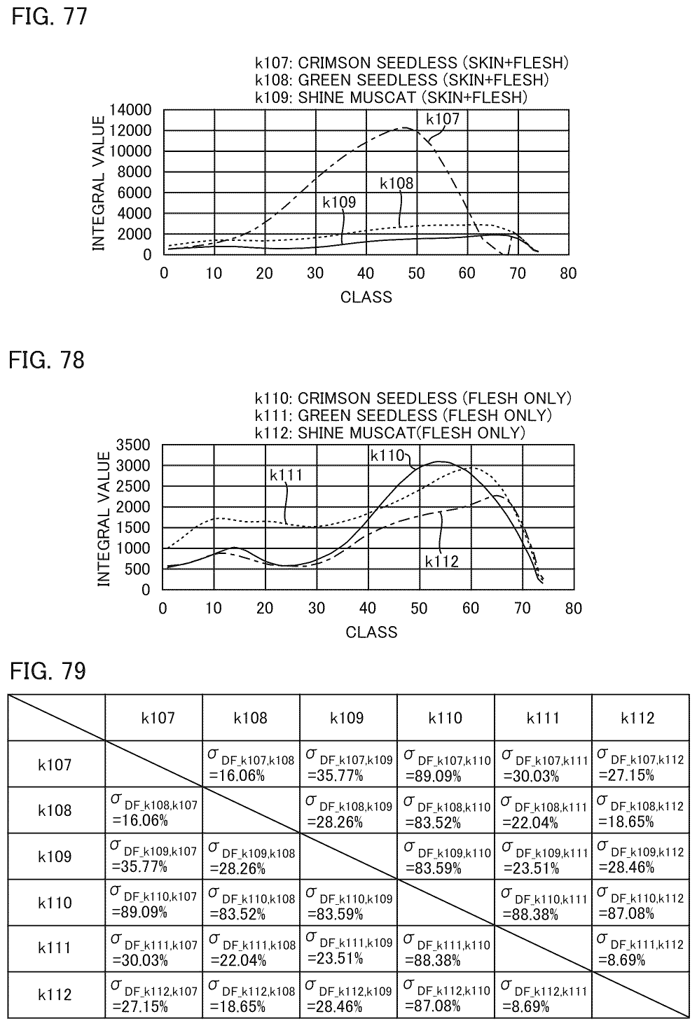

FIG. 77 is a diagram showing integral value spectra of Crimson Seedless (skin+flesh), Green Seedless (skin+flesh) and Shine Muscat (skin+flesh) when the number of integral values in a predetermined potential section in the range of −1362 mV to −2501 mV is 74.

Referring to FIG. 77, each of the curves k107 to k109 is a curve CUR created by the diagnostic device 2 using the above-mentioned method in a predetermined potential section ranging from −1362 mV to −2501 mV. The curve k107 shows the integral value spectrum of Crimson Seedless (skin+flesh), the curve k108 shows the integral value spectrum of Green Seedless (skin+flesh), and the curve k109 shows the integral value spectrum of Shine Muscat (skin+flesh).

FIG. 78 is a diagram showing integral value spectra of Crimson Seedless (flesh only), Green Seedless (flesh only), and Shine Muscat (flesh only) when the number of integral values in a predetermined potential section in the range of −1362 mV to −2501 mV is 74.

Referring to FIG. 78, each of the curves k110 to k112 is a curve CUR generated by the diagnostic device 2 using the above-mentioned method in a predetermined potential section ranging from −1362 mV to −2501 mV. The curve k110 shows the integral value spectrum of Crimson Seedless (flesh only), the curve k111 shows the integral value spectrum of Green Seedless (flesh only), and the curve k112 shows the integral value spectrum of Shine Muscat (flesh only).

In FIGS. 77 and 78, each of classes 1 to 74 consists of a predetermined potential section of 18.1 mV.

FIG. 79 is a diagram showing the result of judgment as to whether the curves k107 to k112 shown in FIG. 77 and FIG. 78 are or not different from each other.

Whether the curves k107 to k112 are or not different from one another is judged by executing a judgment as to whether any two different curves among the curves k107 to k112 are or not different for all combinations of two different curves among the curves k107 to k112.

There are 15 combinations of two different curves among the six curves k107 to k112: (k107, k108), (k107, k109), (k107, k110), (k107, k111), (k107, k112), (k108, k109), (k108, k110), (k108, k111), (k108, k112), (k109, k110), (k109, k111), (k109, k112), (k110, k111), (k110, k112), and (k111, k112).

Referring to FIG. 79, in the 15 standard deviations σ_{DF_k107, k108}, σ_{DF_k107, k109}, . . . , σ_{DF_k111, k112} of differences σ_{DF_k107, k108}, σ_{DF_k107, k109}, . . . , σ_{DF_k111, k112}, the minimum value is 8.69% and the maximum value is 89.09%.

Therefore, all of the 15 standard deviations σ_{DF_k107, k108}, σ_{DF_k107, k109}, . . . , σ_{DF_k111, k112} of the difference are greater than the threshold value σₜₕ (=8%).

Therefore, curves k107 to k112 are mutually different curves, and are fingerprints that represent the features based on integral values for Crimson Seedless (skin+flesh), Green Seedless (skin+flesh), Shine Muscat (skin+flesh), Crimson Seedless (flesh only), Green Seedless (flesh only), and Shine Muscat (flesh only), respectively.

### (II) Number of Integral Values: 37

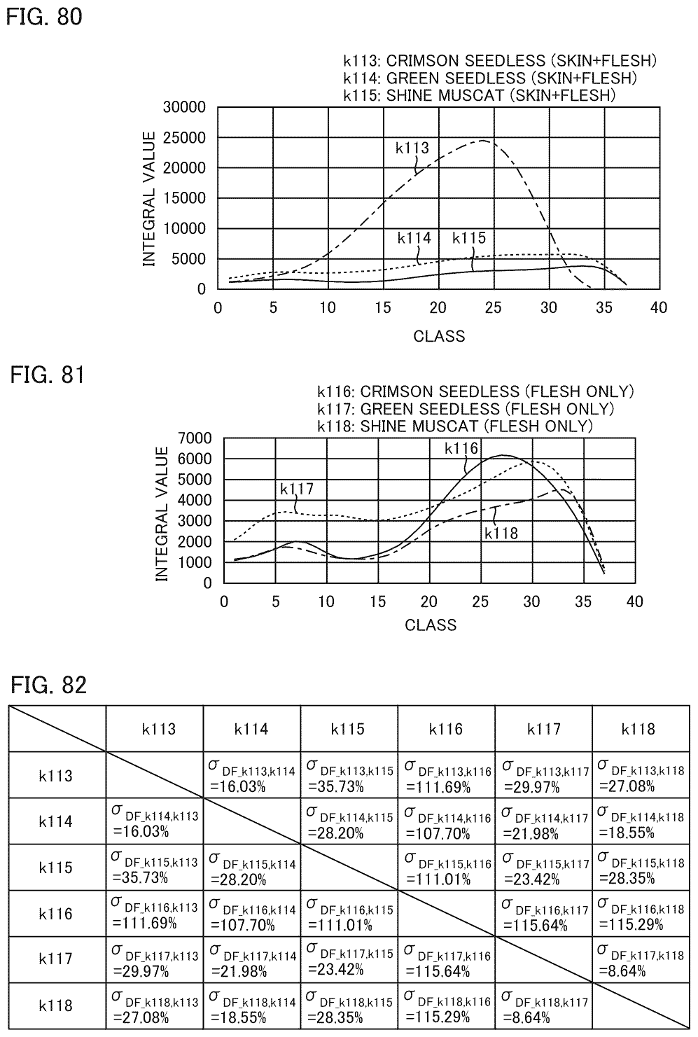

FIG. 80 is a diagram showing integral value spectra of Crimson Seedless (skin+flesh), Green Seedless (skin+flesh) and Shine Muscat (skin+flesh) when the number of integral values in a predetermined potential section in the range of −1362 mV to −2501 mV is 37.

Referring to FIG. 80, each of the curves k113 to k115 is a curve CUR which the diagnostic device 2 created by the method mentioned above in a predetermined potential section in the range of −1362 mV to −2501 mV. The curve k113 shows the integral value spectrum of Crimson Seedless (skin+flesh), the curve k114 shows the integral value spectrum of Green Seedless (skin+flesh), and the curve k115 shows the integral value spectrum of Shine Muscat (skin+flesh).

FIG. 81 is a diagram showing integral value spectra of Crimson Seedless (flesh only), Green Seedless (flesh only), and Shine Muscat (flesh only) when the number of integral values in a s predetermined potential section in the range of −1362 mV to −2501 mV is 37.

Referring to FIG. 81, each of the curves k116 to k118 is a curve CUR which the diagnostic device 2 created by the method mentioned above in a predetermined potential section in the range of −1362 mV to −2501 mV. The curve k116 shows the integral value spectrum of Crimson Seedless (flesh only), the curve k117 shows the integral value spectrum of Green Seedless (flesh only), and the curve k118 shows the integral value spectrum of Shine Muscat (flesh only).

In FIGS. 80 and 81, each of classes 1 to 37 consists of a predetermined potential section of 36.2 mV.

FIG. 82 is a diagram showing the result of judgment as to whether or not the curves k113 to k118 shown in FIGS. 80 and 81 are different from each other.

Whether or not the curves k113 to k118 are different from one another is judged by executing a judgment as to whether or not any two different curves among the curves k113 to k118 are different for all combinations of two different curves among the curves k113 to k118.

There are 15 possible combinations of two different curves among the six curves k113 to k118: (k113, k114), (k113, k115), (k113, k116), (k113, k117), (k113, k118), (k114, k115), (k114, k116), (k114, k117), (k114, k118), (k115, k116), (k115, k117), (k115, k118), (k116, k117), (k116, k118), and (k117, k118).

Referring to FIG. 82, in the 15 standard deviations σ_{DF_k113, k114}, σ_{DF_k113, k115}, . . . , σ_{DF_k117, k118} of differences, the minimum value is 8.64% and the maximum value is 115.64%.

Therefore, all of the 15 standard deviations σ_{DF_k113, k114}, σ_{DF_k113, k115}, . . . , σ_{DF_k117, k118} of differences are greater than the threshold value σₜₕ (=8%).

Therefore, curves k113 to k118 are mutually different curves, and are fingerprints that represent features based on integral values for Crimson Seedless (skin+flesh), Green Seedless (skin+flesh), Shine Muscat (skin+flesh), Crimson Seedless (flesh only), Green Seedless (flesh only), and Shine Muscat (flesh only), respectively.

### (III) Number of Integral Values: 19

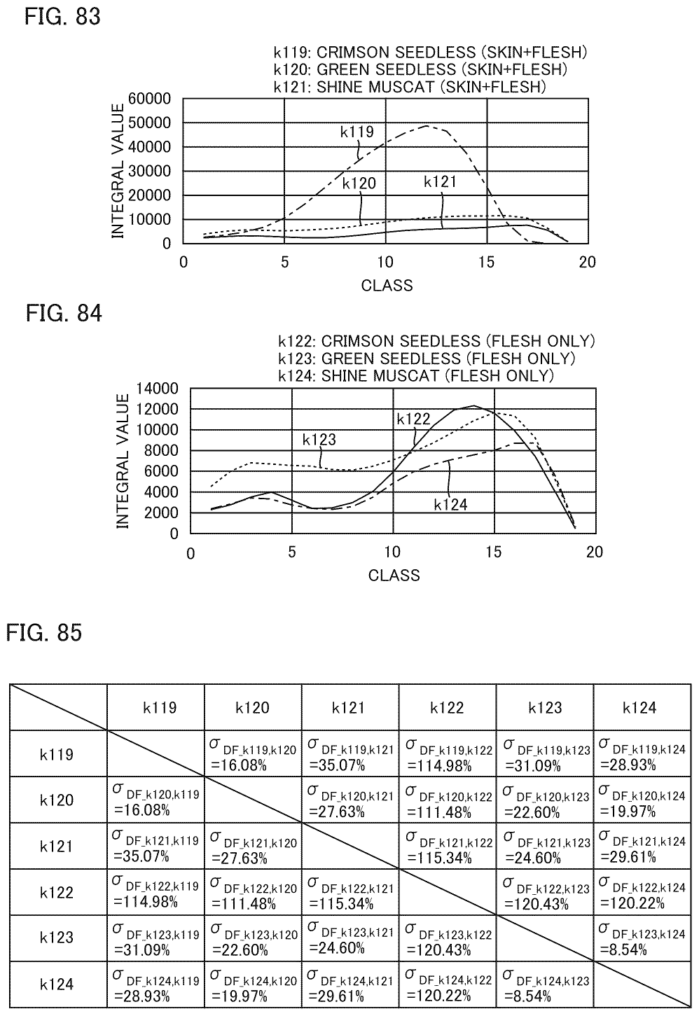

FIG. 83 is a diagram showing integral value spectra of Crimson Seedless (skin+flesh), Green Seedless (skin+flesh) and Shine Muscat (skin+flesh) when the number of integral values in a predetermined potential section in the range of −1362 mV to −2501 mV is 19.

Referring to FIG. 83, each of the curves k119 to k121 is a curve CUR which the diagnostic device 2 created by the method mentioned above in a predetermined potential section in the range of −1362 mV to −2501 mV. Curve k119 shows the integral value spectrum of Crimson Seedless (skin+flesh), curve k120 shows the integral value spectrum of Green Seedless (skin+flesh), and curve k121 shows the integral value spectrum of Shine Muscat (skin+flesh).

FIG. 84 is a diagram showing integral value spectra of Crimson Seedless (flesh only), Green Seedless (flesh only), and Shine Muscat (flesh only) in when the number of integral values in a predetermined potential section in the range of −1362 mV to −2501 mV is 19.

Referring to FIG. 84, each of the curves k122 to k124 is a curve CUR which the diagnostic device 2 created by the method mentioned above in a predetermined potential section in the range of −1362 mV to −2501 mV. Curve k122 shows the integral value spectrum of Crimson Seedless (flesh only), curve k123 shows the integral value spectrum of Green Seedless (flesh only), and curve k124 shows the integral value spectrum of Shine Muscat (flesh only).

In FIGS. 83 and 84, each of classes 1 to 18 is made up of a predetermined potential section of 72.4 mV, and class 19 is made up of a predetermined potential section of 36.2 mV.

FIG. 85 is a diagram showing the result of judgment as to whether or not the curves k119 to k124 shown in FIG. 83 and FIG. 84 are different from each other.

Whether or not the curves k119 to k124 are different from one another is judged by executing a judgment as to whether or not any two different curves among the curves k119 to k124 are different for all combinations of two different curves among the curves k119 to k124.

There are 15 combinations of two different curves among the six curves k119 to k124: (k119, k120), (k119, k121), (k119, k122), (k119, k123), (k119, k124), (k120, k121), (k120, k122), (k120, k123), (k120, k124), (k121, k122), (k121, k123), (k121, k124), (k122, k123), (k122, k124), and (k123, k124).

Referring to FIG. 85, in the 15 standard deviations σ_{DF_k119, k120}, σ_{DF_k119, k121}, . . . σ_{DF_k123, k124} of differences, the minimum value is 8.54% and the maximum value is 120.43%.

Therefore, all of the standard deviations σ_{DF_k119, k120}, σ_{DF_k119, k121}, . . . , σ_{DF_k123, k124} of the 15 differences are greater than the threshold value σₜₕ (=8%).

Therefore, curves k119 to k124 are mutually different curves and are fingerprints representing features based on integral values for Crimson Seedless (skin+flesh), Green Seedless (skin+flesh), Shine Muscat (skin+flesh), Crimson Seedless (flesh only), Green Seedless (flesh only), and Shine Muscat (flesh only).

### (IV) Number of Integral Values: 9

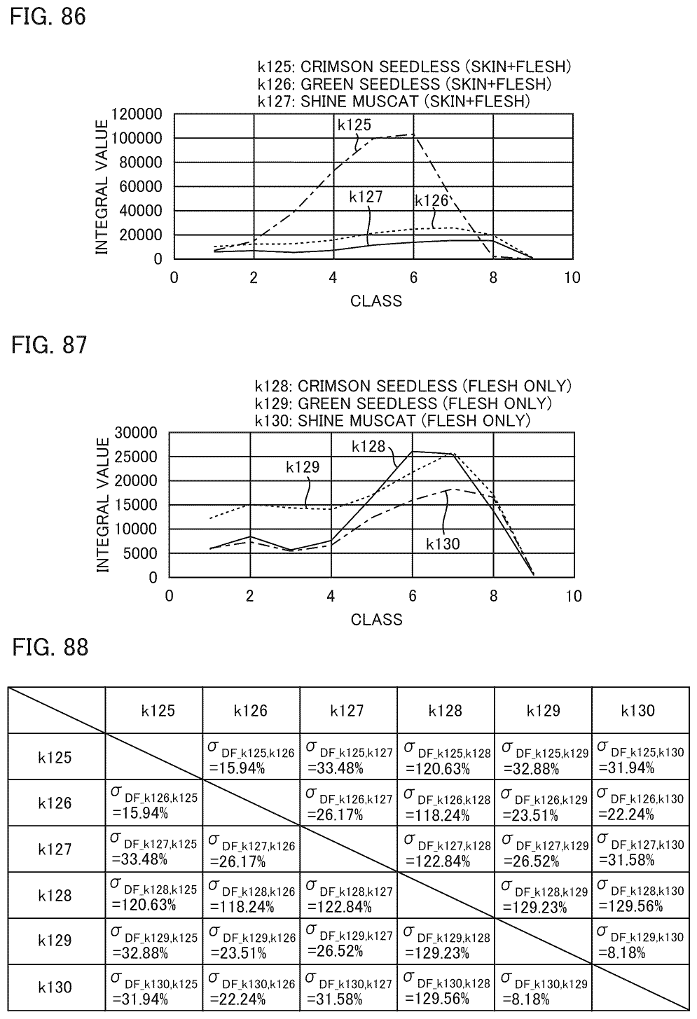

FIG. 86 is a diagram showing integral value spectra of Crimson Seedless (skin+flesh), Green Seedless (skin+flesh) and Shine Muscat (skin+flesh) in when the number of integral values in a predetermined potential section in the range of −1362 mV to −2501 mV is 9.

Referring to FIG. 86, each of the curves k125 to k127 is a curve CUR which the diagnostic device 2 created by the method mentioned above in a predetermined potential section in the range of −1362 mV to −2501 mV. Curve k125 shows the integral value spectrum of Crimson Seedless (skin+flesh), curve k126 shows the integral value spectrum of Green Seedless (skin+flesh), and curve k127 shows the integral value spectrum of Shine Muscat (skin+flesh).

FIG. 87 is a diagram showing integral value spectra of Crimson Seedless (flesh only), Green Seedless (flesh only), and Shine Muscat (flesh only) when the number of integral values in a predetermined potential section in the range of −1362 mV to −2501 mV is 9.

Referring to FIG. 87, each of the curves k128 to k130 is a curve CUR which the diagnostic device 2 created by the method mentioned above in a predetermined potential section in the range of −1362 mV to −2501 mV. Curve k128 shows the integral value spectrum of Crimson Seedless (flesh only), curve k129 shows the integral value spectrum of Green Seedless (flesh only), and curve k130 shows the integral value spectrum of Shine Muscat (flesh only).

In FIGS. 86 and 87, each of classes 1 to 8 is made up of a predetermined potential section of 162.9 mV, and class 9 is made up of a predetermined potential section of 36.2 mV.

FIG. 88 is a diagram showing the result of judgment as to whether the curves k125 to k130 shown in FIG. 86 and FIG. 87 are or not different from each other.

Whether or not the curves k125 to k130 are different from one another is judged by executing a judgment as to whether or not any two different curves among the curves k125 to k130 are different for all combinations of two different curves among the curves k125 to k130.

There are 15 combinations of two different curves among the six curves k125 to k130: (k125, k126), (k125, k127), (k125, k128), (k125, k129), (k125, k130), (k126, k127), (k126, k128), (k126, k129), (k126, k130), (k127, k128), (k127, k129), (k127, k130), (k128, k129), (k128, k130), and (k129, k130).

Referring to FIG. 88, in the 15 standard deviations σ_{DF_k125, k126}, σ_{DF_k125, k127}, . . . , σ_{DF_k129, k130} of differences, the minimum value is 8.18% and the maximum value is 129.56%.

Therefore, all of the 15 standard deviations σ_{DF_k125, k126}, σ_{DF_k125, k127}, . . . , σ_{DF_k129, k130} of the differences are greater than the threshold value σₜₕ (=8).

Therefore, curves k125 to k130 are mutually different curves and are fingerprints that represent features based on integral values for Crimson Seedless (skin+flesh), Green Seedless (skin+flesh), Shine Muscat (skin+flesh), Crimson Seedless (flesh only), Green Seedless (flesh only), and Shine Muscat (flesh only), respectively.

Although not shown in the figures, it was also confirmed that when the number of integral values in a predetermined potential section in the range of −1362 mV to −2501 mV was 25, 15, 13, 11, and 7, the standard deviation of the differences was greater than the threshold value (8%) for all combinations (₆C₂=15) in when selecting two different curves from six curves.

As described above, the index curve for uniquely identifying Crimson Seedless (skin+flesh) consists of a plurality of curves k107, k113, k119, and k125 having different numbers of integral values, the index curve for uniquely identifying Green Seedless (skin+flesh) consists of a plurality of curves k108, k114, k120, and k126 having different numbers of integral values, the index curve for uniquely identifying Shine Muscat (skin+flesh) consists of a plurality of curves k109, k115, k121, and k127 having different numbers of integral values, the index curve for uniquely identifying Crimson Seedless (flesh only) consists of a plurality of curves k110, k116, k122, and k128 having different numbers of integral values, the index curve for uniquely identifying Green Seedless (flesh only) consists of a plurality of curves k111, k117, k123, and k129 having different numbers of integral values, and the index curve for uniquely identifying Shine Muscat (flesh only) consists of a plurality of curves k112, k118, k124, and k130 having different numbers of integral values.

As described above, the “astringency” of grapes is calculated by calculating the Body index (−)th shown in formula (10) and substituting the calculated Body index (−)th into equation (11).

The Body index (−)th is calculated based on the sums of the integral values L(−)_sum_th, M(−)_sum_th, and H(−)_sum_th, as shown in equation (10).

The sum L(−)_sum_th of integral values is the sum of integral values in a predetermined potential section in range from −1362 mV to −2501 mV among a plurality of integral values calculated based on a cyclic voltammogram measured by setting the potential scanning rate to the potential scanning rate V_{r_Low}, the um M(−)_sum_th of integral values is the sum of integral values in a predetermined potential section ranging in range from −1362 mV to −2501 mV among a plurality of integral values calculated based on a cyclic voltammogram measured by setting the potential scanning rate to the potential scanning rate V_{r_Middle}, and the sum H(−)_sum_th of integral values is the sum of integral values in a predetermined potential section in range from −1362 mV to −2501 mV among a plurality of integral values calculated based on a cyclic voltammogram measured by setting the potential scanning rate to the potential scanning rate V_{r_High}.

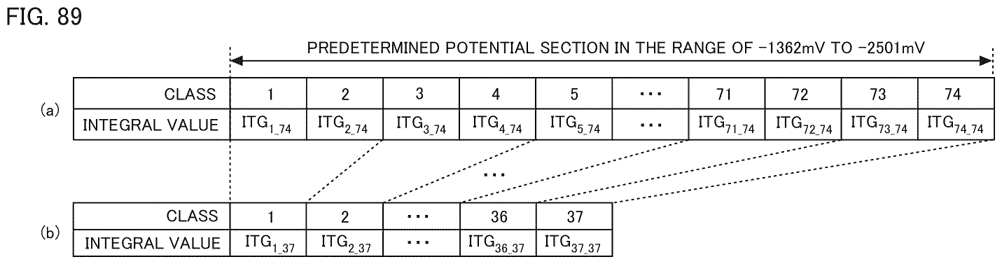

FIG. 89 is a diagram showing the correspondence relationship between the classes and integral values when the number of integral values is 37 and the correspondence relationship between the classes and integral values when the number of integral values is 74.

Referring to FIG. 89, when the number of integral values in a predetermined potential section in the range of −1362 mV to −2501 mV is 74, the integral values in classes 1 to 74 are ITG_{1_74} to ITG_{74_74}, respectively.

When the number of integral values in a predetermined potential section in the range of −1362 mV to −2501 mV is 37, the integral value ITG_{1_37} in class 1 consists of the sum of the integral values ITG_{1_74} and ITG_{2_74} when the number of integral values is 74, the integral value ITG_{2_37} in class 2 consists of the sum of the integral values ITG_{3_74} and ITG_{4_74} when the number of integral values is 74, . . . , the integral value ITG_{36_37} in class 36 consists of the sum of the integral values ITG_{71_74} and ITG_{72_74} when the number of integral values is 74, and the integral value ITG_{37_37} in class 37 consists of the sum of the integral values ITG_{73_74} and ITG_{74_74} when the number of integral values is 74.

As a result, when the number of integral values is 37, the sum of the 37 integral values is equal to the sum of the 74 integral values when the number of integral values is 74.

In this case, although not shown in the figure, when the number of integral values in a predetermined potential section in the range of −1362 mV to −2501 mV is 19, the sum of the 19 integral values is equal to the sum of the 74 integral values when the number of integral values is 74, and the sum of the 9 integral values when the number of integral values in a predetermined potential section in the range of −1362 mV to −2501 mV is 9 is equal to the sum of the 74 integral values when the number of integral values is 74.

Therefore, the sum L(−)_sum_th_₇₄ of integral values that is the sum L(−)_sum_th of integral values in when the number of integral values in a predetermined potential section in the range of −1362 mV to −2501 mV is 74, the sum L(−)_sum_th_₃₇ of integral values that is the sum L(−)_sum_th of integral values in when the number of integral values in a predetermined potential section in the range of −1362 mV to −2501 mV is 37, the sum L(−)_sum_th_₁₉ of integral values that is the sum L(−)_sum_th of integral values in when the number of integral values in a predetermined potential section in the range of −1362 mV to −2501 mV is 19, and the sum L(−)_sum_th_₉ of integral values that is the sum L(−)_sum_th of integral values in when the number of integral values in a predetermined potential section in the range of −1362 mV to −2501 mV is 9, are equal to each other.

Similarly, the sum M(−)_sum_th_₇₄ of integral values that is the sum M(−)_sum_th of integral values in when the number of integral values in a predetermined potential section in the range of −1362 mV to −2501 mV is 74, the sum M(−)_sum_th_₃₇ of integral values that is the sum M(−)_sum_th of integral values in when the number of integral values in a predetermined potential section in the range of −1362 mV to −2501 mV is 37, the sum M(−)_sum_th_₁₉ of integral values that is the sum M(−)_sum_th of integral values in when the number of integral values in a predetermined potential section in the range of −1362 mV to −2501 mV is 19, and the sum M(−)_sum_th_₉ of integral values that is the sum M(−)_sum_th of integral values in when the number of integral values in a predetermined potential section in the range of −1362 mV to −2501 mV is 9, are equal to each other, and the sum H(−)_sum_th_₇₄ of integral values that is the sum H(−)_sum_th of integral values in when the number of integral values in a predetermined potential section in the range of −1362 mV to −2501 mV is 74, the sum H(−)_sum_th_₃₇ of integral values that is the sum H(−)_sum_th of integral values in when the number of integral values in a predetermined potential section in the range of −1362 mV to −2501 mV is 37, the sum H(−)_sum_th_₁₉ of integral values that is the sum H(−)_sum_th of integral values in when the number of integral values in a predetermined potential section in the range of −1362 mV to −2501 mV is 19, and the sum H(−)_sum_th_₉ of integral values that is the sum H(−)_sum_th of integral values in when the number of integral values in a predetermined potential section in the range of −1362 mV to −2501 mV is 9, are equal to each other.

As a result, in a predetermined potential section in the range of −1362 mV to −2501 mV, Body index (−)_{th_74} in when the number of integral values is 74, Body index (−)_{th_37} in when the number of integral values is 37, Body index (−)_{th_19} in when the number of integral values is 19, and Body index (−)_{th_9} in when the number of integral values is 9 are equal to each other.

In this case, even if the number of integral values changes, the coefficient k9 shown in equation (11) does not change, so the “astringency” of grapes in when the number of integral values in a predetermined potential section in the range of −1362 mV to −2501 mV is 74, the “astringency” of grapes in when the number of integral values in a predetermined potential section in the range of −1362 mV to −2501 mV is 37, the “astringency” of grapes in when the number of integral values in a predetermined potential section in the range of −1362 mV to −2501 mV is 19, and the “astringency” of grapes in when the number of integral values in a predetermined potential section in the range of −1362 mV to −2501 mV is 9 are equal to each other.

For the same reason, when the number of integral values in a predetermined potential section in the range of −1362 mV to −2501 mV is 25, 15, 13, 11, and 7, also “the plurality of astringency” of the grapes are equal to each other.

Therefore, the diagnostic device 2 may perform a taste diagnosis of “grapes” by the above-mentioned method based on an integral value spectrum in which the number of integral values in a predetermined potential section in the range of −1362 mV to −2501 mV is any one of 7, 9, 11, 13, 15, 19, 25, 37, and 74.

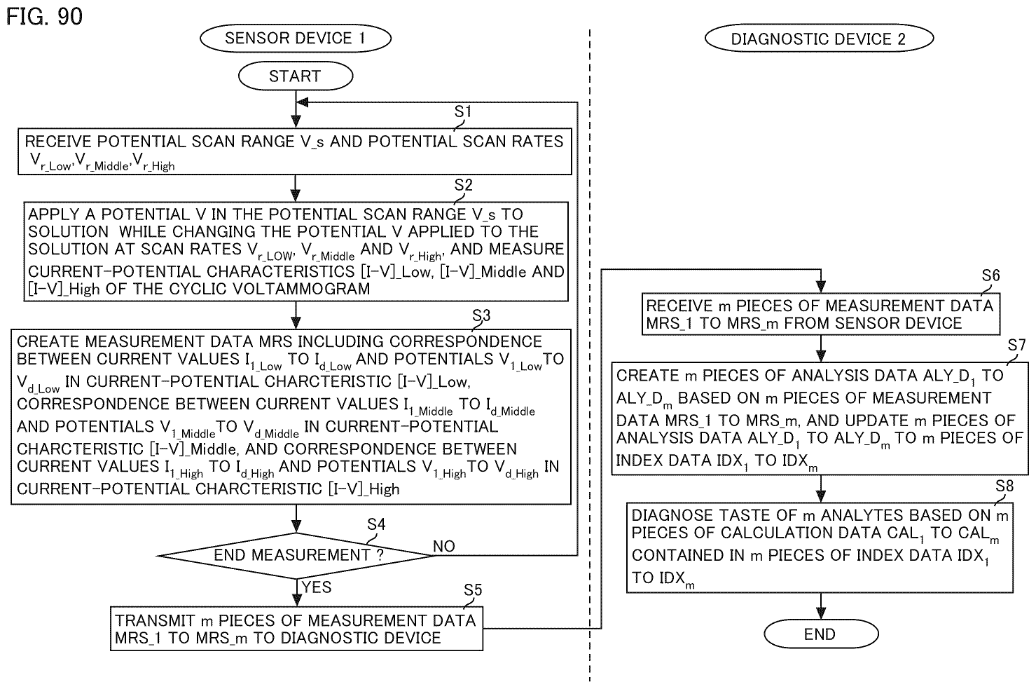

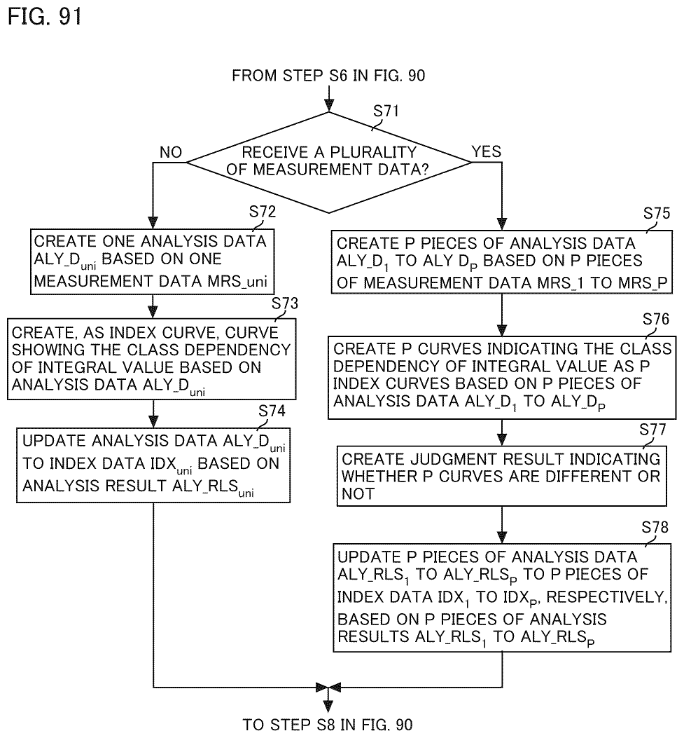

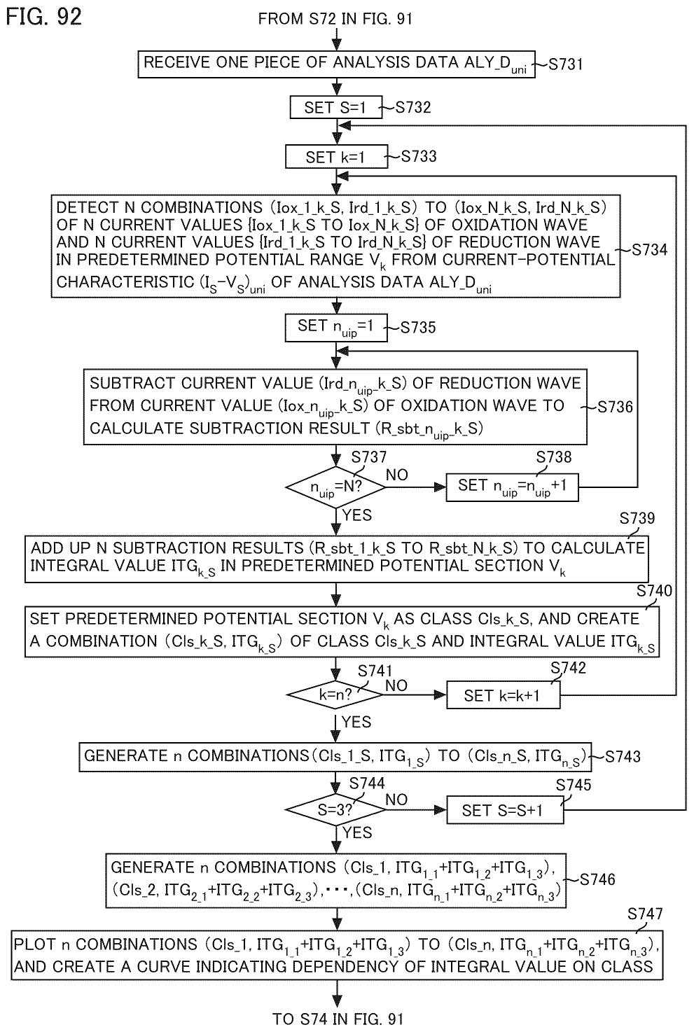

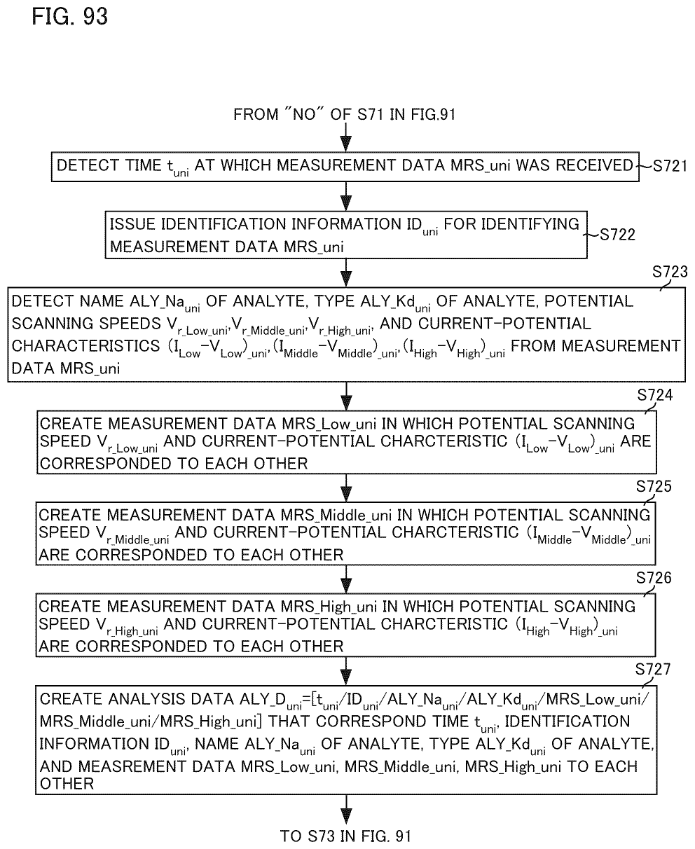

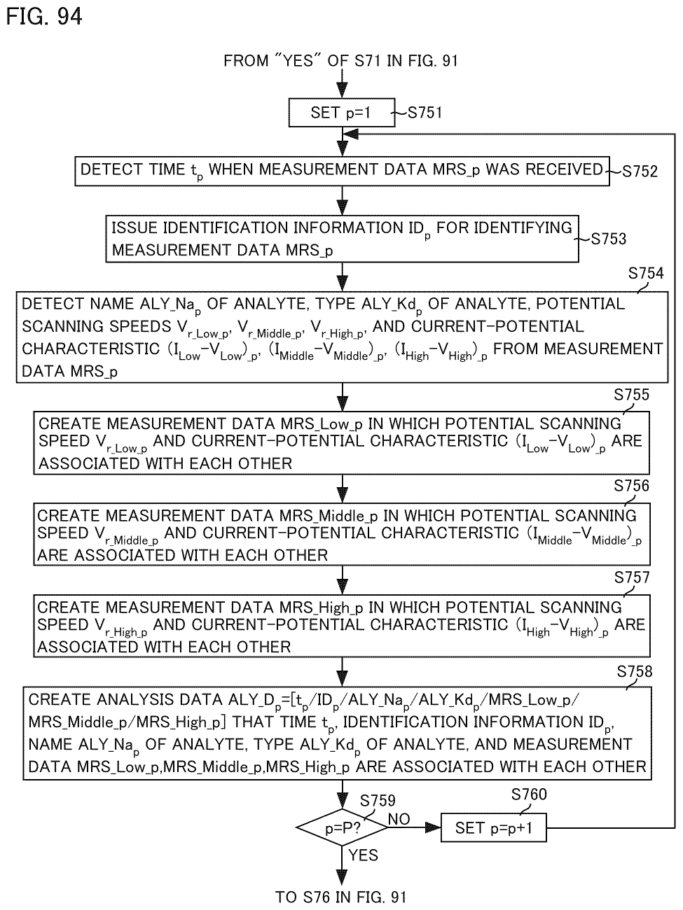

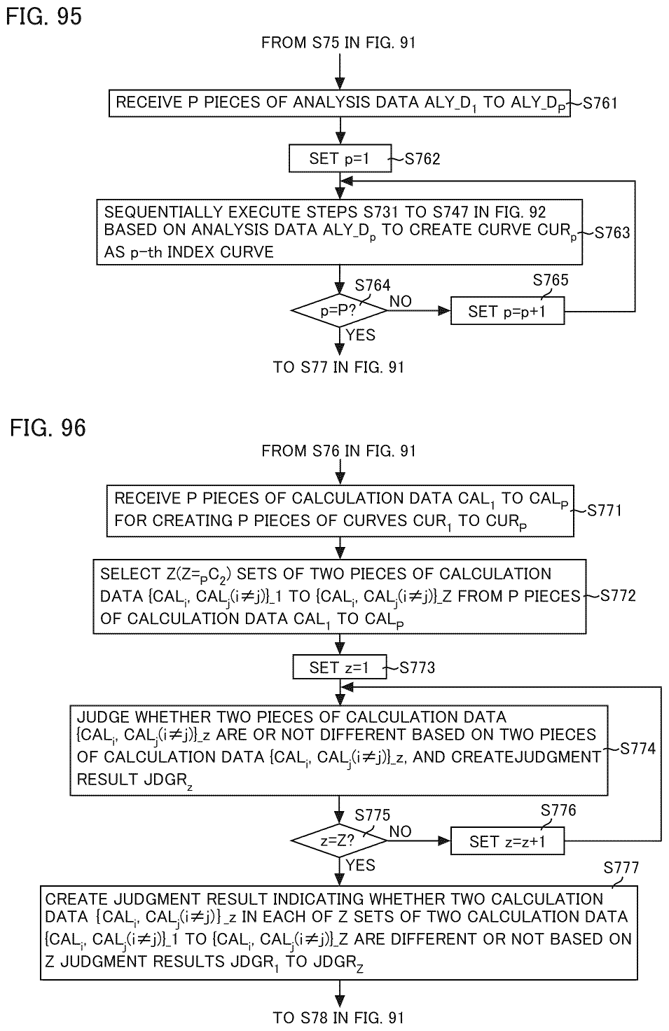

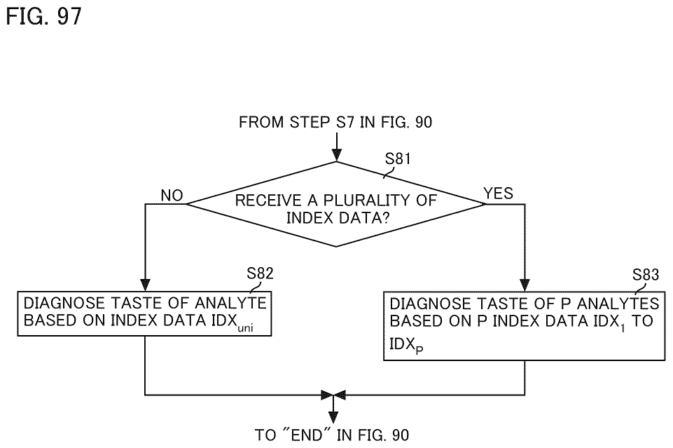

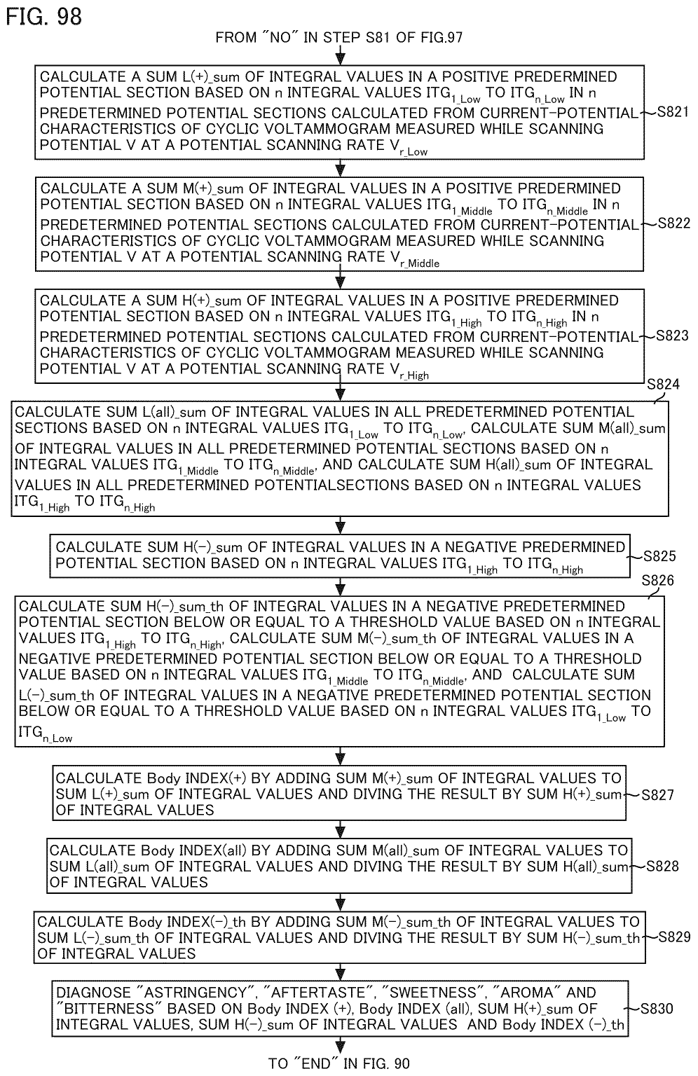

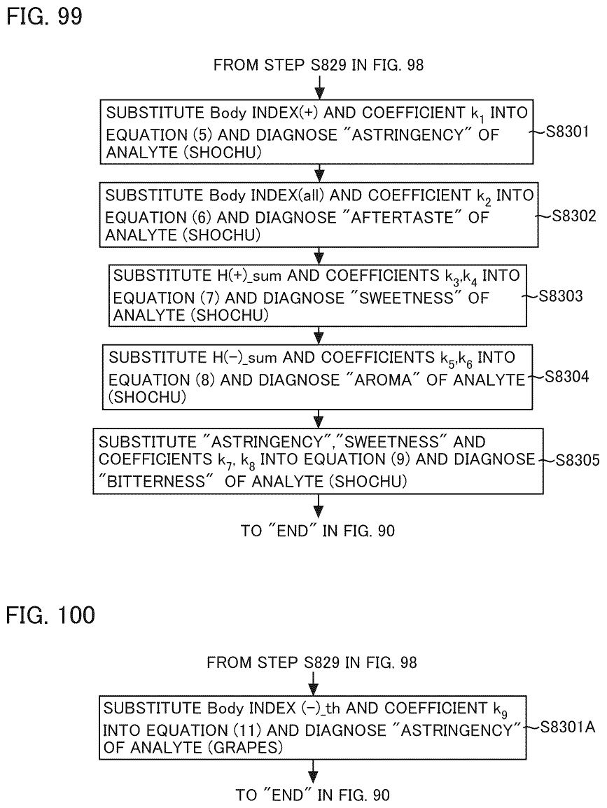

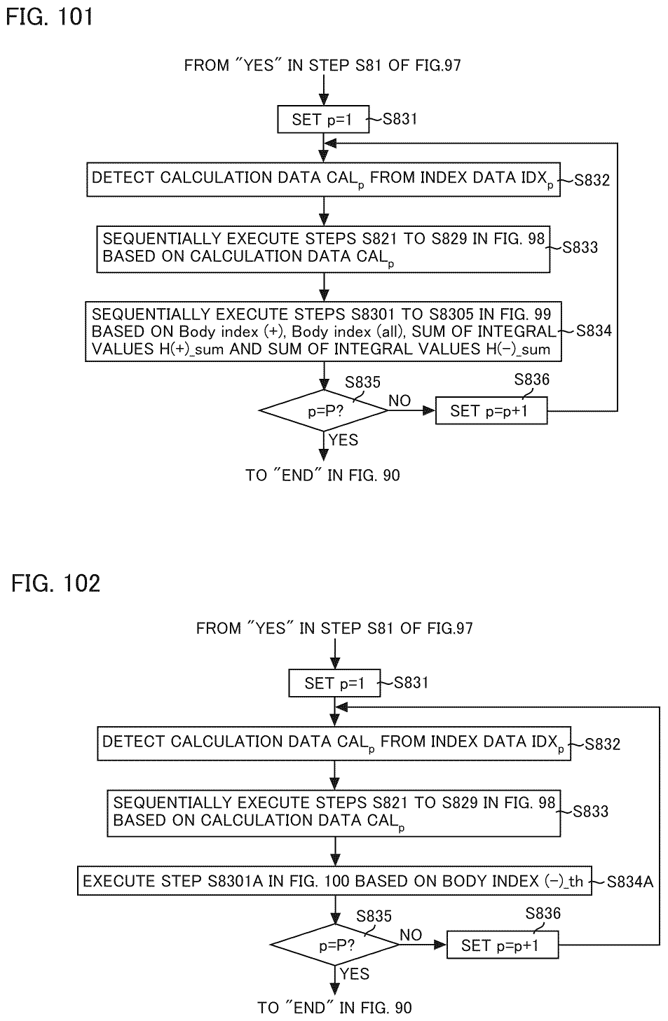

FIG. 90 is a flowchart for explaining the operation of the diagnostic system 10 shown in FIG. 1.

Referring to FIG. 90, when the operation of the diagnostic system 10 is started, the supply unit 121 of the sensor device 1 receives the potential scanning range V_s and the potential scan rates V_{r_Low}, V_{r_Middle}, and V_{r_High} input to the measuring instrument 12 by the user of the sensor device 1 (step S1).

In addition, the supply unit 121 of the sensor device 1 accepts a start signal input to the measuring instrument 12 by the user of the sensor device 1, and the measuring unit 122 of the sensor device 1 accepts an end signal input to the measuring instrument 12 by the user of the sensor device 1.

Then, when the supply unit 121 of the sensor device 1 received the start signal, the supply unit 121 applies a potential V of the potential scanning range V_s to the solution while changing the potential applied to the solution (analyte) at scan speeds of V_{r_Low}, V_{r_Middle}, and V_{r_High}, and the measuring unit 122 measures the potential V of the working electrode 112 based on the potential of the reference electrode 114 and measures the current value I from the counter electrode 113 to measure the current-potential characteristics [I-V]_Low, [I-V]_Middle, and [I-V]_High of the cyclic voltammogram (step S2).

In this case, the supply unit 121 applies a potential V of the potential scanning range V_s to the solution while changing the potential V at the scan speed V_{r_Low}, and the measurement unit 122 measures the potential V of the working electrode 112 based on the potential of the reference electrode 114, and measures the current value I from the counter electrode 113 to measure the current-potential characteristic [I-V]_Low of the cyclic voltammogram.

In addition, the supply unit 121 applies a potential V in a potential scanning range V_s to the solution while changing the potential V at the scan speed V_{r_Middle}, and the measurement unit 122 measures the potential V of the working electrode 112 based on the potential of the reference electrode 114, and measures the current value I from the counter electrode 113 to measure the current-potential characteristic [I-V]_Middle of the cyclic voltammogram.

Furthermore, the supply unit 121 applies a potential V of a potential scanning range V_s to the solution while changing the potential V at the scan speed V_{r_High}, and the measurement unit 122 measures the potential V of the working electrode 112 based on the potential of the reference electrode 114, and measures the current value I from the counter electrode 113 to measure the current-potential characteristic [I-V]_High of the cyclic voltammogram.

After the step S2, the measurement unit 122 creates measurement data MRS including the correspondence between the current values I_{1_Low} to I_{d_Low} and the potentials V_{1_Low} to V_{d_Low} in the current-potential characteristics [I-V]_Low, the correspondence between the current values I_{1_Middle} to I_{d_Middle} and the potentials V_{1_Middle} to V_{d_Middle} in the current-potential characteristics [I-V]_Middle, and the correspondence between the current values I_{1_High} to I_{d_High} and the potentials V_{1_High} to V_{d_High} in the current-potential characteristics [I-V]_High (step S3).

Then, the measuring unit 122 judges whether or not to end the measurement of the cyclic voltammogram of the solution (step S4).

In this case, when the measurement unit 122 receives an end signal input to the measuring instrument 12 by the user of the sensor device 1, the measurement unit 122 judges to end the measurement, and when the measurement unit 122 does not receive the end signal, the measurement unit 122 judges not to end the measurement.

When it is judged in step S4 that the measurement of the cyclic voltammogram of the solution is not to be terminated, the operation of the sensor device 1 transitions to the step S1, and steps S1 to S4 are repeatedly executed until it is judged in the step S4 that the measurement of the cyclic voltammogram of the solution is to be terminated.

In this case, each time it is judged in step S4 that the measurement should not be terminated, the sensor 11 used to measure the cyclic voltammogram is discarded, and a sensor 11 not used to measure the cyclic voltammogram is attached to the measuring instrument 12, and the above-mentioned steps S1 to S4 are executed sequentially.

Then, when it is judged in step S4 that the measurement of the cyclic voltammogram of the solution is to be terminated, the measuring unit 122 of the sensor device 1 outputs m (m is an integer equal to or greater than 1) pieces of measurement data MRS_1 to MRS_m that have been created in when it is judged that the measurement of the cyclic voltammogram of the solution is to be terminated to the transmitting unit 123. The transmitting unit 123 receives the m pieces of measurement data MRS_1 to MRS_m from the measuring unit 122, and transmits the received m pieces of measurement data MRS_1 to MRS_m to the diagnostic device 2 by wired communication or wireless communication (step S5).

The receiving unit 211 of the diagnostic device 2 receives the m pieces of measurement data MRS_1 to MRS_m from the transmitting unit 123 of the sensor device 1 by wired communication or wireless communication (step S6), and outputs the received m pieces of measurement data MRS_1 to MRS_m to the control unit 212.

The control unit 212 of the diagnostic device 2 receives the m pieces of measurement data MRS_1 to MRS_m from the receiving unit 211. Then, the control unit 212 creates m pieces of analysis dataALY_D₁ to ALY_Dₘ based on the m pieces of measurement data MRS_1 to MRS_m, and updates the m pieces of analysis data ALY_D₁ to ALY_Dₘ to m pieces of index data IDX₁ to IDXₘ (step S7).

Then, the control unit 212 outputs the m pieces of index data IDX₁ to IDXₘ to a diagnostic unit (comprised of a calculation unit 216 and a taste diagnostic unit 217).

The diagnostic unit (comprised of the calculation unit 216 and the taste diagnostic unit 217) receives the m pieces of index data IDX₁ to IDXₘ from the control unit 212. The diagnostic unit (comprised of the calculation unit 216 and the taste diagnostic unit 217) then detects m pieces of calculation data CAL₁ to CALₘ from the m pieces of index data IDX₁ to IDXₘ, respectively, and diagnoses the tastes of the m analyte based on the detected m pieces of calculation data CAL₁ to CALₘ (step S8). This completes the operation of the diagnostic system 10.

FIG. 91 is a flowchart for explaining the detailed operation of step S7 in FIG. 90.

Referring to FIG. 91, after the step S6 in FIG. 90, the control unit 212 judges whether a plurality of measurement data have been or not received from the receiving unit 211 (step S71).

In this case, when the control unit 212 has not received the plurality of measurement data from the receiving unit 211, the control unit 212 judges that the control unit 212 has received measurement data MRS_uni from the receiving unit 211, and when the control unit 212 have received the plurality of measurement data from the receiving unit 211, the control unit 212 judges that the control unit 212 has received P pieces of measurement data MRS_1 to MRS_P (multiple measurement data MRS) from the receiving unit 211.

When it is judged in step S71 that the plurality of measurement data have not been received, the control unit 212 creates one piece of analysis data ALY_Dᵤₙᵢ based on one piece of measurement data MRS_uni using the method described above (step S72), stores the created analysis data ALY_Dᵤₙᵢ in the database 22, and outputs the analysis data ALY_Dᵤₙᵢ to the calculation unit 213.

The calculation unit 213 receives the analysis data ALY_Dᵤₙᵢ from the control unit 212. Then, the calculation unit 213 and the creation unit 215 create a curve CURᵤₙᵢ indicating the class dependency of the integral value as an index curve based on the analysis dataALY_Dᵤₙᵢ (step S73).

Then, the creation unit 215 creates an analysis result ALY_RLSᵤₙᵢ=[IDᵤₙᵢ/CALᵤₙᵢ/CURᵤₙᵢ] that associates the identification information IDᵤₙᵢ, the calculation data CALᵤₙᵢ, and the curve CURᵤₙᵢ, and outputs the created analysis result ALY_RLSᵤₙᵢ=[IDᵤₙᵢ/CALᵤₙᵢ CURᵤₙᵢ] to the control unit 212.

The control unit 212 receives the analysis result ALY_RLSᵤₙᵢ from the creation unit 215, updates the analysis data ALY_Dᵤₙᵢ to index data IDXᵤₙᵢ based on the received analysis result ALY_RLSᵤₙᵢ (step S74), and stores the updated index data IDXᵤₙᵢ in the database 22 in place of the analysis data ALY_Dᵤₙᵢ.

On the other hand, when it is judged in step S71 that a plurality of measurement data have been received, the control unit 212 creates P pieces of analysis data ALY_D₁ to ALY_D_{P} based on the P pieces of measurement data MRS_1 to MRS_P by the method described above (step S75), stores the created P pieces of analysis data ALY_D₁ to ALY_D_{P} in the database 22, and outputs the P pieces of analysis data ALY_D₁ to ALY_D_{P} to the calculation unit 213.

The calculation unit 213 receives the P pieces of analysis dataALY_D₁ to ALY_D_{P} from the control unit 212. Then, the calculation unit 213 and the creation unit 215 create P curves CUR₁ to CUR_{P} indicating the class dependency of the integral value based on the P pieces of analysis data ALY_D₁ to ALY_D_{P} as P index curves of the P analytes (step S76).

Then, the judgment unit 214 creates a judgment result (judgment result shown in Table 1) indicating whether the P curves CURᵢ to CUR_{P} are different or not (step S77), and outputs the created judgment result (judgment result shown in Table 1) to the creation unit 215.

When the creation unit 215 receives the judgment results (the judgment results shown in Table 1) from the judgment unit 214, the creation unit 215 adds P curves CUR₁ to CUR_{P} to the P calculation results CAL_RLS₁=[ID₁/CAL₁] to CAL_RLS_{P}=[ID_{P}/CAL_{P}], respectively, to create P analysis results ALY_RLS₁=[ID₁/CAL₁/CUR₁] to ALY_RLS_{P}=[ID_{P}/CAL_{P}/CUR_{P}]. Then, the creation unit 215 outputs the P analysis results ALY_RLS₁ to ALY_RLS_{P} and the judgment results (the judgment results shown in Table 1) to the control unit 212.

Then, when the control unit 212 receives the P analysis results ALY_RLS₁ to ALY_RLS_{P} and the judgment results (the judgment results shown in Table 1) from the creation unit 215, the control unit 212 updates the P analysis data ALY_D₁ to ALY_D_{P} to P index data IDX₁ to IDX_{P}, respectively, based on the P analysis results ALY_RLS₁ to ALY_RLS_{P} (step S78), and stores the updated P index data IDX₁ to IDXₚ in the database 22 in place of the P analysis data ALY_D₁ to ALY_D_{P}, respectively.

In this case, the control unit 212 detects the identification information IDₚ, the calculation data CAL_{P}, and the curve CURₚ from the analysis result ALY_RLSₚ (p is any of 1 to P), reads out the analysis data ALY_Dₚ having the same identification information as the detected identification information IDₚ from the database 22, and updates the analysis data ALY_Dₚ to index data IDXₚ by adding the calculation data CAL_{P} and the curve CURₚ to the read analysis data ALY_Dₚ, performing this process for all of P analysis data ALY_D₁ to ALY_D_{P}.

As a result, the P pieces of analysis data ALY_D₁ to ALY_D_{P} are updated to the P pieces of index data IDX₁ to IDX_{P}, respectively.

Then, the control unit 212 stores the P index data IDX₁ to IDX_{P} in the database 22 in place of the P analysis data ALY_D₁ to ALY_D_{P}, respectively, and stores the judgment result (the judgment result shown in Table 1) in the database 22 in association with the P index data IDX₁ to IDX_{P}.

After step S74 or step S78, the operation of the diagnostic device 2 proceeds to step S8 in FIG. 90.

According to the flowchart shown in FIG. 91, when the diagnostic device 2 judges in step S71 that multiple measurement data have not been received, the diagnostic device 2 creates one curve CURᵤₙᵢ (see step S73), and when the diagnostic device 2 judges in step S71 that multiple measurement data have been received, the diagnostic device 2 creates P curves CUR₁ to CUR_{P} and the judgment result (the judgment result shown in Table 1) indicating whether the P curves CUR₁ to CUR_{P} are or not different (see steps S76 and S77).

Therefore, the diagnostic device 2 can create the curve CURᵤₙᵢ (or P curves CUR₁ to CUR_{P}) as an index curve that serves as an index when identifying the analyte.

FIG. 92 is a flowchart for explaining the detailed operation of step S73 in FIG. 91

In FIG. 92, the potential scanning speed Vᵣ is represented by “S.” Furthermore, “S=1” represents the potential scanning speed V_{r_Low}, “S=2” represents the potential scanning speed V_{r_Middle}, and “S=3” represents the potential scanning speed V_{r_High}.

Referring to FIG. 92, after step S72 in FIG. 91, the calculation unit 213 receives one piece of analysis data ALY_Dᵤₙᵢ from the control unit 212 (step S731).

Then, the calculation unit 213 sets S=1 (step S732) and sets k=1 (step S733). Here, k is an argument representing the predetermined potential section.

After step S733, the calculation unit 213 detects N combinations (Iox_1_k_S, Ird_1_k_S) to (Iox_N_k_S, Ird_N_k_S) of N current values {Iox_1_k_S to Iox_N_k_S} of the oxidation wave and N current values {Ird_1_k_S to Ird_N_k_S} of the reduction wave in the predetermined potential range Vₖ from the current-potential characteristic (I_{S}-V_{S})ᵤₙᵢ of the analysis dataALY_Dᵤₙᵢ (step S734).

Here, N represents the total number of unit potentials (for example, 1 mV) in one predetermined potential section Vₖ.

Also, for example, when one predetermined potential section Vₖ is [0 to 100 mV], since the current-potential characteristic (I_{S}-V_{S})ᵤₙᵢ includes a current value I_{0→100} in when the potential V was scanned from 0 mV to 100 mV and a current value I_{100→0} in when the potential V was scanned from 100 mV to 0 mV, the calculation unit 213 detects the current value I_{0→100} in when the potential V was scanned from 0 mV to 100 mV as [N current values {Iox_1_k_S to Iox_N_k_S} of oxidation wave], and detects the current value I_{100→0} in when the potential V is scanned from 100 mV to 0 mV as [N current values {Ird_1_k_S to Ird_N_k_S} of reduction wave], and N combinations (Iox_1_k_S, Ird_1_k_S) to (Iox_N_k_S, Ird_N_k_S). The same is applied to the case where one predetermined potential section Vₖ is other than [0 to 100 mV].

After step S734, the calculation unit 23 sets nᵤₙᵢp=1 (step S735). Here, nᵤᵢₚ is an argument representing each of the N unit potentials in one predetermined potential section Vₖ.

After step S735, the calculation unit 213 subtracts the current value (Ird_nᵤᵢₚk_S) of the reduction wave from the current value (lox nᵤᵢₚ_k_S) of the oxidation wave to calculate the subtraction result (R_sbt_nᵤᵢₚ_k_S) (step S736).

Then, the calculation unit 213 judges whether or not nᵤᵢₚ=N (step S737).

When it is judged in step S737 that nᵤᵢₚ=N is not satisfied, the calculation unit 213 sets nᵤᵢₚ=nᵤᵢₚ+1 (step S738). Thereafter, the operation of the diagnostic device 2 proceeds to step S736, and steps S736 to S738 are executed repeatedly until it is judged in step S737 that nᵤₙᵢp=N is satisfied.

If it is judged in step S737 that nᵤₙᵢp=N, the calculation unit 213 adds up the N subtraction results (R_sbt_1_k_S to R_sbt_N_k_S) to calculate the integral value ITG_{k_s} in the predetermined potential section Vₖ (step S739).

Thereafter, the calculation unit 213 sets the predetermined potential section Vₖ as the class Cls_k_S, and creates a combination (Cls_k_S, ITG_{k_s}) of the class Cls_k_S and the integral value ITG_{k_s} (step S740).

Then, the calculation unit 213 judges whether or not k=n holds (step S741). Here, n is the total number of the predetermined potential sections Vₖ.

When it is judged in step S741 that k=n is not true, the calculation unit 213 sets k=k+1 (step S742). After that, the operation of the diagnostic device 2 proceeds to step S734, and steps S734 to S742 are repeatedly executed until it is judged in step S741 that k=n is true.

Then, when it is judged in step S741 that k=n is true, the calculation unit 213 generates n combinations (Cls_1_S, ITG_{1_S}) to (Cls__{n_S}, ITG_{n_S}) (step S743).

Then, the calculation unit 213 judges whether S=3 is or not true (step S744).

When it is judged in step S744 that S is not 3, the calculation unit 213 sets S=S+1 (step S745). After that, the operation of the diagnostic device 2 proceeds to step S733. Then, steps S733 to S745 are repeatedly executed until it is judged in step S744 that S=3 is true.

Then, if it is judged in step S744 that S=3 is true, the calculation unit 213 adds up the three integral values ITG_{1_1}, ITG_{1_2}, and ITG_{1_3} in class Cls_1 to calculate the sum ITG_{1_1}+ITG_{1_2}+ITG_{1_3} of the integral values, adds up the three integral values ITG_{2_1}, ITG_{2_2}, and ITG_{2_3} in class Cls_2 to calculate the sum ITG_{2_1}+ITG_{2_2}+ITG_{2_3} of the integral values, and similarly adds up the three integral values ITG. 1, ITG_{n_2}, and ITG_{n_3} in class Cls_n to calculate the sum ITG_{n_1}+ITG_{n_2}+ITG_{n_3} of the integral values.

Then, the calculation unit 213 generates n combinations (Cls_1, ITG_{1_1}+ITG_{1_2}+ITG_{1_3}), (Cls_2, ITG_{2_1}+ITG_{2_2}+ITG_{2_3}), . . . , (Cls_n, ITG_{n_1}+ITG_{n_2}+ITG_{n_3}) (step S746).

Then, the calculation unit 213 creates the calculation data CAL consisting of n combinations (Cls_1, ITG_{1_1}+ITG_{1_2}+ITG_{1_3}), (Cls_2, ITG_{2_1}+ITG_{2_2}+ITG_{2_3}), . . . , (Cls_n, ITG_{n_1}+ITG_{n_2}+ITG_{n_3}) and outputs a calculation result CAL_RLS=[identification information ID/calculation data CAL] in which the calculation data CAL is corresponded to the identification information ID of the analyte to the creation unit 215.

The creation unit 215 receives the calculation result CAL_RLS=[identification information ID/calculated data CAL] from the calculation unit 213. Then, the creation unit 215 detects n combinations (Cls_1, ITG_{1_1}+ITG_{1_2}+ITG_{1_3}), (Cls_2, ITG_{2_1}+ITG_{2_2}+ITG_{2_3}), . . . , (Cls_n, ITG_{n_1}+ITG_{n_2}+ITG_{n_3}) from the calculation data CAL of the calculation result CAL_RLS, plots the detected n combinations (Cls_1, ITG_{1_1}+ITG_{1_2}+ITG_{1_3}), (Cls_2, ITG_{2_1}+ITG_{2_2}+ITG_{2_3}), . . . , (Cls_n, ITG_{n_1}+ITG_{n_2}+ITG_{n_3}) to create a curve CUR indicating the dependency of the integral value on the class (step S747).

After step S747, the operation of the diagnostic device 2 proceeds to step S74 in FIG. 91.

In the flowchart shown in FIG. 92, the calculation unit 213 calculates n integral values ITG_{1_1} to ITG_{n_1} in n classes Cls_1_1 to Cls_n_1 based on the cyclic voltammogram measured at the potential scanning speed V_{r_Low} to create n combinations (Cls_1_1, ITG_{1_1}) to (Cls_n_1, ITG_{n_1}) of classes and integral values, the calculation unit 213 calculates n integral values ITG_{2_1} to ITG_{n_2} in n classes Cls_1_2 to Cls_n_2 based on the cyclic voltammogram measured at the potential scanning speed V_{r_Middle} to create n combinations (Cls_1_2, ITG_{1_2}) to (Cls_n_2, ITG_{n_2}) of classes and integral values, and the calculation unit 213 calculates n integral values ITG₁_3 to ITG. 3 in n classes Cls_1_3 to Cls_n_3 based on the cyclic voltammogram measured at the potential scanning speed V_{r_High} to create n combinations (Cls_1_3, ITG_{1_3}) to (Cls_n_3, ITG_{n_3}) of classes and integral values.

Then, the calculation unit 213 executes for all of n of classes to calculate the sum (ITG_{k_1}+ITG_{k_2}+ITG_{k_3}) of the three integral values ITG_{k_1}, ITG_{k_2}, ITG_{k_3} in one class k (=predetermined potential section Vₖ) based on the n combinations (Cls_1_1, ITG_{1_1}) to (Cls_n_1, ITG_{n_1}), n combinations (Cls_1_2, ITG_{1_2}) to (Cls_n_2, ITG_{n_2}), and n combinations (Cls_1_3, ITG_{1_3}) to (Cls_n_3, ITG₃) to calculate the n sums (ITG_{1_1}+ITG_{1_2}+ITG_{1_3}) to (ITG_{n_1}+ITG_{n_2}+ITG_{n_3}).

Then, the calculation unit 213 generates n combinations (Cls_1, ITG_{1_1}+ITG_{1_2}+ITG_{1_3}), (Cls_2, ITG_{2_1}+ITG_{2_2}+ITG_{2_3}), . . . , (Cls_n, ITG_{n_1}+ITG_{n_2}+ITG_{n_3}) based on n classes Cls_1 to Cls_n and n sums (ITG_{1_1}+ITG_{1_2}+ITG_{1_3}) to (ITG_{n_1}+ITG_{n_2}+ITG_{n_3}) (see step S746), and the calculation unit 213 outputs the generated n combinations (Cls_1, ITG_{1_1}+ITG_{1_2}+ITG_{1_3}), (Cls_2, ITG_{2_1}+ITG_{2_2}+ITG_{2_3}), . . . , (Cls_n, ITG_{n_1}+ITG_{n_2}+ITG_{n_3}) to the creation unit 215.

The creation unit 215 receives n combinations (Cls_1, ITG_{1_1}+ITG_{1_2}+ITG_{1_3}), (Cls_2, ITG_{2_1}+ITG_{2_2}+ITG_{2_3}), . . . , (Cls_n, ITG_{n_1}+ITG_{n_2}+ITG_{n_3}) from the calculation unit 213, and plots the received n combinations (Cls_1, ITG_{1_1}+ITG_{1_2}+ITG_{1_3}), (Cls_2, ITG_{2_1}+ITG_{2_2}+ITG_{2_3}), . . . , (Cls_n, ITG_{n_1}+ITG_{n_2}+ITG_{n_3}) to create a curve CUR indicating the class dependency of the integral value (see step S747).

FIG. 93 is a flowchart for explaining the detailed operation of step S72 in FIG. 91.

Referring to FIG. 93, when it is judged in step S71 of FIG. 91 that a plurality of measurement data have not been received, the control unit 212 of the diagnostic device 2 receives the measurement data MRS_uni from the receiving unit 211. Then, the control unit 212 refers to a timer to detect the time tᵤₙᵢ at which the measurement data MRS_uni was received (step S721), and issues identification information IDᵤₙᵢ for identifying the measurement data MRS_uni (step S722).

Then, the control unit 212 detects the name ALY_Naᵤₙᵢ of the analyte, the type ALY_Kdᵤₙᵢ of the analyte, the potential scanning speeds V_{r_Low_uni}, V_{r_Middle_uni}, V_{r_High_uni}, and the current-potential characteristics (I_{Low}-V_{Low})ᵤₙᵢ, (I_{Middle}-V_{Middle})ᵤₙᵢ, and (I_{High}-V_{High})ᵤₙᵢ from the measurement data MRS_uni (step S723).

Then, the control unit 212 creates measurement data MRS_Low_uni in which the potential scanning speed V_{r_Low_uni} and the current-potential characteristic (I_{Low}-V_{Low})ᵤₙᵢ are corresponded to each other (step S724).

Furthermore, the control unit 212 creates measurement data MRS_Middle_uni in which the potential scanning speed V_{r_Middle_uni} and the current-potential characteristic (I_{Middle}-V_{Middle})ᵤₙᵢ are associated with each other (step S725).

Furthermore, the control unit 212 creates measurement data MRS_High_uni in which the potential scanning speed V_{r_High_uni} and the current-potential characteristic (I _{High}-V _{High})ᵤₙᵢ are associated with each other (step S726).

Then, the control unit 212 creates analysis data ALY_Dᵤₙᵢ=[tᵤₙᵢ/IDᵤₙᵢ/ALY_Naᵤₙᵢ/ALY_Kdᵤₙᵢ/MRS_Low_uni/MRS_Middle_uni/MRS_High_uni] that corresponds the time tᵤₙᵢ, identification information IDᵤₙᵢ, name of the analyte ALY_Naᵤₙᵢ, type of the analyte ALY_Kdᵤₙᵢ, and measurement data MRS_Low_uni, MRS_Middle_uni, and MRS_High_uni to each other (step S727).

After step S727, the operation of the diagnostic device 2 proceeds to step S73 in FIG. 91.

FIG. 94 is a flowchart for explaining the detailed operation of step S75 in FIG. 1.

Referring to FIG. 94, when it is judged in step S71 of FIG. 91 that a plurality of measurement data have been received, the control unit 212 of the diagnostic device 2 receives P pieces of measurement data MRS_1 to MRS_P from the receiving unit 211. Then, the control unit 212 sets p=1 (step S751). Here, p is an argument representing each of the P pieces of measurement data MRS_1 to MRS_P, and p=1 to P.

After step S751, the control unit 212 refers to the timer to detect the time tₚ at when the measurement data MRS_p was received (step S752), and issues identification information IDₚ for identifying the measurement data MRS_p (step S753).

Then, the control unit 212 detects the name ALY_Naₚ of the analyte, the type ALY_Kdₚ of the analyte, the potential scanning speeds V_{r_Low_p}, V_{r_Middle_p}, V_{r_High_p}, and the current-potential characteristics (I_{Low}-V_{Low})ₚ, (I_{Middle}-V_{Middle})ₚ, (I_{High}-V_{High})ₚ from the measurement data MRS_p (step S754).

Then, the control unit 212 creates measurement data MRS_Low_p in which the potential scanning speed V_{r_Low_p} and the current-potential characteristic (I_{Low}-V_{Low})ₚ are associated with each other (step S755).

Furthermore, the control unit 212 creates measurement data MRS_Middle_p in which the potential scanning speed V_{r_Middle_p} and the current-potential characteristic (I _{Middle}-V _{Middle})ₚ are associated with each other (step S756).

Furthermore, the control unit 212 creates measurement data MRS_High_p in which the potential scanning speed V_{r_High_p} and the current-potential characteristic (I _{High}-V _{High})ₚ are associated with each other (step S757).

The control unit 212 then creates analysis data ALY_Dₚ=[tₚ/IDₚ/ALY_Naₚ/ALY_Kdₚ/MRS_Low_p/MRS_Middle_p/MRS_High_p] that the time tₚ, the identification information IDₚ, the name ALY_Naₚ of the analyte, the type ALY_Kdₚ of analyte, and the measurement data MRS_Low_p, MRS_Middle_p, and MRS_High_p are associated with each other (step S758).

Then, the control unit 212 judges whether p=P or not (step S759).

When it is judged in step S759 that p=P is not satisfied, control unit 212 sets p=p+1 (step S760). Thereafter, the operation of diagnostic device 2 proceeds to step S752, and steps S752 to S760 are repeatedly executed until it is judged in step S759 that p=P is satisfied.

Then, if it is judged in step S759 that p=P, the operation of the diagnostic device 2 proceeds to step S76 in FIG. 91.

In the flowchart shown in FIG. 94, when it was judged in step S759 that p=P, P pieces of analysis data ALY_D₁ to ALY_D_{P} have been created.

FIG. 95 is a flowchart for explaining the detailed operation of step S76 in FIG. 91.

Referring to FIG. 95, after step S75 in FIG. 91, the calculation unit 213 receives P pieces of analysis dataALY_D₁ to ALY_D_{P} from the control unit 212 (step S761).

Then, the calculation unit 213 sets p=1 (step S762). Here, p is an argument representing each of the P pieces of analysis data ALY_D₁ to ALY_D_{P}.

After step S762, the calculation unit 213 and the creation unit 215 sequentially execute steps S731 to S747 in FIG. 92 based on the analysis data ALY_Dₚ to create the curve CURₚ as the p-th index curve (step S763).

In this case, in step S731 of FIG. 92, “analysis data ALY_Dᵤₙᵢ,” is replaced with “analysis data ALY_Dₚ”, and in step S734 of FIG. 92, “analysis data ALY_Dᵤₙᵢ,” is replaced with “analysis data ALY_Dₚ”, and “current-potential characteristic (I-V)ᵤₙᵢ” is replaced with “current-potential characteristic (I-V)ₚ”.

After the step 763, the calculation unit 213 judges whether p=P or not (step S764).

When it is judged in step S764 that p=P is not satisfied, the calculation unit 213 sets p=p+1 (step S765). Thereafter, the operation of the diagnostic device 2 proceeds to step S763, and steps S763 to S765 are executed repeatedly until it is judged in step S764 that p=P is satisfied.

Then, if it is judged in step S764 that p=P, the operation of the diagnostic device 2 proceeds to step S77 in FIG. 91.

According to the flowchart shown in FIG. 95, when it is judged in step S764 that p=P, P curves CUR₁ to CUR_{P} have been created as P index curves for P analytes, respectively.

FIG. 96 is a flowchart for explaining the detailed operation of step S77 in FIG. 91.

Referring to FIG. 96, after step S76 in FIG. 91, the judgment unit 214 receives P pieces of calculation data CAL₁ to CAL_{P} for creating P pieces of curves CUR₁ to CUR_{P} from the calculation unit 213 (step S771).

Then, the judgment unit 214 selects Z (Z=_{P}C₂) sets of two pieces of calculation data {CALᵢ, CALⱼ (i≠j)}_1 to {CALᵢ, CALⱼ(i≠j)}_Z from the P pieces of calculation data CAL₁ to CAL_{P} (step S772). Here, the two pieces of calculation data CALᵢ, CALⱼ are different calculation data.

After step S772, the judgment unit 214 sets z=1 (step S773). Here, z is an argument representing each of Z sets of two calculation data {CALᵢ, CALⱼ(i≠j)}_1 to {CALᵢ, CALⱼ(i≠j)}_Z, respectively.

After step S773, the judgment unit 214 judges whether the two pieces of calculation data {CALᵢ, CALⱼ(i≠j)}_z are or not different based on the two pieces of calculation data {CALᵢ, CALⱼ(i≠j)}_z, and creates a judgment result JDGR_{z} (step S774).

In this case, the judgment unit 214 judges whether the two pieces of calculation data {CALᵢ, CALⱼ (i≠j)}_z are or not different by the following method.

Each of the two pieces of calculation data {CALᵢ, CALⱼ(i≠j)}_z has the same configuration as the calculation data CALᵤₙᵢ shown in FIG. 12.

As a result, the calculation data CALᵢ_z includes the sums {ITG_{1_Low}+ITG_{1_Middle}+ITG_{1_High}}ᵢ_Z to {ITG_{n_Low}+ITG_{n_Middle}+ITG_{n_High}}ᵢ_z of n integral values corresponding to the n classes Cls₁ to Clsₙ, respectively. Also, the calculation data CALⱼ_z includes the sums {ITG_{1_Low}+ITG_{1_Middle}+ITG_{1_High}}ⱼ_Z to {ITG_{n_Low}+ITG_{n_Middle}+ITG_{n_High}}ⱼ_z of n integral values corresponding to the n classes Cls₁ to Clsₙ, respectively.

The judgment unit 214 executes, for all of g=1 to n, calculating the difference DF_{g} between the sum {ITG_{g_Low}+ITG_{g_Middle}+ITG_{g_High}}ᵢ_z of the integral values and the sum {ITG_{g_Low}+ITG_{g_Middle}+ITG_{g_High}}ⱼ_z of the integral values in one class Cls_{g} (g=1 to n) to calculate n differences DF₁ to DFₙ.

Then, the judgment unit 214 calculates the standard deviation σ_{i,j} of the n differences DF₁ to DFₙ.

Then, when the standard deviation σ_{i,j} is greater than the threshold value σₜₕ, the judgment unit 214 judges that the two calculated data {CALᵢ, CAL₁(i≠j)}_z are different, and when the standard deviation σ_{i,j} is equal to or less than the threshold value σₜₕ, the judgment unit 214 judges that the two calculated data {CALᵢ, CALⱼ(i≠j)}_z are not different.

Therefore, the judgment result JDGR_{z} includes a judgment result (◯)_that the two of calculation data {CALᵢ, CALⱼ(i≠j)}_z are different or a judgment result (x)_that the two calculation data {CALᵢ, CALⱼ(i≠j)}_z are not different about the two of calculation data {CALᵢ, CALⱼ(i≠j)}_z.

After the step S774, the judgment unit 214 judges whether z=Z or not (step S775). Here, Z represents the total number of Z sets of two pieces of calculation data {CALᵢ, CALⱼ(i≠j)}_1 to {CALᵢ, CALⱼ(i≠j)}_Z.

When it is judged in step S775 that z=Z is not satisfied, the judgment unit 214 sets z=z+1 (step S776). After that, the operation of the judgment unit 214 proceeds to step S774, and steps S774 to S776 are executed repeatedly until it is judged in step S775 that z=Z is satisfied.

Then, when it is judged in step S775 that z=Z is true, the judgment unit 214 creates a judgment result indicating whether the two calculation data {CALᵢ, CALⱼ(i≠j)}_z in each of the Z sets of two calculation data {CALᵢ, CALⱼ (i≠j)}_1 to {CALᵢ, CALⱼ (i≠j)}_Z are or not different based on the Z judgment results JDGR₁ to JDGR_{z} (step S777).

After the step S777, the operation of the diagnostic device 2 proceeds to the step S78 in FIG. 91.

FIG. 97 is a flowchart for explaining the detailed operation of the step S8 in FIG. 90.

Referring to FIG. 97, after the step S7 in FIG. 90, the calculation unit 216 receives m pieces of index data IDX₁ to IDXₘ from the control unit 212.

Then, the calculation unit 216 judges whether a plurality of index data have been or not received from the control unit 212 (step S81).

In this case, when the calculation unit 216 has not been received multiple index data from the control unit 212, the calculation unit 216 judges that the calculation unit 216 has received index data IDXᵤₙᵢ from the control unit 212, and when the calculation unit 216 has received multiple index data from the control unit 212, the calculation unit 216 judges that the calculation unit 216 has received P pieces of index data IDX₁ to IDX_{P} (multiple index data IDX) from the control unit 212.

When it is judged in the step S81 that a plurality of index data have not been received from the control unit 212, the calculation unit 216 and the taste diagnostic unit 217 diagnose the taste of the analyte based on the index data IDXᵤₙᵢ (step S82).

On the other hand, when it is judged in the step S81 that multiple index data have been received from the control unit 212, the calculation unit 216 and the taste diagnostic unit 217 diagnose the taste of the P analytes based on the P index data IDX₁ to IDX_{P} (multiple index data IDX) (step S83).

After the step S82 or the step S83, the operation of the diagnostic device 2 proceeds to “End” in FIG. 90.

FIG. 98 is a flowchart for explaining the detailed operation of the step S82 in FIG. 97.

Referring to FIG. 98, when it is judged in the step S81 of FIG. 97 that multiple index data have not been received, the calculation unit 216 detects calculation data CALᵤₙᵢ from the index data IDXᵤₙᵢ, and calculates a sum L(+)_sum of the integral values in a positive predetermined potential section based on the n integral values ITG_{1_Low} to ITG_{n_Low} of the detected calculation data CALᵤₙᵢ (=n integral values ITG_{1_Low} to ITG_{n_Low} in n predetermined potential sections calculated from the current-potential characteristics of a cyclic voltammogram measured by scanning the potential V at a potential scanning speed V_{r_Low}) (step S821).

In addition, the calculation unit 216 calculates the sum M(+)_sum of the integral values in a positive predetermined potential section based on the n integral values ITG_{1_Middle} to ITG_{n_Middle} (=n integral values ITG_{1_Middle} to ITG_{n_Middle} in n predetermined potential sections calculated from the current-potential characteristics of a cyclic voltammogram measured by scanning the potential V at a potential scanning speed V_{r_Middle}) of the calculation data CALᵤₙᵢ (step S822).

Furthermore, the calculation unit 216 calculates the sum H(+)_sum of the integral values in a positive predetermined potential section based on the n integral values ITG_{1_High} to ITG_{n_High} (=n integral values ITG_{1_High} to ITG_{n_High} in n predetermined potential sections calculated from the current-potential characteristics of a cyclic voltammogram measured by scanning the potential V at a potential scanning speed V_{r_High}) of the calculation data CALᵤₙᵢ (step S823).

Furthermore, the calculation unit 216 calculates the sum L(all)_sum of the integral values in all predetermined potential sections based on the n integral values ITG_{1_Low} to ITG_{n_Low}, calculates the sum M(all)_sum of the integral values in all predetermined potential sections based on the n integral values ITG_{1_Middle} to ITG_{n_Middle}, and calculates the sum H(all)_sum of the integral values in all predetermined potential sections based on the n integral values ITG_{1_High} to ITG_{n_High} (step S824).

Furthermore, the calculation unit 216 calculates the sum H(−)_sum of the integral values in a negative predetermined potential section based on the n integral values ITG_{1_High} to ITG_{n_High} (step S825).

Furthermore, the calculation unit 216 calculates the sum H(−)_sum_th of the integral values in a predetermined negative potential section below or equal to a threshold value Vth based on the n integral values ITG_{1_High} to ITG_{n_High}, calculates the sum M(−)_sum_th of the integral values in a predetermined negative potential section below or equal to a threshold value Vth based on the n integral values ITG_{1_Middle} to ITG_{n_Middle}, and calculates the sum L(−)_sum_th of the integral values in a predetermined negative potential section below or equal to a threshold value Vth based on the n integral values ITG_{1_Low} to ITG_{n_Low} (step S826).

Furthermore, the calculation unit 216 calculates the Body index (+) by dividing the adding result adding the sum M(+)_sum of the integral values to the sum L(+) sum of the integral values by the sum H(+)_sum of the integral values (step S827).

Furthermore, the calculation unit 216 calculates the Body index (all) by dividing the adding result adding the sum M(all)_sum of the integral values to the sum L(all)_sum of the integral values by the sum H(all)_sum of the integral values (step S828).

Furthermore, the calculation unit 216 calculates the Body index (−)_th by dividing the adding result adding the sum M(−)_sum_th of the integral values to the sum L(−)_sum_th of the integral values by the sum H(−)_sum_th of the integral values (step S829).

Then, the calculation unit 216 outputs the Body index (+), the Body index (all), the sum H(+)_sum of the integral values, the sum H(−)_sum of the integral values and the Body index (−)_th to the taste diagnostic unit 217.

The taste diagnostic unit 217 receives the Body index (+), the Body index (all), the sum H(+)_sum of the integral values, the sum H(−)_sum of the integral values, and the Body index (−)_th from the calculation unit 216.

Then, the taste diagnostic unit 217 diagnoses the “astringency”, “aftertaste”, “sweetness”, “aroma” and “bitterness” of the analyte based on Body index (+), Body index (all), the sum H(+)_sum of the integral values, the sum H(−)_sum of the integral values and Body index (−)_th (step S830).

In this case, the taste diagnostic unit 217 diagnoses the “astringency” of the analyte (shochu) using the Body index (+) and diagnoses the “astringency” of the analyte (grapes) using the Body index (−)_th.

Then, after step S830, the taste diagnostic unit 217 creates a taste diagnosis result JDR of the analyte, and outputs the created taste diagnosis result JDR to the control unit 212 and the display unit 218.

Upon receiving by the control unit 212 the taste diagnosis result JDR from the taste diagnostic unit 217, the control unit 212 stores the received taste diagnosis result JDR in the database 22.

Furthermore, upon receiving the taste diagnosis result JDR from the taste diagnostic unit 217, the display unit 218 displays the received taste diagnosis result JDR.

After the step S830, the operation of the diagnostic device 2 proceeds to “End” in FIG. 90.

FIG. 99 is a flowchart for explaining the detailed operation of the step S830 in FIG. 98.

The flowchart shown in FIG. 99 is a flowchart for explaining the detailed operation of step S830 in FIG. 98 in when the analyte is “shochu”.

Referring to FIG. 99, after the step S829 in FIG. 98, the taste diagnostic unit 217 receives the Body index (+), the Body index (all), the sum H(+)_sum of the integral values and the sum H(−)_sum of the integral values from the calculation unit 216.

Then, the taste diagnostic unit 217 substitutes the Body index (+) and coefficient k1 into equation (5) to calculate the astringency ASTG value of the analyte (shochu), and diagnoses the calculated value as the astringency ASTG of the analyte (shochu) (step S8301).

After the step S8301, the taste diagnostic unit 217 substitutes the Body index (all) and coefficient k2 into equation (6) to calculate the value of the aftertaste LNGS of the analyte (shochu), and diagnoses the calculated value as the aftertaste LNGS of the analyte (shochu) (step S8302).

After the step S8302, the taste diagnostic unit 217 substitutes the sum H(+) sum of the integral values and the coefficients k₃ and k₄ into equation (7) to calculate the value of the sweetness SWT, and diagnoses the calculated value as the sweetness SWT of the analyte (shochu) (step S8303).

After the step S8303, the taste diagnostic unit 217 substitutes the sum H(−)_sum of the integral values and the coefficients k₅ and k₆ into equation (8) to calculate the value of the aroma SCT, and diagnoses the calculated value as the aroma SCT of the analyte (shochu) (step S8304).

After the step S8304, the taste diagnostic unit 217 substitutes the astringency ASTG, sweetness SWT and coefficients k₇ and k₈ into equation (9) to calculate the value of bitterness BIT, and diagnoses the calculated value as the bitterness BIT of the analyte (shochu) (step S8305).

Then, after the step S8305, the operation of the diagnostic device 2 proceeds to “End” in FIG. 90.

FIG. 100 is another flowchart for explaining the detailed operation of the step S830 in FIG. 98.

Referring to FIG. 100, after the step S829 in FIG. 98, the taste diagnostic unit 217 receives the Body index (−)_th from the calculation unit 216.

Then, the taste diagnostic unit 217 substitutes the Body index (−)_th and the coefficient k9 into the formula (11) to calculate the value of the astringency ASTG, and diagnoses the calculated value as the astringency ASTG of the analyte (grapes) (step S8301A).

Thereafter, the operation of the diagnostic device 2 proceeds to “End” in FIG. 90.

FIG. 101 is a flowchart for explaining the detailed operation of the step S83 in FIG. 97.

Referring to FIG. 101, when it has been judged in the step S81 of FIG. 97 that a plurality of index data have been received, the calculation unit 216 already has received P pieces of index data IDX₁ to IDX_{P} from the control unit 212.

Then, the calculation unit 216 sets p=1 (step S831), and detects the calculation data CALₚ from the index data IDXₚ (step S832).

Thereafter, the calculation unit 216 sequentially executes the steps S821 to S829 in FIG. 98 based on the calculation data CALₚ of the index data IDXₚ (step S833).

Then, the taste diagnostic unit 217 sequentially executes the steps S8301 to S8305 in FIG. 99 based on the Body index (+), the Body index (all), the sum H(+)_sum of the integral values and the sum H(−)_sum of the integral values (step S834).

Then, the calculation unit 216 judges whether or not p=P (step S835).

When it is judged in step S835 that p is not equal to P, the calculation unit 216 sets p=p+1 (step S836).

Thereafter, the operation of the diagnostic device 2 proceeds to step S832, and steps S832 to S836 are executed repeatedly until it is judged in the step S835 that p is equal to P.

Then, in the step S835, when it was judged that p is equal to P, the taste diagnostic unit 217 creates a diagnosis result JDR_{P} of P tastes for the P analytes, and outputs the created diagnosis result JDR_{P} of P tastes to the control unit 212 and the display unit 218.

When the control unit 212 receives the P taste diagnosis results JDR_{P} from the taste diagnostic unit 217, the control unit 212 stores the received P taste diagnosis results JDR_{P} in the database 22.

Furthermore, upon receiving the P taste diagnosis results JDR_{P} from the taste diagnostic unit 217, the display unit 218 displays the received P taste diagnosis results JDR_{P}.

Then, the operation of the diagnostic device 2 proceeds to “End” in FIG. 90.

FIG. 102 is another flowchart for explaining the detailed operation of the step S83 in FIG. 97.

The flowchart shown in FIG. 102 is the same as the flowchart shown in FIG. 101, except that the step S834 in the flowchart shown in FIG. 101 is replaced with step S834A.

Referring to FIG. 102, when it was judged in the step S81 of FIG. 97 that a plurality of index data were received, the calculation unit 216 has received P pieces of index data IDX₁ to IDX_{P} from the control unit 212.

Then, the above-mentioned steps S831 to S833 are executed sequentially. After the step S833, the taste diagnostic unit 217 executes step S8301A in FIG. 100 based on the Body index (−)_th (step S834A).

Then, the above-mentioned the step S835 is executed, and when it is judged in step S835 that p=P is not satisfied, the above-mentioned the step S836 is executed, and then the operation of the diagnostic device 2 proceeds to the step S832.

Then, the steps S832, S833, S834A, S835, and S836 are repeatedly executed until it is judged in the step S835 that p=P.

Then, in the step S835, when it was judged that p is equal to P, the taste diagnostic unit 217 creates a diagnosis result JDR_{P} of P tastes for the P analytes, and outputs the created diagnosis result JDR_{P} of P tastes to the control unit 212 and the display unit 218.

Upon receiving the P pieces of taste diagnosis results JDR_{P} from the taste diagnostic unit 217, the control unit 212 stores the received P pieces of taste diagnosis results JDR_{P} in the database 22.

Upon receiving the P pieces of taste diagnosis results JDR_{P} from the taste diagnostic unit 217, the display unit 218 displays the received P pieces of taste diagnosis results JDR_{P}.

Then, the operation of the diagnostic device 2 proceeds to “End” in FIG. 90.

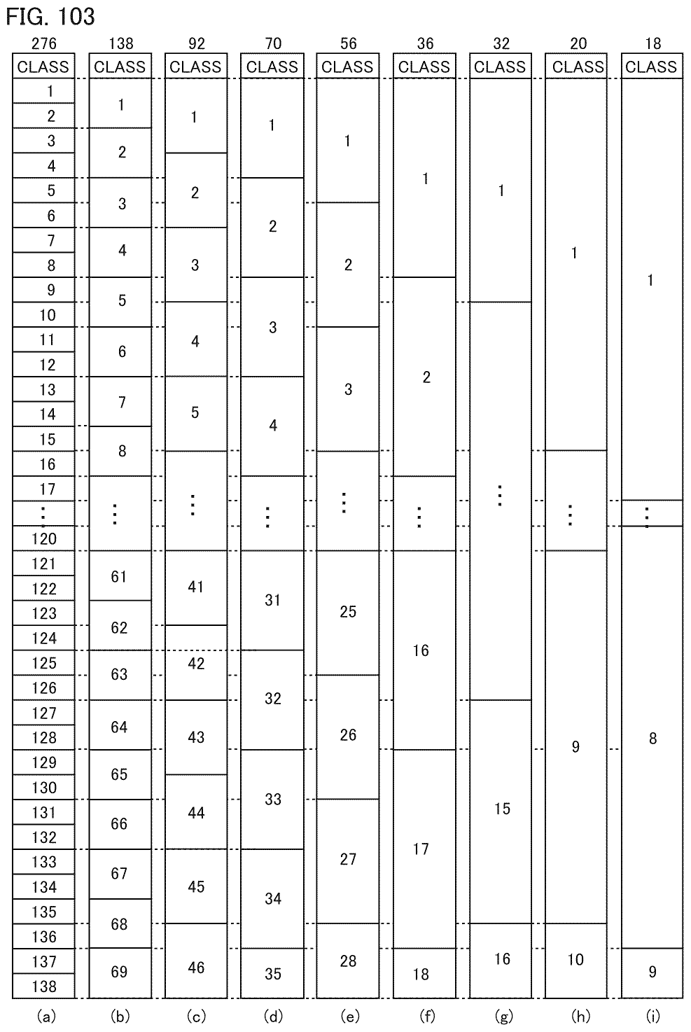

FIG. 103 is a diagram showing the correspondence relationship of classes in a positive predetermined potential section when the number of integral values in when the analyte is “shochu” is 18, 20, 32, 36, 56, 70, 92, 138 and 276.

Referring to FIG. 103, when the analyte is “shochu”, class 1_₁₃₈ in when the number of integral values is 138 is associated with classes 1_₂₇₆ and 2_₂₇₆ in when the number of integral values is 276, class 2_₁₃₈ in when the number of integral values is 138 is associated with classes 3_₂₇₆ and 4_₂₇₆ in when the number of integral values is 276, class 3_₁₃₈ in when the number of integral values is 138 is associated with classes 5_₂₇₆ and 6_₂₇₆ in when the number of integral values is 276, similarly, class 67_₁₃₈ in when the number of integral values is 138 is associated with classes 133_₂₇₆ and 134_₂₇₆ in when the number of integral values is 276, class 68_₁₃₈ in when the number of integral values is 138 is associated with classes 135_₂₇₆ and 136_₂₇₆ in when the number of integral values is 276, class 69_₁₃₈ in when the number of integral values is 138 is associated with classes 137_₂₇₆ and 138_₂₇₆ in when the number of integral values is 276 (See (b) of FIG. 103).

As a result, the integral value ITG_{1_Low_138} in class 1_₁₃₈ in when the number of integral values is 138 consists of the sum (=ITG_{1_Low_276}+ITG_{2_Low_276}) of the integral value ITG_{1_Low_276} in class 1_₂₇₆ and the integral value ITG_{2_Low_276} in class 2_₂₇₆ in when the number of integral values is 276, the integral value ITG_{1_MIddle_138} in class 1_₁₃₈ in when the number of integral values is 138 consists of the sum (=ITG_{1_Middle_276}+ITG_{2_Middle_276}) of the integral value ITG_{1_Middle_276} in class 1_₂₇₆ and the integral value ITG_{2_Middle_276} in class 2_₂₇₆ in when the number of integral values is 276, and the integral value ITG_{1_High_138} in class 1_₁₃₈ in when the number of integral values is 138 consists of the sum (=ITG_{1_High_276}+ITG_{2_High_276}) of the integral value ITG_{1_High_276} in class 1_₂₇₆ and the integral value ITG_{2_High_276} in class 2_₂₇₆ in when the number of integral values is 276.

Furthermore, an integral value ITG_{2_Low_138} in class 2_₁₃₈ in when the number of integral values is 138 is consists of the sum (=ITG_{3_Low_276}+ITG_{4_Low_276}) of an integral value ITG_{3_Low_276} in class 3_₂₇₆ and an integral value ITG_{4_Low_276} in class 4_₂₇₆ in when the number of integral values is 276, an integral value ITG_{2_Middle_138} in class 2_₁₃₈ in when the number of integral values is 138 is consists of the sum (=ITG_{3_Middle_276}+ITG_{4_Middle_276}) of an integral value ITG_{3_Middle_276} in class 3_₂₇₆ and an integral value ITG_{4_Middle_276} in class 4_₂₇₆ in when the number of integral values is 276, and an integral value ITG_{2_High_138} in class 2_₁₃₈ in when the number of integral values is 138 is consists of the sum (=ITG_{3_High_276}+ITG_{4_High_276}) of an integral value ITG_{3_High_276} in class 3_₂₇₆ and an integral value ITG_{4_High_276} in class 4_₂₇₆ in when the number of integral values is 276.

Similarly, an integral value ITG_{69_Low_138} in class 69_₁₃₈ in when the number of integral values is 138 is consists of the sum (=ITG_{137_Low_276}+ITG_{138_Low_276}) of an integral value ITG_{137_Low_276} in class 137_₂₇₆ and an integral value ITG_{138_Low_276} in class 138_₂₇₆ in when the number of integral values is 276, an integral value ITG_{69_Middle_138} in class 69_₁₃₈ in when the number of integral values is 138 is consists of the sum (=ITG_{137_Middle_276}+ITG_{138_Middle_276}) of an integral value ITG_{137_Middle_276} in class 3_₂₇₆ and an integral value ITG_{138_Middle_276} in class 138_₂₇₆ in when the number of integral values is 276, and an integral value ITG_{69_High_138} in class 69_₁₃₈ in when the number of integral values is 138 is consists of the sum (=ITG_{137_High_276}+ITG_{138_High_276}) of an integral value ITG_{137_High_276} in class 137_₂₇₆ and an integral value ITG_{138_High_276} in class 138_₂₇₆ in when the number of integral values is 276.

In addition, when the analyte is “shochu”, class 1_₉₂ in when the number of integral values is 92 is corresponded with classes 1_₂₇₆ to 3_₂₇₆ in when the number of integral values is 276, class 2_₉₂ in when the number of integral values is 92 is corresponded with classes 4_₂₇₆ to 6_₂₇₆ in when the number of integral values is 276, class 3_₉₂ in when the number of integral values is 92 is corresponded with classes 7_₂₇₆ to 9_₂₇₆ in when the number of integral values is 276, similarly, class 45_₉₂ in when the number of integral values is 92 is corresponded with classes 133_₂₇₆ to 135_₂₇₆ in when the number of integral values is 276, and class 46_₉₂ in when the number of integral values is 92 is corresponded with classes 136_₂₇₆ to 138_₂₇₆ in when the number of integral values is 276 (See (c) of FIG. 103).

As a result, an integral value ITG_{1_Low_92} in class 1_₉₂ in when the number of integral values is 92 is composed of the sum (=ITG_{1_Low_276}+ITG_{2_Low_276}+ITG_{3_Low_276}) of an integral value ITG_{1_Low_276} in class 1_₂₇₆, an integral value ITG_{2_Low_276} in class 2_₂₇₆, and an integral value ITG_{3_Low_276} in class 3_₂₇₆ in when the number of integral values is 276, an integral value ITG_{1_Middle_92} in class 1_₉₂ in when the number of integral values is 92 is composed of the sum (=ITG_{1_Middle_276}+ITG_{2_Middle_276}+ITG_{3_Middle_276}) of an integral value ITG_{1_Middle_276} in class 1_₂₇₆, an integral value ITG_{2_Middle_276} in class 2_₂₇₆, and an integral value ITG_{3_Middle_276} in class 3_₂₇₆ in when the number of integral values is 276, and an integral value ITG_{1_High_92} in class 1_₉₂ in when the number of integral values is 92 is composed of the sum (=ITG_{1_High_276}+ITG_{2_High_276}+ITG_{3_High_276}) of an integral value ITG_{1_High_276} in class 1_₂₇₆, an integral value ITG_{2_High_276} in class 2_₂₇₆, and an integral value ITG_{3_High_276} in class 3_₂₇₆ in when the number of integral values is 276.

In addition, an integral value ITG_{2_Low_92} in class 2_₉₂ in when the number of integral values is 92 is composed of the sum (=ITG_{4_Low_276}+ITG_{5_Low_276}+ITG_{6_Low_276}) of an integral value ITG_{4_Low_276} in class 4_₂₇₆, an integral value ITG_{5_Low_276} in class 5_₂₇₆, and an integral value ITG_{6_Low_276} in class 6_₂₇₆ in when the number of integral values is 276, an integral value ITG_{2_Middle_92} in class 2_₉₂ in when the number of integral values is 92 is composed of the sum (=ITG_{4_Middle_276}+ITG_{5_Middle_276}+ITG_{6_Middle_276}) of an integral value ITG_{4_Middle_276} in class 4_₂₇₆, an integral value ITG_{5_Middle_276} in class 5_₂₇₆, and an integral value ITG_{6_Middle_276} in class 6_₂₇₆ in when the number of integral values is 276, and an integral value ITG_{2_High_92} in class 2_₉₂ in when the number of integral values is 92 is composed of the sum (=ITG_{4_High_276}+ITG_{5_High_276}+ITG_{6_High_276}) of an integral value ITG_{4_High_276} in class 4_₂₇₆, an integral value ITG_{5_High_276} in class 5_₂₇₆, and an integral value ITG_{6_High_276} in class 6_₂₇₆ in when the number of integral values is 276.

Similarly, an integral value ITG_{46_Low_92} in class 46_₉₂ in when the number of integral values is 92 is composed of the sum (=ITG_{136_Low_276}+ITG_{137_Low_276}+ITG_{138_Low_276}) of an integral value ITG_{136_Low_276} in class 136_₂₇₆, an integral value ITG_{137_Low_276} in class 137_₂₇₆, and an integral value ITG_{138_Low_276} in class 138_₂₇₆ in when the number of integral values is 276, an integral value ITG_{46_Middle_92} in class 46_₉₂ in when the number of integral values is 92 is composed of the sum (=ITG_{136_Middle_276}+ITG_{137_Middle_276}+ITG_{138_Middle_276}) of an integral value ITG_{136_Middle_276} in class 136_₂₇₆, an integral value ITG_{137_Middle_276} in class 137_₂₇₆, and an integral value ITG_{138_Middle_276} in class 138_₂₇₆ in when the number of integral values is 276, and an integral value ITG_{46_High_92} in class 46_₉₂ in when the number of integral values is 92 is composed of the sum (=ITG_{136_High_276}+ITG_{137_High_276}+ITG_{138_High_276}) of an integral value ITG_{136_High_276} in class 136_₂₇₆, an integral value ITG_{137_High_276} in class 137_₂₇₆, and an integral value ITG_{138_High_276} in class 138_₂₇₆ in when the number of integral values is 276.

Furthermore, when the analyte is “shochu”, class 1_₇₀ in when the number of integral values is 70 is corresponded with classes 1_₂₇₆ to 4_₂₇₆ in when the number of integral values is 276, class 2_₇₀ in when the number of integral values is 70 is corresponded with classes 5_₂₇₆ to 8_₂₇₆ in when the number of integral values is 276, class 3_₇₀ in when the number of integral values is 70 is corresponded with classes 9₂₇₆ to 12_₂₇₆ in when the number of integral values is 276, similarly, class 33_₇₀ in when the number of integral values is 70 is corresponded with classes 129_₂₇₆ to 132_₂₇₆ in when the number of integral values is 276, and class 34_₇₀ in when the number of integral values is 70 is corresponded with classes 133_₂₇₆ to 136_₂₇₆ in when the number of integral values is 276, and class 35_₇₀ in when the number of integral values is 70 is corresponded with classes 137_₂₇₆, 138_₂₇₆ in when the number of integral values is 276, (See (d) of FIG. 103).

Here, the reason why class 35_₇₀ in when the number of integral values is 70 is corresponded with classes 137_₂₇₆ and 138_₂₇₆ in when the number of integral values is 276, because it is necessary to set class 35_₇₀ in when the number of integral values is 70 to the last class in the positive predetermined potential section.

As a result, the integral value ITG_{1_Low_70} in class 1_₇₀ in when the number of integral values is 70 consists of the sum (=ITG_{1_Low_276}+ITG_{2_Low_276}+ITG_{3_Low_276}+ITG_{4_Low_276}) of the integral value ITG_{1_Low_276} in class 1_₂₇₆, the integral value ITG_{2_Low_276} in class 2_₂₇₆, the integral value ITG_{3_Low_276} in class 3_₂₇₆, the integral value ITG_{4_Low_276} in class 4_₂₇₆ in when the number of integral values is 276, the integral value ITG_{1_Middle_70} in class 1_₇₀ in when the number of integral values is 70 consists of the sum (=ITG_{1_Middle_276}+ITG_{2_Middle_276}+ITG_{3_Middle_276}+ITG_{4_Middle_276}) of the integral value ITG_{1_Middle_276} in class 1_₂₇₆, the integral value ITG_{2_Middle_276} in class 2_₂₇₆, the integral value ITG_{3_Middle_276} in class 3_₂₇₆, and the integral value ITG_{4_Middle_276} in class 4_₂₇₆ in when the number of integral values is 276, and the integral value ITG_{1_High_70} in class 1_₇₀ in when the number of integral values is 70 consists of the sum (=ITG_{1_High_276}+ITG_{2_High_276}+ITG_{3_High_276}+ITG_{4_High_276}) of the integral value ITG_{1_High_276} in class 1_₂₇₆, the integral value ITG_{2_High_276} in class 2_₂₇₆, the integral value ITG_{3_High_276} in class 3_₂₇₆, and the integral value ITG_{4_High_276} in class 4_₂₇₆ in when the number of integral values is 276.

Also, the integral value ITG_{2_Low_70} in class 2_₇₀ in when the number of integral values is 70 consists of the sum (=ITG_{5_Low_276}+ITG_{6_Low_276}+ITG_{7_Low_276}+ITG_{8_Low_276}) of the integral value ITG_{5_Low_276} in class 5_₂₇₆, the integral value ITG_{6_Low_276} in class 6_₂₇₆, the integral value ITG_{7_Low_276} in class 7_₂₇₆, and the integral value ITG_{8_Low_276} in class 8_₂₇₆ in when the number of integral values is 276, the integral value ITG_{2_Middle_70} in class 2_₇₀ in when the number of integral values is 70 consists of the sum (=ITG_{5_Low_276}+ITG_{6_Low_276}+ITG_{7_Low_276}+ITG_{8_Low_276}) of the integral value ITG_{5_Middle_276} in class 5_₂₇₆, the integral value ITG_{6_Middle_276} in class 6_₂₇₆, the integral value ITG_{7_Middle_276} in class 7_₂₇₆, and the integral value ITG_{8_Middle_276} in class 8_₂₇₆ in when the number of integral values is 276, and the integral value ITG_{2_High_70} in class 2_₇₀ in when the number of integral values is 70 consists of the sum (=ITG_{5_High_276}+ITG_{6_High_276}+ITG_{7_High_276}+ITG_{8_High_276}) of the integral value ITG_{5_High_276} in class 5_₂₇₆, the integral value ITG_{6_High_276} in class 6_₂₇₆, the integral value ITG_{7_High_276} in class 7_₂₇₆, and the integral value ITG_{8_High_276} in class 8_₂₇₆ in when the number of integral values is 276.

Similarly, the integral value ITG_{34_Low_70} in class 34_₇₀ in when the number of integral values is 70 consists of the sum (=ITG_{133_Low_276}+ITG_{134_Low_276}+ITG_{135_Low_276}+ITG_{136_Low_276}) of the integral value ITG_{133_Low_276} in class 133_₂₇₆, the integral value ITG_{134_Low_276} in class 134_₂₇₆, the integral value ITG_{135_Low_276} in class 135_₂₇₆, and the integral value ITG_{136_Low_276} in class 136_₂₇₆ in when the number of integral values is 276, the integral value ITG_{34_Middle_70} in class 34_₇₀ in when the number of integral values is 70 consists of the sum (=ITG_{5_Low_276}+ITG_{6_Low_276}+ITG_{7_Low_276}+ITG_{8_Low_276}) of the integral value ITG_{133_Middle_276} in class 133_₂₇₆, the integral value ITG_{134_Middle_276} in class 134_₂₇₆, the integral value ITG_{135_Middle_276} in class 135_₂₇₆, and the integral value ITG_{136_Middle_276} in class 136_₂₇₆ in when the number of integral values is 276, and the integral value ITG_{34_High_70} in class 34_₇₀ in when the number of integral values is 70 consists of the sum (=ITG_{133_High_276}+ITG_{134_High_276}+ITG_{135_High_276}+ITG_{136_High_276}) of the integral value ITG_{133_High_276} in class 133_₂₇₆, the integral value ITG_{134_High_276} in class 134_₂₇₆, the integral value ITG_{135_High_276} in class 135_₂₇₆, and the integral value ITG_{136_High_276} in class 136_₂₇₆ in when the number of integral values is 276.

Then, the integral value ITG_{35_Low_70} in class 35_₇₀ in when the number of integral values is 70 is made up of the sum (=ITG_{137_Low_276}+ITG_{138_Low_276}) of the integral value ITG_{137_Low_276} in class 137_₂₇₆ and the integral value ITG_{138_Low_276} in class 138_₂₇₆ in when the number of integral values is 276, the integral value ITG_{35_Middle_70} in class 35_₇₀ in when the number of integral values is 70 is made up of the sum (=ITG_{137_Middle_276}+ITG_{138_Middle_276}) of an integral value ITG_{137_Middle_276} in class 137_₂₇₆ and an integral value ITG_{138_Middle_276} in class 138_₂₇₆ in when the number of integral values is 276, and the integral value ITG_{35_High_70} in class 35_₇₀ in when the number of integral values is 70 is made up of the sum (=ITG_{137_High_276}+ITG_{138_High_276}) of an integral value ITG_{137_High_276} in class 137_₂₇₆ and an integral value ITG_{138_High_276} in class 138_₂₇₆ in when the number of integral values is 276.

Furthermore, when the analyte is “shochu”, class 1_₅₆ in when the number of integral values is 56 is associated with classes 1_₂₇₆ to 5_₂₇₆ in when the number of integral values is 276, class 2_₅₆ in when the number of integral values is 56 is associated with classes 6_₂₇₆ to 10_₂₇₆ in when the number of integral values is 276, similarly, class 26_₅₆ in when the number of integral values is 56 is associated with classes 126_₂₇₆ to 130_₂₇₆ in when the number of integral values is 276, class 27_₅₆ in when the number of integral values is 56 is associated with classes 131_₂₇₆ to 135_₂₇₆ in when the number of integral values is 276, and class 28_₅₆ in when the number of integral values is 56 is associated with classes 136_₂₇₆ to 138_₂₇₆ in when the number of integral values is 276 (See (e) of FIG. 103).

Here, the reason why class 28_₅₆ in when the number of integral values is 56 is associated with classes 136_₂₇₆ to 138_₂₇₆ in when the number of integral values is 276, in order to make class 28_₅₆ in when the number of integral values is 56 to the last class in the positive predetermined potential section.

As a result, the integral value ITG_{1_Low_56} in class 1_₅₆ in when the number of integral values is 56 consists of the sum(=ITG_{1_Low_276}+ITG_{2_Low_276}+ITG_{3_Low_276}+ITG_{4_Low_276}+ITG_{5_Low_276}) from integral value ITG_{1_Low_276} in class 1_₂₇₆ to ITG_{5_Low_276} in class 5_₂₇₆ in when the number of integral values is 276, the integral value ITG_{1_Middle_56} in class 1_₅₆ in when the number of integral values is 56 consists of the sum(=ITG_{1_Middle_276}+ITG_{2_Middle_276}+ITG_{3_Middle_276}+ITG_{4_Middle_276}+ITG_{5_Middle_276}) from integral value ITG_{1_Middle_276} in class 1_₂₇₆ to ITG_{5_Middle_276} in class 5_₂₇₆ in when the number of integral values is 276, and the integral value ITG_{1_High_56} in class 1_₅₆ in when the number of integral values is 56 consists of the sum (=ITG_{1_High_276}+ITG_{2_High_276}+ITG_{3_High_276}+ITG_{4_High_276}+ITG_{5_High_276}) from integral value ITG_{1_High_276} in class 1_₂₇₆ to ITG_{5_High_276} in class 5_₂₇₆ in when the number of integral values is 276.

In addition, the integral value ITG_{2_Low_56} in class 2_₅₆ in when the number of integral values is 56 consists of the sum(=ITG_{6_Low_276}+ITG_{7_Low_276}+ITG_{8_Low_276}+ITG_{9_Low_276}+ITG_{10_Low_276}) from integral values ITG_{6_Low_276} in class 6_₂₇₆ to ITG_{10_Low_276} in class 10_₂₇₆ in when the number of integral values is 276, the integral value ITG_{2_Middle_56} in class 2_₅₆ in when the number of integral values is 56 consists of the sum(=ITG_{6_Middle_276}+ITG_{7_Middle_276}+ITG_{8_Middle_276}+ITG_{9_Middle_276}+ITG_{10_Middle_276}) from integral value ITG_{6_Middle_276} in class 6_₂₇₆ to ITG_{10_Middle_276} in class 10_₂₇₆ in when the number of integral values is 276, and the integral value ITG_{2_High_56} in class 2_₅₆ in when the number of integral values is 56 consists of the sum(=ITG_{6_High_276}+ITG_{7_High_276}+ITG_{8_High_276}+ITG_{9_High_276}+ITG_{10_High_276}) from integral value ITG_{6_High_276} in class 6_₂₇₆ to ITG_{10_High_276} in class 10_₂₇₆ in when the number of integral values is 276.

Similarly, the integral value ITG_{27_Low_56} in class 27_₅₆ in when the number of integral values is 56 consists of the sum(=ITG_{13l_Low_276}+ITG_{132_Low_276}+ITG_{133_Low_276}+ITG_{134_Low_276}+ITG_{135_Low_276}) from integral values ITG_{131_Low_276} in class 131_₂₇₆ ITG_{135_Low_276} in class 135_₂₇₆ in when the number of integral values is 276, the integral value ITG_{27_Middle_56} in class 27_₅₆ in when the number of integral values is 56 consists of the sum (=ITG_{131_Middle_276}+ITG_{132_Middle_276}+ITG_{133_Middle_276}+ITG_{134_Middle_276}+ITG_{135_Middle_276}) from integral value ITG_{131_Middle_276} in class 131_₂₇₆ to ITG_{135_Middle_276} in class 135_₂₇₆ in when the number of integral values is 276, and the integral value ITG_{27_High_56} in class 27_₅₆ in when the number of integral values is 56 consists of the sum ITG_{131_High_276}+ITG_{132_High_276}+ITG_{133_High_276}+ITG_{134_High_276}+ITG_{135_High_276}) from integral values ITG_{131_High_276} in class 131_₂₇₆ to ITG_{135_High_276} in class 135_₂₇₆ in when the number of integral values is 276.

Then, the integral value ITG_{28_Low_56} in class 28_₅₆ in when the number of integral values is 56 consists of the sum(=ITG_{136_Low_276}+ITG_{137_Low_276}+ITG_{138_Low_276}) integral value ITG_{136_Low_276} in class 136_₂₇₆, integral value ITG_{137_Low_276} in class 137_₂₇₆ and integral value ITG_{138_Low_276} in class 138_₂₇₆ in when the number of integral values is 276, the integral value ITG_{28_Middle_56} in class 28_₅₆ in when the number of integral values is 56 consists of the sum(=ITG_{136_Middle_276}+ITG_{137_Middle_276}+ITG_{138_Middle_276}) of integral value ITG_{136_Middle_276} in class 136_₂₇₆, integral value ITG_{137_Middle_276} in class 137_₂₇₆ and integral value ITG_{138_Middle_276} in class 138_₂₇₆ in when the number of integral values is 276, and the integral value ITG_{28_High_56} in class 28_₅₆ in when the number of integral values is 56 consists of the sum(=ITG_{136_High_276}+ITG_{137_High_276}+ITG_{138_High_276}) of integral value ITG_{136_High_276} in class 136_₂₇₆, integral value ITG_{137_High_276} in class 137_₂₇₆ and integral value ITG_{138_High_276} in class 138_₂₇₆ in when the number of integral values is 276.

Furthermore, when the analyte is “shochu”, class 1_₃₆ in when the number of integral values is 36 is associated with classes 1_₂₇₆ to 8_₂₇₆ in when the number of integral values is 276, class 2_₃₆ in when the number of integral values is 36 is associated with classes 9_₂₇₆ to 16_₂₇₆ in when the number of integral values is 276, similarly, class 16_₃₆ in when the number of integral values is 36 is associated with classes 121_₂₇₆ to 128_₂₇₆ in when the number of integral values is 276, class 17_₃₆ in when the number of integral values is 36 is associated with classes 129_₂₇₆ to 136_₂₇₆ in when the number of integral values is 276, and class 18_₃₆ in when the number of integral values is 36 is associated with classes 137_₂₇₆ and 138_₂₇₆ in when the number of integral values is 276 (See (f) of FIG. 103).

Here, the reason why class 18_₃₆ in when the number of integral values is 36 is associated with classes 137_₂₇₆ and 138_₂₇₆ in when the number of integral values is 276 is to make class 18_₃₆ in when the number of integral values is 36 the last class in the positive predetermined potential section.

As a results, the integral value ITG_{1_Low_36} in class 1_₃₆ in when the number of integral values is 36 consists of the sum(=ITG_{1_Low_276}+ITG_{2_Low_276}+ITG_{3_Low_276}+ITG_{4_Low_276}+ITG_{5_Low_276}+ITG_{6_Low_276}+ITG_{7_Low_276}+ITG_{8_Low_276}) from integral value ITG_{1_Low_276} in class 1_₂₇₆ to integral value ITG_{8_Low_276} in class 8_₂₇₆ in when the number of integral values is 276, the integral value ITG_{1_Middle_36} in class 1_₃₆ in when the number of integral values is 36 consists of the sum(=ITG_{1_Middle_276}+ITG_{2_Middle_276}+ITG_{3_Middle_276}+ITG_{4_Middle_276}+ITG_{5_Middle_276}+ITG_{6_Middle_276}+ITG_{7_Middle_276}+ITG_{8_Middle_276}) from integral value ITG_{1_Middle_276} in class 1_₂₇₆ to integral value ITG_{8_Middle_276} in class 8_₂₇₆ in when the number of integral values is 276, and the integral value ITG_{1_High_36} in class 1_₃₆ in when the number of integral values is 36 consists of the sum(=ITG_{1_High_276}+ITG_{2_High_276}+ITG_{3_High_276}+ITG_{4_High_276}+ITG_{5_High_276}+ITG_{6_High_276}+ITG_{7_High_276}+ITG_{8_High_276}) from integral value ITG_{1_High_276} in class 1_₂₇₆ to integral value ITG_{8_High_276} in class 8_₂₇₆ in when the number of integral values is 276.

Similarly, the integral value ITG_{17_Low_36} in class 17_₃₆ in when the number of integral values is 36 consists of the sum(=ITG_{129_Low_276}+ITG_{130_Low_276}+ITG_{13l_Low_276}+ITG_{132_Low_276}+ITG_{133_Low_276}+ITG_{134_Low_276}+ITG_{135_Low_276}+ITG_{136_Low_276}) from integral value ITG_{129_Low_276} in class 129_₂₇₆ to integral value ITG_{136_Low_276} in class 136_₂₇₆ in when the number of integral values is 276, the integral value ITG_{17_Middle_36} in class 17_₃₆ in when the number of integral values is 36 consists of the sum(=ITG_{129_Middle_276}+ITG_{130_Middle_276}+ITG_{131_Middle_276}+ITG_{132_Middle_276}+ITG_{133_Middle_276}+ITG_{134_Middle_276}+ITG_{135_Middle_276}+ITG_{136_Low_276}) from integral value ITG_{129_Middle_276} in class 129_₂₇₆ to integral value ITG_{136_Middle_276} in class 136_₂₇₆ in when the number of integral values is 276, and the integral value ITG_{17_High_36} in class 17_₃₆ in when the number of integral values is 36 consists of the sum (=ITG_{129_High_276}+ITG_{130_High_276}+ITG_{131_High_276}+ITG_{132_High_276}+ITG_{133_High_276}+ITG_{134_High_276}+ITG_{135_High_276}+ITG_{136_High_276}) from integral value ITG_{129_High_276} in class 129_₂₇₆ to integral value ITG_{136_High_276} in class 136_₂₇₆ in when the number of integral values is 276.

In addition, the integral value ITG_{18_Low_36} in class 18_₃₆ in when the number of integral values is 36 consists of the sum(=ITG_{137_Low_276}+ITG_{138_Low_276}) of integral value ITG_{137_Low_276} in class 137_₂₇₆ and integral value ITG_{138_Low_276} in class 138_₂₇₆ in when the number of integral values is 276, the integral value ITG_{18_Middle_36} in class 18_₃₆ in when the number of integral values is 36 consists of the sum(=ITG_{137_Middle_276}+ITG_{138_Middle_276}) of integral value ITG_{137_Middle_276} in class 137_₂₇₆ and integral value ITG_{138_Middle_276} in class 138_₂₇₆ in when the number of integral values is 276, and the integral value ITG_{18_High_36} in class 18_₃₆ in when the number of integral values is 36 consists of the sum(=ITG_{137_High_276}+ITG_{138_High_276}) of integral value ITG_{137_High_276} in class 137_₂₇₆ and integral value ITG_{138_High_276} in class 138_₂₇₆ in when the number of integral values is 276.

Furthermore, when the analyte is “shochu”, class 1_₃₂ in when the number of integral values is 32 is associated with classes 1_₂₇₆ to 9_₂₇₆ in when the number of integral values is 276, similarly, class 15_32 in when the number of integral values is 32 is associated with classes 127_₂₇₆ to 135_₂₇₆ in when the number of integral values is 276, and class 16_₃₂ in when the number of integral values is 32 is associated with classes 136_₂₇₆ to 138_₂₇₆ in when the number of integral values is 276 (See (g) of FIG. 103).

Here, the reason why class 16_₃₂ in when the number of integral values is 32 is associated with classes 136_₂₇₆ to 138_₂₇₆ in when the number of integral values is 276 is to make class 16_₃₆ in when the number of integral values is 32 to the last class in the positive predetermined potential section.

As a result, the integral value ITG_{1_Low_32} in class 1_₃₂ in when the number of integral values is 32 consists of the sum(=ITG_{1_Low_276}+ITG_{2_Low_276}+ITG_{3_Low_276}+ITG_{4_Low_276}+ITG_{5_Low_276}+ITG_{6_Low_276}+ITG_{7_Low_276}+ITG_{8_Low_276}+ITG_{9_Low_276}) from integral value ITG_{1_Low_276} in class 1_₂₇₆ to integral value ITG_{9_Low_276} in class 9_₂₇₆ in when the number of integral values is 276, the integral value ITG_{1_Middle_32} in class 1_₃₂ in when the number of integral values is 32 consists of the sum(=ITG_{1_Middle_276}+ITG_{2_Middle_276}+ITG_{3_Middle_276}+ITG_{4_Middle_276}+ITG_{5_Middle_276}+ITG_{6_Middle_276}+ITG_{7_Middle_276}+ITG_{8_Middle_276}+ITG_{9_Middle_276}) from integral value ITG_{1_Middle_276} in class 1_₂₇₆ to integral value ITG_{9_Middle_276} in class 9_₂₇₆ in when the number of integral values is 276, and the integral value ITG_{1_High_32} in class 1_₃₂ in when the number of integral values is 32 consists of the sum(=ITG_{1_High_276}+ITG_{2_High_276}+ITG_{3_High_276}+ITG_{4_High_276}+ITG_{5_High_276}+ITG_{6_High_276}+ITG_{7_High_276}+ITG_{8_High_276}+ITG_{9_High_276}) from integral value ITG_{1_High_276} in class 1_₂₇₆ to integral value ITG_{9_High_276} in class 9_₂₇₆ in when the number of integral values is 276.

Similarly, the integral value ITG_{15_Low_32} in class 15_₃₂ in when the number of integral values is 32 consists of the sum(=ITG_{127_Low_276}+ITG_{128_Low_276}+ITG_{129_Low_276}+ITG_{130_Low_276}+ITG_{131_Low_276}+ITG_{132_Low_276}+ITG_{133_Low_276}+ITG_{134_Low_276}+ITG_{135_Low_276}) from integral value ITG_{127_Low_276} in class 127_₂₇₆ to integral value ITG_{135_Low_276} in class 135_₂₇₆ in when the number of integral values is 276, the integral value ITG_{15_Middle_32} in class 15_₃₂ in when the number of integral values is 32 consists of the sum (=ITG_{127_Middle_276}+ITG_{128_Middle_276}+ITG_{129_Middle_276}+ITG_{130_Middle_276}+ITG_{131_Middle_276}+ITG_{132_Middle_276}+ITG_{133_Middle_276}+ITG_{134_Middle_276}+ITG_{135_Middle_276}) from integral value ITG_{127_Middle_276} in class 127_₂₇₆ to integral value ITG_{135_Middle_276} in class 135_₂₇₆ in when the number of integral values is 276, and the integral value ITG_{15_High_32} in class 15_₃₂ in when the number of integral values is 32 consists of the sum(=ITG_{127_High_276}+ITG_{128_High_276}+ITG_{129_High_276}+ITG_{130_High_276}+ITG_{131_High_276}+ITG_{132_High_276}+ITG_{133_High_276}+ITG_{134_High_276}+ITG_{135_High_276}) from integral Value ITG_{127_High_276} in class 127_₂₇₆ to integral value ITG_{135_High_276} in class 135_₂₇₆ in when the number of integral values is 276.

Then, the integral value ITG_{16_Low_32} in class 16_₃₂ in when the number of integral values is 32 consists of the sum(=ITG_{136_Low_276}+ITG_{137_Low_276}+ITG_{138_Low_276}) of integral value ITG_{136_Low_276} in class 136_₂₇₆, integral value ITG_{137_Low_276} in class 137_₂₇₆, and integral value ITG_{138_Low_276} in class 138_₂₇₆ in when the number of integral values is 276, the integral value ITG_{16_Middle_32} in class 16_₃₂ in when the number of integral values is 32 consists of the sum(=ITG_{136_Middle_276}+ITG_{137_Middle_276}+ITG_{138_Middle_276}) of integral value ITG_{136_Middle_276} in class 136_₂₇₆, integral value ITG_{137_Middle_276} in class 137_₂₇₆, and integral value ITG_{138_Middle_276} in class 138_₂₇₆ in when the number of integral values is 276, and the integral value ITG_{16_High_32} in class 16_₃₂ in when the number of integral values is 32 consists of the sum(=ITG_{136_High_276}+ITG_{137_High_276}+ITG_{138_High_276}) of integral value ITG_{136_High_276} in class 136_₂₇₆, integral value ITG_{137_High_276} in class 137_₂₇₆, and integral value ITG_{138_High_276} in class 138_₂₇₆ in when the number of integral values is 276.

Furthermore, when the analyte is “shochu”, class 1_₂₀ in when the number of integral values is 20 is associated with classes 1_₂₇₆ to 15_₂₇₆ in when the number of integral values is 276, similarly, class 9_₂₀ in when the number of integral values is 20 is associated with classes 121_₂₇₆ to 135_₂₇₆ in when the number of integral values is 276, and class 10_₂₀ in when the number of integral values is 20 is associated with classes 136_₂₇₆ to 138_₂₇₆ in when the number of integral values is 276 (See (h) of FIG. 103),

Here, the reason why class 10_₂₀ in when the number of integral values is 20 is associated with classes 136_₂₇₆ to 138_₂₇₆ in when the number of integral values is 276 is to make class 10_₂₀ in when the number of integral values is 20 to the last class in the positive predetermined potential section.

As a results, the integral value ITG_{1_Low_20} in class 1_₂₀ in when the number of integral values is 20 consists of the sum(=ITG_{1_Low_276}+ITG_{2_Low_276}+ITG_{3_Low_276}+ITG_{4_Low_276}+ITG_{5_Low_276}+ITG_{6_Low_276}+ITG_{7_Low_276}+ITG_{8_Low_276}+ITG_{9_Low_276}+ , . . . , +ITG_{15_Low_276}) from integral value ITG_{1_Low_276} in class 1_₂₇₆ to integral value ITG_{15_Low_276} in class 15_₂₇₆ in when the number of integral values is 276, the integral value ITG_{1_Middle_20} in class 1_₂₀ in when the number of integral values is 20 consists of the sum(=ITG_{1_Middle_276}+ITG_{2_Middle_276}+ITG_{3_Middle_276}+ITG_{4_Middle_276}+ITG_{5_Middle_276}+ITG_{6_Middle_276}+ITG_{7_Middle_276}+ITG_{8_Middle_276}+ITG_{9_Middle_276}+ , . . . , +ITG_{15_Middle_276}) from integral value ITG_{1_Middle_276} in class 1_₂₇₆ to integral value ITG_{15_Middle_276} in class 15_₂₇₆ in when the number of integral values is 276, and the integral value ITG_{1_High_20} in class 1_₂₀ in when the number of integral values is 20 consists of the sum(=ITG_{1_High_276}+ITG_{2_High_276}+ITG_{3_High_276}+ITG_{4_High_276}+ITG_{5_High_276}+ITG_{6_High_276}+ITG_{7_High_276}+ITG_{8_High_276}+ITG_{9_High_276}+ , . . . , +ITG_{15_High_276}) from integral value ITG_{1_High_276} in class 1_₂₇₆ to integral value ITG_{15_High_276} in class 15_₂₇₆ in when the number of integral values is 276.

Similarly, the integral value ITG_{9_Low_20} in class 9_₂₀ in when the number of integral values is 20 consists of the sum(=ITG_{121_Low_276}+ITG_{122_Low_276}+ITG_{123_Low_276}+ITG_{124_Low_276}+ITG_{125_Low_276}+ITG_{126_Low_276}+ITG_{127_Low_276}+ITG_{128_Low_276}+ITG_{129_Low_276}+ , . . . , +ITG_{135_Low_276}) from integral value ITG_{121_Low_276} in class 121_₂₇₆ to integral value ITG_{135_Low_276} in class 135_₂₇₆ in when the number of integral values is 276, the integral value ITG_{9_Middle_20} in class 9_₂₀ in when the number of integral values is 20 consists of the sum(=ITG_{121_Middle_276}+ITG_{122_Middle_276}+ITG_{123_Middle_276}+ITG_{124_Middle_276}+ITG_{125_Middle_276}+ITG_{126_Middle_276}+ITG_{127_Middle_276}+ITG_{128_Middle_276}+ITG_{129_Middle_276}+ , . . . ,ITG_{135_Middle_276}) from integral value ITG_{121_Middle_276} in class 121_₂₇₆ to integral value ITG_{135_Middle_276} in class 135_₂₇₆ in when the number of integral values is 276, and the integral value ITG_{9_High_20} in class 9_₂₀ in when the number of integral values is 20 consists of the sum(=ITG_{121_High_276}+ITG_{122_High_276}+ITG_{123_High_276}+ITG_{124_High_276}+ITG_{125_High_276}+ITG_{126_High_276}+ITG_{127_High_276}+ITG_{128_High_276}+ITG_{129_High_276}+ , . . . , +ITG_{135_High_276}) from integral value ITG_{121_High_276} in class 121_₂₇₆ to integral value ITG_{135_High_276} in class 135_₂₇₆ in when the number of integral values is 276.

Then, the integral value ITG_{10_Low_20} in class 10_₂₀ in when the number of integral values is 20 consists of the sum(=ITG_{136_Low_276}+ITG_{137_Low_276}+ITG_{138_Low_276}) from integral value ITG_{136_Low_276} in class 136_₂₇₆ to integral value ITG_{138_Low_276} in class 138_₂₇₆ in when the number of integral values is 276, the integral value ITG_{10_Middle_20} in class 10_₂₀ in when the number of integral values is 20 consists of the sum(=ITG_{136_Middle_276}+ITG_{137_Middle_276}+ITG_{138_Middle_276}) from integral value ITG_{136_Middle_276} in class 136_₂₇₆ to integral value ITG_{138_Middle_276} in class 138_₂₇₆ in when the number of integral values is 276, and the integral value ITG_{10_High_20} in class 10_₂₀ in when the number of integral values is 20 consists of the sum(=ITG_{136_High_276}+ITG_{137_High_276}+ITG_{138_High_276}) from integral value ITG_{136_High_276} in class 136_₂₇₆ to integral value ITG_{138_High_276} in class 138_₂₇₆ in when the number of integral values is 276.

Furthermore, when the analyte is “shochu”, class 1_₁₈ in when the number of integral values is 18 is associated with classes 1_₂₇₆ to 17_₂₇₆ in when the number of integral values is 276, similarly, when the analyte is “shochu”, class 8_₁₈ in when the number of integral values is 18 is associated with classes 120_₂₇₆ to 136_₂₇₆ in when the number of integral values is 276, and when the analyte is “shochu”, class 9_₁₈ in when the number of integral values is 18 is associated with classes 137_₂₇₆ and 138_₂₇₆ in when the number of integral values is 276 (See (i) of FIG. 103).

Here, the reason why class 9_₁₈ in when the number of integral values is 18 is associated with classes 137_₂₇₆ and 138_₂₇₆ in when the number of integral values is 276 is to make class 9_₁₈ in when the number of integral values is 18 to the last class in the positive predetermined potential section.

As a result, the integral value ITG_{1_Low_18} in class 1_₁₈ in when the number of integral values is 18 consists of the sum(=ITG_{1_Low_276}+ITG_{2_Low_276}+ITG_{3_Low_276}+ITG_{4_Low_276}+ITG_{5_Low_276}+ITG_{6_Low_276}+ITG_{7_Low_276}+ITG_{8_Low_276}+ITG_{9_Low_276}+ , . . . , +ITG_{17_Low_276}) from integral value ITG_{1_Low_276} in class 1_₂₇₆ to integral value ITG_{17_Low_276} in class 17_₂₇₆ in when the number of integral values is 276, the integral value ITG_{1_Middle_18} in class 1_₁₈ in when the number of integral values is 18 consists of the sum(=ITG_{1_Middle_276}+ITG_{2_Middle_276}+ITG_{3_Middle_276}+ITG_{4_Middle_276}+ITG_{5_Middle_276}+ITG_{6_Middle_276}+ITG_{7_Middle_276}+ITG_{8_Middle_276}+ITG_{9_Middle_276}+ , . . . , +ITG_{17_Middle_276}) from integral value ITG_{1_Middle_276} in class 1_₂₇₆ to integral value ITG_{17_Middle_276} in class 17_₂₇₆ in when the number of integral values is 276, and the integral value ITG_{1_High_18} in class 1_₁₈ in when the number of integral values is 18 consists of the sum(=ITG_{1_High_276}+ITG_{2_High_276}+ITG_{3_High_276}+ITG_{4_High_276}+ITG_{5_High_276}+ITG_{6_High_276}+ITG_{7_High_276}+ITG_{8_High_276}+ITG_{9_High_276}+ , . . . , +ITG_{17_High_276}) from integral value ITG_{1_High_276} in class 1_₂₇₆ to integral value ITG_{17_High_276} in class 17_₂₇₆ in when the number of integral values is 276.

Similarly, the integral value ITG_{8_Low_18} in class 8_₁₈ in when the number of integral values is 18 consists of the sum(=ITG_{120_Low_276}+ITG_{121_Low_276}+ITG_{122_Low_276}+ITG_{123_Low_276}+ITG_{124_Low_276}+ITG_{125_Low_276}+ITG_{126_Low_276}+ITG_{127_Low_276}+ITG_{128_Low_276}+ , . . . , +ITG_{136_Low_276}) from integral value ITG_{120_Low_276} in class 120_₂₇₆ to integral value ITG_{136_Low_276} in class 136_₂₇₆ in when the number of integral values is 276, the integral value ITG_{8_Middle_18} in class 8_₁₈ in when the number of integral values is 18 consists of the sum (=ITG_{120_Middle_276}+ITG_{121_Middle_276}+ITG_{122_Middle_276}+ITG_{123_Middle_276}+ITG_{124_Middle_276}+ITG_{125_Middle_276}+ITG_{126_Middle_276}+ITG_{127_Middle_276}+ITG_{128_Middle_276}+ . . . , +ITG_{136_Middle_276}) from integral value ITG_{120_Middle_276} in class 120_₂₇₆ to integral value ITG_{136_Middle_276} in class 136_₂₇₆ in when the number of integral values is 276, and the integral value ITG_{8_High_18} in class 8_₁₈ in when the number of integral values is 18 consists of the sum(=ITG_{120_High_276}+ITG_{121_High_276}+ITG_{122_High_276}+ITG_{123_High_276}+ITG_{124_High_276}+ITG_{125_High_276}+ITG_{126_High_276}+ITG_{127_High_276}+ITG_{128_High_276}+ , . . . , +ITG_{136_High_276}) from integral value ITG_{120_High_276} in class 120_₂₇₆ to integral value ITG_{136_High_276} in class 136_₂₇₆ in when the number of integral values is 276.

Then, the integral value ITG_{9_Low_18} in class 9_₁₈ in when the number of integral values is 18 consists of the sum(=ITG_{137_Low_276}+ITG_{138_Low_276}) of integral value ITG_{137_Low_276} in class 137_₂₇₆ and integral value ITG_{138_Low_276} in class 138_₂₇₆ in when the number of integral values is 276, the integral value ITG_{9_Middle_18} in class 9_₁₈ in when the number of integral values is 18 consists of the sum(=ITG_{137_Middle_276}+ITG_{138_Middle_276}) of integral value ITG_{137_Middle_276} in class 137_₂₇₆ and integral value ITG_{138_Middle_276} in class 138_₂₇₆ in when the number of integral values is 276, and the integral value ITG_{9_High_18} in class 9_₁₈ in when the number of integral values is 18 consists of the sum(=ITG_{137_High_276}+ITG_{138_High_276}) of integral value ITG_{137_High_276} in class 137_₂₇₆ and integral value ITG_{138_High_276} in class 138_₂₇₆ in when the number of integral values is 276.

As described above, the integral values ITG_{1_Low_138} to ITG_{69_Low_138} in classes 1_₁₃₈ to 69_₁₃₈ in when the number of integral values is 138 are calculated using the integral values ITG_{1_Low_276} to ITG_{138_Low_276} in classes 1_₂₇₆ to 138_₂₇₆ in when the number of integral values is 276, the integral values ITG_{1_Middle_138} to ITG_{69_Low_138} in classes 1_₁₃₈ to 69_₁₃₈ in when the number of integral values is 138 are calculated using the integral values ITG_{1_Middle_276} to ITG_{138_Middle_276} in classes 1_₂₇₆ to 138_₂₇₆ in when the number of integral values is 276, and the integral values ITG_{1_High_138} to ITG_{69_Low_138} in classes 1_₁₃₈ to 69_₁₃₈ in when the number of integral values is 138 are calculated using the integral values ITG_{1_High_276} to ITG_{138_High_276} in classes 1_₂₇₆ to 138_₂₇₆ in when the number of integral values is 276.

Also, the integral values ITG_{1_Low_92} to ITG_{46_Low_92} in classes 1_₉₂ to 46_₉₂ in when the number of integral values is 92, the integral values ITG_{1_Low_70} to ITG_{35_Low_70} in classes 1_₇₀ to 35_₇ in when the number of integral values is 70, the integral values ITG_{1_Low_56} to ITG_{28_Low_56} in classes 1_₅₆ to 28_₅₆ in when the number of integral values is 56, the integral values ITG_{1_Low_36} to ITG_{18_Low_36} in classes 1_₃₆ to 18_₃₆ in when the number of integral values is 36, the integral values ITG_{1_Low_32} to ITG_{16_Low_32} in classes 1_₃₂ to 16_₃₂ in when the number of integral values is 32, the integral values ITG_{1_Low_20} to ITG_{10_Low_20} in classes 1_₂₀ to 10_₂₀ in when the number of integral values is 20, and the integral values ITG_{1_Low_18} to ITG_{9_Low_18} in classes 1_₁₈ to 9_₁₈ in when the number of integral values is 18, similarly, are calculated using the integral values ITG_{1_High_276} to ITG_{138_High_276} in class 1_₂₇₆ to class 138_₂₇₆ in when the number of integral values is 276.

Therefore, classes 1_₁₃₈ to 69_₁₃₈ in when the number of integral values is 138, classes 1_₉₂ to 46_₉₂ in when the number of integral values is 92, classes 1_₇₀ to 35_₇₀ in when the number of integral values is 70, classes 1_₅₆ to 28_₅₆ in when the number of integral values is 56, classes 1_₃₆ to 18_₃₆ in when the number of integral values is 36, classes 1_₃₂ to 16_₃₂ in when the number of integral values is 32, classes 1_₂₀ to 10_₂₀ in when the number of integral values is 20, and classes 1_₁₈ to 9_₁₈ in when the number of integral values is 18 are associated with classes 1_₂₇₆ to 138_₂₇₆ in when the number of integral values is 276, as a results, regardless of whether the number of integral values is 138, 92, 70, 56, 36, 32, 20, or 18, the sum of the integral values in the positive predetermined potential section can be made to match the sum of the integral values in classes 1_₂₇₆ to 138_₂₇₆ (=positive predetermined potential section) in when the number of integral values is 276.

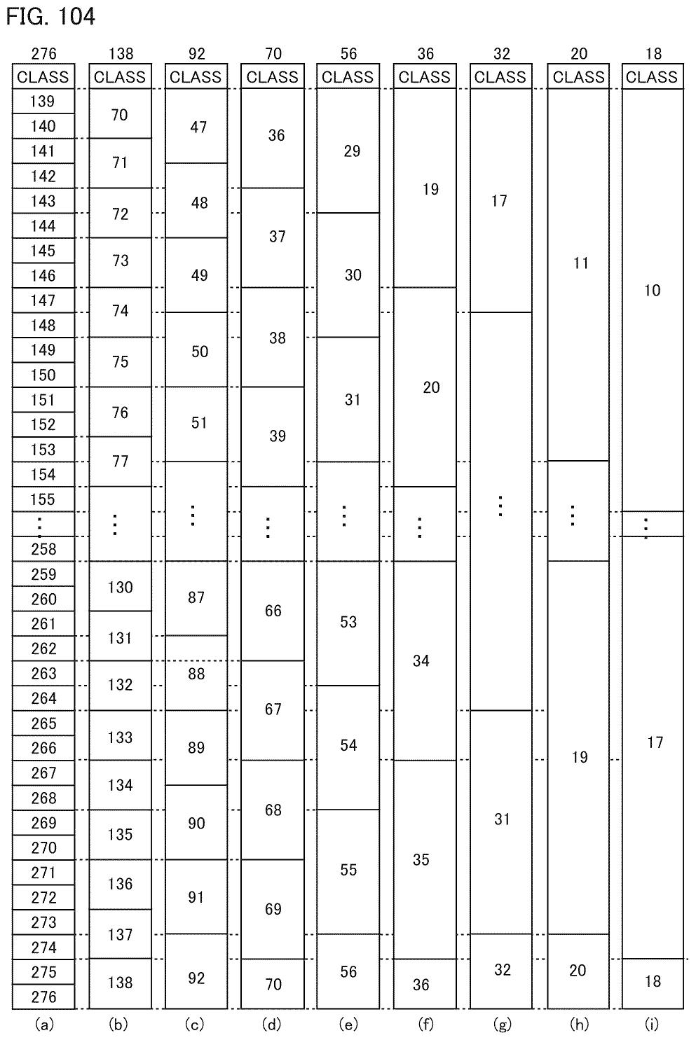

FIG. 104 is a diagram showing the correspondence relationship of classes in a negative predetermined potential range about when the number of integral values in when the analyte is “shochu” is 18, 20, 32, 36, 56, 70, 92, 138 and 276.

Referring to FIG. 104, when the analyte is “shochu”, the class 70_₁₃₈ in when the number of integral values is 138 is corresponded to classes 139_₂₇₆ and 140_₂₇₆ in when the number of integral values is 276, the class 71_₁₃₈ in when the number of integral values is 138 is corresponded to classes 141_₂₇₆ and 142_₂₇₆ in when the number of integral values is 276, the class 72_₁₃₈ in when the number of integral values is 138 is corresponded to classes 143_₂₇₆ and 144_₂₇₆ in when the number of integral values is 276, similarly, the class 136_₁₃₈ in when the number of integral values is 138 is corresponded to classes 271_₂₇₆ and 272_₂₇₆ in when the number of integral values is 276, the class 137_₁₃₈ in when the number of integral values is 138 is corresponded to classes 273_₂₇₆ and 274_₂₇₆ in when the number of integral values is 276, and the class 138_₁₃₈ in when the number of integral values is 138 is corresponded to classes 275_₂₇₆ and 276_₂₇₆ in when the number of integral values is 276 (See (b) of FIG. 104).

As a result, the integral value ITG_{70_Low_138} in class 70_₁₃₈ in when the number of integral values is 138 consists of the sum(=ITG_{139_Low_276}+ITG_{140_Low_276}) of integral value ITG_{139_Low_276} in class 139_₂₇₆ and integral value ITG_{140_Low_276} in class 140_₂₇₆ in when the number of integral values is 276, the integral value ITG_{70_Middle_138} in class 70_₁₃₈ in when the number of integral values is 138 consists of the sum(=ITG_{139_Middle_276}+ITG_{140_Middle_276}) of integral value ITG_{139_Middle_276} in class 139_₂₇₆ and integral value ITG_{140_Middle_276} in class 140_₂₇₆ in when the number of integral values is 276, and he integral value ITG_{70_High_138} in class 70_₁₃₈ in when the number of integral values is 138 consists of the sum(=ITG_{139_High_276}+ITG_{140_High_276}) of integral value ITG_{139_High_276} in class 139_₂₇₆ and integral value ITG_{140_High_276} in class 140_₂₇₆ in when the number of integral values is 276.

Also, the integral value ITG_{71_Low_138} in class 71_₁₃₈ in when the number of integral values is 138 consists of the sum(=ITG_{141_Low_276}+ITG_{142_Low_276}) of integral value ITG_{141_Low_276} in class 141_₂₇₆ and integral value ITG_{142_Low_276} in class 142_₂₇₆ in when the number of integral values is 276, the integral value ITG_{71_Middle_138} in class 71_₁₃₈ in when the number of integral values is 138 consists of the sum(=ITG_{141_Middle_276}+ITG_{142_Middle_276}) of integral value ITG_{141_Middle_276} in class 141_₂₇₆ and integral value ITG_{142_Middle_276} in class 142_₂₇₆ in when the number of integral values is 276, and the integral value ITG_{71_High_138} in class 71_₁₃₈ in when the number of integral values is 138 consists of the sum(=ITG_{141_High_276}+ITG_{142_High_276}) of integral value ITG_{141_High_276} in class 141_₂₇₆ and integral value ITG_{142_High_276} in class 142_₂₇₆ in when the number of integral values is 276.

Similarly, the integral value ITG_{138_Low_138} in class 138_₁₃₈ in when the number of integral values is 138 consists of the sum(=ITG_{275_Low_276}+ITG_{276_Low_276}) of integral value ITG_{275_Low_276} in class 275_₂₇₆ and integral value ITG_{276_Low_276} in class 276_₂₇₆ in when the number of integral values is 276, the integral value ITG_{138_Middle_138} in class 138_₁₃₈ in when the number of integral values is 138 consists of the sum(=ITG_{275_Middle_276}+ITG_{276_Middle_276}) of integral value ITG_{275_Middle_276} in class 275_₂₇₆ and integral value ITG_{276_Middle_276} in class 276_₂₇₆ in when the number of integral values is 276, and the integral value ITG_{138_High_138} in class 138_₁₃₈ in when the number of integral values is 138 consists of the sum(=ITG_{275_High_276}+ITG_{276_High_276}) of integral value ITG_{275_High_276} in class 275_₂₇₆ and integral value ITG_{276_High_276} in class 276_₂₇₆ in when the number of integral values is 276.

Also, when the analyte is “shochu”, the class 47_₉₂ in when the number of integral values is 92 is corresponded to classes 139_₂₇₆ to 141_₂₇₆ in when the number of integral values is 276, the class 48_₉₂ in when the number of integral values is 92 is corresponded to classes 142_₂₇₆ to 144_₂₇₆ in when the number of integral values is 276, the class 49_₉₂ in when the number of integral values is 92 is corresponded to classes 145_₂₇₆ to 147_₂₇₆ in when the number of integral values is 276, similarly, the class 91_₉₂ in when the number of integral values is 92 is corresponded to classes 271_276 to 273_₂₇₆ in when the number of integral values is 276, and the class 92_₉₂ in when the number of integral values is 92 is corresponded to classes 274_₂₇₆ to 276_₂₇₆ in when the number of integral values is 276 (See (c) of FIG. 104).

As a result, the integral value ITG_{47_Low_92} in class 47_₉₂ in when the number of integral values is 92 consists of the sum(=ITG_{139_Low_276}+ITG_{140_Low_276}+ITG_{141_Low_276}) of integral value ITG_{139_Low_276} in class 139_₂₇₆, integral value ITG_{140_Low_276} in class 140_₂₇₆, and integral value ITG_{141_Low_276} in class 141_₂₇₆ in when the number of integral values is 276, the integral value ITG_{47_Middle_92} in class 47_₉₂ in when the number of integral values is 92 consists of the sum(=ITG_{139_Middle_276}+ITG_{140_Middle_276}+ITG_{141_Middle_276}) of integral value ITG_{139_Middle_276} in class 139_₂₇₆, integral value ITG_{140_Middle_276} in class 140_₂₇₆, and integral value ITG_{141_Middle_276} in class 141_₂₇₆ in when the number of integral values is 276, and the integral value ITG_{47_High_92} in class 47_₉₂ in when the number of integral values is 92 consists of the sum(=ITG_{139_High_276}+ITG_{140_High_276}+ITG_{141_High_276}) of integral value ITG_{139_High_276} in class 139_₂₇₆, integral value ITG_{140_High_276} in class 140_₂₇₆, and integral value ITG_{141_High_276} in class 141_₂₇₆ in when the number of integral values is 276.

Also, the integral value ITG_{48_Low_92} in class 48_₉₂ in when the number of integral values is 92 consists of the sum(=ITG_{142_Low_276}+ITG_{143_Low_276}+ITG_{144_Low_276}) of integral value ITG_{142_Low_276} in class 142_₂₇₆, integral value ITG_{143_Low_276} in class 143_₂₇₆, and integral value ITG_{144_Low_276} in class 144_₂₇₆ in when the number of integral values is 276, the integral value ITG_{48_Middle_92} in class 48_₉₂ in when the number of integral values is 92 consists of the sum(=ITG_{142_Middle_276}+ITG_{143_Middle_276}+ITG_{144_Middle_276}) of integral value ITG_{142_Middle_276} in class 142_₂₇₆, integral value ITG_{143_Middle_276} in class 143_₂₇₆, and integral value ITG_{144_Middle_276} in class 144_₂₇₆ in when the number of integral values is 276, and the integral value ITG_{48_High_92} in class 48_₉₂ in when the number of integral values is 92 consists of the sum(=ITG_{142_High_276}+ITG_{143_High_276}+ITG_{144_High_276}) of integral value ITG_{142_High_276} in class 142_₂₇₆, integral value ITG_{143_High_276} in class 143_₂₇₆, and integral value ITG_{144_High_276} in class 144_₂₇₆ in when the number of integral values is 276.

Similarly, the integral value ITG_{92_Low_92} in class 92_₉₂ in when the number of integral values is 92 consists of the sum(=ITG_{274_Low_276}+ITG_{275_Low_276}+ITG_{276_Low_276}) of integral value ITG_{274_Low_276} in class 274_₂₇₆, integral value ITG_{275_Low_276} in class 275_₂₇₆, and integral value ITG_{276_Low_276} in class 276_₂₇₆ in when the number of integral values is 276, the integral value ITG_{92_Middle_92} in class 92_₉₂ in when the number of integral values is 92 consists of the sum(=ITG_{274_Middle_276}+ITG_{275_Middle_276}+ITG_{276_Middle_276}) of integral value ITG_{274_Middle_276} in class 274_₂₇₆, integral value ITG_{275_Middle_276} in class 275_₂₇₆, and integral value ITG_{276_Middle_276} in class 276_₂₇₆ in when the number of integral values is 276, and the integral value ITG_{92_High_92} in class 92_₉₂ in when the number of integral values is 92 consists of the sum(=ITG_{274_High_276}+ITG_{275_High_276}+ITG_{276_High_276}) of integral value ITG_{274_High_276} in class 274_₂₇₆, integral value ITG_{275_High_276} in class 275_₂₇₆, and integral value ITG_{276_High_276} in class 276_₂₇₆ in when the number of integral values is 276.

Furthermore, when the analyte is “shochu”, class 36_₇₀ in when the number of integral values is 70 is associated with classes 139_₂₇₆ to 142_₂₇₆ in when the number of integral values is 276, class 37_₇₀ in when the number of integral values is 70 is associated with classes 143_₂₇₆ to 146_₂₇₆ in when the number of integral values is 276, class 38_₇₀ in when the number of integral values is 70 is associated with classes 147_₂₇₆ to 150_₂₇₆ in when the number of integral values is 276, similarly, class 69₇₀ in when the number of integral values is 70 is associated with classes 271_₂₇₆ to 274_₂₇₆ in when the number of integral values is 276, and class 70_₇₀ in when the number of integral values is 70 is associated with classes 275_₂₇₆ and 276_₂₇₆ in when the number of integral values is 276 (See (d) of FIG. 104).

As a result, the integral value ITG_{36_Low_70} in class 36_₇₀ in when the number of integral values is 70 consists of the sum(=ITG_{139_Low_276}+ITG_{140_Low_276}+ITG_{141_Low_276}+ITG_{142_Low_276}) of integral value ITG_{139_Low_276} in class 139_₂₇₆, integral value ITG_{140_Low_276} in class 140_₂₇₆, integral value ITG_{141_Low_276} in class 141_₂₇₆, and integral value ITG_{142_Low_276} in class 142_₂₇₆ in when the number of integral values is 276, the integral value ITG_{36_Middle_70} in class 36_₇₀ in when the number of integral values is 70 consists of the sum(=ITG_{139_Middle_276}+ITG_{140_Middle_276}+ITG_{141_Middle_276}+ITG_{142_Middle_276}) of integral value ITG_{139_Middle_276} in class 139_₂₇₆, integral value ITG_{140_Middle_276} in class 140_₂₇₆, integral value ITG_{141_Middle_276} in class 141_₂₇₆, and integral value ITG_{142_middle_276} in class 142_₂₇₆ in when the number of integral values is 276, and the integral value ITG_{36_High_70} in class 36_₇₀ in when the number of integral values is 70 consists of the sum(=ITG_{139_High_276}+ITG_{140_High_276}+ITG_{141_High_276}+ITG_{142_High_276}) of integral value ITG_{139_High_276} in class 139_₂₇₆, integral value ITG_{140_High_276} in class 140_₂₇₆, integral value ITG_{141_High_276} in class 141_₂₇₆, and integral value ITG_{142_High_276} in class 142_₂₇₆ in when the number of integral values is 276.

Also, the integral value ITG_{37_Low_70} in class 37_₇₀ in when the number of integral values is 70 consists of the sum(=ITG_{143_Low_276}+ITG_{144_Low_276}+ITG_{145_Low_276}+ITG_{146_Low_276}) of integral value ITG_{143_Low_276} in class 143_₂₇₆, integral value ITG_{144_Low_276} in class 144_₂₇₆, integral value ITG_{145_Low_276} in class 145_₂₇₆, and integral value ITG_{146_Low_276} in class 146_₂₇₆ in when the number of integral values is 276, the integral value ITG_{37_Middle_70} in class 37_₇₀ in when the number of integral values is 70 consists of the sum(=ITG_{143_Middle_276}+ITG_{144_Middle_276}+ITG_{145_Middle_276}+ITG_{146_Middle_276}) of integral value ITG_{143_Middle_276} in class 143_₂₇₆, integral value ITG_{144_Middle_276} in class 144_₂₇₆, integral value ITG_{145_Middle_276} in class 145_₂₇₆, and integral value ITG_{146_Middle_276} in class 146_₂₇₆ in when the number of integral values is 276, the integral value ITG_{37_High_70} in class 37_₇₀ in when the number of integral values is 70 consists of the sum(=ITG_{143_High_276}+ITG_{144_High_276}+ITG_{145_High_276}+ITG_{146_High_276}) of integral value ITG_{143_High_276} in class 143_₂₇₆, integral value ITG_{144_High_276} in class 144_₂₇₆, integral value ITG_{145_High_276} in class 145_₂₇₆, and integral value ITG_{146_High_276} in class 146_₂₇₆ in when the number of integral values is 276.

Similarly, the integral value ITG_{69_Low_70} in class 69_₇₀ in when the number of integral values is 70 consists of the sum(=ITG_{271_Low_276}+ITG_{272_Low_276}+ITG_{273_Low_276}+ITG_{274_Low_276}) of integral value ITG_{271_Low_276} in class 271_₂₇₆, integral value ITG_{272_Low_276} in class 272_₂₇₆, integral value ITG_{273_Low_276} in class 273_₂₇₆, and integral value ITG_{274_Low_276} in class 274_₂₇₆ in when the number of integral values is 276, the integral value ITG_{69_Middle_70} in class 69_₇₀ in when the number of integral values is 70 consists of the sum(=ITG_{271_Middle_276}+ITG_{272_Middle_276}+ITG_{273_Middle_276}+ITG_{274_Middle_276}) of integral value ITG_{271_Middle_276} in class 271_₂₇₆, integral value ITG_{272_Middle_276} in class 272_₂₇₆, integral value ITG_{273_Middle_276} in class 273_₂₇₆, and integral value ITG_{274_Middle_276} in class 274_₂₇₆ in when the number of integral values is 276, and the integral value ITG_{69_High_70} in class 69_₇₀ in when the number of integral values is 70 consists of the sum(=ITG_{271_High_276}+ITG_{272_High_276}+ITG_{273_High_276}+ITG_{274_High_276}) of integral value ITG_{271_High_276} in class 271_₂₇₆, integral value ITG_{272_High_276} in class 272_₂₇₆, integral value ITG_{273_High_276} in class 273_₂₇₆, and integral value ITG_{274_High_276} in class 274_₂₇₆ in when the number of integral values is 276.

Then, the integral value ITG_{70_Low_70} in class 70_₇₀ in when the number of integral values is 70 consists of the sum(=ITG_{275_Low_276}+ITG_{276_Low_276}) of integral value ITG_{275_Low_276} in class 275_₂₇₆ and integral value ITG_{276_Low_276} in class 276_₂₇₆ in when the number of integral values is 276, the integral value ITG_{70_Middle_70} in class 70_₇₀ in when the number of integral values is 70 consists of the sum(=ITG_{275_Middle_276}+ITG_{276_Middle_276}) of integral value ITG_{275_Middle_276} in class 275_₂₇₆ and integral value ITG_{276_Middle_276} in class 276_₂₇₆ in when the number of integral values is 276, and the integral value ITG_{70_High_70} in class 70_₇₀ in when the number of integral values is 70 consists of the sum(=ITG_{275_High_276}+ITG_{276_High_276}) of integral value ITG_{275_High_276} in class 275_₂₇₆ and integral value ITG_{276_High_276} in class 276_₂₇₆ in when the number of integral values is 276.

Furthermore, when the analyte is “shochu”, class 29_₅₆ in when the number of integral values is 56 is associated with classes 139_₂₇₆ to 143_₂₇₆ in when the number of integral values is 276, class 30_₅₆ in when the number of integral values is 56 is associated with classes 144_₂₇₆ to 148_₂₇₆ in when the number of integral values is 276, similarly, class 55_₅₆ in when the number of integral values is 56 is associated with classes 269_₂₇₆ to 273_₂₇₆ in when the number of integral values is 276, class 56_₅₆ in when the number of integral values is 56 is associated with classes 274_₂₇₆ to 276_₂₇₆ in when the number of integral values is 276 (See (e) of FIG. 104).

As a result, the integral value ITG_{29_Low_56} in the class 29₅₆ in when the number of integral values is 56 consists of the sum(=ITG_{139_Low_276}+ITG_{140_Low_276}+ITG_{141_Low_276}+ITG_{142_Low_276}+ITG_{143_Low_276}) of integral value ITG_{139_Low_276} in class 139_₂₇₆, integral value ITG_{140_Low_276} in class 140_₂₇₆, integral value ITG_{141_Low_276} in class 141_₂₇₆, integral value ITG_{142_Low_276} in class 142_₂₇₆, and integral value ITG_{143_Low_276} in class 143_₂₇₆ in when the number of integral values is 276, the integral value ITG_{29_Middle_56} in the class 29_₅₆ in when the number of integral values is 56 consists of the sum (=ITG_{139_Middle_276}+ITG_{140_Middle_276}+ITG_{141_Middle_276}+ITG_{142_Middle_276}+ITG_{143_Middle_276}) of integral value ITG_{139_Middle_276} in class 139_₂₇₆, integral value ITG_{140_Middle_276} in class 140_₂₇₆, integral value ITG_{141_Middle_276} in class 141_₂₇₆, integral value ITG_{142_Middle_276} in class 142_₂₇₆, and integral value ITG_{143_Middle_276} in class 143_₂₇₆ in when the number of integral values is 276, and the integral value ITG_{29_High_56} in the class 29_₅₆ in when the number of integral values is 56 consists of the sum (=ITG_{139_High_276}+ITG_{140_High_276}+ITG_{141_High_276}+ITG_{142_High_276}+ITG_{143_High_276}) of integral value ITG_{139_High_276} in class 139_₂₇₆, integral value ITG_{140_High_276} in class 140_₂₇₆, integral value ITG_{141_High_276} in class 141_₂₇₆, integral value ITG_{142_High_276} in Class 142_₂₇₆, and integral value ITG_{143_High_276} in class 143_₂₇₆ in when the number of integral values is 276.

Also, the integral value ITG_{30_Low_56} in the class 30_₅₆ in when the number of integral values is 56 consists of the sum(=ITG_{144_Low_276}+ITG_{145_Low_276}+ITG_{146_Low_276}+ITG_{147_Low_276}+ITG_{148_Low_276}) of integral value ITG_{144_Low_276} in class 144_₂₇₆, integral value ITG_{145_Low_276} in class 145_₂₇₆, integral value ITG_{146_Low_276} in class 146_₂₇₆, integral value ITG_{147_Low_276} in class 147_₂₇₆, and integral value ITG_{148_Low_276} in class 148_₂₇₆ in when the number of integral values is 276, the integral value ITG_{30_Middle_56} in the class 30_₅₆ in when the number of integral values is 56 consists of the sum(=ITG_{144_Middle_276}+ITG_{145_Middle_276}+ITG_{146_Middle_276}+ITG_{147_Middle_276}+ITG_{148_Middle_276}) of integral value ITG_{144_Middle_276} in class 144_₂₇₆, integral value ITG_{145_Middle_276} in class 145_₂₇₆, integral value ITG_{146_Middle_276} in class 146_₂₇₆, integral value ITG_{147_Middle_276} in class 147_₂₇₆, and integral value ITG_{148_Middle_276} in class 148_₂₇₆ in when the number of integral values is 276, and the integral value ITG_{30_High_56} in the class 30_₅₆ in when the number of integral values is 56 consists of the sum(=ITG_{144_High_276}+ITG_{145_High_276}+ITG_{146_High_276}+ITG_{147_High_276}+ITG_{148_High_276}) of integral value ITG_{144_High_276} in class 144_₂₇₆, integral value ITG_{145_High_276} in class 145_₂₇₆, integral value ITG_{146_High_276} in class 146_₂₇₆, integral value ITG_{147_High_276} in class 147_₂₇₆, and integral value ITG_{148_High_276} in class 148_₂₇₆ in when the number of integral values is 276.

Similarly, the integral value ITG_{55_Low_56} in the class 55_₅₆ in when the number of integral values is 56 consists of the sum(=ITG_{269_Low_276}+ITG_{270_Low_276}+ITG_{271_Low_276}+ITG_{272_Low_276}+ITG_{273_Low_276}) of integral value ITG_{269_Low_276} in class 269_₂₇₆, integral value ITG_{270_Low_276} in class 270_₂₇₆, integral value ITG_{271_Low_276} in class 271_₂₇₆, integral value ITG_{272_Low_276} in class 272_₂₇₆, and integral value ITG_{273_Low_276} in class 273_₂₇₆ in when the number of integral values is 276, the integral value ITG_{55_Middle_56} in the class55₅₆ in when the number of integral values is 56 consists of the sum(=ITG_{269_Middle_276}+ITG_{270_Middle_276}+ITG_{271_Middle_276}+ITG_{272_Middle_276}+ITG_{273_Middle_276}) of integral value ITG_{269_Middle_276} in class 269_₂₇₆, integral value ITG_{270_Middle_276} in class 270_₂₇₆, integral value ITG_{271_Middle_276} in class 271_₂₇₆, integral value ITG_{272_Middle_276} in class 272_₂₇₆, and integral value ITG_{273_Middle_276} in class 273_₂₇₆ in when the number of integral values is 276, and the integral value ITG_{55_High_56} in the class 55_₅₆ in when the number of integral values is 56 consists of the sum(=ITG_{269_High_276}+ITG_{270_High_276}+ITG_{271_High_276}+ITG_{272_High_276}+ITG_{273_High_276}) of integral value ITG_{269_High_276} in class 269_₂₇₆, integral value ITG_{270_High_276} in class 270_₂₇₆, integral value ITG_{271_High_276} in class 271_₂₇₆, integral value ITG_{272_High_276} in class 272_₂₇₆, and integral value ITG_{273_High_276} in class 273_₂₇₆ in when the number of integral values is 276.

Then, the integral value ITG_{56_Low_56} in the class56_₅₆ in when the number of integral values is 56 consists of the sum(=ITG_{274_Low_276}+ITG_{275_Low_276}+ITG_{276_Low_276}) of integral value ITG_{274_Low_276} in class 274_₂₇₆, integral value ITG_{275_Low_276} in class 275_₂₇₆, and integral value ITG_{276_Low_276} in class 276_₂₇₆ in when the number of integral values is 276, the integral value ITG_{56_Middle_56} in the class 56_₅₆ in when the number of integral values is 56 consists of the sum(=ITG_{274_Middle_276}+ITG_{275_Middle_276}+ITG_{276_Middle_276}) of integral value ITG_{274_Middle_276} in class 274_₂₇₆, integral value ITG_{275_Middle_276} in class 275_₂₇₆, and integral value ITG_{276_Middle_276} in class 276_₂₇₆ in when the number of integral values is 276, and the integral value ITG_{56_High_56} in the class 56_₅₆ in when the number of integral values is 56 consists of the sum(=ITG_{274_High_276}+ITG_{275_High_276}+ITG_{276_High_276}) of integral Value ITG_{274_High_276} in class 274_₂₇₆, integral value ITG_{275_High_276} in class 275_₂₇₆, and integral value ITG_{276_High_276} in class 276_₂₇₆, in when the number of integral values is 276.

Furthermore, when the analyte is “shochu”, the class 19_₃₆ in when the number of integral values is 36 is associated with classes 139_₂₇₆ to 146_₂₇₆ in when the number of integral values is 276, the class 20_₃₆ in when the number of integral values is 36 is associated with classes 147_₂₇₆ to 154_276 in when the number of integral values is 276, similarly, the class 34_₃₆ in when the number of integral values is 36 is associated with classes 259_₂₇₆ to 266_₂₇₆ in when the number of integral values is 276, the class 35_₃₆ in when the number of integral values is 36 is associated with classes 267_₂₇₆ to 274_₂₇₆ in when the number of integral values is 276, and the class 36_₃₆ in when the number of integral values is 36 is associated with classes 275_₂₇₆ and 276_₂₇₆ in when the number of integral values is 276 (See (f) of FIG. 104).

As a result, the integral value ITG_{19_Low_36} in the class 19_₃₆ in when the number of integral values is 36 consists of the sum(=ITG_{139_Low_276}+ITG_{140_Low_276}+ , . . . , +ITG_{146_Low_276}) from integral value ITG_{139_Low_276} in class 139_₂₇₆ to integral value ITG_{146_Low_276} in class 146_₂₇₆ in when the number of integral values is 276, the integral value ITG_{19_Middle_36} in the class 19_₃₆ in when the number of integral values is 36 consists of the sum(=ITG_{139_Middle_276}+ITG_{140_Middle_276}+ , . . . , +ITG_{146_Middle_276}) from integral value ITG_{139_Middle_276} in class 139_₂₇₆ to integral value ITG_{146_Middle_276} in class 146_₂₇₆ in when the number of integral values is 276, and the integral value ITG_{19_High_36} in the class 19_₃₆ in when the number of integral values is 36 consists of the sum(=ITG_{139_High_276}+ITG_{140_High_276}+ , . . . , +ITG_{146_High_276}) from integral value ITG_{139_High_276} in class 139_₂₇₆ to integral value ITG_{146_High_276} in class 146_₂₇₆ in when the number of integral values is 276.

Also, the integral value ITG_{20_Low_36} in the class 20_₃₆ in when the number of integral values is 36 consists of the sum(=ITG_{147_Low_276}+ITG_{148_Low_276}+ , . . . , +ITG_{154_Low_276}) from integral value ITG_{147_Low_276} in class 147_₂₇₆ to integral value ITG_{154_Low_276} in class 154_₂₇₆ in when the number of integral values is 276, the integral value ITG_{20_Middle_36} in the class 20_₃₆ in when the number of integral values is 36 consists of the sum (=ITG_{147_Middle_276}+ITG_{148_Middle_276}+ , . . . , +ITG_{154_Middle_276}) from integral value ITG_{147_Middle_276} in class 147_₂₇₆ to integral value ITG_{154_Middle_276} in class 154_₂₇₆ in when the number of integral values is 276, and the integral value ITG_{20_High_36} in the class 20_₃₆ in when the number of integral values is 36 consists of the sum (=ITG_{147_High_276}+ITG_{148_High_276}+ . . . , +ITG_{154_High_276}) from integral value ITG_{147_High_276} in class 147_₂₇₆ to integral value ITG_{154_High_276} in class 154_₂₇₆ in when the number of integral values is 276.

Similarly, the integral value ITG_{35_Low_36} in the class 35_₃₆ in when the number of integral values is 36 consists of the sum(=ITG_{267_Low_276}+ITG_{268_Low_276}+ , . . . , +ITG_{274_Low_276}) from integral value ITG_{267_Low_276} in class 267_₂₇₆ to integral value ITG_{274_Low_276} in class 274_₂₇₆ in when the number of integral values is 276, the integral value ITG_{35_Middle_36} in the class 35_₃₆ in when the number of integral values is 36 consists of the sum(=ITG_{267_Middle_276}+ITG_{268_Middle_276}+ , . . . , +ITG_{274_Middle_276}) from integral value ITG_{267_Middle_276} in class 267_₂₇₆ to integral value ITG_{274_Middle_276} in class 274_₂₇₆ in when the number of integral values is 276, and the integral value ITG_{35_High_36} in the class 35_₃₆ in when the number of integral values is 36 consists of the sum(=ITG_{267_High_276}+ITG_{268_High_276}+ , . . . , +ITG_{274_High_276}) from integral value ITG_{267_High_276} in class 267_₂₇₆ to integral value ITG_{274_High_276} in class 274_₂₇₆ in when the number of integral values is 276.

Then, the integral value ITG_{36_Low_36} in the class 36_₃₆ in when the number of integral values is 36 consists of the sum(=ITG_{275_Low_276}+ITG_{276_Low_276}) of integral value ITG_{275_Low_276} in class 275_₂₇₆ and integral value ITG_{276_Low_276} in class 276_₂₇₆ in when the number of integral values is 276, the integral value ITG_{36_Middle_36} in the class 36_₃₆ in when the number of integral values is 36 consists of the sum(=ITG_{275_Middle_276}+ITG_{276_Middle_276}) of integral value ITG_{275_Middle_276} in class 275_₂₇₆ and integral value ITG_{276_Middle_276} in class 276_₂₇₆ in when the number of integral values is 276, and the integral value ITG_{36_High_36} in the class 36_₃₆ in when the number of integral values is 36 consists of the sum(=ITG_{275_High_276}+ITG_{276_High_276}) of integral value ITG_{275_High_276} in class 275_₂₇₆ and integral value ITG_{276_High_276} in class 276_₂₇₆ in when the number of integral values is 276.

Furthermore, when the analyte is “shochu”, the class 17_₃₂ in when the number of integral values is 32 is associated with classes 139_₂₇₆ to 147_₂₇₆ in when the number of integral values is 276, similarly, the class 31_₃₂ in when the number of integral values is 32 is associated with classes 265_₂₇₆ to 273_₂₇₆ in when the number of integral values is 276, the class 32_₃₂ in when the number of integral values is 32 is associated with classes 274_₂₇₆ to 276_₂₇₆ in when the number of integral values is 276 (See (g) if FIG. 104).

As a result, the integral value ITG_{17_Low_32} in the class 17₃₂ in when the number of integral values is 32 consists of the sum(=ITG_{139_Low_276}+ITG_{140_Low_276}+ , . . . , +ITG_{147_Low_276}) from integral value ITG_{139_Low_276} in class 139_₂₇₆ to integral value ITG_{147_Low_276} in class 147_₂₇₆ in when the number of integral values is 276, the integral value ITG_{17_Middle_32} in the class 17_₃₂ in when the number of integral values is 32 consists of the sum(=ITG_{139_Middle_276}+ITG_{140_Middle_276}+ , . . . , +ITG_{147_Middle_276}) from integral value ITG_{139_Middle_276} in class 139_₂₇₆ to integral value ITG_{147_Middle_276} in class 147_₂₇₆ in when the number of integral values is 276, and the integral value ITG_{17_High_32} in the class 17_₃₂ in when the number of integral values is 32 consists of the sum(=ITG_{139_High_276}+ITG_{140_High_276}+ , . . . , +ITG_{147_High_276}) from integral value ITG_{139_High_276} in class 139_₂₇₆ to integral value ITG_{147_High_276} in class 147_₂₇₆ in when the number of integral values is 276.

Similarly, the integral value ITG_{31_Low_32} in the class 31_₃₂ in when the number of integral values is 32 consists of the sum(=ITG_{265_Low_276}+ITG_{266_Low_276}+ , . . . , +ITG_{273_Low_276}) from integral value ITG_{265_Low_276} in class 265_₂₇₆ to integral value ITG_{273_Low_276} in class 273_₂₇₆ in when the number of integral values is 276, the integral value ITG_{31_Middle_32} in the class 31_₃₂ in when the number of integral values is 32 consists of the sum (=ITG_{265_Middle_276}+ITG_{266_Middle_276}+ , . . . , +ITG_{273_Middle_276}) from integral value ITG_{265_Middle_276} in class 265_₂₇₆ to integral value ITG_{273_Middle_276} in class 273_₂₇₆ in when the number of integral values is 276, and the integral value ITG_{31_High_32} in the class 31_₃₂ in when the number of integral values is 32 consists of the sum (=ITG_{265_High_276}+ITG_{266_High_276}+ , . . . , +ITG_{273_High_276}) from integral value ITG_{265_High_276} in class 265_₂₇₆ to integral value ITG_{273_High_276} in class 273_₂₇₆ in when the number of integral values is 276.

Then, the integral value ITG_{32_Low_32} in the class 32_₃₂ in when the number of integral values is 32 consists of the sum(=ITG_{274_Low_276}+ITG_{275_Low_276}+ITG_{276_Low_276}) from integral value ITG_{274_Low_276} in class 274_₂₇₆ to integral value ITG_{276_Low_276} in class 276_₂₇₆ in when the number of integral values is 276, the integral value ITG_{32_Middle_32} in the class 32_₃₂ in when the number of integral values is 32 consists of the sum (=ITG_{274_Middle_276}+ITG_{275_Middle_276}+ITG_{276_Middle_276}) from integral value ITG_{274_Middle_276} in class 274_₂₇₆ to integral value ITG_{276_Middle_276} in class 276_₂₇₆ in when the number of integral values is 276, and the integral value ITG_{32_High_32} in the class 32_₃₂ in when the number of integral values is 32 consists of the sum(=ITG_{274_High_276}+ITG_{275_High_276}+ITG_{276_High_276}) from integral value ITG_{274_High_276} in class 274_₂₇₆ to integral value ITG_{276_High_276} in class 276_₂₇₆ in when the number of integral values is 276.

Furthermore, when the analyte is “shochu”, the class 11_₂₀ in when the number of integral values is 20 is associated with classes 139_₂₇₆ to 153_₂₇₆ in when the number of integral values is 276, similarly, the class 19_₂₀ in when the number of integral values is 20 is associated with classes 259_₂₇₆ to 273_₂₇₆ in when the number of integral values is 276, and the class 20_₂₀ in when the number of integral values is 20 is associated with classes 274_₂₇₆ to 276_₂₇₆ in when the number of integral values is 276 (See (h) of FIG. 104).

As a result, the integral value ITG_{11_Low_20} in the class 11_₂₀ in when the number of integral values is 20 consists of the sum(=ITG_{139_Low_276}+ITG_{140_Low_276}+ , . . . , ITG_{153_Low_276}) from integral value ITG_{139_Low_276} in class 139_₂₇₆ to integral value ITG_{153_Low_276} in class 153_₂₇₆ in when the number of integral values is 276, the integral value ITG_{11_Middle_20} in the class 11_₂₀ in when the number of integral values is 20 consists of the sum(=ITG_{139_Middle_276}+ITG_{140_Middle_276}+ , . . . ,ITG_{153_Middle_276}) from integral value ITG_{139_Middle_276} in class 139_₂₇₆ to integral value ITG_{153_Middle_276} in class 153_₂₇₆ in when the number of integral values is 276, and the integral value ITG_{11_High_20} in the class 11_₂₀ in when the number of integral values is 20 consists of the sum(=ITG_{139_High_276}+ITG_{140_High_276}+ , . . . ,ITG_{153_High_276}) from integral value ITG_{139_High_276} in class 139_₂₇₆ to integral value ITG_{153_High_276} in class 153_₂₇₆ in when the number of integral values is 276.

Similarly, the integral value ITG_{19_Low_20} in the class 19_₂₀ in when the number of integral values is 20 consists of the sum(=ITG_{259_Low_276}+ITG_{260_Low_276}+ , . . . , +ITG_{273_Low_276}) from integral value ITG_{259_Low_276} in class 259_₂₇₆ to integral value ITG_{273_Low_276} in class 273_₂₇₆ in when the number of integral values is 276, the integral value ITG_{19_Middle_20} in the class 19_₂₀ in when the number of integral values is 20 consists of the sum(=ITG_{259_Middle_276}+ITG_{260_Middle_276}+ , . . . , +ITG_{273_Middle_276}) from integral value ITG_{259_Middle_276} in class 259_₂₇₆ to integral value ITG_{273_Middle_276} in class 273_₂₇₆ in when the number of integral values is 276, and the integral value ITG_{19_High_20} in the class 19_₂₀ in when the number of integral values is 20 consists of the sum(=ITG_{259_High_276}+ITG_{260_High_276}+ , . . . , +ITG_{273_High_276}) from integral value ITG_{259_High_276} in class 259_₂₇₆ to integral value ITG_{273_High_276} in class 273_₂₇₆ in when the number of integral values is 276.

Then, the integral value ITG_{20_Low_20} in the class 20_₂₀ in when the number of integral values is 20 consists of the sum(=ITG_{274_Low_276}+ITG_{275_Low_276}+ITG_{276_Low_276}) from integral value ITG_{274_Low_276} in class 274_₂₇₆ to integral value ITG_{276_Low_276} in class 276_₂₇₆ in when the number of integral values is 276, the integral value ITG_{20_Middle_20} in the class 20_₂₀ in when the number of integral values is 20 consists of the sum(=ITG_{274_Middle_276}+ITG_{275_Middle_276}+ITG_{276_Middle_276}) from integral value ITG_{274_Middle_276} in class 274_₂₇₆ to integral value ITG_{276_Middle_276} in class 276_₂₇₆ in when the number of integral values is 276, and the integral value ITG_{20_High_20} in the class 20_₂₀ in when the number of integral values is 20 consists of the sum(=ITG_{274_High_276}+ITG_{275_High_276}+ITG_{276_High_276}) from integral value ITG_{274_High_276} in class 274_₂₇₆ to integral value ITG_{276_High_276} in class 276_₂₇₆ in when the number of integral values is 276.

Furthermore, when the analyte is “shochu”, the class 10_₁₈ in when the number of integral values is 18 is associated with classes 139_₂₇₆ to 155_₂₇₆ in when the number of integral values is 276, similarly, the class 17_₁₈ in when the number of integral values is 18 is associated with classes 258_₂₇₆ to 274_₂₇₆ in when the number of integral values is 276, and the class 18_₁₈ in when the number of integral values is 18 is associated with classes 275_₂₇₆ and 276_₂₇₆ in when the number of integral values is 276 (See (i) of FIG. 104).

As a result, the integral value ITG_{10_Low_18} in the class 10_₁₈ in when the number of integral values is 18 consists of the sum(=ITG_{139_Low_276}+ITG_{140_Low_276}+ , . . . ,ITG_{155_Low_276}) from integral value ITG_{139_Low_276} in class 139_₂₇₆ to integral value ITG_{155_Low_276} in class 155_₂₇₆ in when the number of integral values is 276, the integral value ITG_{10_Middle_18} in the class 10_₁₈ in when the number of integral values is 18 consists of the sum(=ITG_{139_Middle_276}+ITG_{14_Middle_276}+ , . . . ,ITG_{155_Middle_276}) from integral value ITG_{139_Middle_276} in class 139_₂₇₆ to integral value ITG_{155_Middle_276} in class 155_₂₇₆ in when the number of integral values is 276, and the integral value ITG_{10_High_18} in the class 10_₁₈ in when the number of integral values is 18 consists of the sum(=ITG_{139_High_276}+ITG_{140_High_276}+ , . . . ,ITG_{155_High_276}) from integral value ITG_{139_High_276} in class 139_₂₇₆ to integral value ITG_{155_High_276} in class 155_₂₇₆ in when the number of integral values is 276.

Similarly, the integral value ITG_{17_Low_18} in the class 17_₁₈ in when the number of integral values is 18 consists of the sum(=ITG_{258_Low_276}+ITG_{259_Low_276}+ , . . . ,ITG_{274_Low_276}) from integral value ITG_{258_Low_276} in class 258_₂₇₆ to integral value ITG_{274_Low_276} in class 274_₂₇₆ in when the number of integral values is 276, the integral value ITG_{17_Middle_18} in the class 17_₁₈ in when the number of integral values is 18 consists of the sum(=ITG_{258_Middle_276}+ITG_{259_Middle_276}+ , . . . ,ITG_{274_Middle_276}) from integral value ITG_{258_Middle_276} in class 258_₂₇₆ to integral value ITG_{274_Middle_276} in class 274_₂₇₆ in when the number of integral values is 276, and the integral value ITG_{17_High_18} in the class 17_₁₈ in when the number of integral values is 18 consists of the sum(=ITG_{258_High_276}+ITG_{259_High_276}+ , . . . ,ITG_{274_High_276}) from integral value ITG_{258_High_276} in class 258_₂₇₆ to integral value ITG_{274_High_276} in class 274_₂₇₆ in when the number of integral values is 276.

Then, the integral value ITG_{18_Low_18} in the class 18_₁₈ in when the number of integral values is 18 consists of the sum(=ITG_{275_Low_276}+ITG_{276_Low_276}) of integral value ITG_{275_Low_276} in class 275_₂₇₆ and integral value ITG_{276_Low_276} in class 276_₂₇₆ in when the number of integral values is 276, the integral value ITG_{18_Middle_18} in the class 18_₁₈ in when the number of integral values is 18 consists of the sum(=ITG_{275_Middle_276}+ITG_{276_Middle_276}) of integral value ITG_{275_Middle_276} in class 275_₂₇₆ and integral value ITG_{276_Middle_276} in class 276_₂₇₆ in when the number of integral values is 276, and the integral value ITG_{18_High_18} in the class 18_₁₈ in when the number of integral values is 18 consists of the sum(=ITG_{275_High_276}+ITG_{276_High_276}) of integral value ITG_{275_High_276} in class 275_₂₇₆ and integral value ITG_{276_High_276} in class 276_₂₇₆ in when the number of integral values is 276.

According to the correspondence of the classes shown in FIG. 103, by associating “classes 1_₁₃₈ to 69_₁₃₈ in when the number of integral values is 138, classes 1_₉₂ to 46_₉₂ in when the number of integral values is 92, classes 1_₇₀ to 35_₇₀ in when the number of integral values is 70, classes 1_₅₆ to 28_₅₆ in when the number of integral values is 56, classes 1_₃₆ to 18_₃₆ in when the number of integral values is 36, classes 1_₃₂ to 16_₃₂ in when the number of integral values is 32, and classes 1_₂₀ to 10_₂₀ in when the number of integral values is 20 and classes 1_₁₈ to 9_₁₈ in when the number of integral values is 18” with “classes 1_₂₇₆ to 138_₂₇₆ in when the number of integral values is 276”, it is possible to match “the sum of the integral values in the positive predetermined potential section” to “the sum of the integral values in classes 1_₂₇₆ to 138_₂₇₆ (=the positive predetermined potential section) in when the number of integral values is 276” even if the number of integral values is any one of 138, 92, 70, 56, 36, 32, 20, and 18.

Also, according to the correspondence of the classes shown in FIG. 104, by associating “classes 70_₁₃₈ to 138_₁₃₈ in when the number of integral values is 138, classes 47_₉₂ to 92_₉₂ in when the number of integral values is 92, classes 36_₇₀ to 70_₇₀ in when the number of integral values is 70, classes 29_₅₆ to 56_₅₆ in when the number of integral values is 56, classes 19_₃₆ to 36_₃₆ in when the number of integral values is 36, classes 17_₃₂ to 32_₃₂ in when the number of integral values is 32, and classes 11_₂₀ to 20_₂₀ in when the number of integral values is 20 and classes 10_₁₈ to 18_₁₈ in when the number of integral values is 18” with “classes 139_₂₇₆ to 276_₂₇₆ in when the number of integral values is 276”, it is possible to match “the sum of the integral values in the negative predetermined potential section” to “the sum of the integral values in classes 139_₂₇₆ to 276_₂₇₆ (=the negative predetermined potential section) in when the number of integral values is 276” even if the number of integral values is any one of 138, 92, 70, 56, 36, 32, 20, and 18.

As a result, by associating “classes 1_₁₃₈ to 138_₁₃₈ in when the number of integral values is 138, classes 1_₉₂ to 92_₉₂ in when the number of integral values is 92, classes 1_₇₀ to 70_₇₀ in when the number of integral values is 70, classes 1_₅₆ to 56_₅₆ in when the number of integral values is 56, classes 1_₃₆ to 36_₃₆ in when the number of integral values is 36, classes 1₃₂ to 32₃₂ in when the number of integral values is 32, classes 1_₂₀ to 20_₂₀ when in the number of integral values is 20 and classes 1_₁₈ to 18_₁₈ in when the number of integral values is 18” with “classes 1_₂₇₆ to 276_₂₇₆ in when the number of integral values is 276”, it is possible to match “the sum of the integral values in the all predetermined potential section (=the positive predetermined potential section+the negative predetermined potential section)” to “the sum of the integral values in classes 1_₂₇₆ to 276_₂₇₆ in when the number of integral values is 276” even if the number of integral values is any one of 138, 92, 70, 56, 36, 32, 20, and 18.

Therefore, the taste diagnosis results for shochu in when the number of integral values is any of 18, 20, 32, 36, 56, 70, 92, and 138 coincide with the taste diagnosis result (=the taste diagnosis result shown in FIG. 29) for shochu in when the number of integral values is 276.

Therefore, the diagnostic device 2 may perform a taste diagnosis of “shochu” using the above-mentioned method based on the correspondence (see FIG. 12) between the classes and the integral values in when the number of integral values is any of 18, 20, 32, 36, 56, 70, 92, 138 and 276.

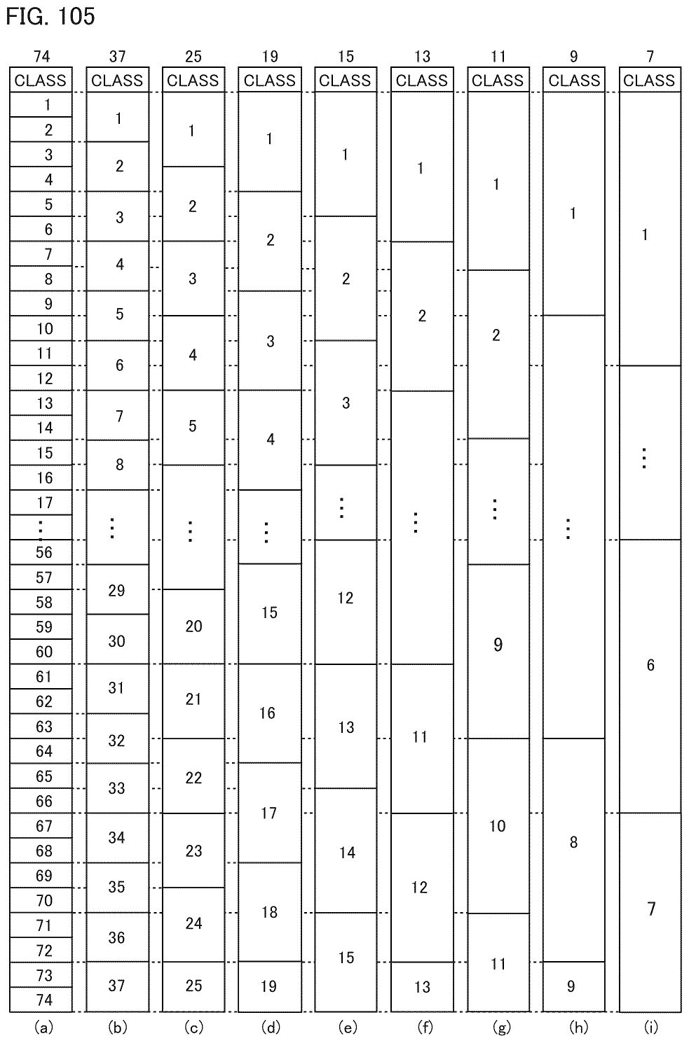

FIG. 105 is a diagram showing the correspondence relationship of classes in the case that the number of integral values in when the analyte is “grapes” is 7, 9, 11, 13, 15, 19, 25, 37 and 74.

Referring to FIG. 105, when the analyte is “grapes”, class 1_₃₇ in when the number of integral values is 37 is corresponded to classes 1_₇₄ and 2_₇₄ in when the number of integral values is 74, class 2_₃₇ in when the number of integral values is 37 is corresponded to classes 3_₇₄ and 4_₇₄ in when the number of integral values is 74, class 3_₃₇ in when the number of integral values is 37 is corresponded to classes 5_₇₄ and 6_₇₄ in when the number of integral values is 74, similarly, class 36_₃₇ in when the number of integral values is 37 is corresponded to classes 71_₇₄ and 72_₇₄ in when the number of integral values is 74, and class 37_₃₇ in when the number of integral values is 37 is corresponded to classes 73_₇₄ and 74_₇₄ in when the number of integral values is 74 (See (b) of FIG. 105).

As a result, an integral value ITG_{1_Low_37} in class 1_₃₇ in when the number of integral values is 37 consists of the sum (=ITG_{1_Low_74}+ITG_{2_Low_74}) of an integral value ITG_{1_Low_74} in class 1_₇₄ and an integral value ITG_{2_Low_74} in class 2_₇₄ in when the number of integral values is 74, an integral value ITG_{1_Middle_37} in class 1_₃₇ in when the number of integral values is 37 consists of the sum (=ITG_{1_Middle_74}+ITG_{2_Middle_74}) of an integral value ITG_{1_Middle_74} in class 1_₇₄ and an integral value ITG_{2_Middle_74} in class 2_₇₄ in when the number of integral values is 74, and an integral value ITG_{1_High_37} in class 1_₃₇ in when the number of integral values is 37 consists of the sum (=ITG_{1_High_74}+ITG_{2_High_74}) of an integral value ITG_{1_High_74} in class 1_₇₄ and an integral value ITG_{2_High_74} in class 2_₇₄ in when the number of integral values is 74.

Also, an integral value ITG_{2_Low_37} in class 2_₃₇ in when the number of integral values is 37 consists of the sum (=ITG_{3_Low_74}+ITG_{4_Low_74}) of an integral value ITG_{3_Low_74} in class 3_₇₄ and an integral value ITG_{4_Low_74} in class 4_₇₄ in when the number of integral values is 74, an integral value ITG_{2_Middle_37} in class 2_₃₇ in when the number of integral values is 37 consists of the sum (=ITG_{3_Middle_74}+ITG_{4_Middle_74}) of an integral value ITG_{3_Middle_74} in class 3_₇₄ and an integral value ITG_{4_Middle_74} in class 4_₇₄ in when the number of integral values is 74, and an integral value ITG_{2_High_37} in class 2_₃₇ in when the number of integral values is 37 consists of the sum (=ITG_{3_high_74}+ITG_{4_High_74}) of an integral value ITG_{3_High_74} in class 3_₇₄ and an integral value ITG_{4_High_74} in class 4_₇₄ in when the number of integral values is 74.

Similarly, an integral value ITG_{37_Low_37} in class 37_₃₇ in when the number of integral values is 37 consists of the sum (=ITG_{73_Low_74}+ITG_{74_Low_74}) of an integral value ITG_{73_Low_74} in class 73_₇₄ and an integral value ITG_{74_Low_74} in class 74_₇₄ in when the number of integral values is 74, an integral value ITG_{3_Middle_37} in class 3_₃₇ in when the number of integral values is 37 consists of the sum (=ITG_{73_Middle_74}+ITG_{74_Middle_74}) of an integral value ITG_{73_Middle_74} in class 73_₇₄ and an integral value ITG_{74_Middle_74} in class 74_₇₄ in when the number of integral values is 74, and an integral value ITG_{3_High_37} in class 3_₃₇ in when the number of integral values is 37 consists of the sum (=ITG_{73_High_74}+ITG_{74_High_74}) of an integral value ITG_{73_High_74} in class 73_₇₄ and an integral value ITG_{74_High_74} in class 74_₇₄ in when the number of integral values is 74.

Also, when the analyte is “grapes”, class 1_₂₅ in when the number of integral values is 25 corresponds to classes 1_₇₄ to 3_₇₄ in when the number of integral values is 74, class 2_₂₅ in when the number of integral values is 25 corresponds to classes 4_₇₄ to 6_₇₄ in when the number of integral values is 74, class 3_₂₅ in when the number of integral values is 25 corresponds to classes 7_₇₄ to 9_₇₄ in when the number of integral values is 74, similarly, class 24_₂₅ in when the number of integral values is 25 corresponds to classes 70_₇₄ to 72_₇₄ in when the number of integral values is 74, and class 25_₂₅ in when the number of integral values is 25 corresponds to classes 73_₇₄ and 74_₇₄ in when the number of integral values is 74 (See (c) of FIG. 105).

As a result, an integral value ITG_{1_Low_25} in class 1_₂₅ in when the number of integral values is 25 consists of the sum (=ITG_{1_Low_74}+ITG_{2_Low_74}+ITG_{3_Low_74}) of an integral value ITG_{1_Low_74} in class 1_₇₄, an integral value ITG_{2_Low_74} in class 2_₇₄, and an integral value ITG_{3_Low_74} in class 3_₇₄ in when the number of integral values is 74, an integral value ITG_{1_Middle_25} in class 1_₂₅ in when the number of integral values is 25 consists of the sum (=ITG_{1_Middle_74}+ITG_{2_Middle_74}+ITG_{3_Middle_74}) of an integral value ITG_{1_Middle_74} in class 1_₇₄, an integral value ITG_{2_Middle_74} in class 2_₇₄, and an integral value ITG_{3_Middle_74} in class 3_₇₄ in when the number of integral values is 74, and an integral value ITG_{1_High_25} in class 1_₂₅ in when the number of integral values is 25 consists of the sum (=ITG_{1_High_74}+ITG_{2_High_74}+ITG_{3_Low_74}) of an integral value ITG_{1_High_74} in class 1_₇₄, an integral value ITG_{2_High_74} in class 2_₇₄, and an integral value ITG_{3_High_74} in class 3_₇₄ in when the number of integral values is 74.

Also, an integral value ITG_{2_Low_25} in class 2_₂₅ in when the number of integral values is 25 consists of the sum (=ITG_{4_Low_74}+ITG_{5_Low_74}+ITG_{6_Low_74}) of an integral value ITG_{4_Low_74} in class 4_₇₄, an integral value ITG_{5_Low_74} in class 5_₇₄, and an integral value ITG_{6_Low_74} in class 6_₇₄ in when the number of integral values is 74, an integral value ITG_{2_Middle_25} in class 2_₂₅ in when the number of integral values is 25 consists of the sum (=ITG_{4_Middle_74}+ITG_{5_Middle_74}+ITG_{6_Middle_74}) of an integral value ITG_{4_Middle_74} in class 4_₇₄, an integral value ITG_{5_Middle_74} in class 5_₇₄, and an integral value ITG_{6_Middle_74} in class 6_₇₄ in when the number of integral values is 74, and an integral value ITG_{2_High_25} in class 2_₂₅ in when the number of integral values is 25 consists of the sum (=ITG_{4_High_74}+ITG_{5_High_74}+ITG_{6_High_74}) of an integral value ITG_{4_High_74} in class 4_₇₄, an integral value ITG_{5_High_74} in class 5_₇₄, and an integral value ITG_{6_High_74} in class 6_₇₄ in when the number of integral values is 74.

Similarly, an integral value ITG_{24_Low_25} in class 24_₂₅ in when the number of integral values is 25 consists of the sum (=ITG_{70_Low_74}+ITG_{71_Low_74}+ITG_{72_Low_74}) of an integral value ITG_{70_Low_74} in class 70_₇₄, an integral value ITG_{71_Low_74} in class 71_₇₄, and an integral value ITG_{72_Low_74} in class 72_₇₄ in when the number of integral values is 74, an integral value ITG_{24_Middle_25} in class 24_₂₅ in when the number of integral values is 25 consists of the sum (=ITG_{70_Middle_74}+ITG_{71_Middle_74}+ITG_{72_Middle_74}) of an integral value ITG_{70_Middle_74} in class 70_₇₄, an integral value ITG_{71_Middle_74} in class 71_₇₄, and an integral value ITG_{72_Middle_74} in class 72_₇₄ in when the number of integral values is 74, and an integral value ITG_{24_High_25} in class 24_₂₅ in when the number of integral values is 25 consists of the sum (=ITG_{70_High_74}+ITG_{71_High_74}+ITG_{72_High_74}) of an integral value ITG_{70_High_74} in class 70_₇₄, an integral value ITG_{71_High_74} in class 71_₇₄, and an integral value ITG_{72_High_74} in class 72_₇₄ in when the number of integral values is 74.

Also, an integral value ITG_{25_Low_25} in class 25_₂₅ in when the number of integral values is 25 consists of the sum (=ITG_{73_Low_74}+ITG_{74_Low_74}) of an integral value ITG_{73_Low_74} in class 73_₇₄, and an integral value ITG_{74_Low_74} in class 74_₇₄ in when the number of integral values is 74, an integral value ITG_{25_Middle_25} in class 25_₂₅ in when the number of integral values is 25 consists of the sum (=ITG_{73_Middle_74}+ITG_{74_Middle_74}) of an integral value ITG_{73_Middle_74} in class 73_₇₄ and an integral value ITG_{74_Middle_74} in class 74_₇₄ in when the number of integral values is 74, and an integral value ITG_{25_High_25} in class 25_₂₅ in when the number of integral values is 25 consists of the sum (=ITG_{73_High_74}+ITG_{74_High_74}) of an integral value ITG_{73_High_74} in class 73_₇₄, and an integral value ITG_{74_High_74} in class 74_₇₄ in when the number of integral values is 74.

Furthermore, when the analyte is “grapes”, class 1_₁₉ in when the number of integral values is 19 corresponds to classes 1_₇₄ to 4_₇₄ in when the number of integral values is 74, class 2_₁₉ in when the number of integral values is 19 corresponds to classes 5_₇₄ to 8_₇₄ in when the number of integral values is 74, class 3_₁₉ in when the number of integral values is 19 corresponds to classes 9_₇₄ to 12_₇₄ in when the number of integral values is 74, similarly, class 18_₁₉ in when the number of integral values is 19 corresponds to classes 69_₇₄ to 72_₇₄ in when the number of integral values is 74, and class 19_₁₉ in when the number of integral values is 19 corresponds to classes 73_₇₄ and 74_₇₄ in when the number of integral values is 74 (See (d) of FIG. 105).

As a result, an integral value ITG_{1_Low_19} in class 1_₁₉ in when the number of integral values is 19 consists of the sum (=ITG_{1_Low_74}+ITG_{2_Low_74}+ITG_{3_Low_74}+ITG_{4_Low_74}) from an integral value ITG_{1_Low_74} in class 1_₇₄ to an integral value ITG_{4_Low_74} in class 4_₇₄ in when the number of integral values is 74, an integral value ITG_{1_Middle_19} in class 1_₁₉ in when the number of integral values is 19 consists of the sum (=ITG_{1_Middle_74}+ITG_{2_Middle_74}+ITG_{3_Middle_74}+ITG_{4_Middle_74}) from an integral value ITG_{1_Middle_74} in class 1_₇₄ to an integral value ITG_{4_Middle_74} in class 4_₇₄ in when the number of integral values is 74, and an integral value ITG_{1_High_19} in class 1_₁₉ in when the number of integral values is 19 consists of the sum (=ITG_{1_High_74}+ITG_{2_High_74}+ITG_{3_High_74}+ITG_{4_High_74}) from an integral value ITG_{1_High_74} in class 1_₇₄ to an integral value ITG_{4_High_74} in class 4_₇₄ in when the number of integral values is 74.

Also, an integral value ITG_{2_Low_19} in class 2_₁₉ in when the number of integral values is 19 consists of the sum (=ITG_{5_Low_74}+ITG_{6_Low_74}+ITG_{7_Low_74}+ITG_{8_Low_74}) from an integral value ITG_{5_Low_74} in class 5_₇₄ to an integral value ITG_{8_Low_74} in class 8_₇₄ in when the number of integral values is 74, an integral value ITG_{2_Middle_19} in class 2_₁₉ in when the number of integral values is 19 consists of the sum (=ITG_{5_Middle_74}+ITG_{6_Middle_74}+ITG_{7_Middle_74}+ITG_{8_Middle_74}) from an integral value ITG_{5_Middle_74} in class 5_₇₄ to an integral value ITG_{8_Middle_74} in class 8_₇₄ in when the number of integral values is 74, and an integral value ITG_{2_High_19} in class 2_₁₉ in when the number of integral values is 19 consists of the sum (=ITG_{5_High_74}+ITG_{6_High_74}+ITG_{7_High_74}+ITG_{8_High_74}) from an integral value ITG_{5_High_74} in class 5_₇₄ to an integral value ITG_{8_High_74} in class 8_₇₄ in when the number of integral values is 74.

Similarly, an integral value ITG_{18_Low_19} in class 18_₁₉ in when the number of integral values is 19 consists of the sum (=ITG_{69_Low_74}+ITG_{70_Low_74}+ITG_{71_Low_74}+ITG_{72_Low_74}) from an integral value ITG_{69_Low_74} in class 69_₇₄ to an integral value ITG_{72_Low_74} in class 72_₇₄ in when the number of integral values is 74, an integral value ITG_{18_Middle_19} in class 18_₁₉ in when the number of integral values is 19 consists of the sum (=ITG_{69_Middle_74}+ITG_{70_Middle_74}+ITG_{71_Middle_74}+ITG_{72_Middle_74}) from an integral value ITG_{69_Middle_74} in class 69_₇₄ to an integral value ITG_{72_Middle_74} in class 72_₇₄ in when the number of integral values is 74, and an integral value ITG_{18_High_19} in class 18_₁₉ in when the number of integral values is 19 consists of the sum (=ITG_{69_High_74}+ITG_{70_High_74}+ITG_{71_High_74}+ITG_{72_High_74}) from an integral value ITG_{69_High_74} in class 69_₇₄ to an integral value ITG_{72_High_74} in class 72_₇₄ in when the number of integral values is 74.

And, an integral value ITG_{19_Low_19} in class 19_₁₉ in when the number of integral values is 19 consists of the sum (=ITG_{73_Low_74}+ITG_{74_Low_74}) of an integral value ITG_{73_Low_74} in class 73_₇₄ and an integral value ITG_{74_Low_74} in class 74_₇₄ in when the number of integral values is 74, an integral value ITG_{19_Middle_19} in class 19_₁₉ in when the number of integral values is 19 consists of the sum (=ITG_{73_Middle_74}+ITG_{74_Middle_74}) of an integral value ITG_{73_Middle_74} in class 73_₇₄ and an integral value ITG_{74_Middle_74} in class 74_₇₄ in when the number of integral values is 74, and an integral value ITG_{19_High_19} in class 19_₁₉ in when the number of integral values is 19 consists of the sum (=ITG_{73_High_74}+ITG_{74_High_74}) of an integral value ITG_{73_High_74} in class 73_₇₄ and an integral value ITG_{74_High_74} in class 74_₇₄ in when the number of integral values is 74.

Furthermore, when the analyte is “grapes”, class 1_₁₅ in when the number of integral values is 15 corresponds to classes 1₇₄ to 5_₇₄ in when the number of integral values is 74, class 2₁₅ in when the number of integral values is 15 corresponds to classes 6_₇₄ to 10_₇₄ in when the number of integral values is 74, class 3_₁₅ in when the number of integral values is 15 corresponds to classes 11_₇₄ to 15_₇₄ in when the number of integral values is 74, similarly, class 14₁₅ in when the number of integral values is 15 corresponds to classes 66_₇₄ to 70_₇₄ in when the number of integral values is 74, and class 15_₁₅ in when the number of integral values is 15 corresponds to classes 71_₇₄ to 74_₇₄ in when the number of integral values is 74 (See of (e) in FIG. 105).

As a result, an integral value ITG_{1_Low_15} in class 1_₁₅ in when the number of integral values is 15 consists of the sum (=ITG_{1_Low_74}+ITG_{2_Low_74}+ITG_{3_Low_74}+ITG_{4_Low_74}+ITG_{5_Low_74}) from an integral value ITG_{1_Low_74} in class 1_₇₄ to an integral value ITG_{5_Low_74} in class 5_₇₄ in when the number of integral values is 74, an integral value ITG_{1_Middle_15} in class 1_₁₅ in when the number of integral values is 15 consists of the sum (=ITG_{1_Middle_74}+ITG_{2_Middle_74}+ITG_{3_Middle_74}+ITG_{4_Middle_74}+ITG_{5_Middle_74}) from an integral value ITG_{1_Middle_74} in class 1_₇₄ to an integral value ITG_{5_Middle_74} in class 5_₇₄ in when the number of integral values is 74, and an integral value ITG_{1_High_15} in class 1_₁₅ in when the number of integral values is 15 consists of the sum (=ITG_{1_High_74}+ITG_{2_High_74}+ITG_{3_High_74}+ITG_{4_High_74}+ITG_{5_High_74}) from an integral value ITG_{1_High_74} in class 1_₇₄ to an integral value ITG_{5_High_74} in class 5_₇₄ in when the number of integral values is 74.

Also, an integral value ITG_{2_Low_15} in class 2_₁₅ in when the number of integral values is 15 consists of the sum (=ITG_{6_Low_74}+ITG_{7_Low_74}+ITG_{8_Low_74}+ITG_{9_Low_74}+ITG_{10_Low_74}) from an integral value ITG_{6_Low_74} in class 6_₇₄ to an integral value ITG_{10_Low_74} in class 10_₇₄ in when the number of integral values is 74, an integral value ITG_{2_Middle_15} in class 2_₁₅ in when the number of integral values is 15 consists of the sum (=ITG_{6_Middle_74}+ITG_{7_Middle_74}+ITG_{8_Middle_74}+ITG_{9_Middle_74}+ITG_{10_Middle_74}) from an integral value ITG_{6_Middle_74} in class 6_₇₄ to an integral value ITG_{10_Middle_74} in class 10_₇₄ in when the number of integral values is 74, and an integral value ITG_{2_High_15} in class 2_₁₅ in when the number of integral values is 15 consists of the sum (=ITG_{6_High_74}+ITG_{7_High_74}+ITG_{8_High_74}+ITG_{9_High_74}+ITG_{10_High_74}) from an integral value ITG_{6_High_74} in class 6_₇₄ to an integral value ITG_{10_High_74} in class 10_₇₄ in when the number of integral values is 74.

Similarly, an integral value ITG_{14_Low_15} in class 14_₁₅ in when the number of integral values is 15 consists of the sum (=ITG_{66_Low_74}+ITG_{67_Low_74}+ITG_{68_Low_74}+ITG_{69_Low_74}+ITG_{70_Low_74}) from an integral value ITG_{66_Low_74} in class 66_₇₄ to an integral value ITG_{70_Low_74} in class 70_₇₄ in when the number of integral values is 74, an integral value ITG_{14_Middle_15} in class 14_₁₅ in when the number of integral values is 15 consists of the sum (=ITG_{66_Middle_74}+ITG_{67_Middle_74}+ITG_{68_Middle_74}+ITG_{69_Middle_74}+ITG_{70_Middle_74}) from an integral value ITG_{66_Middle_74} in class 66_₇₄ to an integral value ITG_{70_Middle_74} in class 70_₇₄ in when the number of integral values is 74, and an integral ITG_{14_High_15} in class 14_₁₅ in when the number of integral values is 15 consists of the sum (=ITG_{66_High_74}+ITG_{67_High_74}+ITG_{68_High_74}+ITG_{69_High74}+ITG_{70_High_74}) from an integral value ITG_{66_High_74} in class 66_₇₄ to an integral value ITG_{70_High_74} in class 70_₇₄ in when the number of integral values is 74.

And, an integral value ITG_{15_Low_15} in class 15_₁₅ in when the number of integral values is 15 consists of the sum (=ITG_{71_Low_74}+ITG_{72_Low_74}+ITG_{73_Low_74}+ITG_{74_Low_74}) from an integral value ITG_{71_Low_74} in class 71_₇₄ to an integral value ITG_{74_Low_74} in class 74_₇₄ in when the number of integral values is 74, an integral value ITG_{15_Middle_15} in class 15_₁₅ in when the number of integral values is 15 consists of the sum (=ITG_{71_Middle_74}+ITG_{72_Middle_74}+ITG_{73_Middle_74}+ITG_{74_Middle_74}) from an integral value ITG_{71_Middle_74} in class 71_₇₄ to an integral value ITG_{74_Middle_74} in class 74_₇₄ in when the number of integral values is 74, and an integral value ITG_{15_High_15} in class 15_₁₅ in when the number of integral values is 15 consists of the sum (=ITG_{71_High_74}+ITG_{72_High_74}+ITG_{73_High_74}+ITG_{74_High_74}) from an integral value ITG__{71_High_74} in class 71_₇₄ to an integral value ITG_{74_High_74} in class 74_₇₄ in when the number of integral values is 74.

Furthermore, when the analyte is “grapes”, class 1_₁₃ in when the number of integral values is 13 corresponds to classes 1_₇₄ to 6_₇₄ in when the number of integral values is 74, class 2_₁₃ in when the number of integral values is 13 corresponds to classes 7_₇₄ to 12_₇₄ in when the number of integral values is 74, similarly, class 12_₁₃ in when the number of integral values is 13 corresponds to classes 67_₇₄ to 72_₇₄ in when the number of integral values is 74, and class 13_₁₃ in when the number of integral values is 13 corresponds to classes 73_₇₄ and 74_₇₄ in when the number of integral values is 74 (See (f) of FIG. 105).

As a result, an integral value ITG_{1_Low_13} in class 1_₁₃ in when the number of integral values is 13 consists of the sum (=ITG_{1_Low_74}+ITG_{2_Low_74}+ITG_{3_Low_74}+ . . . , +ITG_{6_Low_74}) from an integral value ITG_{1_Low_74} in class 1_₇₄ to an integral value ITG_{6_Low_74} in class 6_₇₄ in when the number of integral values is 74, an integral value ITG_{1_Middle_13} in class 1_₁₃ in when the number of integral values is 13 consists of the sum (=ITG_{1_Middle_74}+ITG_{2_Middle_74}+ITG_{3_Middle_74}+ , . . . , +ITG_{6_Middle_74}) from an integral value ITG_{1_Middle_74} in class 1_₇₄ to an integral value ITG_{6_Middle_74} in class 6_₇₄ in when the number of integral values is 74, and an integral value ITG_{1_High_13} in class 1_₁₃ in when the number of integral values is 13 consists of the sum (=ITG_{1_High_74}+ITG_{2_High_74}+ITG_{3_High_74}+ , . . . , +ITG_{6_High_74}) from an integral value ITG_{1_High_74} in class 1_₇₄ to an integral value ITG_{6_High_74} in class 6_₇₄ in when the number of integral values is 74.

Also, an integral value ITG_{2_Low_13} in class 2_₁₃ in when the number of integral values is 13 consists of the sum (=ITG_{7_Low_74}+ITG_{8_Low_74}+ITG_{9_Low_74}+ , . . . , +ITG_{12_Low_74}) from an integral value ITG_{7_Low_74} in class 7_₇₄ to an integral value ITG_{12_Low_74} in class 12_₇₄ in when the number of integral values is 74, an integral value ITG_{2_Middle_13} in class 2_₁₃ in when the number of integral values is 13 consists of the sum (=ITG_{7_Middle_74}+ITG_{8_Middle_74}+ITG_{9_Middle_74}+ , . . . , +ITG_{12_Middle_74}) from an integral value ITG_{7_Middle_74} in class 7_₇₄ to an integral value ITG_{12_Middle_74} in class 12_₇₄ in when the number of integral values is 74, and an integral value ITG_{2_High_13} in class 2_₁₃ in when the number of integral values is 13 consists of the sum (=ITG_{7_High_74}+ITG_{8_High_74}+ITG_{9_High_74}+ , . . . , +ITG_{12_High_74}) from an integral value ITG_{7_High_74} in class 7_₇₄ to an integral value ITG_{12_High_74} in class 12_₇₄ in when the number of integral values is 74.

Similarly, an integral value ITG_{12_Low_13} in class 12_₁₃ in when the number of integral values is 13 consists of the sum (=ITG_{67_Low_74}+ITG_{68_Low_74}+ITG_{69_Low_74}+ , . . . , +ITG_{72_Low_74}) from an integral value ITG_{67_Low_74} in class 67_₇₄ to an integral value ITG_{72_Low_74} in class 72_₇₄ in when the number of integral values is 74, an integral value ITG_{12_Middle_13} in class 12_₁₃ in when the number of integral values is 13 consists of the sum (=ITG_{67_Middle_74}+ITG_{68_Middle_74}+ITG_{69_Middle_74}+ , . . . , +ITG_{72_Middle_74}) from an integral value ITG_{67_Middle_74} in class 67_₇₄ to an integral value ITG_{72_Middle_74} in class 72_₇₄ in when the number of integral values is 74, and an integral value ITG_{12_High_13} in class 12_₁₃ in when the number of integral values is 13 consists of the sum (=ITG_{67_High_74}+ITG_{68_High_74}+ITG_{69_High_74}+ , . . . , +ITG_{72_High_74}) from an integral value ITG_{67_High_74} in class 67_₇₄ to an integral value ITG_{72_High_74} in class 72_₇₄ in when the number of integral values is 74.

And, an integral value ITG_{13_Low_13} in class 13_₁₃ in when the number of integral values is 13 consists of the sum (=ITG_{73_Low_74}+ITG_{74_Low_74}) of an integral value ITG_{73_Low_74} in class 73_₇₄ and an integral value ITG_{74_Low_74} in class 74_₇₄ in when the number of integral values is 74, an integral value ITG_{13_Middle_13} in class 13_₁₃ in when the number of integral values is 13 consists of the sum (=ITG_{73_Middle_74}+ITG_{74_Middle_74}) of an integral value ITG_{73_Middle_74} in class 73_₇₄ and an integral value ITG_{74_Middle_74} in class 74_₇₄ in when the number of integral values is 74, and an integral value ITG_{13_High_13} in class 13_₁₃ in when the number of integral values is 13 consists of the sum (=ITG_{73_High_74}+ITG_{74_High_74}) of an integral value ITG_{73_High_74} in class 73_₇₄ and an integral value ITG_{74_High_74} in class 74_₇₄ in when the number of integral values is 74.

Furthermore, when the analyte is “grapes”, class 1_₁₁ in when the number of integral values is 11 corresponds to classes 1_₇₄ to 7_₇₄ in when the number of integral values is 74, class 2_₁₁ in when the number of integral values is 11 corresponds to classes 8_₇₄ to 14_₇₄ in when the number of integral values is 74, similarly, class 10_₁₁ in when the number of integral values is 11 corresponds to classes 64_₇₄ to 70_₇₄ in when the number of integral values is 74, and class 11_₁₁ in when the number of integral values is 11 corresponds to classes 71_₇₄ to 74_₇₄ in when the number of integral values is 74 (See (g) of FIG. 105).

As a result, an integral value ITG_{1_Low_11} in class 1_₁₁ in when the number of integral values is 11 consists of the sum (=ITG_{1_Low_74}+ITG_{2_Low_74}+ITG_{3_Low_74}+ , . . . , +ITG_{7_Low_74}) from an integral value ITG_{1_Low_74} in class 1_₇₄ to an integral value ITG_{7_Low_74} in class 7_₇₄ in when the number of integral values is 74, an integral value ITG_{1_Middle_11} in class 1_₁₁ in when the number of integral values is 11 consists of the sum (=ITG_{1_Middle_74}+ITG_{2_Middle_74}+ITG_{3_Middle_74}+ , . . . , +ITG_{7_Middle_74}) from an integral value ITG_{1_Middle_74} in class 1_₇₄ to an integral value ITG_{7_Middle_74} in class 7_₇₄ in when the number of integral values is 74, and an integral value ITG_{1_High_11} in class 1_₁₁ in when the number of integral values is 11 consists of the sum (=ITG_{1_High_74}+ITG_{2_High_74}+ITG_{3_High_74}+ , . . . , +ITG_{7_High_74}) from an integral value ITG_{1_High_74} in class 1_₇₄ to an integral value ITG_{7_High_74} in class 7_₇₄ in when the number of integral values is 74.

Also, an integral value ITG_{2_Low_11} in class 2_₁₁ in when the number of integral values is 11 consists of the sum (=ITG_{8_Low_74}+ITG_{9_Low_74}+ITG_{10_Low_74}+ , . . . , +ITG_{14_Low_74}) from an integral value ITG_{8_Low_74} in class 8_₇₄ to an integral value ITG_{14_Low_74} in class 14_₇₄ in when the number of integral values is 74, an integral value ITG_{2_Middle_11} in class 2_₁₁ when the number of integral values is 11 consists of the sum (=ITG_{8_Middle_74}+ITG_{9_Middle_74}+ITG_{10_Middle_74}+ , . . . , +ITG_{14_Middle_74}) from an integral value ITG_{8_Middle_74} in class 8_₇₄ to an integral value ITG_{14_Middle_74} in class 14_₇₄ in when the number of integral values is 74, and an integral value ITG_{2_High_11} in class 2_₁₁ in when the number of integral values is 11 consists of the sum (=ITG_{8_High_74}+ITG_{9_High_74}+ITG_{10_High_74}+ , . . . , +ITG_{14_High_74}) from an integral value ITG_{8_High_74} in class 8_₇₄ to an integral value ITG_{14_High_74} in class 14_₇₄ in when the number of integral values is 74.

Similarly, an integral value ITG_{10_Low_11} in class 10_₁₁ in when the number of integral values is 11 consists of the sum (=ITG_{64_Low_74}+ITG_{65_Low_74}+ITG_{66_Low_74}+ , . . . , +ITG_{70_Low_74}) from an integral value ITG_{64_Low_74} in class 64_₇₄ to an integral value ITG_{70_Low_74} in class 70_₇₄ in when the number of integral values is 74, an integral value ITG_{10_Middle_11} in class 10_₁₁ in when the number of integral values is 11 consists of the sum (=ITG_{64_Middle_74}+ITG_{65_Middle_74}+ITG_{66_Middle_74}+ , . . . , +ITG_{70_Middle_74}) from an integral value ITG_{64_Middle_74} in class 64_₇₄ to an integral value ITG_{70_Middle_74} in class 70_₇₄ in when the number of integral values is 74, an integral value ITG_{10_High_11} in class 10_₁₁ in when the number of integral values is 11 consists of the sum (=ITG_{64_High_74}+ITG_{65_High_74}+ITG_{66_High_74}+ , . . . , +ITG_{70_High_74}) from an integral value ITG_{64_High_74} in class 64_₇₄ to an integral value ITG_{70_High_74} in class 70_₇₄ in when the number of integral values is 74.

And, an integral value ITG_{1_Low_11} in class 11_₁₁ in when the number of integral values is 11 consists of the sum (=ITG_{71_Low_74}+ITG_{72_Low_74}+ITG_{73_Low_74}+ITG_{74_Low_74}) from an integral value ITG_{71_Low_74} in class 71_₇₄ to an integral value ITG_{74_Low_74} in class 74_₇₄ in when the number of integral values is 74, an integral value ITG_{11_Middle_11} in class 11_₁₁ in when the number of integral values is 11 consists of the sum (=ITG_{71_Middle_74}+ITG_{72_Middle_74}+ITG_{73_Middle_74}+ITG_{74_Middle_74}) from an integral value ITG_{71_Middle_74} in class 71_₇₄ to an integral value ITG_{74_Middle_74} in class 74_₇₄ in when the number of integral values is 74, and an integral value ITG_{11_High_11} in class 11_₁₁ in when the number of integral values is 11 consists of the sum (=ITG_{71_High_74}+ITG_{72_High_74}+ITG_{73_High_74}+ITG_{74_High_74}) from an integral value ITG_{71_High_74} in class 71_₇₄ to an integral value ITG_{74_High_74} in class 74_₇₄ in when the number of integral values is 74.

Furthermore, when the analyte is “grapes”, class 1_₉ in when the number of integral values is 9 corresponds to classes 1_₇₄ to 9_₇₄ in when the number of integral values is 74, similarly, class 8_₉ in when the number of integral values is 9 corresponds to classes 64_₇₄ to 72_₇₄ in when the number of integral values is 74, and class 9_₉ in when the number of integral values is 9 corresponds to classes 73_₇₄ and 74_₇₄ in when the number of integral values is 74 (See (h) of FIG. 105).

As a result, an integral value ITG_{1_Low_9} in class 1_₉ in when the number of integral values is 9 consists of the sum (=ITG_{1_Low_74}+ITG_{2_Low_74}+ITG_{3_Low_74}+ , . . . , +ITG_{9_Low_74}) from an integral value ITG_{1_Low_74} in class 1_₇₄ to an integral value ITG_{9_Low_74} in class 9_₇₄ in when the number of integral values is 74, an integral value ITG_{1_Middle_9} in class 1_₉ in when the number of integral values is 9 consists of the sum (=ITG_{1_Middle_74}+ITG_{2_Middle_74}+ITG_{3_Middle_74}+ , . . . , +ITG_{9_Middle_74}) from an integral value ITG_{1_Middle_74} in class 1_₇₄ to an integral value ITG_{9_Middle_74} in class 9_₇₄ in when the number of integral values is 74, and an integral value ITG_{1_High_9} in class 1_₉ in when the number of integral values is 9 consists of the sum (=ITG_{1_High_74}+ITG_{2_High_74}+ITG_{3_High_74}+ , . . . , +ITG_{9_High_74}) from an integral value ITG_{1_High_74} in class 1_₇₄ to an integral value ITG_{9_High_74} in class 9_₇₄ in when the number of integral values is 74.

Similarly, an integral value ITG_{8_Low_9} in class 8_₉ in when the number of integral values is 9 consists of the sum (=ITG_{64_Low_74}+ITG_{65_Low_74}+ITG_{66_Low_74}+ , . . . , +ITG_{72_Low_74}) from an integral value ITG_{64_Low_74} in class 64_₇₄ to an integral value ITG_{72_Low_74} in class 72_₇₄ when the number of integral values is 74, an integral value ITG_{8_Middle_9} in class 8_₉ in when the number of integral values is 9 consists of the sum (=ITG_{64_Middle_74}+ITG_{65_Middle_74}+ITG_{66_Middle_74}+ , . . . , +ITG_{72_Middle_74}) from an integral value ITG_{64_Middle_74} in class 64_₇₄ to an integral value ITG_{72_Middle_74} in class 72_₇₄ in when the number of integral values is 74, and an integral value ITG_{8_High_9} in class 8_₉ in when the number of integral values is 9 consists of the sum (=ITG_{64_High_74}+ITG_{65_High_74}+ITG_{66_High_74}+ , . . . , +ITG_{72_High_74}) from an integral value ITG_{64_High_74} in class 64_₇₄ to an integral value ITG_{72_High_74} in class 72_₇₄ in when the number of integral values is 74.

And, an integral value ITG_{9_Low_9} in class 9_₉ in when the number of integral values is 9 consists of the sum (=ITG_{73_Low_74}+ITG_{74_Low_74}) of an integral value ITG_{73_Low_74} in class 73_₇₄ and an integral value ITG_{74_Low_74} in class 74_₇₄ in when the number of integral values is 74, an integral value ITG_{9_Middle_9} in class 9_₉ in when the number of integral values is 9 consists of the sum (=ITG_{73_Middle_74}+ITG_{74_Middle_74}) of an integral value ITG_{73_Middle_74} in class 73_₇₄ and an integral value ITG_{74_Middle_74} in class 74_₇₄ in when the number of integral values is 74, and an integral value ITG_{9_High_9} in class 9_₉ in when the number of integral values is 9 consists of the sum (=ITG_{73_High_74}+ITG_{74_High_74}) of an integral value ITG_{73_High_74} in class 73_₇₄ and an integral value ITG_{74_High_74} in class 74_₇₄ in when the number of integral values is 74.

Furthermore, when the analyte is “grapes”, class 1_₇ in when the number of integral values is 7 corresponds to classes 1_₇₄ to 11_₇₄ in when the number of integral values is 74, similarly, class 6_₇ in when the number of integral values is 7 corresponds to classes 56_₇₄ to 66_₇₄ in when the number of integral values is 74, and class 7_₇ in when the number of integral values is 7 corresponds to classes 67_₇₄ to 74_₇₄ in when the number of integral values is 74 (See (i) of FIG. 105).

As a result, an integral value ITG_{1_Low_7} in class 1_₇ in when the number of integral values is 7 consists of the sum (=ITG_{1_Low_74}+ITG_{2_Low_74}+ITG_{3_Low_74}+ , . . . , +ITG_{1_Low_74}) from an integral value ITG_{1_Low_74} in class 1_₇₄ to an integral value ITG_{11_Low_74} in class 11_₇₄ in when the number of integral values is 74, an integral value ITG_{1_Middle_7} in class 1_₇ in when the number of integral values is 7 consists of the sum (=ITG_{1_Middle_74}+ITG_{2_Middle_74}+ITG_{3_Middle_74}+ , . . . , +ITG_{11_Middle_74}) from an integral value ITG_{1_Middle_74} in class 1_₇₄ to an integral value ITG_{11_Middle_74} in class 11_₇₄ in when the number of integral values is 74, and an integral value ITG_{1_High_7} in class 1_₇ in when the number of integral values is 7 consists of the sum (=ITG_{1_High_74}+ITG_{2_High_74}+ITG_{3_High_74}+ , . . . , +ITG_{11_High_74}) from an integral value ITG_{1_High_74} in class 1_₇₄ to an integral value ITG_{11_High_74} in class 11_₇₄ in when the number of integral values is 74.

Similarly, an integral value ITG_{6_Low_7} in class 6_₇ in when the number of integral values is 7 consists of the sum (=ITG_{56_Low_74}+ITG_{57_Low_74}+ITG_{58_Low_74}+ , . . . , +ITG_{66_Low_74}) from an integral value ITG_{56_Low_74} in class 56_₇₄ to an integral value ITG_{66_Low_74} in class 66_₇₄ in when the number of integral values is 74, an integral value ITG_{6_Middle_7} in class 6_₇ in when the number of integral values is 7 consists of the sum (=ITG_{56_Middle_74}+ITG_{57_Middle_74}+ITG_{58_Middle_74}+ , . . . , +ITG_{66_Middle_74}) from an integral value ITG_{56_Middle_74} in class 56_₇₄ to an integral value ITG_{66_Middle_74} in class 66_₇₄ in when the number of integral values is 74, and an integral value ITG_{6_High_7} in class 6_₇ in when the number of integral values is 7 consists of the sum (=ITG_{56_High_74}+ITG_{57_High_74}+ITG_{58_High_74}+ , . . . , +ITG_{66_High_74}) from an integral value ITG_{56_High_74} in class 56_₇₄ to an integral value ITG_{66_High_74} in class 66_₇₄ in when the number of integral values is 74.

And, an integral value ITG_{7_Low_7} in class 7_₇ in when the number of integral values is 7 consists of the sum (=ITG_{67_Low_74}+ITG_{68_Low_74}+ITG_{69_Low_74}+ , . . . , +ITG_{74_Low_74}) from an integral value ITG_{67_Low_74} in class 67_₇₄ to an integral value ITG_{74_Low_74} in class 74_₇₄ in when the number of integral values is 74, an integral value ITG_{7_Middle_7} in class 7_₇ in when the number of integral values is 7 consists of the sum (=ITG_{67_Middle_74}+ITG_{68_Middle_74}+ITG_{69_Middle_74}+ , . . . , +ITG_{74_Middle_74}) from an integral value ITG_{67_Middle_74} in class 67_₇₄ to an integral value ITG_{74_Middle_74} in class 74_₇₄ in when the number of integral values is 74, and an integral value ITG_{7_High_7} in class 7_₇ in when the number of integral values is 7 consists of the sum (=ITG_{67_High_74}+ITG_{68_High_74}+ITG_{69_High_74}+ , . . . , +ITG_{74_High_74}) from an integral value ITG_{67_High_74} in class 67_₇₄ to an integral value ITG_{74_High_74} in class 74_₇₄ in when the number of integral values is 74.

As a result, by associating each of “classes 1_₃₇ to 37_₃₇ in when the number of integral values is 37, classes 1_₂₅ to 25_₂₅ in when the number of integral values is 25, classes 1_₁₉ to 19_₁₉ in when the number of integral values is 19, classes 1_₁₅ to 15₁₅ in when the number of integral values is 15, classes 1₁₃ to 13₁₃ in when the number of integral values is 13, classes 1_₁ to 11_₁₁ in when the number of integral values is 11, classes 1_₉ to 9_₉ in when the number of integral values is 9, and classes 1_₇ to 7_₇ in when the number of integral values is 7,” with “classes 1_₇₄ to 74_₇₄ in when the number of integral values is 74”, it is possible to match “the sum of integral values in a predetermined potential range including a potential equal to or lower than the threshold value Vth” to “the sum of the integral values in a predetermined potential section including a potential equal to or lower than the threshold value Vth in when the number of integral values is 74” even if the number of integral values is any one of 37, 25, 19, 15, 13, 11, 9, and 7.

Therefore, the taste diagnosis result of grape in when the number of integral values is any one of 7, 9, 11, 13, 15, 19, 25, and 37 coincides with the taste diagnosis result of grape (=the taste diagnosis result shown in FIG. 35) in when the number of integral values is 74.

Therefore, the diagnostic device 2 may perform a taste diagnosis of “grapes” using the above-mentioned method based on the correspondence (see FIG. 12) between the classes and the integral values in when the number of integral values is any of 7, 9, 11, 13, 15, 19, 25, 37, and 74. [0936][Taste diagnosis of “Shochu” in when the number of integral values is multiple] When the diagnostic device 2 diagnoses the taste of shochu, the control unit 212 stores the calculation data CALᵤₙᵢ and the curve CURᵤₙᵢ in the analysis data ALY_Dᵤₙᵢ read out from the database 22 to update the analysis data ALY_Dᵤₙᵢ to index data IDXᵤₙᵢ, and outputs the updated index data IDXᵤₙᵢ to the calculation unit 216. In addition, the control unit 212 determines the number n_{TGC1} of integral values used for the taste diagnosis of shochu to be any one of 18, 20, 32, 36, 56, 70, 92, and 138, and outputs the determined number n_{TGC1} of integral values to the calculation unit 216.

Here, the index data IDXᵤₙᵢ has a configuration of IDXᵤₙᵢ=[time tᵤₙᵢ/identification information IDᵤₙᵢ/name ALY_Naᵤₙᵢ of analyte/type ALY_Kdᵤₙᵢ of analyte/current-potential characteristic (I-V)ᵤₙᵢ/calculation data CALᵤₙᵢ/curve CURᵤₙᵢ].

The calculation unit 216 receives the number n_{TGC1} of integral values used in the taste diagnosis of Shochu and the index data IDXᵤₙᵢ from the control unit 212, and detects the calculation data CALᵤₙᵢ from the received index data IDXᵤₙᵢ.

Here, it is assumed that n in the calculation data CALᵤₙᵢ received from the control unit 212 is set to n=276. It is also assumed that the calculation unit 216 holds in advance the “diagram showing the correspondence relationship of classes” shown in FIGS. 103 and 104.

The calculation unit 216 refers to the “diagram showing the correspondence relationship of classes” and converts the 276 integral values ITG_{1_Low} to ITG_{276_Low}, ITG_{1_Middle} to ITG_{276_Middle}, and ITG_{1_High} to ITG_{276_High} into n_{TGC1} of integral values ITG_{1_Low} to ITG_{nTGC1_Low}, ITG_{1_Middle} to ITG_{nTGC1_Middle}, and ITG_{1_High} to ITG_{nTGC1_High}, respectively.

Then, the calculation unit 216 associates the classes Cls₁ to Cls_{nTGC1} with the n_{TGC1} integral values ITG_{1_Low} to ITG_{nTGC1_Low}, ITG_{1_Middle} to ITG_{nTGC1_Middle}, and ITG_{1_High} to ITG to output them to the taste diagnostic unit 217.

The taste diagnostic unit 217 receives the correspondence between the classes Cls₁ to Cls_{nTGC1} and the n_{TGC1} integral values ITG_{1_Low} to ITG_{nTGC1_Low}, ITG_{1_Middle} to ITG_{nTGC1_Middle}, and ITG_{1_High} to ITG_{nTGC1_High} from the calculation unit 216, and the n_{TGC1} of ITG_{1_High} to ITG_{nTGC1_High}, executes sequentially the steps S821 to S825, S827, S828, and S830 shown in FIG. 98 (including the flowchart shown in FIG. 99) to diagnose the taste of shochu based on the correspondence between the classes Cls₁ to Cls_{nTGC1} and the n_{TGC1} Of integral values ITG_{1_Low} to ITG_{nTGC1_Low}, the n_{TGC1} of ITG_{1_Middle} to ITG_{nTGC1_Middle}, and the n_{TGC1} Of ITG_{1_High} to ITG.

In this case, “n” in steps S821 to S825 in FIG. 98 is set to “n_{TGC1}.”

In FIGS. 103 and 104, when the number of integral values is 276, the 276 classes are all composed of the same predetermined potential section, when the number of integral values is 138, the 138 classes are all composed of the same predetermined potential section, and when the number of integral values is 92, the 92 classes are all composed of the same predetermined potential section.

On the other hand, when the number of integral values is 70, classes 1 to 34 and 36 to 69 are composed of the same predetermined potential section, and classes 35 and 70 are composed of predetermined potential sections smaller than the predetermined potential sections in each of classes 1 to 34 and 36 to 69. The same is also true for the cases where the number of integral values is 56, 36, 32, 20, and 18.

Therefore, when the analyte is “shochu”, the n_{TGC1} pieces of integral values used for taste diagnosis consists of multiple integral values in the same predetermined potential section (=See 276 integral values, 138 integral values, and 92 integral values), or consists of multiple integral values including integral values in a first predetermined potential section and integral values in a second predetermined potential section smaller than the first predetermined potential section (=See 70 integral values, 56 integral values, 36 integral values, 32 integral values, 20 integral values, and 18 integral values).

[Taste diagnosis of “grapes” in when the number of integral values is multiple] When the diagnostic device 2 diagnoses the taste of “grapes”, the calculation unit 216 receives the index data IDXᵤₙᵢ from the control unit 212 as described above. Here, it is assumed that n in the calculation data CALᵤₙᵢ contained in the index data IDXᵤₙᵢ is set to n=74 and that the calculation unit 216 holds in advance the “diagram showing the correspondence relationship of classes” shown in FIG. 105.

The calculation unit 216 also receives from the control unit 212 the number nTGC2 of integral values composed of any one of 7, 9, 11, 13, 15, 19, 25, and 37.

The calculation unit 216 refers to the “diagram showing the correspondence relationship of classes” to convert the 74 integral values ITG_{1_Low} to ITG₇₄_Low, ITG_{1_Middle} to ITG_{74_Middle}, and ITG_{1_High} to ITG_{74_High} into n_{TGC2} integral values ITG_{1_Low} to ITG_{nTGC2_Low}, ITG_{1_Middle} to ITG_{nTGC2_Middle}, and ITG_{1_High} to ITG_{nTGC2_High}, respectively.

Then, the calculation unit 216 associates the classes Cls₁ to Cls_{nTGC2} with the n_{TGC2} integral values ITG_{1_Low} to ITG_{nTGC2_Low}, ITG_{1_Middle} to ITG_{nTGC2_Middle}, and ITG_{1_High} to ITG_{nTGC2_High} to output them to the taste diagnostic unit 217.

The taste diagnostic unit 217 receives the correspondence relationship between the classes Cls₁ to Cls_{nTGC2} and the n_{TGC2} integral values ITG_{1_Low} to ITG_{nTGC2_Low}, ITG_{1_Middle} to ITG_{nTGC2_Middle}, and ITG_{1_High} to ITG_{nTGC2_High} from the calculation unit 216, and executes sequentially steps S826 and S830 (including the flowchart shown in FIG. 100) shown in FIG. 98 in sequence to diagnose the taste of “grapes” based on the correspondence relationship between the classes Cls₁ to Cls_{nTGC2} and the n_{TGC2} integral values ITG_{1_Low} to ITG_{nTGC2_Low}, ITG_{1_Middle} to ITG_{nTGC2_Middle}, and ITG_{1_High} to ITG_{nTGC2_High}.

In this case, “n” in step S826 of FIG. 98 is set to “n_{TGC2}”.

In FIG. 105, when the number of integral values is 74, the 74 classes consist of mutually the same predetermined potential section, and when the number of integral values is 37, the 37 classes consist of mutually the same predetermined potential section.

On the other hand, when the number of integral values is 25, classes 1 to 24 are composed of the same predetermined potential section, and class 25 is composed of a predetermined potential section smaller than the predetermined potential sections in each of classes 1 to 24. The same is true for the cases where the number of integral values is 19, 15, 13, 11, 9, and 7.

Therefore, when the analyte is “grapes,” the two n_{TGC2} integral values used for taste diagnosis consist of a plurality of integral values in the same predetermined potential section (see=74 integral values and 37 integral values), or consist of a plurality of integral values including integral values in a first predetermined potential section and a plurality of integral values in a second predetermined potential section smaller than the first predetermined potential section (see=25 integral values, 19 integral values, 15 integral values, 13 integral values, 11 integral values, 9 integral values, and 7 integral values).

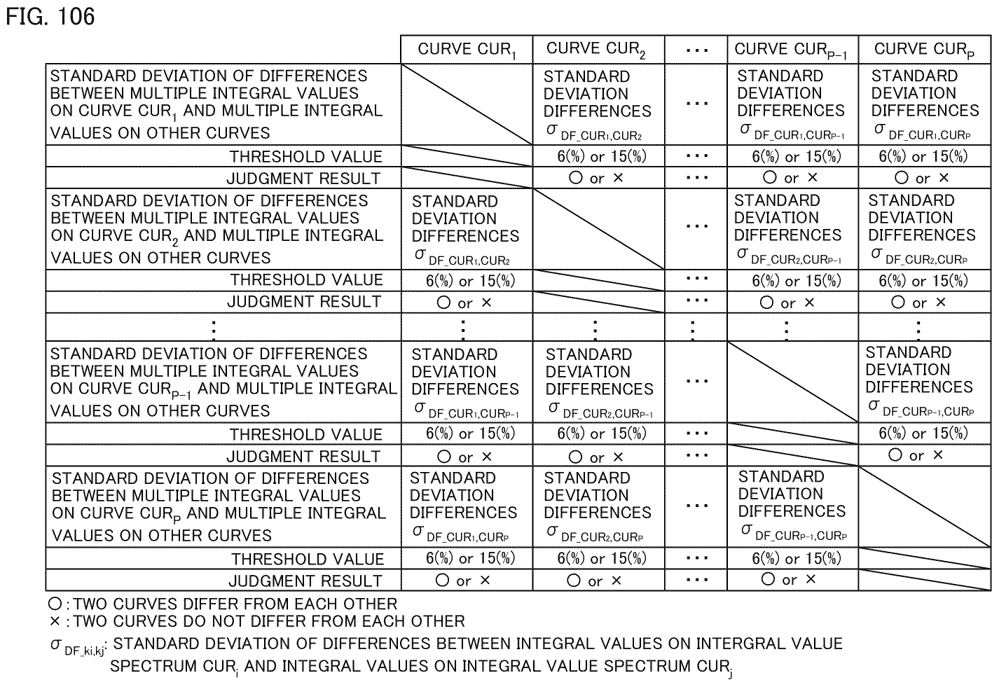

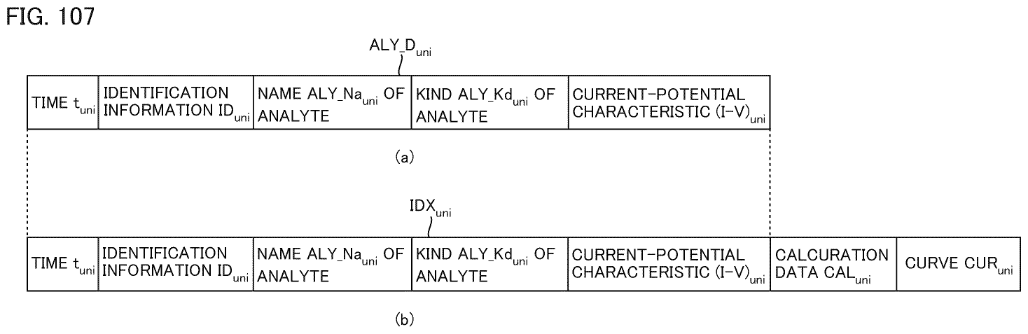

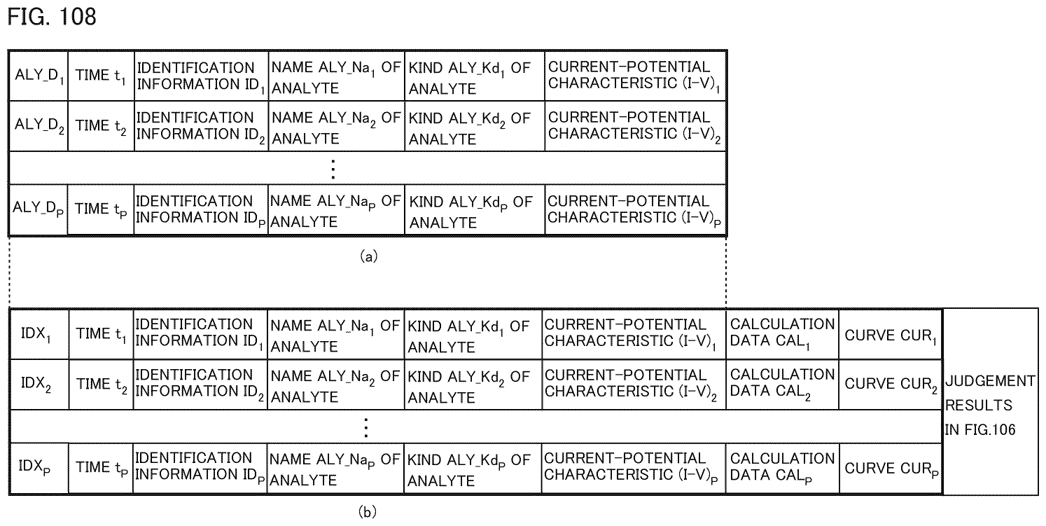

FIG. 106 is a conceptual diagram of a judgment result indicating whether _{P}C₂ pairs of two curves CURᵢ, CURⱼ are or not different.

Referring to FIG. 106, in _{P}C₂ pairs of the two curves CURᵢ, CURⱼ (i≠j), the threshold value σₜₕ(CURᵢ, CURⱼ) for judging whether the two curves CURᵢ, CURⱼ(i≠j; i=1 to P, j=1 to P) are or not different is, for example, 6% or 15%. Here, _{P}C₂ is the number of combinations of two different curves CURᵢ, CURⱼ (i≠j) in when extracting the two different curves CURᵢ, CURⱼ (i≠j) from the P curves CUR₁ to CUR_{P}.

When a judgment result is the judgment result (◯) that all of the _{P}C₂ pairs of two curves CURᵢ, CURⱼ are different, the P curves CUR₁ to CUR_{P} are judged to be different from each other. In this case, the P curves CUR₁ to CUR_{P} each represent a feature amount by an integral value (=the sum ITG_{ST} of the integral values shown in FIG. 12) for the P analytes.

On the other hand, when at least one pair of two curves CURᵢ, CURⱼ in “the _{P}C₂ pairs of two curves CURᵢ, CURⱼ” do not differ, the P curves CUR₁ to CUR_{P} are not different from one another.

And, when at least one pair of two curves CURᵢ, CURⱼ among the ₚC₂ pairs of two curves CURᵢ, CURⱼ are not different, by referring to the judgment result shown in FIG. 106, it is possible to easily understand the two curves CURᵢ, CURⱼ that differ and the two curves CUR, CUR, that do not differ.

Furthermore, when all of _{P}C₂ pairs of two curves CURᵢ, CURⱼ are not different (x), the P curves CUR₁ to CUR_{P} are judged to be not different from one another.

The judgment unit 214 creates, in step S77 of FIG. 91, the judgment result shown in FIG. 106. Then, the judgment unit 214 outputs the judgment result shown in FIG. 106 to the creation unit 215.

The creation unit 215 receives P calculation results CAL_RLS₁=[ID₁/CAL₁] to CAL_RLS_{P}=[ID_{P}/CAL] from the calculation unit 213 and the judgment result shown in FIG. 106 from the judgment unit 214, and then adds curves CUR₁ to CUR_{P} to the P calculation results CAL_RLS₁=[ID₁/CAL₁] to CAL_RLS_{P}=[ID_{P}/CAL_{P}], respectively, to create P analysis results ALY_RLS₁=[ID₁/CAL₁/CUR₁] to ALY_RLS_{P}=[ID_{P}/CAL_{P}/CUR_{P}].

Then, the creation unit 215 outputs the P analysis results ALY_RLS₁=[ID₁/CAL₁/CUR₁] to ALY_RLS_{P}=[ID_{P}/CAL_{P}/CUR_{P}] and the judgment results shown in FIG. 106 to the control unit 212.

In FIG. 106, a threshold value σₜₕ(CURᵢ, CURⱼ) of 5% is used when it is judged whether the 20 curves k27 to k46 for Shochu of No. 1 to No. 20 are or not different from one another. The threshold value σ_{th(CUR}_{ᵢ}_{, CUR}_{ⱼ}₎ of 5% is used similary in the case where it is judged whether the 20 curves k47 to k66 are or not different from one another for Shochu of No. 1 to No. 20, in the case where it is judged whether the 20 curves k67 to k86 are or not different from one another for Shochu of No. 1 to No. 20, and in the case where when it is judged whether the 20 curves k87 to k106 are or not different from one another for Shochu of No. 1 to No. 20.

Also, in FIG. 106, a threshold value σ_{th(cuR}_{₁}_{, CUR}_{ⱼ}₎ of 8% is used when it is judged whether the six curves k107 to k112 are or not different from one another, when it is judged whether the six curves k113 to k118 are or not different from one another, when it is judged whether the six curves k119 to k124 are or not different from one another, and when it is judged whether the six curves k125 to k130 are or not different from one another, for grapes.

FIG. 107 is a conceptual diagram showing the update from the analysis data ALY_Dᵤₙᵢ to the index data IDXᵤₙᵢ in step S74 of FIG. 91.

Referring to FIG. 107, in step S72 of FIG. 91, the control unit 212 of the diagnostic device 2 creates analysis dataALY_Dᵤₙᵢ in which includes the time tᵤₙᵢ, identification information IDᵤₙᵢ, name ALY_Naᵤₙᵢ of the analyte, type ALY_Kdᵤₙᵢ of analyte and current-potential characteristic (I-V)ᵤₙᵢ based on the measurement data MRS_uni (see (a) of FIG. 107).

Then, the control unit 212 receives the analysis result ALY_RLSᵤₙᵢ=[IDᵤₙᵢ/CALᵤₙᵢ/CURᵤₙᵢ] from the creation unit 215 and reads out the analysis data ALY_Dᵤₙᵢ having the same identification information as the identification information IDᵤₙᵢ in the analysis result ALY_RLSᵤₙᵢ from the database 22 after outputting the analysis data ALY_Dᵤₙᵢ to the calculation unit 213.

Then, the control unit 212 detects the calculation data CALᵤₙᵢ and the curve CURᵤₙᵢ from the analysis result ALY_RLSᵤₙᵢ=[IDᵤₙᵢ/CALᵤₙᵢ/CURᵤₙᵢ], adds the detected calculation data CALᵤₙᵢ and the curve CURᵤₙᵢ to the analysis data ALY_Dᵤₙᵢ to create the index data IDXᵤₙᵢ, and updates the analysis data ALY_Dᵤₙᵢ to the index data IDXᵤₙᵢ (see FIG. 107(b)). In this case, the calculation data CALᵤₙᵢ has the structure shown in FIG. 12.

Then, the control unit 212 stores the index data IDXᵤₙᵢ in the database 22 in place of the analysis data ALY_Dᵤₙᵢ.

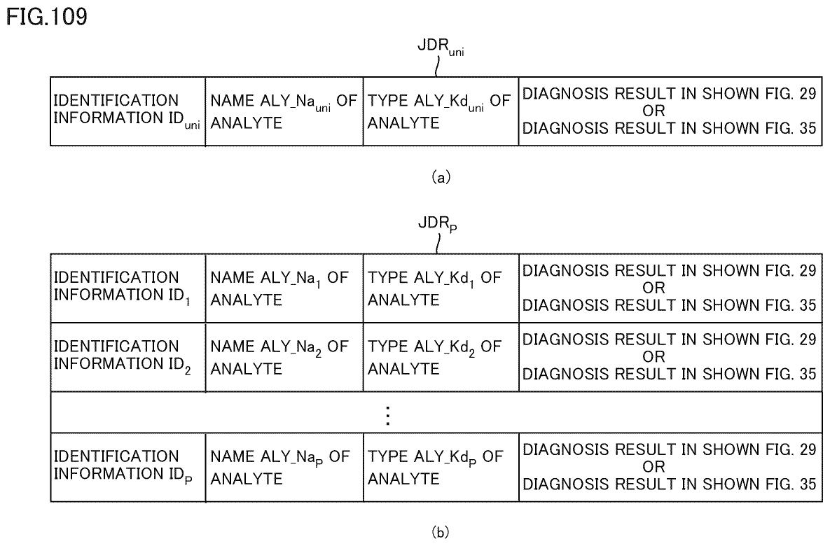

FIG. 108 is a conceptual diagram showing the updating of from P pieces of analysis data ALY_D₁ to ALY_D_{P} to P pieces of index data IDX₁ to IDX_{P} in step S78 of FIG. 91.

Referring to FIG. 108, in step S75 of FIG. 91, the control unit 212 of the diagnostic device 2 creates analysis dataALY_D₁ including time t₁, identification information ID₁, name ALY_Na₁ of the analyte, kind ALY_Kd₁ of the analyte, and current-potential characteristic (I-V)₁, analysis data ALY_D₂ including time t₂, identification information ID₂, name ALY_Na₂ of the analyte, kind ALY_Kd₂ of the analyte, and current-potential characteristic (I-V)₂, . . . , analysis data ALY_D_{P} including time tₚ, identification information ID_{P}, name ALY_Na_{P} of the analyte, kind ALY_Kd_{P} of the analyte, and current-potential characteristic (I-V)_{P}, based on P pieces of measurement data MRS_1 to MRS_P (see (a) of FIG. 108). That is, the control unit 212 creates P pieces of analysis data ALY_D₁ to ALY_D_{P} based on the P pieces of measurement data MRS_1 to MRS_P.

Then, the control unit 212 reads out, from the database 22, P pieces of analysis data ALY_D₁ to ALY_D_{P} which have the same identification information as the P identification information ID₁ to ID_{P}, respectively, in the P analysis results ALY_RLS₁ to ALY_RLS_{P} when receiving P analysis results ALY_RLS₁=[ID₁/CAL₁/CUR₁] to ALY_RLS_{P}=[ID_{P}/CAL_{P}/CUR_{P}] from the creation unit 215 after the control unit 212 outputs P pieces of analysis data ALY_D₁ to ALY_D_{P} to the calculation unit 213.

Then, the control unit 212 detects the calculation data CALₚ and the curve CURₚ from the analysis result ALY_RLSₚ=[IDₚ/CALₚ/CURₚ] (p is any of 1 to P), adds the detected calculation data CALₚ and curve CURₚ to the analysis data ALY_Dₚ to create index data IDXₚ, and performing, for all P pieces of analysis data ALY_D₁ to ALY_D_{P}, updating the analysis data ALY_Dₚ to the index data IDXₚ, thereby updating the P pieces of analysis data ALY_D₁ to ALY_D_{P} to P pieces of index data IDX₁ to IDX_{P}, respectively (see (b) of FIG. 108).

Then, the control unit 212 associates the judgment result (the judgment result shown in FIG. 106) with the P index data IDX₁ to IDXₚ (see (b) of FIG. 108).

In response, the control unit 2₂ stores the P index data IDX₁ to IDX_{P} in the database 22 instead of the P analysis data ALY_D₁ to ALY_D_{P}, and stores the judgment result (the judgment result shown in FIG. 106) in the database 22 in association with the P index data IDX₁ to IDX_{P}.

FIG. 109 is a conceptual diagram showing the results of a taste diagnosis. FIG. 109 (a) shows the results of a taste diagnosis for one analyte, and FIG. 109 (b) shows the results of a taste diagnosis for P analytes.

Referring to (a) of FIG. 109, the taste diagnosis result JDRᵤₙᵢ is configured to correspond to the identification information IDᵤₙᵢ of the analyte, the name ALY_Naᵤₙᵢ of the analyte, the type ALY_Kdᵤₙᵢ of the analyte, and the diagnosis result shown in FIG. 29 or the diagnosis result shown in FIG. 35.

Referring to (b) of FIG. 109, the taste diagnosis result JDR_{P} includes [the identification information ID₁/the name ALY_Na₁/the type ALY_Kd₁/the diagnosis result shown in FIG. 29 or the diagnosis result shown in FIG. 35] corresponding to the identification information ID₁ of the analyte, the name ALY_Na₁ of the analyte, the type ALY_Kd₁ of the analyte, and the diagnosis result shown in FIG. 29 or the diagnosis result shown in FIG. 35., [the identification information ID₂/the name ALY_Na₂/the type ALY_Kd₂/the diagnosis result shown in FIG. 29 or the diagnosis result shown in FIG. 35] corresponding to the identification information ID₂ of the analyte, the name ALY_Na₂ of the analyte, the type ALY_Kd₂ of the analyte, and the diagnosis result shown in FIG. 29 or the diagnosis result shown in FIG. 35, . . . , [the identification information ID_{P}/the name ALY_Na_{P}/the type ALY_Kd_{P}/the diagnosis result shown in FIG. 29 or the diagnosis result shown in FIG. 35] corresponding to the identification information ID_{P} of the analyte, the name ALY_Na_{P} of the analyte, the type ALY_Kd_{P} of the analyte, and the diagnosis result shown in FIG. 29 or the diagnosis result shown in FIG. 35.

The taste diagnostic unit 217 creates a taste diagnosis result JDRᵤₙᵢ and a taste diagnosis result JDR_{P}, stores the created taste diagnosis result JDRᵤₙᵢ₁ and taste diagnosis result JDR_{P} in the database 22 and outputs them to the display unit 218.

When the display unit 218 receives the taste diagnosis result JDRᵤₙᵢ from the taste diagnostic unit 217, the display unit 218 displays the taste diagnosis result JDRᵤₙᵢ, and when the display unit 218 receives the taste diagnosis result JDR_{P} from the taste diagnostic unit 217, the display unit 218 displays the taste diagnosis result JDR_{P}.

In an embodiment of the present invention, the operation of the diagnostic device 2 may be performed by software. In this case, the diagnostic device 2 includes a CPU (Central Processing Unit), a ROM (Read Only Memory), and a RAM (Random Access Memory).

The ROM stores a program Prog_A consisting of steps S6 to S8 (including the flowcharts shown in FIGS. 91 to 99, and FIG. 101) shown in FIG. 90, or a program Prog_B consisting of steps S6 to S8 (including the flowcharts shown in FIGS. 91 to 98, FIG. 100, and FIG. 102) shown in FIG. 90.

The CPU reads out the program Prog_A or the program Prog_B from the ROM, and executes the program Prog_A which is read out or the program Prog_B which is read out to diagnose the taste of the analyte.

In this case, the RAM temporarily stores the standard deviations σ_{DF_CUR}_{ᵢ}_{, CUR}_{ⱼ} (i≠j, i=1 to P, j=1 to P) of differences, the standard deviations σ_{DF_DFF}_{ᵢ}_{, DFF}_{ⱼ} (i≠j, i=1 to P, j=1 to P) of differences, the threshold value σₜₕ, the sums L(+)_sum, M(+)_sum, H(+)_sum, L(all)_sum, M(all)_sum, H(all)_sum, H(−)_sum, L(−)_sum_th, M(−)_sum_th, H(−)_sum_th of integral values, Body index (+), Body index (all), Body index (−)_th, and etc.

Therefore, the program Prog_A or the program Prog_B is a program for causing a computer (CPU) to execute a diagnosis of the taste of the analyte.

### Second Embodiment

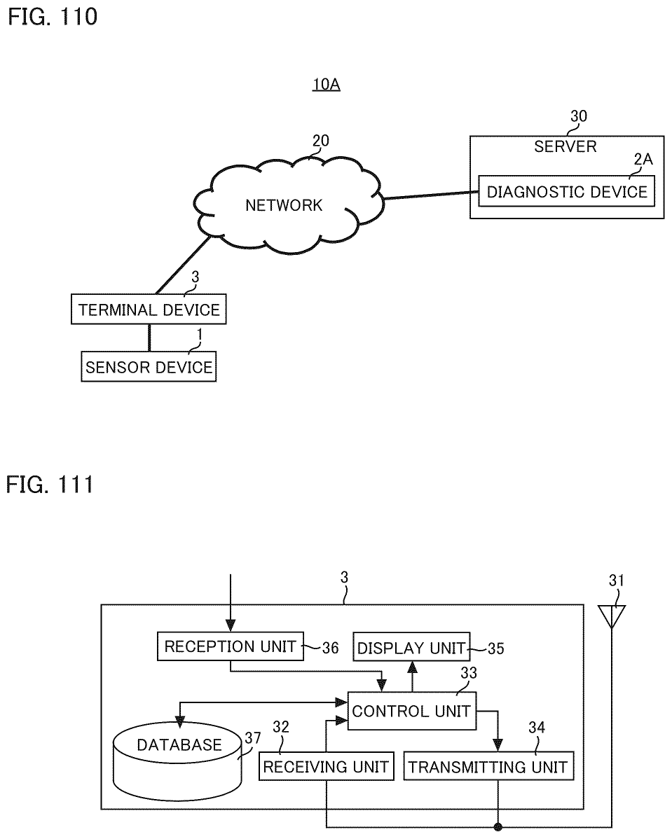

FIG. 110 is a schematic diagram of a diagnostic system according to embodiment 2. Referring to FIG. 110, a diagnostic system 10A according to embodiment 2 includes a sensor device 1, a diagnostic device 2A, and a terminal device 3.

In the embodiment 2, the sensor device 1 and the terminal device 3 are arranged in, for example, restaurants such as Japanese restaurants, Chinese restaurants, and Western restaurants, sake breweries that brew shochu, and liquor stores that sell shochu, etc. Furthermore, the diagnostic device 2A is arranged in the server 30.

The terminal device 3 receives measurement data MRS_uni (consisting of the measurement data MRS shown in FIG. 6) or P pieces of measurement data MRS_1 to MRS_P (each of the measurement data MRS_1 to MRS_P is composed of the measurement data MRS shown in FIG. 6) from the sensor device 1 via wireless communication or wired communication.

Then, the terminal device 3 transmits the measurement data MRS_uni (consisting of the measurement data MRS shown in FIG. 6) to the diagnostic device 2A via the network 20.

In addition, the terminal device 3 receives the diagnosis result of the taste of the analyte (diagnosis result JDRᵤₙᵢ or diagnosis result JDR_{P} shown in FIG. 109) from the diagnostic device 2A via the network 20, and displays the received diagnosis result of the taste of the analyte (diagnosis result JDRᵤₙᵢ or diagnosis result JDR_{P} shown in FIG. 109).

The diagnostic device 2A receives the measurement data MRS_uni (comprised of the measurement data MRS shown in FIG. 6) from the terminal device 3 via the network 20.

Then, the diagnostic device 2A creates the above-mentioned analysis data ALY_Dᵤₙᵢ based on the measurement data MRS_uni, creates calculation data CALᵤₙᵢ based on the analysis data ALY_Dᵤₙᵢ and creates a curve CURᵤₙᵢ based on the calculation data CALᵤₙᵢ.

Then, the diagnostic device 2A updates the analysis data ALY_Dᵤₙᵢ to the index data IDXᵤₙᵢ, diagnoses the taste of the analyte based on the updated index data IDXᵤₙᵢ to create a diagnosis result JDRᵤₙᵢ by the method described in the first embodiment. Then, the diagnostic device 2A transmits the diagnosis result JDRᵤₙᵢ to the terminal device 3 via the network 20.

Moreover, the diagnostic device 2A receives P pieces of measurement data MRS_1 to MRS_P (each of the measurement data MRS_1 to MRS_P is made up of the measurement data MRS shown in FIG. 6) from the terminal device 3 via the network 20.

Then, the diagnostic device 2A creates the above-mentioned P pieces of analysis data ALY_D₁ to ALY_D_{P} based on the P pieces of measurement data MRS_1 to MRS_P, creates P pieces of calculation data CAL₁ to CAL_{P} based on the P pieces of analysis data ALY_D₁ to ALY_D_{P}, respectively, and creates P pieces of curves CUR₁ to CUR_{P} based on the P pieces of calculation data CAL₁ to CAL_{P}, respectively. Also, the diagnostic device 2A creates a judgment result (the judgment result shown in FIG. 106) indicating whether the P curves CUR₁ to CUR_{P} are different or not., respectively,

Then, the diagnostic device 2A updates the P pieces of analysis dataALY_D₁ to ALY_D_{P} to P pieces of index data IDX₁ to IDX_{P}, respectively, using the method described in embodiment 1, and diagnoses the taste of the P analytes based on the updated P pieces of index data IDX₁ to IDX_{P} to create a diagnosis result JDR_{P}.

Then, diagnostic device 2A transmits the diagnosis result JDR_{P} to the terminal device 3 via network 20.

FIG. 111 is a schematic diagram of the terminal device 3 shown in FIG. 110. Referring to FIG. 111, the terminal device 3 includes an antenna 31, a receiving unit 32, a control unit 33, a transmitting unit 34, a display unit 35, a reception unit 36, and a database 37.

The receiving unit 32 receives the measurement data MRS_uni or the P pieces of measurement data MRS_1 to MRS_P from the sensor device 1 via the antenna 31 by wireless communication, or receives the measurement data MRS_uni or the P pieces of measurement data MRS_1 to MRS_P from the sensor device 1 by wired communication. The receiving unit 32 is connected to the sensor device 1 through a wired cable in the case of receiving the measurement data MRS_uni or the P pieces of measurement data MRS_1 to MRS_P from the sensor device 1 through wired communication.

Then, the receiving unit 32 outputs the measurement data MRS_uni or the P pieces of measurement data MRS_1 to MRS_P to the control unit 33.

In addition, the receiving unit 32 receives the taste diagnosis result JDRᵤₙᵢ (or the taste diagnosis result JDR_{P}) from the diagnostic device 2A via the network 20 and the antenna 31, and outputs the received taste diagnosis result JDRᵤₙᵢ (or the taste diagnosis result JDR_{P}) to the control unit 33.

The control unit 33 has a built-in timer. Then, when the control unit 33 receives the measurement data MRS_uni from the receiving unit 32, the control unit 33 refers to the timer and detects the time tᵤₙᵢ in when the measurement data MRS_uni was received from the receiving unit 32. Then, the control unit 33 store the measurement data MRS_uni in the database 37 by associating the measurement data MRS_uni to the time tᵤₙᵢ.

Then, the control unit 33 outputs the measurement data MRS_uni to the transmitting unit 34.

On the other hand, the control unit 33 refers to the timer to detect the times t₁ to t_{P} at which the P pieces of measurement data MRS_1 to MRS_P were received, respectively, when the control unit 33 has received the P pieces of measurement data MRS_1 to MRS_P from the receiving unit 32. Then, the control unit 33 associates the P pieces of measurement data MRS_1 to MRS_P with the times t₁ to tₚ, respectively, to store the P pieces of measurement data MRS_1 to MRS_P in the database 37.

Then, the control unit 33 outputs the P pieces of measurement data MRS_1 to MRS_P to the transmitting unit 34.

Furthermore, when the control unit 33 received the taste diagnosis result JDRᵤₙᵢ from the receiving unit 32, the control unit 33 stores the received taste diagnosis result JDRᵤₙᵢ in association with [time tᵤₙᵢ/measurement data MRS_uni] in the database 37.

On the other hand, when the control unit 33 received the taste diagnosis result JDR_{P} from the receiving unit 32, the control unit 33 stores the received taste diagnosis result JDR_{P} in association with [time t₁/measurement data MRS_1] to [time tₚ/measurement data MRS_P] in the database 37.

Furthermore, the control unit 33 detects the diagnosis result JDRᵤₙᵢ associated with the name ALY_Naᵤₙᵢ of the analyte from the database 37 based on the name ALY_Naᵤₙᵢ of the analyte, and outputs the name ALY_Naᵤₙᵢ of the analyte and the diagnosis result JDRᵤₙᵢ to the display unit 35 when the control unit 33 received a request RQTᵤₙᵢ from the reception unit 36 to display the name ALY_Naᵤₙᵢ of the analyte and the diagnosis result JDRᵤₙᵢ associated with the name ALY_Naᵤₙᵢ.

Furthermore, the control unit 33 detects the q (q is an integer satisfying 1≤q≤P) names ALY_Na₁ to ALY_Na_{q} of the q analytes and the q diagnosis results JDR₁ to JDR_{q} associated with the q names ALY_Na₁ to ALY_Na_{q} of the q analytes, respectively, from the database 37 based on the q names ALY_Na₁ to ALY_Na_{q} and outputs the q names ALY_Na₁ to ALY_Na_{q} of the q analytes and the q diagnosis results JDR₁ to JDR_{q} to the display unit 35 when the control unit 33 received, from the reception unit 36, a request RQT_{q} to display q names ALY_Na₁ to ALY_Na_{q} out of the P names ALY_Na₁ to ALY_Na_{P} of the P analytes and the q diagnosis results JDR₁ to JDR_{q} associated with the q names ALY_Na₁ to ALY_Na_{q}, respectively.

In this case, in the request RQT_{q}, q types ALY_Kd₁ to ALY_Kd_{q} of the q analytes may be used instead of the q names ALY_Na₁ to ALY_Na_{q} of the q analytes.

When the transmitting unit 34 receives the measurement data MRS_uni from the control unit 33, the transmitting unit 34 transmits the measurement data MRS_uni to the diagnostic device 2A via the antenna 31 and the network 20.

On the other hand, when the transmitting unit 34 received the P pieces of measurement data MRS_1 to MRS_P from the control unit 33, the transmitting unit 34 transmits the P pieces of measurement data MRS_1 to MRS_P via the antenna 31 and the network 20 to the diagnostic device 2A.

When the display unit 35 received the taste diagnosis result JDRᵤₙᵢ from the control unit 33, the display unit 35 displays the received taste diagnosis result JDRᵤₙᵢ.

Furthermore, when the display unit 35 received taste diagnosis results (=q diagnosis results JDR₁ to JDR_{q}) from the control unit 33, the display unit 35 displays the received taste diagnosis results (=q diagnosis results JDR₁ to JDR_{q}).

The reception unit 36 receives a request RQTᵤₙᵢ to display the name ALY_Naᵤₙᵢ of the analyte and the diagnosis result JDRᵤₙᵢ associated with the name ALY_Naᵤₙᵢ, and outputs the received request RQTᵤₙᵢ to the control unit 33.

In addition, the reception unit 36 receives a request RQT_{q} to display q (q is an integer satisfying 1≤q≤P) names ALY_Na₁ to ALY_Na_{q} out of the P names ALY_Na₁ to ALY_Na_{P} of the P analytes, and q diagnosis results JDR₁ to JDR_{q} respectively associated with the q names ALY_Na₁ to ALY_Na_{q}, and outputs the received request RQT_{q} to the control unit 33.

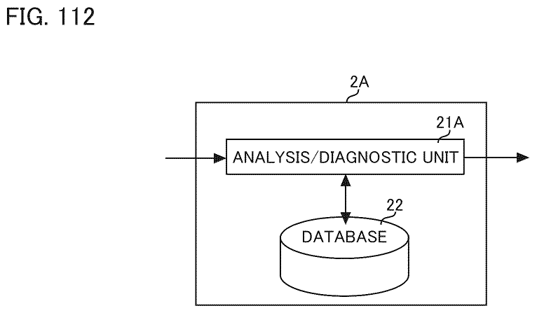

FIG. 112 is a schematic diagram of the diagnostic device 2A shown in FIG. 110. Referring to FIG. 112, the diagnostic device 2A is the same as the diagnostic device 2 shown in FIG. 7 except that the analysis/diagnostic unit 21 of the diagnostic device 2 shown in FIG. 7 is replaced with an analysis/diagnostic unit 21A.

The analysis/diagnostic unit 21 A receives the measurement data MRS from the terminal device 3 via the network 20.

Then, the analysis/diagnostic unit 21A diagnoses the taste of the analyte (shochu or grapes) using the method described above based on the measurement data MRS, stores the diagnosed taste diagnosis result JDR in the database 22 in association with the analyte (shochu or grapes), and transmits the taste diagnosis result JDR of the analyte (shochu or grapes) to the terminal device 3 via the network 20.

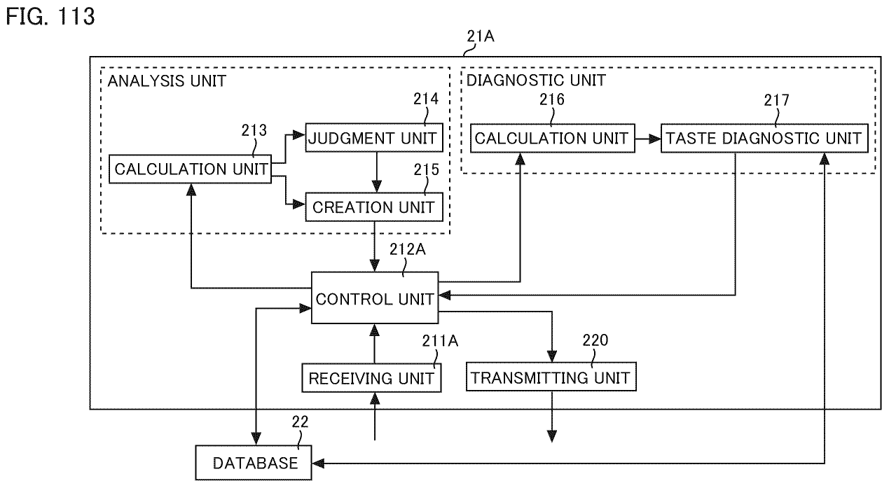

FIG. 113 is a schematic diagram of the analysis/diagnostic unit 21A shown in FIG. 112. Referring to FIG. 113, the analysis/diagnostic unit 21A changes the receiving unit 211 of the analysis/diagnostic unit 21 shown in FIG. 8 to a receiving unit 211A, changes the display unit 218 and the reception unit 219 of the analysis/diagnostic unit 21 shown in FIG. 8 to a transmitting unit 220, and changes the control unit 212 of the analysis/diagnostic unit 21 shown in FIG. 8 to a control unit 212A, otherwise the analysis/diagnostic unit 21A is the same as the analysis/diagnostic unit 21 shown in FIG. 8.

The receiving unit 211A receives the measurement data MRS_uni or P pieces of measurement data MRS_1 to MRS_P from the terminal device 3 via the network 20, and outputs the received measurement data MRS_uni or P pieces of measurement data MRS_1 to MRS_P to the control unit 212A.

The control unit 212A receives the measurement data MRS_uni or P pieces of measurement data MRS_1 to MRS_P from the receiving unit 211A. Then, the control unit 212A stores the measurement data MRS_uni or the P pieces of measurement data MRS_1 to MRS_P in the database 22.

In addition, when the control unit 212A received the taste diagnosis result JDRᵤₙᵢ of the analyte from the taste diagnostic unit 217, the control unit 212A stores the received taste diagnosis result JDRᵤₙᵢ of the analyte in the database 22 and outputs the taste diagnosis result JDRᵤₙᵢ of the analyte to the transmitting unit 220.

Furthermore, when the control unit 212A received a diagnosis result JDR_{P} of the taste of the analyte from the taste diagnostic unit 217, the control unit 212A stores the received diagnosis result JDR_{P} of the taste of the analyte in the database 22 and outputs the diagnosis result JDR_{P} of the taste of the analyte to the transmitting unit 220.

Otherwise, the control unit 212A performs the same functions as the control unit 212 described above.

When the transmitting unit 220 received the diagnosis result JDRᵤₙᵢ of the taste of the analyte from the control unit 212A, the transmitting unit 220 transmits the diagnosis result JDRᵤₙᵢ of the taste of the analyte to the terminal device 3 via the network 20.

Furthermore, when the transmitting unit 220 receives the diagnosis result JDR_{P} of the taste of the analyte from the control unit 212A, the transmitting unit 220 transmits the diagnosis result JDR_{P} of the taste of the analyte to the terminal device 3 via the network 20.

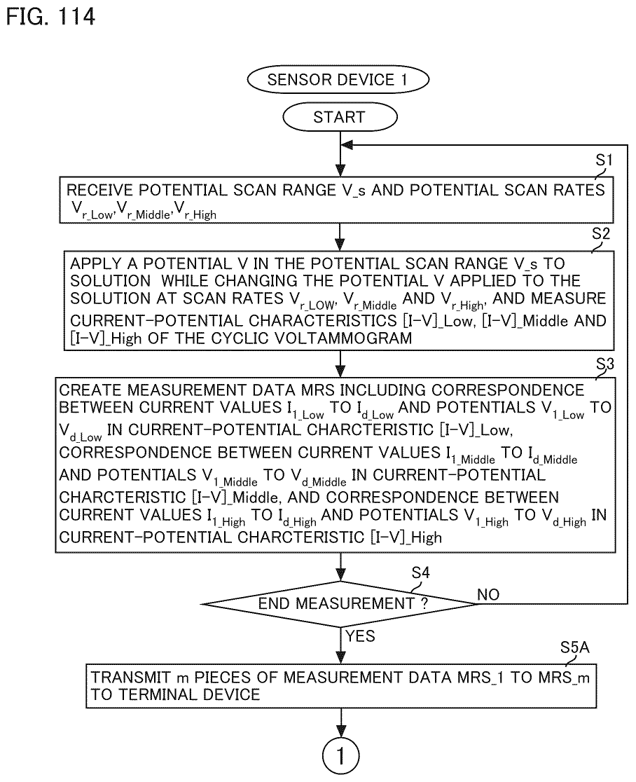

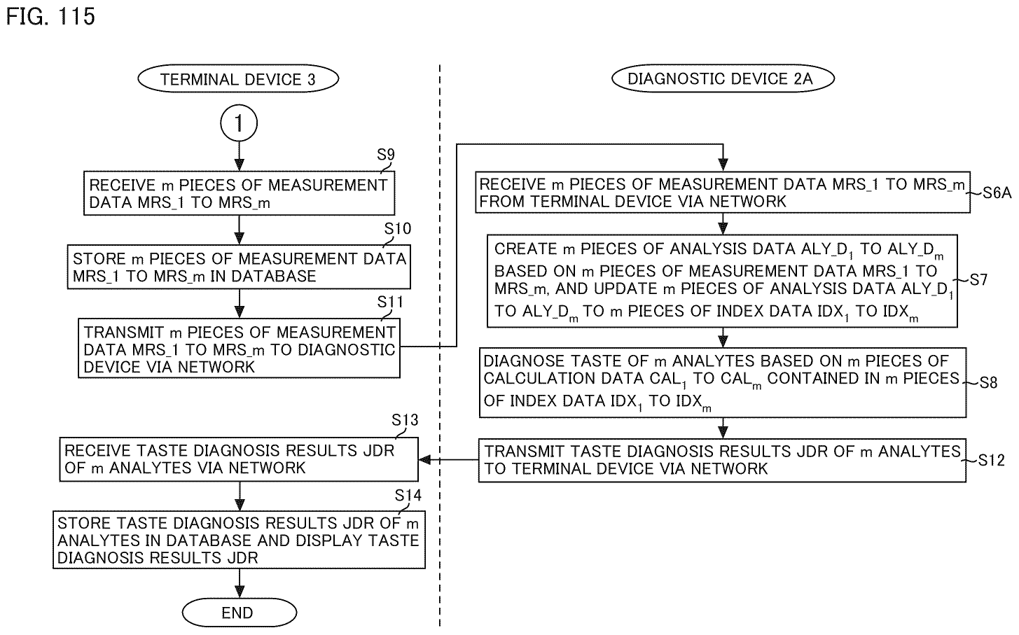

FIGS. 114 and 115 are first and second flowcharts, respectively, for explaining the operation of the diagnostic system 10A shown in FIG. 110.

The flowcharts shown in FIGS. 114 and 115 are the same as the flowchart shown in FIG. 90, except that step S5 of the flowchart shown in FIG. 90 is replaced with step S5A, step S6 of the flowchart shown in FIG. 90 is replaced with step S6A, and steps S9 to S14 are added.

Referring to FIG. 114, when the operation of diagnostic system 10A is started, steps S1 to S4 described above are sequentially executed.

Then, when it is judged in step S4 that the measurement has ended, the sensor device 1 transmits the m pieces of measurement data MRS_1 to MRS_m to the terminal device 3 by wireless communication or wired communication (step S5A).

Referring to FIG. 115, after the step S5A, the terminal device 3 receives m pieces of measurement data MRS_1 to MRS_m from the sensor device 1 by wireless communication or wired communication (step S9).

Then, the terminal device 3 stores the m pieces of measurement data MRS_1 to MRS_m in the database 37 (step S10), and transmits the m pieces of measurement data MRS_1 to MRS_m to the diagnostic device 2A via the network 20 (step S11).

The diagnostic device 2A receives the m pieces of measurement data MRS_1 to MRS_m from the terminal device 3 via the network 20 (step S6A).

After the step S6A, the above-mentioned steps S7 and S8 are sequentially executed. Then, after the step S8, the diagnostic device 2A transmits the taste diagnosis results JDR of the m analytes to the terminal device 3 via the network 20 (step S12).

The terminal device 3 receives the taste diagnosis results JDR of the m analytes from the diagnostic device 2A via the network 20 (step S13).

Then, the terminal device 3 stores the taste diagnosis results JDR of the m analytes in the database 37 and displays the taste diagnosis results JDR (step S14). This completes the operation of the diagnostic system 10A.

In the embodiment of the present invention, the operation of the diagnostic device 2A may be executed by software. In this case, the diagnostic device 2A includes a CPU, a ROM and a RAM.

The ROM stores a program Prog_C consisting of steps S6A, S7, S8, and S12 (including the flowcharts shown in FIG. 91 to FIG. 99, and FIG. 101) shown in FIG. 115, or a program Prog_D consisting of steps S6A, S7, S8, and S12 (including the flowcharts shown in FIG. 91 to FIG. 98, FIG. 100, and FIG. 102) shown in FIG. 115.

The CPU reads out the program Prog_C or the program Prog_D from the ROM, and executes the read out program Prog_C or the read out program Prog_D to diagnose the taste of the analyte.

In this case, the RAM temporarily stores the standard deviations σ_{DF_CUR}_{ᵢ}_{, CUR}_{ⱼ} (i≠j, i=1 to P, j=1 to P) of differences, the standard deviations σ_{DF_DFF}_{ᵢ}_{, DFF}_{ⱼ} (i≠j, i=1 to P, j=1 to P) of differences, the threshold value σₜₕ, the sums of integral values L(+)_sum, M(+)_sum, H(+)_sum, L(all)_sum, M(all)_sum, H(all)_sum, H(−)_sum, L(−)_sum_th, M(−)_sum_th, H(−)_sum_th, Body index (+), Body index (all), and Body index (−)_th, etc.

Therefore, the program Prog_C or the program Prog_D is a program for causing a computer (CPU) to execute a diagnosis of the taste of analyte.

In the embodiment of the present invention, the operation of the terminal device 3 may be executed by software. In this case, the terminal device 3 includes a CPU, a ROM, and a RAM.

The ROM stores a program Prog_E consisting of steps S9 to S11, S13, and S14 shown in FIG. 115.

The CPU reads out the program Prog_E from the ROM, executes the read out program Prog_E to receive m pieces of measurement data MRS_1 to MRS_m from the sensor device 1, transmits the received m pieces of measurement data MRS_1 to MRS_m to the diagnostic device 2A, and also receives a taste diagnosis result JDR of the analyte from the diagnostic device 2A to store the taste diagnosis result JDR in the database 37, and displays the taste diagnosis result JDR.

In this case, the RAM temporarily stores the m pieces of measurement data MRS_1 to MRS_m and the taste diagnosis result JDR.

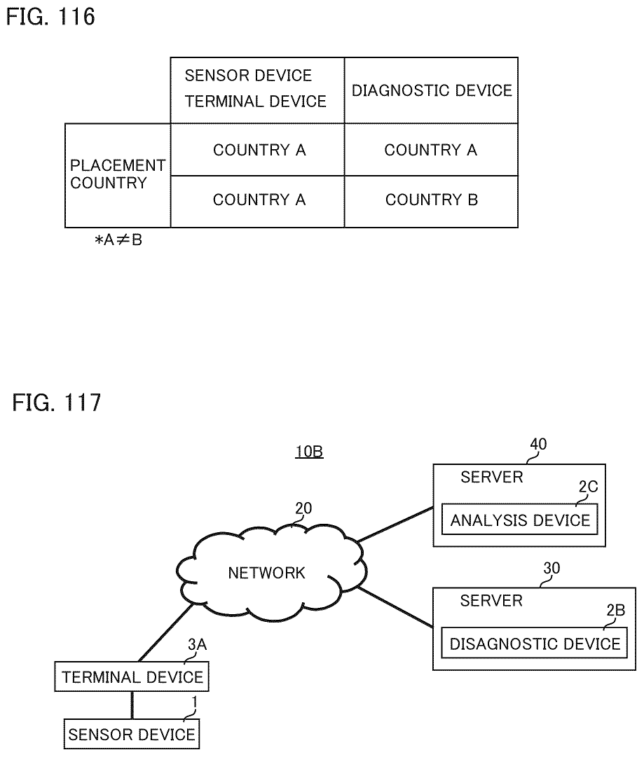

FIG. 116 is a diagram for explaining the locations of the sensor device 1, the terminal device 3, and the diagnostic device 2A.

Referring to FIG. 116, the sensor device 1, the terminal device 3 and the diagnostic device 2A are placed in country A.

In addition, the sensor device 1 and the terminal device 3 are placed in country A, and the diagnostic device 2A is placed in country B which is different from country A.

When the sensor device 1, terminal device 3 and diagnostic device 2A are located in country A, the diagnostic device 2A may receive measurement data MRS_uni or P pieces of measurement data MRS_1 to MRS_P from a terminal device 5 (terminal device having the same configuration as the terminal device 3) located in country C, which is different from country A, via the network 20, diagnose the taste of the analyte based on the received measurement data MRS_uni or P pieces of measurement data MRS_1 to MRS_P, and may transmit a diagnosis result JDR of the taste of the analyte to the terminal device 5 (terminal device having the same configuration as the terminal device 3) via the network 20.

In addition, when the sensor device 1 and the terminal device 3 are located in country A, and the diagnostic device 2A is located in country B which is different from country A, the diagnostic device 2A may receive measurement data MRS_uni or P pieces of measurement data MRS_1 to MRS_P from a terminal device 6 (a terminal device having the same configuration as the terminal device 3) located in country B, diagnose the taste of the analyte based on the received measurement data MRS_uni or P pieces of measurement data MRS_1 to MRS_P, and transmit a taste diagnosis result JDR of the analyte to the terminal device 6 (a terminal device having the same configuration as the terminal device 3) via the network 20.

Furthermore, when the sensor device 1 and the terminal device 3 are located in country A, and the diagnostic device 2A is located in country B which is different from country A, the diagnostic device 2A may receive measurement data MRS_uni or P pieces of measurement data MRS_1 to MRS_P from a terminal device 7 (a terminal device having the same configuration as the terminal device 3) located in country D which is different from countries A and B, diagnose the taste of the analyte based on the received measurement data MRS_uni or P pieces of measurement data MRS_1 to MRS_P, and transmit a diagnosis result JDR of the taste of the analyte to the terminal device 7 (a terminal device having the same configuration as the terminal device 3) via the network 20.

The rest of the description of the second embodiment is the same as that of the first embodiment.

### Embodiment 3

FIG. 117 is a schematic diagram of a diagnostic system according to embodiment 3. Referring to FIG. 117, a diagnostic system 10B according to embodiment 3 includes a sensor device 1, a diagnostic device 2B, an analysis device 2C, and a terminal device 3A.

In the third embodiment, the sensor device 1 and the terminal device 3A are arranged in, for example, restaurants such as Japanese restaurants, Chinese restaurants, and Western restaurants, sake breweries that brew shochu, and liquor stores that sell shochu, etc. Furthermore, the diagnostic device 2B is arranged in a server 30, and the analysis device 2C is arranged in a server 40.

The terminal device 3A receives measurement data MRS_uni (consisting of the measurement data MRS shown in FIG. 6) or P pieces of measurement data MRS_1 to MRS_P (each of the measurement data MRS_1 to MRS_P is composed of the measurement data MRS shown in FIG. 6) from the sensor device 1 via wireless communication or wired communication.

Then, the terminal device 3A transmits the measurement data MRS_uni (consisting of the measurement data MRS shown in FIG. 6) or P pieces of measurement data MRS_1 to MRS_P (each of the measurement data MRS_1 to MRS_P is composed of the measurement data MRS shown in FIG. 6) to the analysis device 2C via the network 20.

Thereafter, the terminal device 3A receives the index data IDXᵤₙᵢ or the P pieces of index data IDX₁ to IDX_{P} from the analysis device 2C via the network 20.

Then, the terminal device 3A stores the index data IDXᵤₙᵢ or the P pieces of index data IDX₁ to IDX_{P} in the database 37, and transmits the index data IDXᵤ, or the P pieces of index data IDX₁ to IDXₚ to the diagnostic device 2B via the network 20.

Then, the terminal device 3A receives the taste diagnosis result (diagnosis result JDRᵤₙᵢ or diagnosis result JDR_{P} shown in FIG. 109) of the analyte from the diagnostic device 2B via the network 20, stores the received taste diagnosis result (diagnosis result JDRᵤₙᵢ or diagnosis result JDR_{P} shown in FIG. 109) of the analyte in the database 37, and displays the taste diagnosis result (diagnosis result JDRᵤₙᵢ or diagnosis result JDR_{P} shown in FIG. 109) of the analyte.

The analysis device 2C receives measurement data MRS_uni (consisting of the measurement data MRS shown in FIG. 6) or P pieces of measurement data MRS_1 to MRS_P (each of the measurement data MRS_1 to MRS_P is composed of the measurement data MRS shown in FIG. 6) from the terminal device 3A via the network 20.

Then, the analysis device 2C creates analysis data ALY_Dᵤₙᵢ based on the measurement data MRS_uni (consisting of the measurement data MRS shown in FIG. 6) using the same method as the diagnostic device 2 in embodiment 1, and updates the created analysis data ALY_Dᵤₙᵢ to index data IDXᵤₙᵢ.

Then, the analysis device 2C transmits the index data IDXᵤₙᵢ to the terminal device 3A via the network 20.

In addition, the analysis device 2C creates P pieces of analysis data ALY_D₁ to ALY_D_{P}, respectively, based on P pieces of measurement data MRS_1 to MRS_P (each of the measurement data MRS_1 to MRS_P is composed of the measurement data MRS shown in FIG. 6) using the same method as the diagnostic device 2 in embodiment 1, and updates the created P pieces of analysis data ALY_D₁ to ALY_D_{P} to P pieces of index data IDX₁ to IDX_{P}, respectively.

Then, the analysis device 2C transmits the P pieces of index data IDX₁ to IDXₚ to the terminal device 3A via the network 20.

The diagnostic device 2B receives the index data IDXᵤₙᵢ or the P pieces of index data IDX₁ to IDX_{P} from the terminal device 3A via the network 20.

Then, the diagnostic device 2B diagnoses the taste of the analyte by the method explained in the first embodiment based on the index data IDXᵤₙᵢ, and creates a diagnosis result JDRᵤₙᵢ of the taste of the analyte.

Furthermore, the diagnostic device 2B diagnoses the taste of the P analytes by the method explained in the first embodiment based on the P index data IDX₁ to IDX_{P}, and creates a diagnosis result JDR_{P} of the taste of the P analytes.

Then, the diagnostic device 2B transmits the diagnosis result JDRᵤₙᵢ of the taste of the analyte or the diagnosis result JDR_{P} of the taste of the P analytes to the terminal device 3A via the network 20.

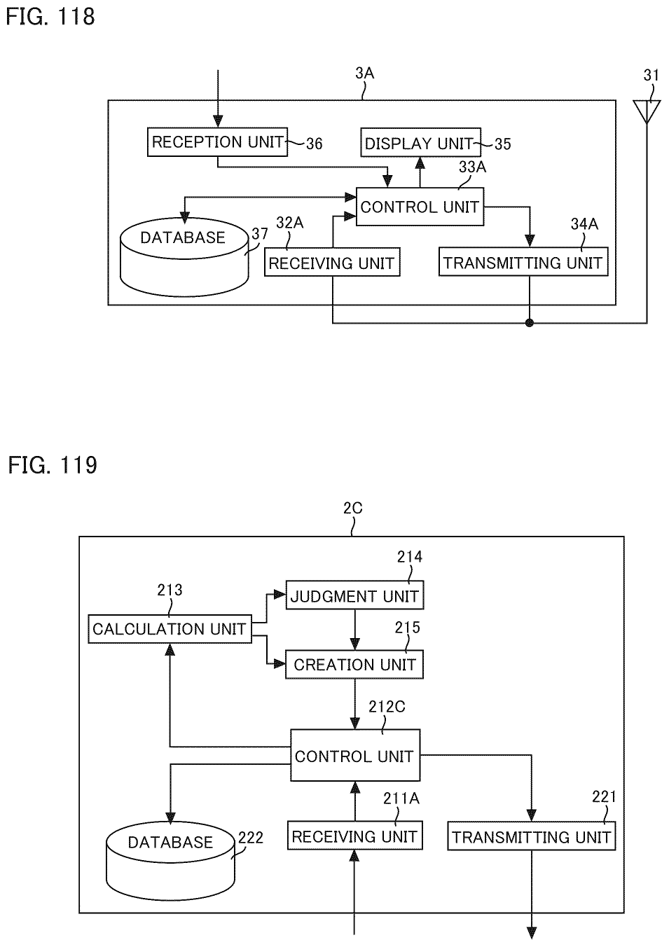

FIG. 118 is a schematic diagram of the terminal device 3A shown in FIG. 117. Referring to FIG. 118, the terminal device 3A is the same as the terminal device 3, except that the receiving unit 32, the control unit 33, and the transmitting unit 34 of the terminal device 3 shown in FIG. 111 are replaced with a receiving unit 32A, a control unit 33A, and a transmitting unit 34A, respectively.

The receiving unit 32A receives the index data IDXᵤₙᵢ or the P pieces of index data IDX₁ to IDX_{P} from the analysis device 2C via the network 20 and the antenna 31, and outputs the received index data IDXᵤₙᵢ or the P pieces of index data IDX₁ to IDX_{P} to the control unit 33A.

Otherwise, the receiving unit 32A performs the same functions as the receiving unit 32 described above.

When the control unit 33A receives the index data IDXᵤₙᵢ or the P pieces of index data IDX₁ to IDX_{P} from the receiving unit 32A, the control unit 33A stores the received index data IDXᵤₙᵢ or the P pieces of index data IDX₁ to IDX_{P} in the database 37 and outputs the index data IDXᵤₙᵢ or the P pieces of index data IDX₁ to IDX_{P} to the transmitting unit 34A.

Otherwise, the control unit 33A performs the same functions as the control unit 33 described above.

When the transmitting unit 34A receives the index data IDXᵤₙᵢ or the P pieces of index data IDX₁ to IDX_{P} from the control unit 33A, the transmitting unit 34A transmits the index data IDXᵤₙᵢ or the P pieces of index data IDX₁ to IDX_{P} to the diagnostic device 2B via the antenna 31 and the network 20.

Otherwise, the transmitting unit 34A performs the same functions as the transmitting unit 34 described above.

FIG. 119 is a schematic diagram of the analysis device 2C shown in FIG. 117. Referring to FIG. 119, in the analysis device 2C, the calculation unit 216 and the taste diagnostic unit 217 of the analysis/diagnostic unit 21A shown in FIG. 113 are deleted, the control unit 212A is changed to a control unit 212C, the transmitting unit 220 is changed to a transmitting unit 221, and a database 222 is added.

Of the operations of the control unit 212 described above, the control unit 212C executes the following: creation of analysis data ALY_Dᵤₙᵢ storage of the analysis data ALY_Dᵤₙᵢ in database 222, creation of P pieces of analysis data ALY_D₁ to ALY_D_{P}, storage of the P pieces of analysis data ALY_D₁ to ALY_D_{P} in database 222, output of the analysis data ALY_Dᵤₙᵢ to the calculation unit 213, output of the P pieces of analysis data ALY_D₁ to ALY_D_{P} to the calculation unit 213, updating of the analysis data ALY_Dᵤₙᵢ to index data IDXᵤₙᵢ, updating of the P pieces of analysis data ALY_D₁ to ALY_D_{P} to P pieces of index data IDX₁ to IDX_{P}, respectively, storage of index data IDXᵤₙᵢ in database 222, and storage of the P pieces of index data IDX₁ to IDX_{P} in database 222.

In addition, the control unit 212C outputs the index data IDXᵤₙᵢ or the P index data IDX₁ to IDX_{P} to the transmitting unit 221.

When the transmitting unit 221 receives the index data IDXᵤₙᵢ from the control unit 212C, the transmitting unit 221 transmits the index data IDXᵤₙᵢ via the network 20 to the terminal device 3A.

Furthermore, when the transmitting unit 221 received the P pieces of index data IDX₁ to IDXₚ from the control unit 212C, the transmitting unit 221 transmits the P pieces of index data IDX₁ to IDX_{P} via the network 20 to the terminal device 3A.

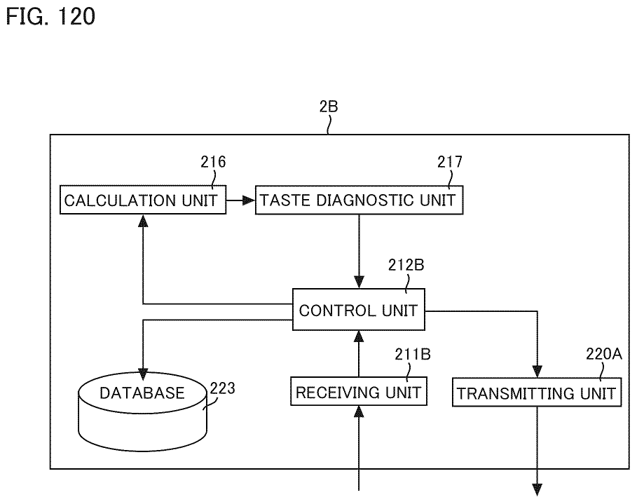

FIG. 120 is a schematic diagram of diagnostic device 2B shown in FIG. 117. Referring to FIG. 120, diagnostic device 2B is obtained by deleting calculation unit 213, judgment unit 214, and creation unit 215 of analysis/diagnostic unit 21A shown in FIG. 113, changing receiving unit 211A to receiving unit 211B, changing control unit 212A to control unit 212B, changing transmitting unit 220 to transmitting unit 220A, and adding database 223.

The receiving unit 211B receives the index data IXDᵤₙᵢ from the terminal device 3A via the network 20, and outputs the received index data IXDᵤₙᵢ to the control unit 212B.

Furthermore, the receiving unit 211B receives P pieces of index data IXD₁ to IXD_{P} from the terminal device 3A via the network 20, and outputs the received P pieces of index data IXD₁ to IXD_{P} to the control unit 212B.

When the control unit 212B received the index data IXDᵤₙᵢ from the receiving unit 211B, the control unit 212B stores the index data IXDᵤₙᵢ in the database 223 and outputs the index data IXDᵤₙᵢ to the calculation unit 216.

Furthermore, when the control unit 212B received the P pieces of index data IXD₁ to IXD_{P} from the receiving unit 211B, the control unit 212B stores the P pieces of index data IXD₁ to IXD_{P} in the database 223 and outputs the P pieces of index data IXD₁ to IXD_{P} to the calculation unit 216.

Furthermore, when the control unit 212B received the taste diagnosis result JDRᵤₙᵢ of the analyte from the taste diagnostic unit 217, the control unit 212B stores the received taste diagnosis result JDRᵤₙᵢ₁ in the database 223 and outputs the taste diagnosis result JDRᵤₙᵢ to the transmitting unit 220A.

Furthermore, when the control unit 212B received the taste diagnosis result JDR_{P} of the analyte from the taste diagnostic unit 217, the control unit 212B stores the received taste diagnosis result JDR_{P} in the database 223 and outputs the taste diagnosis result JDR_{P} to the transmitting unit 220A.

When the transmitting unit 220A received the taste diagnosis result JDRᵤₙᵢ from the control unit 212B, the transmitting unit 220A transmits the taste diagnosis result JDRᵤₙᵢ to the terminal device 3A via the network 20.

Furthermore, when the transmitting unit 220A received the taste diagnosis result JDR_{P} from the control unit 212B, the transmitting unit 220A transmits the taste diagnosis result JDR_{P} to the terminal device 3A via the network 20.

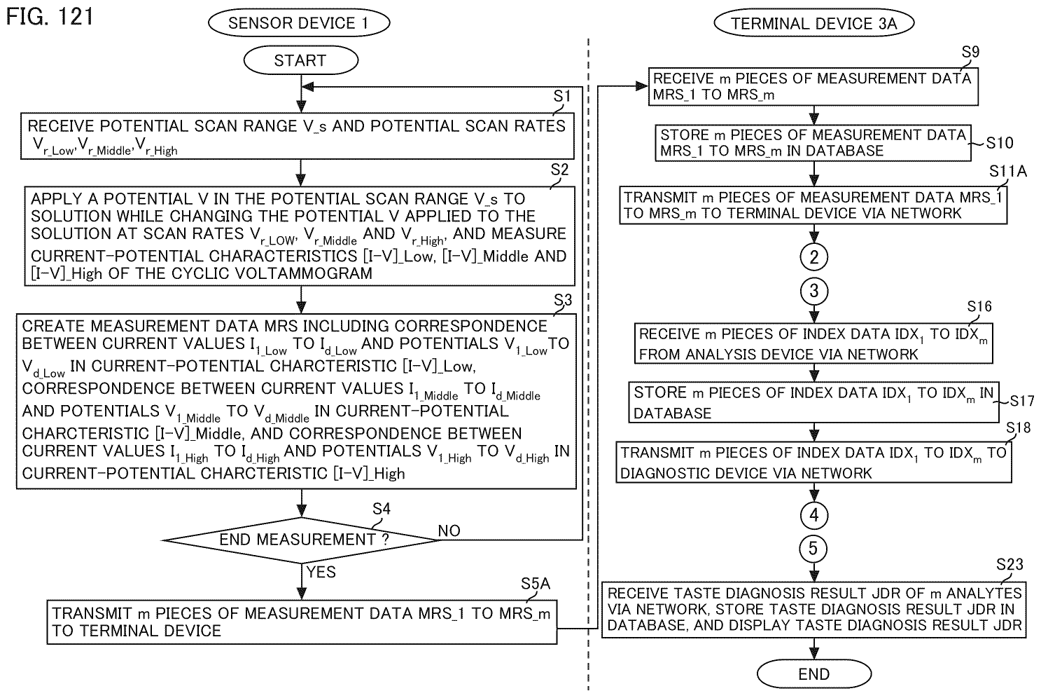

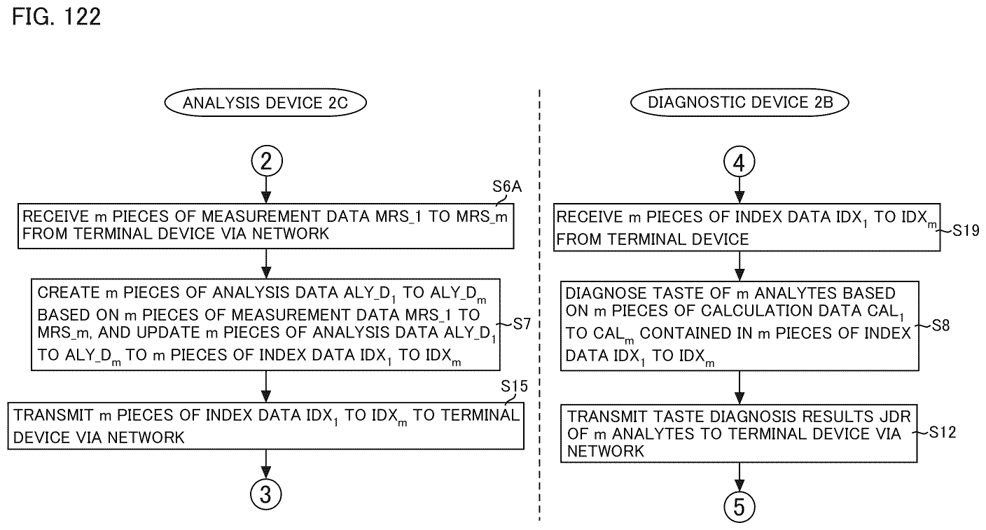

FIGS. 121 and 122 are first and second flowcharts, respectively, for explaining the operation of the diagnostic system 10B shown in FIG. 117.

Referring to FIG. 121, when the operation of diagnostic system 10B is started, the sensor device 1 sequentially executes steps S1 to S4 and S5A described above.

After the step S5A, the terminal device 3A sequentially executes the steps S9 and S10 described above.

After the step S10, the terminal device 3A transmits m pieces of measurement data MRS_1 to MRS_m to analysis device 2C via network 20 (step S11A).

Referring to FIG. 122, after step S11A in FIG. 121, analysis device 2C sequentially executes steps S6A and S7 described above.

After the step S7, the analysis device 2C transmits the m pieces of index data IDX₁ to IDXₘ to the terminal device 3A via the network 20 (step S15).

Referring to FIG. 121, after the step S15 in FIG. 122, the terminal device 3A receives m pieces of index data IDX₁ to IDXₘ from analysis device 2C via network 20 (step S16).

Then, the terminal device 3A stores the m pieces of index data IDX₁ to IDXₘ in the database 37 (step S17).

Thereafter, the terminal device 3A transmits the m pieces of index data IDX₁ to IDXₘ to the diagnostic device 2B via the network 20 (step S18).

Referring to FIG. 122, after step S18 in FIG. 121, the diagnostic device 2B receives m pieces of index data IDX₁ to IDXₘ from terminal device 3A via network 20 (step S19).

Thereafter, the diagnostic device 2B sequentially executes the above-mentioned steps S8 and S12.

Referring to FIG. 121, after the step S12 of FIG. 122, the terminal device 3A receives the taste diagnosis results JDR of m analytes via the network 20, stores the taste diagnosis results JDR in the database 37, and displays the taste diagnosis results JDR (step S23).

This completes the operation of the diagnostic system 10B.

In the embodiment of the present invention, the operation of the diagnostic device 2B may be executed by software. In this case, the diagnostic device 2B includes a CPU, a ROM and a RAM.

The ROM stores a program Prog_F consisting of steps S19, S8, and S12 (including the flowcharts shown in FIGS. 97 to 99 and 101) shown in FIG. 122, or a program Prog_G consisting of steps S19, S8, and S12 (including the flowcharts shown in FIGS. 97, 98, 100, and 102) shown in FIG. 119.

The CPU reads out the program Prog_F or the program Prog_G from the ROM, and executes the read out program Prog_F or the program Prog_G to diagnose the taste of the analyte.

In this case, the RAM temporarily stores the sums L(+)_sum, M(+)_sum, H(+)_sum, L(all)_sum, M(all)_sum, H(all)_sum, H(−)_sum, L(−)_sum_th, M(−)_sum_th, H(−)_sum_th of integrated values, Body index (+), Body index (all), Body index (−)_th, and etc.

Therefore, the program Prog_F or the program Prog_G is a program for causing a computer (CPU) to execute a diagnosis of the taste of analyte.

In the embodiment of the present invention, the operation of the analysis device 2C may be executed by software. In this case, the analysis device 2C includes a CPU, a ROM, and a RAM.

The ROM stores a program Prog_H consisting of steps S6A, S7, and S15 (including the flowcharts shown in FIGS. 91 to 96) shown in FIG. 122.

The CPU reads out the program Prog_H from the ROM, and executes the read out program Prog_H to create m pieces of analysis data ALY_D₁ to ALY_Dₘ based on m pieces of measurement data MRS_1 to MRS_m, and update the m pieces of analysis data ALY_D₁ to ALY_Dₘ to m pieces of index data IDX₁ to IDXₘ.

In this case, the RAM temporarily stores m pieces of measurement data MRS_1 to MRS_m and m pieces of analysis data ALY_D₁ to ALY_Dₘ.

Furthermore, in the embodiment of the present invention, the operation of the terminal device 3A may be executed by software. In this case, the terminal device 3A includes a CPU, a ROM and a RAM.

The ROM stores a program Prog_I consisting of steps S9, S10, S11A, S16 to S18, and S23 shown in FIG. 121.

The CPU reads out the program Prog_I from the ROM and executes the read out program Prog_I to receive m pieces of measurement data MRS_1 to MRS_m from the sensor device 1, and transmits the received m pieces of measurement data MRS_1 to MRS_m to the analysis device 2C, receives m pieces of index data IDX₁ to IDXₘ from the analysis device 2C to store them in the database 37, and transmits the m pieces of index data IDX₁ to IDXₘ to the diagnostic device 2B, receives taste diagnosis results JDR of the m analytes from the diagnostic device 2B to store them in the database 37 and displays the taste diagnosis results JDR.

In this case, the RAM temporarily stores m pieces of measurement data MRS_1 to MRS_m, m pieces of index data IDX₁ to IDXₘ, and a taste diagnosis result JDR.

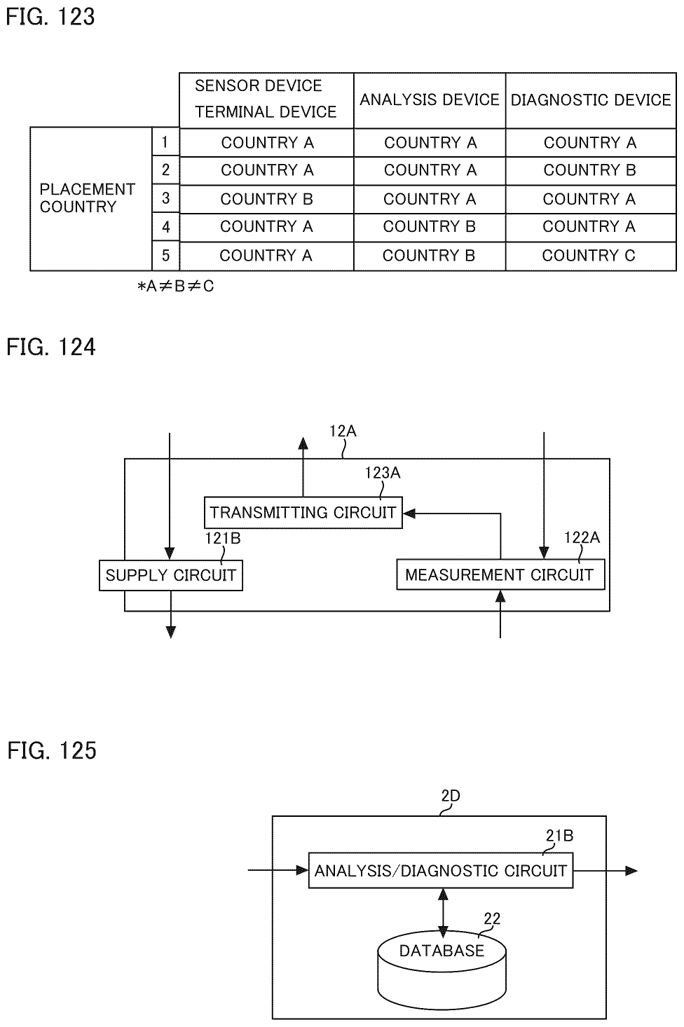

FIG. 123 is a diagram for explaining the locations of the sensor device 1, the terminal device 3A, the analysis device 2C, and the diagnostic device 2B. In FIG. 123, country A, country B, and country C are different from each other.

Referring to FIG. 123, the sensor device 1, the terminal device 3A, the analysis device 2C and the diagnostic device 2B are placed in country A.

In addition, the sensor device 1, the terminal device 3A and the analysis device 2C are located in country A, and the diagnostic device 2B is located in country B.

Furthermore, the sensor device 1 and the terminal device 3A are located in country B, and the analysis device 2C and the diagnostic device 2B are located in country A.

Furthermore, the sensor device 1 and the terminal device 3A are located in country A, the analysis device 2C is located in country B, and the diagnostic device 2B is located in country A.

Furthermore, the sensor device 1 and the terminal device 3A are located in country A, the analysis device 2C is located in country B, and the diagnostic device 2B is located in country C.

The “1” of deployment country is a pattern in which the sensor device 1, the terminal device 3A, the analysis device 2C, and the diagnostic device 2B are all deployed in the same country.

In this case, the diagnostic device 2B may receive index data IDXᵤₙᵢ or P pieces of index data IDX₁ to IDX_{P} from a terminal device 5 (a terminal device having the same configuration as the terminal device 3) located in a country different from country A via the network 20, diagnose the taste of the analyte based on the received index data IDXᵤₙᵢ or P pieces of index data IDX₁ to IDX_{P}, and may transmit a diagnosis result JDR of the taste of the analyte to the terminal device 5 (a terminal device having the same configuration as the terminal device 3) via the network 20.

The “2” of deployment country is a pattern in which the sensor device 1, the terminal device 3A and the analysis device 2C are deployed in the same country, and the diagnostic device 2B is deployed in a country different from that of the sensor device 1, the terminal device 3A and the analysis device 2C.

In this case, the diagnostic device 2B may diagnose the taste of the analyte based on the index data IDX received from an analysis device (an analysis device different from the analysis device 2C) located in country B, or may diagnose the taste of the analyte based on the index data IDX received from an analysis device (an analysis device different from analysis device 2C) located in country D which is different from countries A and B.

The “3” of deployment country is a pattern in which the analysis device 2C and the diagnostic device 2B are deployed in the same country, and the sensor device 1 and the terminal device 3A are deployed in a country different from that of the analysis device 2C and the diagnostic device 2B.

In this case, the diagnostic device 2B may diagnose the taste of the analyte based on the index data IDX received from an analysis device (an analysis device different from analysis device 2C) located in country A, or may diagnose the taste of the analyte based on the index data IDX received from an analysis device (an analysis device different from analysis device 2C) located in a country other than country A.

The “4” of deployment country is a pattern in which the sensor device 1, the terminal device 3A and the diagnostic device 2B are deployed in the same country, and the analysis device 2C is deployed in a country different from that of the sensor device 1, the terminal device 3A and the diagnostic device 2B.

In this case, the diagnostic device 2B may diagnose the taste of the analyte based on the index data IDX received from an analysis device (an analysis device different from analysis device 2C) located in country A, or may diagnose the taste of the analyte based on the index data IDX received from an analysis device (an analysis device different from analysis device 2C) located in a country other than country A and country B.

The “5” of deployment is a pattern in which the sensor device 1, the terminal device 3A, the analysis device 2C, and the diagnostic device 2B are deployed in different countries each other.

In this case, the diagnostic device 2B may diagnose the taste of the analyte based on the index data IDX received from an analysis device (an analysis device different from analysis device 2C) located in country C, or may diagnose the taste of the analyte based on the index data IDX received from an analysis device (an analysis device different from analysis device 2C) located in a country other than country C.

The rest of the description of the third embodiment is the same as that in the first embodiment.

FIG. 124 is another schematic diagram of the measuring instrument 12 shown in FIG. 4. The measuring instrument 12 shown in FIG. 4 may be a measuring instrument 12A shown in FIG. 124.

Referring to FIG. 124, a measuring instrument 12A has the supply unit 121, the measuring unit 122 and the transmitting unit 123 of the measuring instrument 12 shown in FIG. 4 replaced with a supply circuit 121B, a measuring circuit 122A and a transmitting circuit 123A, respectively.

A supply circuit 121B, a measurement circuit 122A, and a transmitting circuit 123A execute the same operations as the supply unit 121, the measurement unit 122, and the transmitting unit 123 described above, respectively.

FIG. 125 is another schematic diagram of the diagnostic device 2 shown in FIG. 7. The diagnostic device 2 shown in FIG. 7 may be a diagnostic device 2D shown in FIG. 125.

Referring to FIG. 125, in a diagnostic device 2D, the analysis/diagnostic unit 21 of the diagnostic device 2 shown in FIG. 7 is replaced with an analysis/diagnostic circuit 21B.

Analysis/diagnostic circuitry 21B then performs the same operations as analysis/diagnostic unit 21 described above.

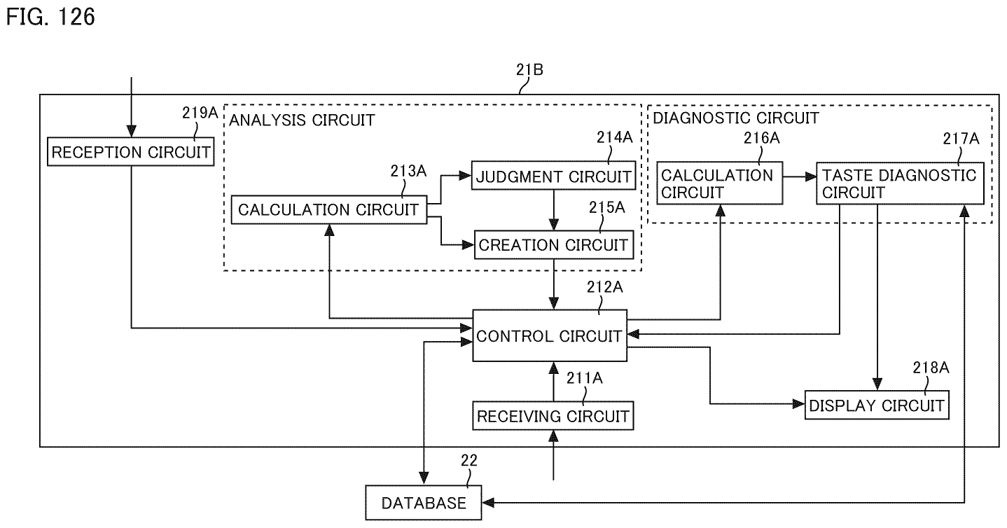

FIG. 126 is a schematic diagram of the analysis/diagnostic circuit 21B shown in FIG. 125. Referring to FIG. 126, the analysis/diagnostic circuit 21B is obtained by replacing the receiving unit 211, the control unit 212, the calculation unit 213, the judgment unit 214, the creation unit 215, the calculation unit 216, the taste diagnostic unit 217, the display unit 218 and the reception unit 219 of the analysis/diagnostic unit 21 shown in FIG. 8 with a receiving circuit 211A, a control circuit 212A, an calculation circuit 213A, a judgment circuit 214A, a creation circuit 215A, an calculation circuit 216A, a taste diagnostic circuit 217A, a display circuit 218A and a reception circuit 219A, respectively.

The receiving circuit 211A, the control circuit 212A, the calculation circuit 213A, the judgment circuit 214A, the creation circuit 215A, the calculation circuit 216A, the taste diagnostic circuit 217A, the display circuit 218A and the reception circuit 219A perform the same operations as the receiving unit 211, the control unit 212, the calculation unit 213, the judgment unit 214, the creation unit 215, the calculation unit 216, the taste diagnostic unit 217, the display unit 218 and the receiving unit 219 described above, respectively.

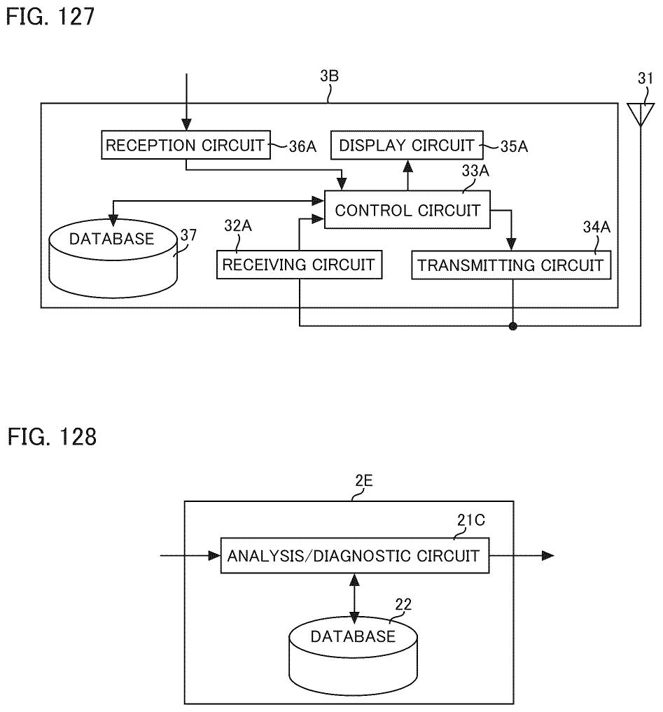

FIG. 127 is another schematic diagram of the terminal device 3 shown in FIG. 111. The terminal device 3 shown in FIG. 111 may be a terminal device 3B shown in FIG. 127.

Referring to FIG. 127, the terminal device 3B has the receiving unit 32, the control unit 33, the transmitting unit 34, the display unit 35 and the receiving unit 36 of terminal device 3 shown in FIG. 111 replaced with a receiving circuit 32A, a control circuit 33A, a transmitting circuit 34A, a display circuit 35A and a reception circuit 36A, respectively.

The receiving circuit 32A, the control circuit 33A, the transmitting circuit 34A, the display circuit 35A and the reception circuit 36A perform the same operations as the receiving unit 32, the control unit 33, the transmitting unit 34, the display unit 35 and the reception unit 36 described above, respectively.

FIG. 128 is another schematic diagram of the diagnostic device 2A shown in FIG. 112. The diagnostic device 2A shown in FIG. 112 may be constituted by a diagnostic device 2E shown in FIG. 128.

Referring to FIG. 128, a diagnostic device 2E has an analysis/diagnostic circuit 21C replacing the analysis/diagnostic unit 21A shown in FIG. 112.

The analysis/diagnostic circuitry 21C then performs the same operations as analysis/diagnostic unit 21A described above.

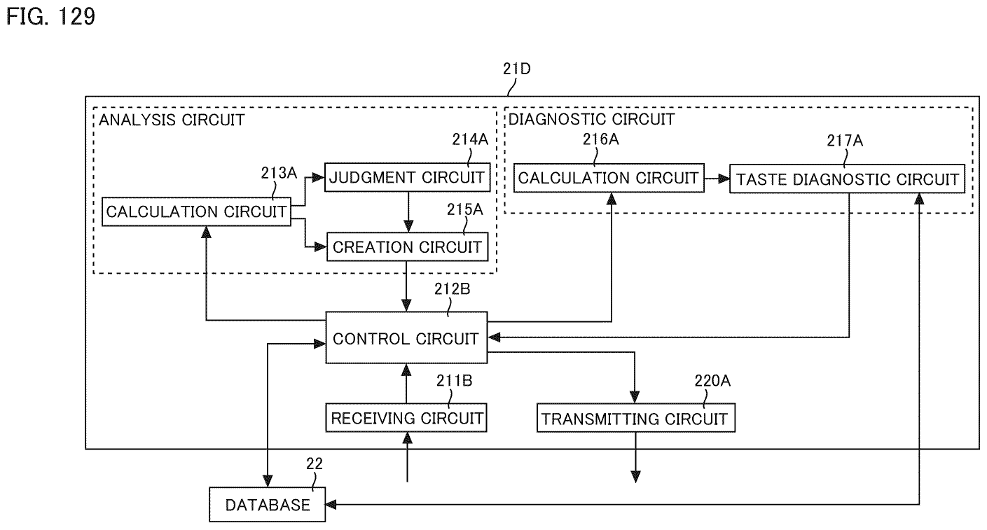

FIG. 129 is another schematic diagram of the analysis/diagnostic unit 21A shown in FIG. 113. The analysis/diagnostic unit 21A shown in FIG. 113 may be composed of an analysis/diagnostic circuit 21D shown in FIG. 129.

Referring to FIG. 129, the analysis/diagnostic circuit 21D has the receiving unit 211A, control unit 212A, the calculation unit 213, the judgment unit 214, the creation unit 215, the calculation unit 216, the taste diagnostic unit 217 and the transmitting unit 220 of the analysis/diagnostic unit 21A shown in FIG. 113 replaced with a receiving circuit 211B, a control circuit 212B, an calculation circuit 213A, a judgment circuit 214A, a creation circuit 215A, an calculation circuit 216A, a taste diagnostic circuit 217A and a transmitting circuit 220A, respectively.

The receiving circuit 211B, the control circuit 212B, the calculation circuit 213A, the judgment circuit 214A, the creation circuit 215A, the calculation circuit 216A, the taste diagnostic circuit 217A and the transmitting circuit 220A respectively perform the same operations as the receiving unit 211A, the control unit 212A, the calculation unit 213, the judgment unit 214, the creation unit 215, the calculation unit 216, the taste diagnostic unit 217 and the transmitting unit 220 described above.

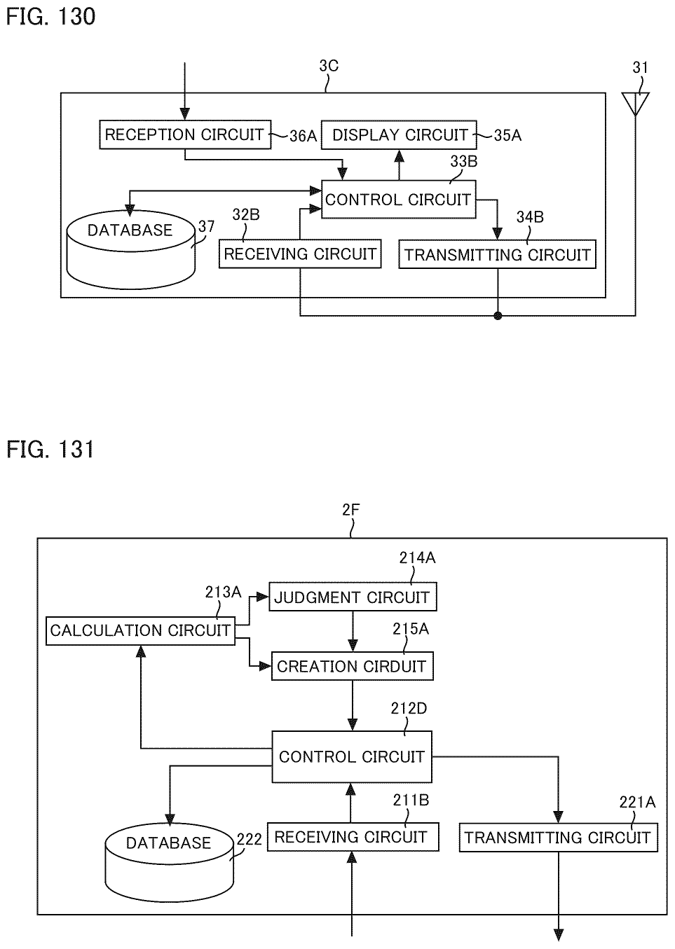

FIG. 130 is another schematic diagram of the terminal device 3A shown in FIG. 118. The terminal device 3A shown in FIG. 118 may be constituted by the terminal device 3C shown in FIG. 130.

Referring to FIG. 130, terminal device 3C is obtained by replacing the receiving unit 32A, the control unit 33A, the transmitting unit 34A, the display unit 35 and the reception unit 36 of the terminal device 3A shown in FIG. 118 with a receiving circuit 32B, a control circuit 33B, a transmitting circuit 34B, a display circuit 35A and a reception circuit 36A, respectively.

The receiving circuit 32B, the control circuit 33B, the transmitting circuit 34B, the display circuit 35A and the reception circuit 36A respectively perform the same operations as the receiving unit 32A, the control unit 33A, the transmitting unit 34A, the display unit 35 and the reception unit 36 described above.

FIG. 131 is another schematic diagram of the analysis device 2C shown in FIG. 119. The analysis device 2C shown in FIG. 119 may be constituted by the analysis device 2F shown in FIG. 131.

Referring to FIG. 131, the analysis device 2F has the receiving unit 211A, the control unit 212C, the calculation unit 213, the judgment unit 214, the creation unit 215 and the transmitting unit 221 of the analysis device 2C shown in FIG. 119 replaced with a receiving circuit 211B, a control circuit 212D, a calculation circuit 213A, a judgment circuit 214A, a creation circuit 215A and a transmitting circuit 221A, respectively.

The receiving circuit 211B, the control circuit 212D, the calculation circuit 213A, the judgment circuit 214A, the creation circuit 215A and the transmitting circuit 221A execute the same operations as the receiving unit 211A, the control unit 212C, the calculation unit 213, the judgment unit 214, the creation unit 215 and the transmitting unit 221 described above, respectively.

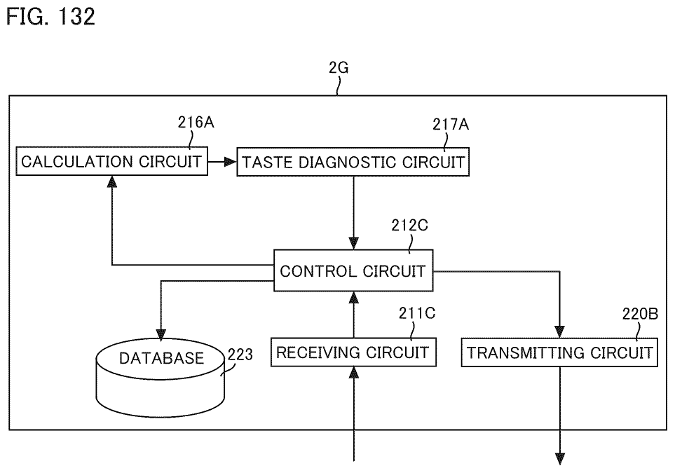

FIG. 132 is another schematic diagram of the diagnostic device 2B shown in FIG. 120. The diagnostic device 2B shown in FIG. 120 may be constituted by a diagnostic device 2G shown in FIG. 132.

Referring to FIG. 132, in a diagnostic device 2G, the receiving unit 211B, the control unit 212B, the calculation unit 216, the taste diagnostic unit 217 and the transmitting unit 220A of the diagnostic device 2B shown in FIG. 120 are replaced with a receiving circuit 211C, a control circuit 212C, a calculation circuit 216A, a taste diagnostic circuit 217A and a transmitting circuit 220B, respectively.

The receiving circuit 211C, the control circuit 212C, the calculation circuit 216A, the taste diagnostic circuit 217A and the transmitting circuit 220B respectively execute the same operations as the receiving unit 211B, the control unit 212B, the calculation unit 216, the taste diagnostic unit 217 and the transmitting unit 220A described above.

The diagnostic device 2 in the first embodiment, the diagnostic device 2A in the second embodiment, and the diagnostic device 2B in the third embodiment described above are common each other in comprising of the calculation unit 216 calculating the sum SMT__{Low(+)} of the integral values ITG_{1_Low(+)} to ITG_{x_Low(+)}, the sum SMT__{Middle(+)} of the integral values ITG_{1_Middle(+)} to ITG_{x_Middle(+)}, the sum SMT__{High(+)} of x integral values ITG_{1_High(+)} to ITG_{x_High(+)}, the sum SMT__{Low(all)} of the integral values ITG_{1_Low(all)} to ITG_{n_Low(all)}, the sum SMT__{Middle(all)} of the integral values ITG_{1_Middle(all)} to ITG_{n_Middle(all)}, the sum SMT__{High(all)} of the integral values ITG_{1_High(all)} to ITG_{n_High(all)}, and the sum H(−)_sum of integral values, and the taste diagnostic unit 217 diagnosing the taste (“astringency”, “aftertaste”, “sweetness”, “aroma” and “bitterness”) of the analyte based on the sums SMT__{Low(+)}, SMT__{Middle(+)}, SMT__{High(+)}, SMT__{Low(all)}, SMT__{Middle(all)}, SMT__{High(all)} and H(−)_sum.

Therefore, according to an embodiment of the present invention, the diagnostic device may comprises of a first calculation unit calculating a first sum (L(+)_sum) which is a sum of first integral values in a positive predetermined potential section based on a plurality of first integral values in a plurality of predetermined potential sections calculated using the current-potential characteristic of a first cyclic voltammogram measured while changing the potential at a first potential scanning rate, calculating a second sum (M(+)_sum) which is a sum of second integral values in a positive predetermined potential section based on a plurality of second integral values in a plurality of predetermined potential sections calculated using the current-potential characteristic of a second cyclic voltammogram measured while changing the potential at a second potential scanning rate faster than the first potential scanning rate, calculating a third sum (H(+)_sum) which is the sum of the third integral values in a positive predetermined potential section is calculated based on the plurality of third integral values in the plurality of predetermined potential sections calculated using the current-potential characteristics of a third cyclic voltammogram measured while changing the potential at a third potential scanning speed faster than the second potential scanning speed, calculating a fourth sum (H(−)_sum) which is the sum of the third integral values in the negative predetermined potential section based on the plurality of third integral values in the plurality of predetermined potential sections, calculating a fifth sum (L(all))_sum) which is the sum of the first integral values in all of the predetermined potential sections based on the plurality of first integral values in the plurality of predetermined potential sections, calculating a sixth sum (M(all))_sum) which is the sum of the second integral values in all of the predetermined potential sections based on the plurality of second integral values in the plurality of predetermined potential sections, and calculating a seventh sum (H(all))_sum which is the sum of the third integral values in all of the predetermined potential sections based on the plurality of third integral values in the plurality of predetermined potential sections and, a taste diagnostic unit diagnosing the taste of the analyte based on the first sum (L(+)_sum), the second sum (M(+)_sum), the third sum (H(+)_sum), the fourth sum (H(−)_sum), the fifth sum (L(all))_sum), the sixth sum (M(all))_sum and the seventh sum (H(all))_sum).

Also, according to an embodiment of the present invention, the program causes execute to computer a first step in which a first calculation unit calculates a first sum (L(+)_sum) which is a sum of first integral values in a positive predetermined potential section based on a plurality of first integral values in a plurality of predetermined potential sections calculated using a current-potential characteristic of a first cyclic voltammogram measured while changing the potential at a first potential scanning rate, calculates a second sum (M(+)_sum) which is a sum of second integral values in a positive predetermined potential section based on a plurality of second integral values in a plurality of predetermined potential sections calculated using a current-potential characteristic of a second cyclic voltammogram measured while changing the potential at a second potential scanning rate which is faster than the first potential scanning rate, calculates a third sum (H(+)_sum) which is the sum of the third integral values in a positive predetermined potential section based on the plurality of third integral values in the plurality of predetermined potential sections calculated using the current-potential characteristics of a third cyclic voltammogram measured while changing the potential at a third potential scanning speed faster than the second potential scanning speed, calculates a fourth sum (H(−)_sum) which is the sum of the third integral values in a negative predetermined potential section based on a plurality of third integral values in the plurality of predetermined potential sections, calculates a fifth sum (L(all))_sum) which is the sum of the first integral values in all predetermined potential sections based on a plurality of first integral values in the plurality of predetermined potential sections, calculates a sixth sum (M(all)_sum) which is sum of the second integral values in all predetermined potential sections based on a plurality of second integral values in the plurality of predetermined potential sections, and calculates a seventh sum (H(all)_sum) which is sum of the third integral values in all predetermined potential sections based on a plurality of third integral values in the plurality of predetermined potential sections and a second step in which the taste diagnostic unit diagnostics the taste of analyte based on the first sum (L(+)_sum), the second sum (M(+) sum), the third sum (H(+)_sum), the fourth sum (H(−)_sum), the fifth sum (L(all))_sum), the sixth sum (M(all))_sum) and the seventh sum (H(all))_sum).

In an embodiment of the present invention, the scanning speed V_{r_Low} of the potential V constitutes a “first potential scanning speed”, the scanning speed V_{r_Middle} of the potential V constitutes a “second potential scanning speed” that is faster than the first potential scanning speed, and the scanning speed V_{r_High} of the potential V constitutes a “third potential scanning speed” that is faster than the second potential scanning speed.

Furthermore, in an embodiment of the present invention, the sum L(+) sum of the integral values constitutes a “first sum” that is the sum of the first integral values in a positive predetermined potential section based on a plurality of first integral values in a plurality of predetermined potential sections calculated using the current-potential characteristics of a first cyclic voltammogram measured while changing the potential at a potential scan speed V_{r_Low}.

Here, the plurality of first integral values may be any of n¹ (n¹₁ is the number of integral values when the integral value is calculated using the smallest specified potential section, and is composed of an addition result obtained by adding “1” to the integer obtained by rounding down the decimal point of the division result obtained by dividing a positive potential section by the smallest specified potential section when the decimal point of the division result is not zero) first integral values, n¹₂ (n¹₂<n¹₁) first integral values, n¹₃ (n¹₃<n¹₂) first integral values, . . . , and n¹_{b} (n¹_{b}<n¹_{b-1}, b is an integer of 2 or more) first integral values.

Furthermore, in an embodiment of the present invention, the sum M(+)_sum of the integral values constitutes a “second sum” that is the sum of the second integral values in a positive predetermined potential section based on a plurality of second integral values in a plurality of predetermined potential sections calculated using the current-potential characteristics of a second cyclic voltammogram measured while changing the potential at the potential scan speed V_{r_Middle}.

Here, the plurality of second integral values may be any of n²₁ (n²₁ is the number of integral values when the integral value is calculated using the smallest specified potential section, and is composed of an addition result obtained by adding “1” to the integer obtained by rounding down the decimal point of the division result obtained by dividing a positive potential section by the smallest specified potential section when the decimal point of the division result is not zero) second integral values, n²₂ (n²₂<n²₁) second integral values, n²₃ (n²₃<n²₂) second integral values, . . . , and n²_{b} (n²_{b}<n²_{b-1}, b is an integer of 2 or more) second integral values.

Furthermore, in an embodiment of the present invention, the sum H(+)_sum of the integral values constitutes a “third sum” that is the sum of the third integral values in a positive predetermined potential section based on a plurality of third integral values in a plurality of predetermined potential sections calculated using the current-potential characteristics of a third cyclic voltammogram measured while changing the potential at the potential scan speed V_{r_High}.

Here, the plurality of third integral values may be any of n³₁ (n³₁ is the number of integral values when the integral value is calculated using the smallest specified potential section, and is composed of an addition result obtained by adding “1” to the integer obtained by rounding down the decimal point of the division result obtained by dividing a positive potential section by the smallest specified potential section when the decimal point of the division result is not zero) third integral values, n³₂ (n³₂<n³₁) third integral values, n³₃ (n³₃<n³₂) third integral values, . . . , and n³_{b} (n³_{b}<n³_{b-1}, b is an integer of 2 or more) third integral values.

Furthermore, in an embodiment of the present invention, the sum H(−)_sum of the integral values constitutes a “fourth sum” which is the sum of the third integral values in a negative predetermined potential section based on a plurality of third integral values in a plurality of predetermined potential sections calculated using the current-potential characteristics of a third cyclic voltammogram measured while changing the potential at the potential scan speed V_{r_High}.

Here, the plurality of third integral values may be any of n³₁ (n³₁ is the number of integral values when the integral value is calculated using the smallest specified potential section, and is composed of an addition result obtained by adding “1” to the integer obtained by rounding down the decimal point of the division result obtained by dividing a positive potential section by the smallest specified potential section when the decimal point of the division result is not zero) third integral values, n³₂ (n³₂<n³₁) third integral values, n³₃ (n³₃<n³₂) third integral values, . . . , and n³_{b} (n³_{b}<n³_{b-1}, b is an integer of 2 or more) third integral values.

Furthermore, in an embodiment of the present invention, the sum L(all)_sum of the integral values constitutes a “fifth sum” which is the sum of the first integral values in all predetermined potential sections based on a plurality of first integral values in a plurality of predetermined potential sections calculated using the current-potential characteristics of the first cyclic voltammogram measured while changing the potential at the potential scan speed V_{r_Low}.

Here, the plurality of third integral values may be any of n¹₁ (n¹₁ is the number of integral values when the integral value is calculated using the smallest specified potential section, and is composed of an addition result obtained by adding “1” to the integer obtained by rounding down the decimal point of the division result obtained by dividing a positive potential section by the smallest specified potential section when the decimal point of the division result is not zero) first integral values, n¹₂ (n¹₂<n¹₁) first integral values, n¹₃ (n¹₃<n¹₂) first integral values, . . . , and n¹_{b} (n¹_{b}<n¹_{b-1}, b is an integer of 2 or more) first integral values.

Furthermore, in an embodiment of the present invention, the sum M(all)_sum of the integral values constitutes a “sixth sum” which is the sum of the second integral values in all of the predetermined potential sections based on a plurality of second integral values in a plurality of predetermined potential sections calculated using the current-potential characteristics of the second cyclic voltammogram measured while changing the potential at the potential scan rate V_{r_Middle}.

Here, the plurality of third integral values may be any of n²₁ (n²₁ is the number of integral values when the integral value is calculated using the smallest specified potential section, and is composed of an addition result obtained by adding “1” to the integer obtained by rounding down the decimal point of the division result obtained by dividing a positive potential section by the smallest specified potential section when the decimal point of the division result is not zero) second integral values, n²₂ (n²₂<n²₁) second integral values, n²₃ (n²₃<n²₂) second integral values, . . . , and n²_{b} (n²_{b}<n²_{b-1}, b is an integer of 2 or more) second integral values.

Furthermore, in an embodiment of the present invention, the sum H(all)_sum of the integral values constitutes a “seventh sum” which is the sum of the third integral values in all of the predetermined potential sections based on a plurality of third integral values in a plurality of predetermined potential sections calculated using the current-potential characteristics of the third cyclic voltammogram measured while changing the potential at the potential scan speed V_{r_High}.

Here, the plurality of third integral values may be any of n³₁ (n³₁ is the number of integral values when the integral value is calculated using the smallest specified potential section, and is composed of an addition result obtained by adding “1” to the integer obtained by rounding down the decimal point of the division result obtained by dividing a positive potential section by the smallest specified potential section when the decimal point of the division result is not zero) third integral values, n³₂ (n³₂<n³₁) third integral values, n³₃ (n³₃<n³₂) third integral values, . . . , and n³_{b} (n³_{b}<n³_{b-1}, b is an integer of 2 or more) third integral values.

Furthermore, in an embodiment of the present invention, the sum L(−)_sum_th of the integral values constitutes an “eighth sum” which is the sum of the first integral values in a plurality of negative predetermined potential sections consisting of negative potentials below or equal to a threshold value based on the first cyclic voltammogram measured while changing the potential at the potential scanning speed V_{r_Low}.

Here, the “sum of first integral values in a plurality of negative predetermined potential sections consisting of negative potentials below or equal to a threshold value” may be any one of the sum of w¹₁ (w¹₁ is the sum of an integer obtained by rounding down the decimal point of a division result obtained by dividing a negative potential section below a threshold value by the smallest predetermined potential section when the decimal point of the division result is not zero, and then adding “1” to the integer) first integral values, the sum of w¹₂ (w¹₂<w¹₁) first integral values, the sum of w¹₃ (w¹₃<w¹₂) first integral values, . . . , and the sum of w¹_{b} (w¹_{b}<w¹_{b-1}, b is an integer equal to or greater than 2) first integral values.

Furthermore, in an embodiment of the present invention, the sum M(−)_sum_th of the integral values constitutes a “ninth sum” which is the sum of the second integral values in a plurality of negative predetermined potential sections consisting of negative potentials below or equal to a threshold value based on a second cyclic voltammogram measured while changing the potential at a potential scanning speed V_{r_Middle}.

Here, the “sum of the second integral values in a plurality of negative predetermined potential sections consisting of negative potentials below or equal to a threshold value” may be any one of the sum of w²₁ (w²₁ is an addition result obtained by adding “1” to an integer obtained by rounding down the decimal point of a division result obtained by dividing a negative potential section below or equal to a threshold value by the smallest predetermined potential section when the decimal point of the division result is not zero) second integral values, the sum of w²₂ (w²₂<w²₁) second integral values, the sum of w²₃ (w²₃<w²₂) second integral values, . . . , and the sum of w²_{b} (w²_{b}<w²_{b-1}, b is an integer equal to or greater than 2) second integral values.

Furthermore, in this embodiment of the present invention, the sum H(−)_sum_th of the integral values constitutes a “tenth sum” which is the sum of the third integral values in a plurality of negative predetermined potential sections consisting of negative potentials below or equal to a threshold value based on a third cyclic voltammogram measured while changing the potential at the potential scanning speed V_{r_High}.

Here, the “sum of the third integral values in a plurality of negative predetermined potential sections consisting of negative potentials below or equal to the threshold value” may be any one of a sum of w³₁ (w³₁ is an addition result obtained by adding “1” to an integer obtained by rounding down the decimal point of a division result obtained by dividing a negative potential section below or equal to the threshold value by the smallest predetermined potential section when the decimal point of the division result is not zero) third integral values, a sum of w³₂ (w³₂<w³₁) third integral values, a sum of w³₃ (w³₃<w³₂) third integral values, . . . , and a sum of w³b (w³_{b}<w³_{b-1}, b is an integer equal to or greater than 2) third integral values.

Furthermore, in an embodiment of the present invention, the Body Index (+) constitutes a “first factor” which is a factor resulting from the diffusion coefficient of a component of the analyte when a positive potential is applied to the analyte.

Furthermore, in an embodiment of the present invention, the Body Index (all) constitutes a “second factor” which is a factor resulting from the diffusion coefficient of the components of the analyte when positive and negative potentials are applied to the analyte.

Furthermore, in an embodiment of the present invention, the Body index (−)_th constitutes a “third factor” which is a factor resulting from the diffusion coefficient of the components of the analyte when a negative potential below or equal to a threshold value is applied to the analyte.

Furthermore, in this embodiment of the present invention, each of the sums {ITG_{1_Low}+ITG_{1_Middle}+ITG_{1_High}}, {ITG_{2_Low}+ITG_{2_Middle}+ITG_{2_High}}, . . . , {ITG_{n_1_Low}+ITG_{n_1_Middle}+ITG_{n_1_High}}, {ITG_{n_Low}+ITG_{n_Middle}+ITG_{n_High}} of the integral values shown in FIG. 12 constitutes a “total integral value.”

The embodiments disclosed herein are to be considered illustrative in all respects and not restrictive. The scope of the invention is defined by the appended claims, not by the foregoing description of the embodiments, and it is intended that all modifications fall within the scope of claims and their equivalents.

### INDUSTRIAL APPLICABILITY

The present invention is applicable to a diagnostic device, a diagnostic system using the same, and a program for causing a computer to execute.

1. A diagnostic device comprising:
a first calculation circuit configured to calculate a first sum (L(+)_sum) which is a sum of first integral values in a positive predetermined potential section based on a plurality of first integral values in a plurality of predetermined potential sections calculated using a current-potential characteristics of a first cyclic voltammogram measured while changing a potential at a first potential scanning rate, calculate a second sum (M(+) sum) which is a sum of second integral values in the positive predetermined potential section based on a plurality of second integral values in the plurality of predetermined potential sections calculated using a current-potential characteristic of a second cyclic voltammogram measured while changing the potential at a second potential scanning rate faster than the first potential scanning rate, calculate a third sum (H(+)_sum) which is a sum of third integral values in the positive predetermined potential section based on a plurality of third integral values in the plurality of predetermined potential sections calculated using a current-potential characteristic of a third cyclic voltammogram measured while changing the potential at a third potential scanning rate faster than the second potential scanning rate, calculate a fourth sum (H(−)_sum) which is a sum of the third integral values in a negative predetermined potential sections based on the plurality of third integral values in the plurality of predetermined potential sections, calculate a fifth sum (L(all)_sum) which is a sum of the first integral values in all of the predetermined potential sections based on the plurality of first integral values in the plurality of predetermined potential sections, calculate a sixth sum (M(all)_sum) which is a sum of the second integral values in all of the predetermined potential sections based on the plurality of second integral values in the plurality of predetermined potential sections, and calculate a seventh sum (H(all)_sum) which is a sum of the third integral values in all of the predetermined potential sections based on the plurality of third integral values in the plurality of predetermined potential sections, and
a taste diagnostic circuit configured to diagnose a taste of a first analyte based on the first sum (L(+)_sum), the second sum (M(+)_sum), the third sum (H(+)_sum), the fourth sum (H(−)_sum), the fifth sum (L(all))_sum), the sixth sum (M(all))_sum and the seventh sum (H(all))_sum).

2. The diagnostic device according to claim 1, wherein the first calculation circuit further calculates a first factor (Body index (+)) which is a factor attributable to a diffusion coefficient of a component of the first analyte when a positive potential is applied to the first analyte based on the first sum (L(+)_sum), the second sum (M(+)_sum) and the third sum (H(+)_sum) and calculates a second factor (Body index (all)) which is a factor attributable to the diffusion coefficient of the component of the first analyte when positive and negative potentials are applied to the first analyte based on the fifth sum (L(all)_sum), the sixth sum (M(all))_sum, and the seventh sum (H(all)_sum), and
the taste diagnostic circuit diagnoses “astringency” of the first analyte based on the first factor (Body index (+)), diagnoses “aftertaste” of the first analyte based on the second factor (Body Index (all)), diagnoses “sweetness” of the first analyte based on the third sum (H(+)_sum), diagnoses “aroma” of the first analyte based on the fourth sum (H(−)_sum), and diagnoses “bitterness” of the first analyte based on the “astringency” of the first analyte and the “sweetness” of the first analyte.

3. The diagnostic device according to claim 2, wherein the taste diagnostic circuit diagnoses the multiplication result obtained by multiplying a coefficient k₁ to the first factor (Body index (+)) as the “astringency” of the first analyte, diagnoses the multiplication result obtained by multiplying a coefficient k₂ to the second factor (Body Index (all)) as the “aftertaste” of the first analyte, diagnoses as the “sweetness” of the first analyte the result of multiplying a coefficient k₄ to the result of dividing the third sum (H(+)_sum) by a coefficient k₃, diagnoses as the “aroma” of the first analyte the result of multiplying a coefficient k₆ to the result of dividing the fourth sum (H(−)_sum) by a coefficient k₅, and diagnoses as the “bitterness” of the first analyte the result obtained by subtracting the result of multiplying a coefficient k₈ to the “sweetness” of the first analyte from the result obtained by multiplying a coefficient k₇ to the “astringency” of the first analyte.

4. The diagnostic device according to claim 3, wherein the taste diagnostic circuit performs a regression analysis using the first factor (Body index (+)) as an explanatory variable and the “astringency” as a response variable to obtain a regression equation, and determines a value multiplied to the first factor (Body index (+)) in the obtained regression equation as the value of the coefficient k₁, performs a regression analysis using the second factor (Body index (all)) as an explanatory variable and the “aftertaste” as a response variable to obtain a regression equation, and determines a value multiplied to the second factor (Body index (all)) which is an explanatory variable in the obtained regression equation as a value of the coefficient k₂, performs a regression analysis using the third sum (H(+)_sum) as an explanatory variable and the “sweetness” as a response variable to obtain a regression equation, and determines a value dividing the explanatory variable (=the third sum (H(+)_sum)) as a value of the coefficient k₃ in the obtained regression equation, and determines a value multiplied to the explanatory variable (=the third sum (H(+)_sum)) as a value of the coefficient k₄, performs a regression analysis using the fourth sum (H(−)_sum) as an explanatory variable and the “aroma” as a response variable to obtain a regression equation, and determines that a value dividing the explanatory variable (the fourth sum (H(−)_sum)) as a value of the coefficient k₅ and determines a value multiplied to the explanatory variable (the fourth sum (H(−)_sum)) as a value of the coefficient k₆ in the obtained regression equation, and performs a regression analysis using the “astringency” and the “sweetness” as explanatory variables and the “bitterness” as a response variable to obtain a regression equation, and determines a value multiplied to the “astringency” as a value of the coefficient k₇ and determines a value multiplied to the “sweetness” as a value of the coefficient k₈ in the obtained regression equation.

5. The diagnostic device according to claim 3, wherein the taste diagnostic circuit updates the values of the coefficients k₁ to k₈ when the taste diagnostic circuit diagnosed the “astringency”, the “aftertaste”, the “sweetness”, the “aroma” and the “bitterness” of v (v is an integer of 1 or more) of the first analytes, and diagnoses the “astringency”, the “aftertaste”, the “sweetness”, the “aroma” and the “bitterness” of the first analyte using the updated values of the coefficients k₁ to k₈.

6. The diagnostic device according to claim 1, wherein the plurality of first integral values are any of n¹₁ (n¹₁ is the number of integral values calculated using the smallest predetermined potential section, and is an addition result obtained by adding “1” to an integer obtained by rounding down the decimal point of a division result obtained by dividing a positive potential section by the smallest predetermined potential section when the decimal point of the division result is not zero) first integral values, n¹₂ (n¹₂<n¹₁) first integral values, n¹₃ (n¹₃<n¹₂) first integral values, . . . , and n¹₁ (n¹_{b}<n¹_{b-1}, b is an integer equal to or greater than 2) first integral values,
the plurality of second integral values are any of n²₁ (n²₁ is the number of integral values calculated using the smallest predetermined potential section, and is an addition result obtained by adding “1” to an integer obtained by rounding down the decimal point of a division result obtained by dividing a positive potential section by the smallest predetermined potential section when the decimal point of the division result is not zero) second integral values, n²₂ (n²₂<n²₁) second integral values, n²₃ (n²₃<n²₂) second integral values, . . . , and n²_{b} (n²_{b}<n²_{b-1}, b is an integer equal to or greater than 2) second integral values, and
the plurality of third integral values are any of n³₁ (n³₁ is the number of integral values calculated using the smallest predetermined potential section, and is composed of an addition result obtained by adding “1” to an integer obtained by rounding down the decimal point of a division result obtained by dividing a positive potential section by the smallest predetermined potential section when the decimal point of the division result is not zero) third integral values, n³₂ (n³₂<n³₁) third integral values, n³₃ (n³₃<n³₂) third integral values, . . . , and n³_{b} (n³_{b}<n³_{b-1}, b is an integer equal to or greater than 2) third integral values.

7. The diagnostic device according to claim 1, wherein the first calculation circuit further calculates an eighth sum (L(−)_sum_th) which is a sum of the first integral values in a plurality of negative predetermined potential sections consisting of negative potentials equal to or lower than a threshold value based on a plurality of first integral values in a plurality of predetermined potential sections calculated using the current-potential characteristic of the first cyclic voltammogram, calculates a ninth sum (M(−)_sum_th) which is a sum of the second integral values in the plurality of negative predetermined potential sections consisting of negative potentials equal to or lower than the threshold value based on a plurality of second integral values in the plurality of predetermined potential sections calculated using the current-potential characteristic of the second cyclic voltammogram, calculates a tenth sum (H(−)_sum_th) which is a sum of the third integral values in the plurality of negative predetermined potential sections consisting of negative potentials equal to or lower than the threshold value based on the plurality of third integral values in the plurality of predetermined potential sections calculated using the current-potential characteristic of the third cyclic voltammogram, and calculates a third factor (Body index (−)_th) which is a factor attributable to a diffusion coefficient of a component of a second analyte when a negative potential equal to or less than the threshold value is applied to the second analyte based on the eighth sum (L(−)_sum_th), the ninth sum (M(−)_sum_th) and the tenth sum (H(−)_sum_th), and
the taste diagnostic circuit further diagnoses the “astringency” of the second analyte based on the third factor (Body index (−)_th).

8. The diagnostic device according to claim 7, wherein the taste diagnostic circuit diagnoses the multiplication result obtained by multiplying a coefficient k9 to the third factor (Body index (−)_th) as the “astringency” of the second analyte.

9. The diagnostic device according to claim 8, wherein the taste diagnostic circuit performs a regression analysis using the third factor (Body index (−)_th) as an explanatory variable and the “astringency” as a response variable to obtain a regression equation, and determines a value multiplied to the third factor (Body index (−)_th) in the obtained regression equation as a value of the coefficient k9.

10. The diagnostic device according to claim 8, wherein the taste diagnostic circuit updates the value of the coefficient k9 and diagnoses the “astringency” of the second analyte using the updated value of the coefficient k9 when the taste diagnostic circuit diagnosed the “astringency” of v (v is an integer equal to or greater than 1) of the second analyte(s).

11. The diagnostic device according to claim 7, wherein the sum of the first integral values in a plurality of negative predetermined potential sections consisting of negative potentials equal to or less than the threshold value is any one of a sum of w¹ (w¹ is an addition result obtained by adding “1” to an integer obtained by rounding down a decimal point of a division result obtained by dividing a negative potential section equal to or less than the threshold value by a minimum predetermined potential section when the decimal point of the division result is not zero) first integral values, a sum of w¹₂ (w¹₂<w¹₁) first integral values, a sum of w¹₃(w¹₃<w¹₂) first integral values, . . . , and a sum of w¹_{b} (w¹_{b}<w¹_{b-1}, b is an integer equal to or greater than 2) first integral values,
the sum of the second integral values in the plurality of negative predetermined potential sections consisting of negative potentials equal to or less than the threshold value is any one of a sum of w²₁ (w²₁ is an addition result obtained by adding “1” to an integer obtained by rounding down a decimal point of a division result obtained by dividing a negative potential section equal to or less than a threshold value by a smallest predetermined potential section when the decimal point of the division result is not zero.) second integral values, a sum of w²₂ (w²₂<w²₁) second integral values, a sum of w²₃ (w²₃<w²₂) second integral values, . . . , and a sum of w²_{b} (w²_{b}<w²_{b-1}, b is an integer equal to or greater than 2) second integral values, and
the sum of the third integral values in the plurality of negative predetermined potential sections consisting of negative potentials equal to or less than the threshold value is any one of a sum of w³₁ (w³₁ is an addition result obtained by adding “1” to an integer obtained by rounding down a decimal point of a division result obtained by dividing a negative potential section equal to or less than a threshold value by a minimum predetermined potential section when the decimal point of the division result is not zero.) third integral values, a sum of w³₂ (w³₂<w³₁) third integral values, a sum of w³₃ (w³₃<w³₂) third integral values, . . . , and a sum of w³_{b} (w³_{b}<w³_{b-1}, b is an integer equal to or greater than 2) third integral values.

12. The diagnostic device according to claim 1, wherein further comprising a second calculation circuit calculates the plurality of first integral values in the plurality of predetermined potential sections based on the current-potential characteristics of the first cyclic voltammogram, calculates the plurality of second integral values in the plurality of predetermined potential sections based on the current-potential characteristics of the second cyclic voltammogram, and calculates the plurality of third integral values in the plurality of predetermined potential sections based on the current-potential characteristics of the third cyclic voltammogram, and
the first calculation circuit calculates the first sum (L(+) sum) and the fifth sum (L(all)_sum) based on the plurality of first integral values calculated by the second calculation circuit, calculates the second sum (M(+)_sum) and the sixth sum (M(all)_sum) based on the plurality of second integral values calculated by the second calculation circuit, and calculates the third sum (H(+) sum), the fourth sum (H(−)_sum), and the seventh sum (H(all)_sum) based on the plurality of third integral values calculated by the second calculation circuit.

13. The diagnostic device according to claim 12, wherein further comprising a creation circuit configured to create, as a feature amount of the first analyte or the second analyte, a curve indicating the dependency of the plurality of sum integral values on the plurality of predetermined potential sections based on the plurality of predetermined potential sections and a plurality of sum integral values respectively corresponding to the plurality of predetermined potential sections, and
the second calculation circuit further calculates the total integral value which is a sum of the first integral value, the second integral value, and the third integral value in one of the predetermined potential sections for all of the plurality of predetermined potential sections to calculate the plurality of total integral values, and outputs the plurality of predetermined potential sections and the plurality of total integral values respectively associated with the plurality of predetermined potential sections to the creation circuit.

14. The diagnostic device according to claim 13, wherein calculation data includes the plurality of predetermined potential sections and the plurality of total integral values respectively associated with the plurality of predetermined potential sections,
further comprising a judgment circuit judging whether P (P is an integer equal to or greater than 2) pieces of “the plurality of total integral values” included in P pieces of the calculation data are or not different from each other, and
the creation circuit creates P pieces of the curves when the judgment circuit judges that the P pieces of “the plurality of total integral values” are different from each other.

15. A diagnostic system comprising the diagnostic device according to claim 1.

16. A program causing a computer to execute:
a first step in which a first calculation circuit calculates a first sum (L(+)_sum) which is a sum of first integral values in a positive predetermined potential section based on a plurality of first integral values in a plurality of predetermined potential sections calculated using a current-potential characteristics of a first cyclic voltammogram measured while changing a potential at a first potential scanning rate, calculates a second sum (M(+)_sum) which is a sum of second integral values in the positive predetermined potential section based on a plurality of second integral values in the plurality of predetermined potential sections calculated using a current-potential characteristic of a second cyclic voltammogram measured while changing the potential at a second potential scanning rate faster than the first potential scanning rate, calculates a third sum (H(+)_sum) which is a sum of third integral values in the positive predetermined potential section based on a plurality of third integral values in the plurality of predetermined potential sections calculated using a current-potential characteristic of a third cyclic voltammogram measured while changing the potential at a third potential scanning rate faster than the second potential scanning rate, calculates a fourth sum (H(−)_sum) which is a sum of the third integral values in a negative predetermined potential sections based on the plurality of third integral values in the plurality of predetermined potential sections, calculates a fifth sum (L(all)_sum) which is a sum of the first integral values in all of the predetermined potential sections based on the plurality of first integral values in the plurality of predetermined potential sections, calculates a sixth sum (M(all)_sum) which is sum of the second integral values in all of the predetermined potential sections based on the plurality of second integral values in the plurality of predetermined potential sections, and calculates a seventh sum (H(all)_sum) which is sum of the third integral values in all of the predetermined potential sections based on the plurality of third integral values in the plurality of predetermined potential sections and,
a second step in which a taste diagnostic circuit diagnoses a taste of a first analyte based on the first sum (L(+)_sum), the second sum (M(+)_sum), the third sum (H(+)_sum), the fourth sum (H(−)_sum), the fifth sum (L(all))_sum), the sixth sum (M(all))_sum and the seventh sum (H(all))_sum).

17. The program causing a computer to execute according to claim 16, wherein the first calculation circuit, in the first step, further calculates a first factor (Body index (+)), which is a factor attributable to a diffusion coefficient of a component of the first analyte when a positive potential is applied to the first analyte based on the first sum (L(+)_sum), the second sum (M(+)_sum) and the third sum (H(+)_sum), and calculates a second factor (Body index (all)) which is a factor attributable to the diffusion coefficient of the component of the first analyte when positive and negative potentials are applied to the first analyte based on the fifth sum (L(all))_sum, the sixth sum (M(all))_sum, and the seventh sum (H(all))_sum, and
the taste diagnostic circuit, in the second step, diagnoses “astringency” of the first analyte based on the first factor (Body index (+)), diagnoses “aftertaste” of the first analyte based on the second factor (Body index (all)), diagnoses “sweetness” of the first analyte based on the third sum (H(+)_sum), diagnoses “aroma” of the first analyte based on the fourth sum (H(−)_sum), and diagnoses “bitterness” of the first analyte based on the “astringency” of the first analyte and the “sweetness” of the first analyte.

18. The program causing a computer to execute according to claim 17, wherein the taste diagnostic circuit, in the second step, diagnoses the multiplication result obtained by multiplying a coefficient k₁ to the first factor (Body index (+)) as the “astringency” of the first analyte, diagnoses the multiplication result obtained by multiplying a coefficient k₂ to the second factor (Body index (all)) as the “aftertaste” of the first analyte, diagnoses the result of multiplying a coefficient k₄ to the result of dividing the third sum (H(+)_sum) by a coefficient k₃ as the “sweetness” of the first analyte, diagnoses the result of multiplying a coefficient k₆ to the result of dividing the fourth sum (H(−)_sum) by a coefficient k₅ as the “aroma” of the first analyte, and diagnoses as the “bitterness” of the first analyte a subtraction result subtracting the multiplication result obtained by multiplying a coefficient k₈ to the “sweetness” of the first analyte from the multiplication result obtained by multiplying a coefficient k₇ to the “astringency” of the first analyte.

19. The program causing a computer to execute according to claim 18, wherein the taste diagnostic circuit, in the second step, performs a regression analysis using the first factor (Body index (+)) as an explanatory variable and the “astringency” as a response variable to obtain a regression equation, and determines as s value of the coefficient k₁ a value multiplied to the first factor (Body index (+)) in the obtained regression equation, performs a regression analysis using the second factor (Body index (all)) as an explanatory variable and the “aftertaste” as a response variable to obtain a regression equation, and determines as a value of the coefficient k₂ a value multiplied to the second factor (Body index (all)) which is the explanatory variable in the obtained regression equation,
performs a regression analysis using the third sum (H(+)_sum) as an explanatory variable and the “sweetness” as a response variable to obtain a regression equation, and determines, as a value of the coefficient k₃, a value dividing the explanatory variable (=the third sum (H(+)_sum)) in the obtained regression equation, and determines a value multiplied to the explanatory variable (=the third sum (H(+)_sum)) as a value of the coefficient k₄, performs a regression analysis using the fourth sum (H(−)_sum) as an explanatory variable and the “aroma” as a response variable to obtain a regression equation, and determines a value dividing the explanatory variable (the fourth sum (H(−)_sum)) in the obtained regression equation as a value of the coefficient k₅, and determines a value multiplied to the explanatory variable (the fourth sum (H(−)_sum)) as a value of the coefficient k₆,
and performs a regression analysis using the “astringency” and the “sweetness” as explanatory variables and the “bitterness” as a response variable to obtain a regression equation, determines a value multiplied to the “astringency” in the obtained regression equation as a value of the coefficient k₇, and determines a value multiplied to the “sweetness” as a value of the coefficient k₈.

20. The program causing a computer to execute according to claim 18, wherein the taste diagnostic circuit updates values of the coefficients k₁ to k₈, and diagnoses the “astringency”, the “aftertaste”, the “sweetness”, the “aroma” and the “bitterness” of the first analyte using the updated values of the coefficients k₁ to k₈ when the taste diagnostic circuit, in the second step, diagnosed the “astringency”, the “aftertaste”, the “sweetness”, the “aroma” and the “bitterness” of v (v is an integer of 1 or more) first analytes.

21. The program causing a computer to execute according to claim 16, wherein the plurality of first integral values are any one of n¹₁ (n¹₁ is the number of integral values calculated using the smallest predetermined potential section, and is an addition result obtained by adding “1” to an integer obtained by rounding down the decimal point of a division result obtained by dividing a positive potential section by the smallest predetermined potential section when the decimal point of the division result is not zero) first integral values, n¹₂ (n¹₂<n¹₁) first integral values, n¹₃ (n¹₃<n¹₂) first integral values, . . . , and n¹_{b} (n¹_{b}<n¹_{b-1}, b is an integer equal to or greater than 2) first integral values,
the plurality of second integral values are any of n²₁ (n²₁ is the number of integral values calculated using the smallest predetermined potential section, and is an addition result obtained by adding “1” to an integer obtained by rounding down the decimal point of a division result obtained by dividing a positive potential section by the smallest predetermined potential section when the decimal point of the division result is not zero) second integral values, n²₂ (n²₂<n²₁) second integral values, n²₃ (n²₃<n²₂) second integral values, . . . , and n²_{b} (n²_{b}<n²_{b-1}, b is an integer equal to or greater than 2) second integral values, and
the plurality of third integral values are any of n³₁ (n³₁ is the number of integral values calculated using the smallest predetermined potential section, and is composed of an addition result obtained by adding “1” to an integer obtained by rounding down the decimal point of a division result obtained by dividing a positive potential section by the smallest predetermined potential section when the decimal point of the division result is not zero) third integral values, n³₂ (n³₂<n³₁) third integral values, n³₃ (n³₃<n³₂) third integral values, . . . , and n³_{b} (n³_{b}<n³_{b-1}, b is an integer equal to or greater than 2) third integral values.

22. The program causing a computer to execute according to claim 16, wherein the first calculation circuit, in the first step, further calculates an eighth sum (L(−)_sum_th) which is a sum of the first integral values in a plurality of negative predetermined potential sections consisting of negative potentials equal to or less than a threshold value based on a plurality of first integral values in a plurality of predetermined potential sections calculated using the current-potential characteristic of the first cyclic voltammogram, calculates a ninth sum (M(−)_sum_th) which is a sum of the second integral values in a plurality of negative predetermined potential sections consisting of negative potentials equal to or lower than the threshold value based on a plurality of second integral values in the plurality of predetermined potential sections calculated using the current-potential characteristic of the second cyclic voltammogram, calculates a tenth sum (H(−)_sum_th) which is a sum of the third integral values in the plurality of negative predetermined potential sections consisting of negative potentials equal to or lower than the threshold value based on a plurality of third integral values in the plurality of predetermined potential sections calculated using the current-potential characteristic of the third cyclic voltammogram, and calculates a third factor (Body index (−)_th) which is a factor attributable to a diffusion coefficient of a component of a second analyte when a negative potential equal to or less than the threshold value is applied to the second analyte based on the eighth sum (L(−)_sum_th), the ninth sum (M(−)_sum_th) and the tenth sum (H(−)_sum_th), and
the taste diagnostic circuit, in the second step, further diagnoses the “astringency” of the second analyte based on the third factor (Body index (−)_th).

23. The program causing a computer to execute according to claim 22, wherein the taste diagnostic circuit, in the second step, diagnoses, as the “astringency” of the second analyte, the multiplication result obtained by multiplying the coefficient k₉ to the third factor (Body index (−)_th).

24. The program causing a computer to execute according to claim 23, wherein the taste diagnostic circuit, in the second step, performs a regression analysis using the third factor (Body index (−)_th) as an explanatory variable and the “astringency” as a response variable to obtain a regression equation, and determines a value multiplied to the third factor (Body index (−)_th) in the obtained regression equation as a value of the coefficient k₉.

25. The program causing a computer to execute according to claim 23, wherein the taste diagnostic circuit updates the value of the coefficient k₉ and diagnoses the “astringency” of the second analyte using the updated value of the coefficient k₉ when the taste diagnostic circuit, in the second step, diagnosed the “astringency” of v (v is an integer equal to or greater than 1) second analytes.

26. The program causing a computer to execute according to claim 22, wherein the sum of the first integral values in a plurality of negative predetermined potential sections consisting of negative potentials equal to or less than the threshold value is any one of a sum of w¹₁ (w¹₁ is an addition result obtained by adding “1” to an integer obtained by rounding down a decimal point of a division result obtained by dividing a negative potential section equal to or less than the threshold value by a minimum predetermined potential section when the decimal point of the division result is not zero) first integral values, a sum of w¹₂ (w¹₂<w¹₁) first integral values, a sum of w¹₃ (w¹₃<w¹₂) first integral values, . . . , and a sum of w¹_{b} (w¹_{b}<w¹_{b-1}, b is an integer equal to or greater than 2) first integral values,
the sum of the second integral values in the plurality of negative predetermined potential sections consisting of negative potentials equal to or less than the threshold value is any one of a sum of w²₁ (w²₁ is an addition result obtained by adding “1” to an integer obtained by rounding down a decimal point of a division result obtained by dividing a negative potential section equal to or less than a threshold value by a smallest predetermined potential section when the decimal point of the division result is not zero) second integral values, a sum of w²₂ (w²₂<w²₁) second integral values, a sum of w²₃ (w²₃<w²₂) second integral values, . . . , and a sum of w²_{b} (w²_{b}<w²_{b-1}, b is an integer equal to or greater than 2) second integral values, and
the sum of the third integral values in the plurality of negative predetermined potential sections consisting of negative potentials equal to or less than the threshold value is any one of a sum of w³₁ (w³₁ is an addition result obtained by adding “1” to an integer obtained by rounding down a decimal point of a division result obtained by dividing a negative potential section equal to or less than a threshold value by a minimum predetermined potential section when the decimal point of the division result is not zero) third integral values, a sum of w³₂ (w³₂<w³₁) third integral values, a sum of w³₃ (w³₃<w³₂) third integral values, . . . and a sum of w³b (w³_{b}<w³_{b-1}, b is an integer equal to or greater than 2) third integral values.

27. The program causing a computer to execute according to claim 16, wherein further causing a computer to execute a third step in which a second calculation circuit calculates the plurality of first integral values in the plurality of predetermined potential sections based on the current-potential characteristic of the first cyclic voltammogram, calculates the plurality of second integral values in the plurality of predetermined potential sections based on the current-potential characteristic of the second cyclic voltammogram, and calculates the plurality of third integral values in the plurality of predetermined potential sections based on the current-potential characteristic of the third cyclic voltammogram, and the first calculation circuit, in the first step, calculates the first sum (L(+)_sum) and the fifth sum (L(all)_sum) based on the plurality of first integral values calculated by the second calculation circuit, calculates the second sum (M(+)_sum) and the sixth sum (M(all)_sum) based on the plurality of second integral values calculated by the second calculation circuit, and calculates the third sum (H(+)_sum), the fourth sum (H(−)_sum), and the seventh sum (H(all)_sum) based on the plurality of third integral values calculated by the second calculation circuit.

28. The program causing a computer to execute according to claim 27, wherein further causing a computer to execute a fourth step in which a creation circuit creates, as a feature amount of the first analyte or the second analyte, a curve showing the dependency of the plurality of total integral values on the plurality of predetermined potential sections based on the plurality of predetermined potential sections and a plurality of total integral values respectively corresponding to the plurality of predetermined potential sections, and
the second calculation circuit further, in the third step, executes to calculate the total integral value that is a sum of the first integral value, the second integral value, and the third integral value in one of the predetermined potential sections for all of the plurality of predetermined potential sections to calculate the plurality of total integral values, and outputs the plurality of predetermined potential sections and the plurality of total integral values respectively associated with the plurality of predetermined potential sections to the creation circuit.

29. The program causing a computer to execute according to claim 28, wherein calculation data includes the plurality of predetermined potential sections and the plurality of total integral values respectively corresponding to the plurality of predetermined potential sections,
the program causes the computer to execute further a fifth step in which a judgment circuit judges whether P (P is an integer equal to or greater than 2) pieces of [the plurality of sum integral values] included in P pieces of the calculation data are or not different from each other, and
the creation circuit creates P pieces of the curves in the fourth step when the judgment circuit judged in the fifth step that the P pieces of [the plurality of total integral values] are different from each other.
